(19) United States
(12) Patent Application Publication
(10) Pub. No.: US 2012/0213781 A1
(43) Pub. Date: Aug. 23, 2012

(54) Monovalent and Multivalent Multispecific Complexes and Uses Thereof

(75) Inventor: David HILBERT, Bethesda, MD (US)

(73) Assignee: Zyngenia, Inc., Gaithersburg, MD (US)

(21) Appl. No.: 13/371,379

(22) Filed: Feb. 10, 2012

Related U.S. Application Data
(60) Provisional application No. 61/442,106, filed on Feb. 11, 2011.

(51) Int. Cl. A61K 39/395; C12P 21/06; C12N 1/21; C07H 21/04; C12N 5/10; C12N 15/63; A61K 38/17; G01N 33/53; C12N 1/19

(52) U.S. Cl. 424/134.1; 435/7.1; 435/69.6; 435/325; 435/348; 435/352; 435/354; 435/358; 435/365; 435/367; 435/419; 435/252.31; 435/252.33; 435/254.2; 435/254.21; 435/254.23; 435/320.1; 530/353; 536/23.4

(57) ABSTRACT

Monovalent and multivalent multispecific complexes including ELP-MRD fusion proteins containing one or more modular recognition domains (MRDs) that bind target antigens are described. The use of these monovalent and multivalent multispecific complexes (e.g., ELP-MRD fusion proteins) in diagnostic, prognostic, and therapeutic applications and methods of making these complexes are also described.

### CROSS-REFERENCE TO RELATED APPLICATIONS

This application claims the benefit of U.S. Provisional Appl. No. 61/442,106, filed Feb. 11, 2011, which is hereby incorporated by reference in its entirety.

### REFERENCE TO A SEQUENCE LISTING SUBMITTED ELECTRONICALLY VIA EFS-WEB

The content of the electronically submitted sequence listing (Name: sequencelisting_ascii.txt, Size: 54,655 bytes; and Date of Creation: Feb. 10, 2012) is herein incorporated by reference in its entirety.

### BACKGROUND OF THE INVENTION

### Field of the Invention

This invention relates generally to monovalent and multivalent and multispecific complexes, including complexes that contain elastin-like polypeptides (ELP) and modular recognition domains (MRD). The invention also generally relates to the diagnostic, prognostic and therapeutic uses and methods of making these monovalent and multivalent multispecific complexes.

The development of multispecific molecules that bind two or more therapeutic targets offers a novel and promising approach for treating cancer, immune system related disorders, infectious disease, and other diseases or disorders. Studies of bispecific antibodies that simultaneously bind two tumor antigens associated with cell proliferation/survival signaling pathways have provided support for this approach.

Technologies that have created multispecific, and/or multivalent molecules include bispecific antibodies, dAbs, diabodies, T and Abs, nanobodies, BiTEs, SMIPs, darpins, DNLs, Affibodies, Duocalins, Adnectins, Fynomers, Kunitz Domains, Albu-dabs, DARTs, DVD-IG, Covx-bodies, peptibodies, scFv-Igs, SVD-Igs, dAb-Igs, Knobs-in-Holes, DuoBodies™ and triomAbs. Although each of these molecules may bind one or more epitopes, they each present challenges with respect to the retention of typical Ig function (e.g., half-life, effector function), production (e.g., yield, purity), valency and/or simultaneous binding.

Many of the smaller, Ig subdomain- and non-Ig-domain-based multispecific molecules have been found to possess advantages over the full-length or larger IgG-like molecules for clinical applications, including tumor radio-imaging and targeting, because of better tissue penetration and faster clearance from the circulation. In contrast, larger IgG-like molecules are often preferred for other in vivo applications because the Fc domain confers long serum half-life and supports secondary immune function, such as antibody-dependent cellular cytotoxicity and complement-mediated cytotoxicity. Unlike their fragment counterparts, engineering and production of recombinant IgG-like multispecific, multivalent molecules have been technically challenging due to their large size (150-200 kDa) and structural complexity.

Peptibodies have also been used to create multivalent therapeutic and diagnostic compositions. Peptibodies are essentially ligand-binding peptide fusions with antibody Fc regions that rely on the Fc component of the fusion protein to increase circulatory half-life and improve the pharmacokinetic properties of the peptides.

The basic units or regions of protein interaction in multivalent and often multispecific proteins that are involved in contacting and recognizing another molecule are often referred to as target-binding sites. Target-binding sites may consist of linear sequences of amino acids or discontinuous sequences of amino acids that collectively form the target-binding sites.

Peptides derived from phage display libraries often retain their binding characteristics when linked to other molecules. Specific peptides of this type and other polypeptide sequences that have target-binding sites can be treated as modular specificity blocks that can, independently or in combination with other protein scaffolds, create a single molecule with binding specificities for several defined targets. Examples of targets bound by polypeptide sequences that have target-binding sites include, integrins, (e.g., αvβ3, αvβ5), vascular endothelial growth factor (VEGF; see e.g., U.S. Pat. No. 6,660,843), Ang2 (see e.g., U.S. Pat. No. 7,138,370), and type 1 insulin-like growth factor-1 receptor (IGF1R). As an alternative to the construction of bi- and multi-functional antibodies through the combination of antibody variable domains, polypeptide sequences that have one or more target-binding sites selected from, for example, peptide display libraries, may offer a highly versatile and modular approach for the construction of multivalent and multifunctional therapeutic, prognostic and diagnostic compositions.

Elastin-like polypeptides (ELPs) are repetitive artificial polypeptides derived from recurring units of amino acid sequences found in the hydrophobic domain of tropoelastin, the soluble precursor of the extracellular matrix protein elastin. The most highly characterized ELPs contain pentapeptide repeats having the general motif of (VPGXG)ₙ, where X, the so called “guest residue,” can be any amino acid except proline, and n represents the number of pentapeptide repeats in the ELP. ELPs composed of VPGXG pentapeptide repeats exhibit an inverse temperature phase transition in which the ELPs are soluble in aqueous solution at temperatures below their inverse transition temperature (Tₜ) and undergo an aqueous demixing phase transition above their Tₜ, resulting in aggregation of the ELP and the formation of an insoluble, polymer-rich “coacervate” phase. This phase transition is reversible, and the ELP redissolves when the solution temperature is lowered below the Tₜ. The transition temperature of the ELP can be modified by altering the identity of the guest residue and/or number of pentapeptide repeats in the ELP. Generally, the addition of hydrophobic guest members to the ELP lowers the Tₜ, whereas the incorporation of ionized or polar guest residues raises the Tₜ of the ELP. Additionally, the change in transition temperature for ELPs often scales with the hydrophobic index of guest residue (see, Urry et al., J. Am. Chem. Soc. 113:4346-4348 (1991), which is herein incorporated by reference). In addition to temperature respondent phase transition, ELP phase transition can be triggered by changes in salt concentrations (e.g., kosmotropic salts), pH, redox, light and ligand binding status (see, e.g., Meyer et al., Nat. Biotechnol. 17:112-115 (1999); Cho et al., J. Phys. Chem. B. 112:13765-13771 (2008); Urry et al., Chemnt. Ed. 32:819-841 (1993); Yin et al., Biomacromolecules 7:1381-1385 (2006); Urry et al., Biochem. Biophys. Res. Comm. 188:611-617 (1992); and Shimoboji et al., PNAS 99:16592-16596 (2002), each of which is herein incorporated by reference).

Many ELP fusion proteins retain the phase transition responsiveness of the ELP components of these fusion proteins. This phase transition responsiveness appears to be limited to molecules that are physically connected to the ELP and the Tₜ of the ELP fusion protein typically differs from that of the ELP alone. This fusion delta Tₜ effect is generally associated with a decrease in fusion protein Tₜ when the ELP is fused with a hydrophobic protein and an increase in fusion protein Tₜ when the fusion partner is hydrophilic. Generally, the fraction of hydrophobic surface area of the fusion partner is linearly correlated with the change in Tₜ of the fusion protein (see, e.g., Trabbic-Carlson et al., Protein Eng. Des. Sel. 17:57-66 (2004), herein incorporated by reference).

The sequence dependent transition of ELP fusion proteins has been exploited in many settings including protein purification, depot drug delivery, hydrogel formation, and tissue engineering.

Therapeutic antibodies represent the most rapidly growing sector of the pharmaceutical industry. Treatment with bispecific antibodies and defined combinations of monoclonal antibodies is expected to show therapeutic advantages over established and emerging antibody monotherapy regimens. However, the cost of developing and producing such therapies has limited their consideration as viable treatments for most indications. There is, therefore, a great need for developing alternative monovalent and multivalent multispecific complexes and having substantially reduced production costs and comparable or superior therapeutic properties compared to conventional bispecific antibodies and combinations of monoclonal antibodies.

### BRIEF SUMMARY OF THE INVENTION

The present invention relates to monovalent and multivalent multispecific (MMM) complexes, including elastin-like peptides (ELP) and modular recognition domain (MRD) fusion proteins. The multivalent complexes comprise direct or indirect fusion of at least one ELP and at least one MRD, and the fusion can be direct or indirect. The invention encompasses MMM complexes (e.g., ELP-MRD fusion proteins) that contain a single, or alternatively, multiple MRDs. MRDs in a single MMM complex (e.g., ELP-MRD fusion protein) can have the same or different amino acid sequences and can occur in tandem or in different locations within the MMM complex (e.g., ELP-MRD fusion protein). In some embodiments, a complex comprises an ELP-MRD fusion protein as provided in U.S. Application No. 61/442,106, filed Feb. 11, 2011, which is herein incorporated by reference.

In various embodiments, MRDs in the MMM complexes (e.g., ELP-MRD fusion proteins) bind secreted, membrane associated, or intracellular targets; natural or synthetic carrier molecules (e.g., proteins such as human serum albumin), components of a patient's immune effector system, including cytotoxins, lipid or carbohydrate containing molecules; other MRD proteins; and/or compositions that do not naturally occur in a patient. The invention also encompasses MMM complexes, such as ELP-MRD fusion proteins that contain a single, or alternatively, multiple ELPs. The ELPs in the MMM complexes (e.g., ELP-MRD fusion proteins) can have the same or different amino acid sequences and can occur in tandem or in different locations within the MMM complex (e.g., ELP-MRD fusion protein). In some embodiments, the MMM complex (e.g., ELP-MRD fusion protein) comprises 1, 2, 3, 4, 5 or more antibody fragments or domains (e.g., antibody variable domains, or ScFvs or single binding domains (e.g., dab)) In some embodiments, the MMM complex (e.g., ELP-MRD fusion protein) comprises 1, 2, 3, 4, 5 or more cytotoxic agents. In some embodiments, the MMM complex (e.g., ELP-MRD fusion protein) comprises 1, 2, 3, 4, 5 or more cytotoxic agents. The antibody fragments, domains, therapeutic compounds, and/or other cytotoxic agents according to these embodiments, can occur in tandem or in different locations within the MMM complex (e.g., ELP-MRD fusion protein. The invention also encompasses variants and derivatives of the MMM complexes (e.g., ELP-MRD fusion proteins). Nucleic acids encoding MMM complexes (e.g., ELP-MRD fusion proteins) and variants and derivatives of the MMM complexes are encompassed by the invention. Nucleic acids encoding MMM complex (e.g., ELP-MRD fusion protein) ELP, MRD, linker, antibody fragment, therapeutic protein and cytotoxic agent components of the MMM complexes are also encompassed by the invention, as are nucleic acids encoding fragments and derivatives of these components. The invention additionally encompasses methods of making and using MMM complexes (e.g., ELP-MRD fusion proteins). Therapeutic, diagnostic and prognostic uses of MRDs and MMM complexes (e.g., ELP-MRD fusion proteins) are also encompassed by the invention.

MRDs and MMM complexes (e.g., ELP-MRD fusion proteins) can bind to the same epitope of a target, different epitopes on the same target, and/or different targets. In some embodiments the invention encompasses MMM complex (e.g., ELP-MRD fusion proteins) that are multivalent and multispecific. Thus, for example, in some embodiments, the MMM complexes (e.g., ELP-MRD fusion proteins) bind two or more targets and have two or more binding sites for each of the targets bound by the MMM complex. In other embodiments, the MMM complexes (e.g., ELP-MRD fusion proteins) have one (or more) single binding sites for one (or more) target(s) and multiple binding sites for other targets. Thus in some embodiments, the MMM complexes (e.g., ELP-MRD fusion proteins) are monovalent, multivalent and multispecific. Moreover, in further embodiments, the MMM complexes (e.g., ELP-MRD fusion proteins) have a single binding site for one target and a single binding site for another target, but do not have multiple binding sites for any target. Thus, in some embodiments, the MMM complexes (e.g., ELP-MRD fusion proteins) are monovalent and multispecific.

MRDs and MMM complexes (e.g., ELP-MRD fusion proteins) can possess activities such as target binding, catalytic activity, the ability to bind, link, or otherwise associate with therapeutic agents or prodrugs, and the ability to serve as reactive sites for linking or associating the MMM complex (e.g., ELP-MRD fusion protein) with additional moieties, and/or other modifications. In some embodiments, the MMM complexes (e.g., ELP-MRD fusion proteins) has one or more effector functions. The MMM complexes (e.g., ELP-MRD fusion proteins) can have advantageous manufacturing, formulation, biological, therapeutic, diagnostic or prognostic functionality.

The targets bound by MRDs and MMM complexes (e.g., ELP-MRD fusion proteins) of the invention can be any target of manufacturing, formulation, therapeutic, diagnostic, or prognostic relevance or value. In some embodiments, the invention encompasses MMM complex (e.g., ELP-MRD fusion proteins) comprising at least 1, 2, 3, 4, or 5 MRDs that bind to a validated target. In additional embodiments, the invention encompasses MMM complexes (e.g., ELP-MRD fusion proteins) comprising at least 1, 2, 3, 4, or 5 MRDs that bind to the same target site on the same antigen. In other embodiments, the MMM complexes (e.g., ELP-MRD fusion protein) comprise at least 1, 2, 3, 4, or 5 MRDs that bind to a different target site on the same antigen. In additional embodiments, MMM complexes (e.g., ELP-MRD fusion proteins) of the invention comprise at least 1, 2, 3, 4, 5 MRDs, each of which binds to a different antigen. In further embodiments, the MMM complexes (e.g., ELP-MRD fusion proteins) comprise an additional component including, for example, an antibody fragment or domain (e.g., and ScFv), that binds to a target of manufacturing, formulation, therapeutic, diagnostic, or prognostic relevance or value.

Methods of using MMM complexes (e.g., ELP-MRD fusion proteins) in diagnostic and therapeutic applications are also provided. Embodiments, relating to the use of MMM complexes of the invention include, but are not limited to methods of treating or preventing a disease, disorder, or injury comprising administering a therapeutically effective amount of an MMM complex (e.g., an ELP-MRD fusion protein) to a subject in need thereof. In some embodiments, the disease, disorder or injury is cancer. In an additional embodiment, undesired angiogenesis is inhibited. In another embodiment, angiogenesis is modulated. In yet another embodiment, tumor growth is inhibited. In other embodiments, the disease, disorder or injury is a disorder of the skeletal system (e.g., osteoporosis), cardiovascular system, nervous system, or an infectious disease. In a further embodiment, the disorder of the immune system is inflammation. In another embodiment, the disorder of the immune system is an autoimmune disease. In an additional embodiment, the disorder of the immune system is a member selected from the group consisting of: rheumatoid arthritis, Crohn's disease, systemic lupus erythematous, inflammatory bowel disease, psoriasis, diabetes, ulcerative colitis, and multiple sclerosis. In a further embodiment, the disease, disorder or injury is a metabolic disease. In another embodiment, the disease, disorder, or injury is an infectious disease. In specific embodiments, the infectious disease is human immunodeficiency virus (HIV) infection or AIDS, botulism, anthrax, or clostridium difficile. In a further embodiment, the disease, disorder, or injury is neurological. In specific embodiments, the neurological disease, disorder or injury is pain. particular embodiments, the pain is acute pain or chronic pain.

In one embodiment, the invention is directed to treating a disease or disorder by administering a therapeutically effective amount of a MMM multispecific complex to a patient in need thereof. In a further embodiment, the invention is directed to treating a disease or disorder by administering a therapeutically effective amount of an MMM complex (e.g., an ELP-MRD fusion protein) to a patient in need thereof.

In one embodiment, the MMM complex (e.g., ELP-MRD fusion protein) contains 2 binding sites for 3 or more targets. In an additional embodiment, the MMM complex contains 2 binding sites for 4 or more targets. In another embodiment, the MMM complex contains 2 binding sites for 5 or more targets. According to some embodiments, at least 1, 2, 3, 4, 5 or more of the targets bound by the MMM complex (e.g., ELP-MRD fusion protein) are located on a cell surface. According to some embodiments, at least 1, 2, 3, 4, 5 or more of the targets bound by the MMM complex (e.g., ELP-MRD fusion protein) are soluble targets (e.g., chemokines, cytokines, and growth factors). In additional embodiments, the MMM complex binds 1, 2, 3, 4, 5 or more of the targets described herein.

In additional embodiments, one or more of the targets bound by the MMM complex (e.g., ELP-MRD fusion protein) are associated with cancer. In some embodiments, the monovalent and multivalent multispecific complex (e.g., ELP-MRD fusion protein) binds 1, 2, 3, 4, 5 or more tumor antigens. In further embodiment the targets bound by the MMM complex are associated with 1, 2, 3, 4, 5 or more different signaling pathways or modes of action associated with cancer.

In additional embodiments, the targets bound by the MMM complex (e.g., ELP-MRD fusion protein) are associated with a disease or disorder of the immune system. In further embodiments, the targets bound by the MMM complex (e.g., ELP-MRD fusion protein) are associated with 1, 2, 3, 4, 5 or more different signaling pathways or modes of action associated with a disease or disorder of the immune system.

In other embodiments, the targets bound by the MMM complex (e.g., ELP-MRD fusion protein) are associated with a disease or disorder of the skeletal system (e.g., osteoporosis), cardiovascular system, nervous system, or an infectious disease. In a further embodiment, the targets bound by the MMM complex (e.g., ELP-MRD fusion protein) are associated with 1, 2, 3, 4, 5 or more different signaling pathways or modes of action associated with a disease or disorder of the skeletal system (e.g., osteoporosis), cardiovascular system, nervous system, or an infectious disease. In a further embodiment, the MMM complex (e.g., ELP-MRD fusion protein) binds 1, 2, 3, 4, 5 or more of the targets described herein.

In additional embodiments, the MMM complex (e.g., ELP-MRD fusion protein) binds (1) a target on a cell or tissue of interest (e.g., a tumor associated antigen on a tumor cell, an immune cell, a diseased cell or an infectious agent) and (2) a target on an effector cell. According to one embodiment, the binding of one or more targets by the MMM complex directs an immune response to a cell, tissue, infectious agent, or other location of interest in a subject. In some embodiments, the effector cell is a leukocyte, such as a T cell or natural killer cell. In other embodiments, the effector cell is an accessory cell, such as a myeloid cell or a dendritic cell.

In additional embodiments, the MMM complex (e.g., ELP-MRD fusion protein) binds (1) a target on a cell or tissue of interest (e.g., a tumor associated antigen on a tumor cell, an immune cell, a diseased cell or an infectious agent) and (2) a target on a leukocyte, such as a T-cell receptor molecule. According to one embodiment, the binding of one or more targets by the MMM complex (e.g., ELP-MRD fusion protein) directs an immune response to an infectious agent, cell, tissue, or other location of interest in a subject. For example, in some embodiments, the monovalent and multivalent multispecific complex binds a target on the surface of a T cell. In particular embodiments, the complex binds a CD3 target selected from CD3 delta, CD3 epsilon, CD3 gamma, CD3 zeta, TCR alpha, TCR beta, and multimers of proteins in the CD3 (TCR) complex. In specific embodiments, the MMM complex (e.g., ELP-MRD fusion protein) binds CD3. In other embodiments, the MMM complex (e.g., ELP-MRD fusion protein) binds CD2. In additional embodiments, the MMM complex (e.g., ELP-MRD fusion protein) binds a target expressed on a natural killer cell. Thus, in some embodiments, the MMM complex (e.g., ELP-MRD fusion protein) binds a target selected from: CD2, CD56, NKG2D, and CD161.

In some embodiments, the MMM complex (e.g., ELP-MRD fusion protein) binds a target expressed on an accessory cell (e.g., a myeloid cell). In some embodiments, the MMM complex (e.g., ELP-MRD fusion protein) binds a target selected from: CD64 (i.e., Fc gamma R1), an MHC class 2 and its invariant chain, TLR1, TLR2, TLR4, TLR5, and TLR6.

In further embodiments, the MMM complex (e.g., ELP-MRD fusion protein) has a single binding site (i.e., is monovalent) for a target. In some embodiments, the MMM complex (e.g., ELP-MRD fusion protein) has a single binding site for a target on a leukocyte, such as a T-cell (e.g., CD3), and multiple binding sites (i.e., is multivalent) for a target on a cell or tissue of interest (e.g., a tumor associated antigen on a tumor cell, such as a target disclosed herein). In further embodiments, the MMM complex (e.g., ELP-MRD fusion protein) contains single binding sites for 2 different targets (i.e., monovalently binds more than one different target). In particular embodiments, the cell or tissue of interest is a cancer cell, immune cell, diseased cell, or an infectious agent.

In some embodiments, an MMM complex (e.g., an ELP-MRD fusion protein) has a single binding site for CD3. In further embodiments, the MMM complex (e.g., ELP-MRD fusion protein) has a single binding site for CD3 and multiple binding sites for 1, 2, 3, 4, 5 or more different targets (e.g., a tumor antigen or other target disclosed herein). In additional embodiments, the MMMM complex (e.g., ELP-MRD fusion protein) has a single binding site for CD3 and a single binding site for a different target (i.e., monovalently binds CD3 and a different target). In other embodiments, an MMM complex (e.g., an ELP-MRD fusion protein) has a single binding site for CD3 epsilon. In some embodiments, the MMM complex (e.g., ELP-MRD fusion protein) has a single binding site for CD3 epsilon and multiple binding sites for 1, 2, 3, 4, 5 or more different targets (e.g., a tumor antigen or other target disclosed herein). In additional embodiments, the MMM complex (e.g., ELP-MRD fusion protein) has a single binding site for CD3 epsilon and a single binding site for a different target (i.e., monovalently binds CD3 epsilon and a different target). In some embodiments, the MMM complex (e.g., ELP-MRD fusion protein) has multiple binding sites for a target on a cancer cell or tissue selected from breast cancer, colorectal cancer, endometrial cancer, kidney (renal cell) cancer, lung cancer, melanoma, Non-Hodgkin Lymphoma, leukemia, prostate cancer, bladder cancer, pancreatic cancer, and thyroid cancer. In additional embodiments, the MMM complex (e.g., ELP-MRD fusion protein) has multiple binding sites for a target on a neurological tumor. In particular embodiments, the neurological tumor is a glioma (e.g., a glioblastoma, glioblastoma multiforme (GBM), and astrocytoma), ependymoma, oligodendroglioma, neurofibroma, sarcoma, medulloblastoma, primitive neuroectodermal tumor, pituitary adenoma, neuroblastoma or cancer of the meninges (e.g., meningioma, meningiosarcoma and gliomatosis).

The invention is also directed to methods of treating a disease or disorder by administering a therapeutically effective amount of an MMM complex (e.g., an ELP-MRD fusion protein) that has a single binding site for a target (i.e., that monovalently binds a target) to a subject in need thereof. In some embodiments, the administered MMM complex (e.g., ELP-MRD fusion protein) has a single binding site for a target on a leukocyte such as a T-cell (e.g., CD3). In further embodiments, the administered MMM complex (e.g., ELP-MRD fusion protein) has a single binding site for a target on a leukocyte such as a T-cell (e.g., CD3) and multiple binding sites for (i.e., is capable of multivalently binding) a target located on a cell or tissue of interest (e.g., a tumor antigen on a tumor cell). In further embodiments, the MMM complex (e.g., ELP-MRD fusion protein) has a single binding site for a target on a leukocyte (e.g., CD3) and a single binding site for a different target. In some embodiments, the cell of interest is a tumor cell from a cancer selected from breast cancer, colorectal cancer, endometrial cancer, kidney (renal cell) cancer, lung cancer, melanoma, Non-Hodgkin Lymphoma, leukemia, prostate cancer, bladder cancer, pancreatic cancer, and thyroid cancer. In additional embodiments, the monovalent and multivalent multispecific complex has multiple binding sites for a target on a neurological tumor. In particular embodiments, the neurological tumor is a glioma (e.g., a glioblastoma, glioblastoma multiforme (GBM), and astrocytoma), ependymoma, oligodendroglioma, neurofibroma, sarcoma, medulloblastoma, primitive neuroectodermal tumor, pituitary adenoma, neuroblastoma or cancer of the meninges (e.g., meningioma, meningiosarcoma and gliomatosis).

In further embodiments, the invention is directed to treating a disease or disorder by administering to a subject in need thereof, a therapeutically effective amount of a monovalent and multivalent multispecific complex that has a single binding site for a target (i.e., that monovalently binds a target) and multiple binding sites for 1, 2, 3, 4, 5 or more different targets. In further embodiments, the monovalent and multivalent multispecific complex has single binding sites for 2 different targets. In some embodiments, the monovalent and multivalent multispecific complex has multiple binding sites for a target on a cancer cell or tissue selected from breast cancer, colorectal cancer, endometrial cancer, kidney (renal cell) cancer, lung cancer, melanoma, Non-Hodgkin Lymphoma, leukemia, prostate cancer, bladder cancer, pancreatic cancer, and thyroid cancer. In additional embodiments, the monovalent and multivalent multispecific complex has multiple binding sites for a target on a neurological tumor. In particular embodiments, the neurological tumor is a glioma (e.g., a glioblastoma, glioblastoma multiforme (GBM), and astrocytoma), ependymoma, oligodendroglioma, neurofibroma, sarcoma, medulloblastoma, primitive neuroectodermal tumor, pituitary adenoma, neuroblastoma or cancer of the meninges (e.g., meningioma, meningiosarcoma and gliomatosis).

In additional embodiments, the invention is directed to treating a disease or disorder by administering to a subject in need thereof, a therapeutically effective amount of an MMM complex (e.g., an ELP-MRD fusion protein) that has a single binding site for CD3 (e.g., CD3 epsilon) that monovalently binds CD3 and multiple binding sites for 1, 2, 3, 4, 5 or more different targets located on a cell or tissue of interest (e.g., a tumor antigen on a tumor cell). In some embodiments, the administered MMM complex has a single binding site for CD3 (e.g., CD3 epsilon) and a single binding site for a different target and also has multiple binding sites for a target located on a cell or tissue of interest (e.g., a tumor antigen on a tumor cell). In some embodiments, the MMM complex (e.g., ELP-MRD fusion protein) has multiple binding sites for a target on a cancer cell selected from breast cancer, colorectal cancer, endometrial cancer, kidney (renal cell) cancer, lung cancer, melanoma, Non-Hodgkin Lymphoma, leukemia, prostate cancer, bladder cancer, pancreatic cancer, and thyroid cancer. In additional embodiments, the MMM complex (e.g., an ELP-MRD fusion protein) has multiple binding sites for a target on a neurological tumor. In particular embodiments, the neurological tumor is a glioma (e.g., a glioblastoma, glioblastoma multiforme (GBM), and astrocytoma), ependymoma, oligodendroglioma, neurofibroma, sarcoma, medulloblastoma, primitive neuroectodermal tumor, pituitary adenoma, neuroblastoma or cancer of the meninges (e.g., meningioma, meningiosarcoma and gliomatosis).

In further embodiments, the MMM (e.g., an ELP-MRD fusion protein) has a single binding site for (i.e., monovalently binds) a cell surface target that requires multimerization for signaling. In some embodiments, the MMM complex (e.g., ELP-MRD fusion protein) has a single binding site for a growth factor receptor. In other embodiments, the MMM complex (e.g., ELP-MRD fusion protein) has a single binding site for a TNF receptor superfamily member. In additional embodiments, the MMM complex (e.g., an ELP-MRD fusion protein) additionally has a single binding site for a different target (i.e., monovalently binds more than one different target).

In additional embodiments, the MMM complex (e.g., ELP-MRD fusion protein) binds a target associated with an endogenous blood brain barrier (BBB) receptor mediated transport system (e.g., the insulin receptor, transferrin receptor, leptin receptor, lipoprotein receptor, and the IGF receptor mediated transport systems) and is capable of crossing to the brain (cerebrospinal fluid) side of the BBB. In some embodiments, the MMM complex has 2 or more binding sites for a target antigen associated with an endogenous BBB receptor mediated transport system. In additional embodiments, the MMM complex has a single binding site for a target associated with an endogenous BBB receptor mediated transport system. In further embodiments, the MMM complex additionally binds 1, 2, 3, 4, 5, or more targets located on the brain side of the BBB. In particular embodiments, the MMM complex (e.g., ELP-MRD fusion protein) binds 1, 2, 3, 4, 5, or more targets associated with a neurological disease or disorder. In another embodiment, the MMM complex is administered to a subject to treat a brain cancer, metastatic cancer of the brain, or primary cancer of the brain. In a further embodiment, the MMM complex is administered to a subject to treat brain injury, stroke, spinal cord injury, or to manage pain.

In additional embodiments, targets bound by the MMM complex (e.g., an MRD-ELP fusion protein) are associated with a disease or disorder of the skeletal system (e.g., osteoporosis), cardiovascular system, nervous system, or an infectious disease. In a further embodiment targets bound by the MMM complex (e.g., an ELP-MRD fusion protein) are associated with 1, 2, 3, 4, 5 or more different signaling pathways or modes of action associated with one or more of the above diseases or disorders. In a further embodiment, the MMM complex (e.g., an ELP-MRD fusion protein) binds 1, 2, 3, 4, 5 or more of the targets described herein.

In a further embodiment, the MMM complex (e.g., an ELP-MRD fusion protein) contains binding sites for 3 or more targets. In an additional embodiment, the MMM complex (e.g., an ELP-MRD fusion protein) contains 2 binding sites for 4 or more targets. In an additional embodiment, the MMM complex (e.g., an ELP-MRD fusion protein) contains 2 binding sites for 5 or more targets.

In one embodiment, the MMM complex (e.g., an MRD-ELP fusion protein) contains 2 binding sites for 3 or more targets. In an additional embodiment, the MMM complex (e.g., MRD-ELP fusion protein) contains 2 binding sites for 4 or more targets. In another embodiment, the MMM complex (e.g., MRD-ELP fusion protein) contains 2 binding sites for 5 or more targets. According to some embodiments, at least 1, 2, 3, 4, 5 or more of the targets bound by the MMM complex (e.g., ELP-MRD fusion protein) are located on a cell surface. According to some embodiments, at least 1, 2, 3, 4, 5 or more of the targets bound by the MMM complex (e.g., an ELP-MRD fusion protein) are soluble targets (e.g., chemokines, cytokines, and growth factors). In additional embodiments, the MMM complex (e.g., an ELP-MRD fusion protein) binds 1, 2, 3, 4, 5, or more of the targets described herein.

In additional embodiments, the targets bound by the MMM complex (e.g., MRD-ELP fusion protein) are associated with cancer. In a further embodiment the targets bound by the MMM complex (e.g., ELP-MRD fusion protein) are associated with 1, 2, 3, 4, 5 or more different signaling pathways or modes of action associated with cancer.

In additional embodiments, a target bound by the MMM complex (e.g., MRD-ELP fusion protein) is associated with a disease or disorder of the immune system. In a further embodiment the targets bound by the MMM complex (e.g., ELP-MRD fusion protein) are associated with 1, 2, 3, 4, 5 or more different signaling pathways or modes of action associated with a disease or disorder of the immune system.

In additional embodiments, a target bound by the MMM complex (e.g., MRD-ELP fusion protein) is associated with a disease or disorder of the skeletal system, cardiovascular system, nervous system, or an infectious disease. In a further embodiment a target bound by the MMM complex (e.g., ELP-MRD fusion protein) is associated with 1, 2, 3, 4, 5, or more different signaling pathways or modes of action associated with one or more of the above diseases or disorders. In another embodiment, the MMM complex (e.g., an ELP-MRD fusion protein) binds 1, 2, 3, 4, or more of the targets described herein.

The MMM complexes of the invention (e.g., MRD-ELP fusion proteins) provide the ability to selectively bind multiple targets (e.g., receptors and microenvironment associated targets) having for example, different, overlapping, or redundant mechanisms of action associated with the etiology or pathophysiology of a disease or disorder. In additional embodiments, the invention encompasses an MMM complex (e.g., an ELP-MRD fusion protein) that is covalently or otherwise associated with a cytotoxic agent. According to some embodiments, the cytoxic agent is covalently attached to an MMM complex (e.g., an ELP-MRD fusion protein) by a linker. In additional embodiments, the cytotoxic agent is a chemotherapeutic agent, growth inhibitory agent, toxin (e.g., an enzymatically active toxin of bacterial, fungal, plant, or animal origin, or fragments thereof), radioactive isotope (i.e., a radioconjugate), or prodrug. The complexes of the invention are optionally linked to the cytotoxic agent by a linker. In particular embodiments, a linker attaching the monovalent and multivalent multispecific complex and the cytotoxic agent is cleavable by a protease. In particular embodiments, a linker attaching the MMM complex (e.g., ELP-MRD fusion protein) and the cytotoxic agent is cleavable under low pH or reducing conditions. Methods of using ELP-MRD complex-cytoxic agent compositions (e.g., ELP-MRD fusion protein-cytotoxic agent conjugates) are also encompassed by the invention.

In additional embodiments, the MMM complex (e.g., ELP-MRD fusion protein) is covalently or otherwise associated with a cytotoxic agent selected from, a toxin, a chemotherapeutic agent, a drug moiety (e.g., a chemotherapeutic agent or prodrug), an antibiotic, a radioactive isotope, a chelating ligand (e.g., DOTA, DOTP, DOTMA, DTPA and TETA), and a nucleolytic enzyme. In particular embodiments, the cytotoxic agent is a member selected from: auristatin, dolostantin, MMAE, MMAF, a maytansinoid derivative (e.g., the DM1 (N(2′)-deacetyl-N(2′)-(3-mercapto-1-oxopropyl)-maytansine), DM3 (N(2′)-deacetyl-N2-(4-mercapto-1-oxopentyl)-maytansine) and DM4 (N(2′)-deacetyl-N2-(4-mercapto-4-methyl-1-oxopentyl)-maytansine).

Methods of treatment or prevention comprising administering an additional therapeutic agent along with MMM complexes of the invention, such as ELP-MRD fusion proteins, are also provided.

### BRIEF DESCRIPTION OF THE FIGURES

FIG. 1: Schematic representation of exemplary ELP-MRD fusion protein. MRDs can be arrayed as tandem repeats interspersed throughout an ELP-based scaffold, fused to the N-terminus, and/or fused to the C-terminus of the ELP scaffold. The ELP backbone can be comprised of repeating structural units. Modifications and other modular components, such as therapeutics, can be introduced by direct or indirect attachment to the side functional groups of amino acids within ELPs, MRDs and/or or other components of the MMM complex, including the N- and C-termini.

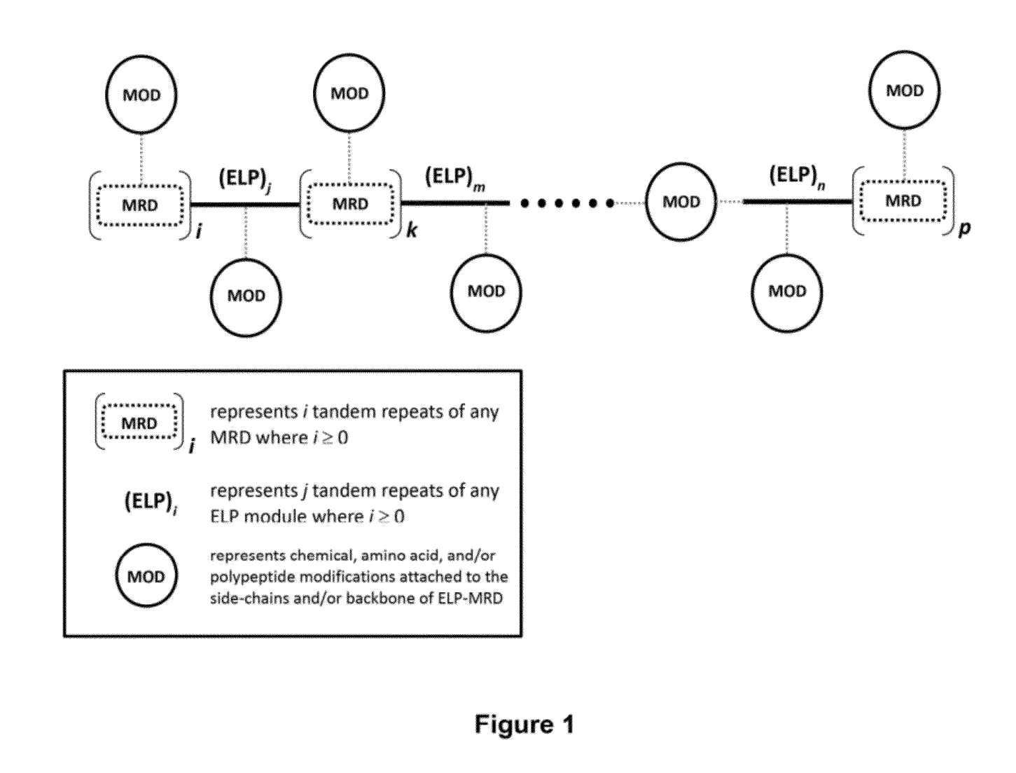

FIG. 2: Schematic representation of ELP-MRD fusion construct generation by recursive directional ligation and plasmid reconstruction.

FIG. 3: (A) Non-reducing SDS-PAGE of purified ELP-MRD fusion protein fractions from cobalt resin columns for three constructs. Lane M: Molecular weight marker (Novex Sharp Prestained Protein Standard). (B) Representative Western blot of purified ELP-MRD fusion protein fractions.

FIG. 4: Non-reducing SDS-PAGE analysis of purified and buffer-exchanged ELP-MRD fusion protein.

FIG. 5: Binding data for tetravalent and monovalent ELP-MRD fusion proteins as measured by ELISA on recombinant human angiopoietin-2 (rhAng2) coated and uncoated microplate wells.

FIG. 6: Binding data for a bispecific ELP-MRD fusion protein as measured by ELISA on rhAng-2-coated, rhVEGF165-coated, and uncoated wells.

FIG. 7: Binding data for an ELP-MRD fusion protein displaying an internal constrained MRD as measured by ELISA on rhAng-2-coated and uncoated wells.

FIG. 8: Binding data for the HER2-targeted ELP-MRD fusion protein as measured by FACS analysis on SKBR3 (HER2+) and MDAMB231 (HER2−) cells.

FIG. 9: Pharmacokinetic data for ANGa-ELP₂(160)-10×His fusion protein in mice.

### DETAILED DESCRIPTION OF THE INVENTION

The following provides a description of ELP-MRD fusions comprising at least 1, 2, or more modular recognition domains (MRDs). The linkage of one or more MRDs to at least one ELP results in a multivalent and potentially multispecific molecule that provides distinct diagnostic and therapeutic advantages over conventional compositions. In addition, the MMM complex (e.g., ELP-MRD fusion proteins) can readily be manufactured using conventional recombinant expression systems and techniques. The target(s) bound by one or more of the MMM complex (e.g., ELP-MRD fusion proteins) of the invention can be any suitable target that confers a desired property to the MMM complex (e.g., ELP-MRD fusion proteins). MRDs and other components of MMM complexes (e.g., ELP-MRD fusion proteins) can be operably linked to the amino or carboxyl terminus of the ELP, and the attachment can be direct or indirect (e.g., through a chemical or polypeptide linker). Compositions of ELP-MRD fusions, nucleic acids encoding ELP-MRD fusions, methods of recombinantly producing MMM complexes (e.g., ELP-MRD fusion proteins), methods of designing and manufacturing ELP-MRD fusions, and methods of using ELP-MRD fusions are among the embodiments, also encompassed by the invention and described in the sections below.

Standard techniques can be used for producing MMM complexes (e.g., ELP-MRD fusion proteins), including recombinant DNA molecule and protein production, as well as for tissue culture and cell transformation. Enzymatic reactions and purification techniques are typically performed according to the manufacturer's specifications or as commonly accomplished using or routinely modifying known procedures such as, those set forth in Sambrook et al. (Molecular Cloning: A Laboratory Manual. Cold Spring Harbor Laboratory Press, Cold Spring Harbor, N.Y. (1989)); PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila, et al., Nucleic Acids Res. 19:967 (1991); Eckert, et al., PCR Methods and Applications 1:17 (1991); PCR (eds. McPherson et al., IRL Press, Oxford); Harlow et al., Antibodies: A Laboratory Manual, (Cold Spring Harbor Laboratory Press, 2nd ed. 1988) and R. Kontermann and S. Dubel (eds.), “The Antibody Engineering Lab Manual” (Springer Verlag, Heidelberg/New York, 2000) (the contents of each of which are herein incorporated by reference), or as described herein. Unless specific definitions are provided, the nomenclature utilized in connection with, and the laboratory procedures and techniques of analytical chemistry, synthetic organic chemistry, and medicinal and pharmaceutical chemistry described herein, are those known and used in the art. Additionally, standard techniques can be used for chemical syntheses, chemical analyses, recombinant production, purification, pharmaceutical preparation, formulation, delivery, and treatment of patients.

The section headings used herein are for organizational purposes only and are not to be construed as in any way limiting of the subject matter described.

### I. Definitions

The term “ELP” is used herein to refer to an elastin-like polypeptide. ELPs are repeating peptide sequences that can have characteristics found to exist in the elastin protein. Among these repeating peptide sequences are polytetra-, polypenta-, polyhexa-, polyhepta-, polyocta, and polynonapeptides. More information about ELPs can be found in the following references which are herein incorporated by reference in their entireties: U.S. Pat. No. 6,852,834 and U.S. Patent Publication Nos. 2005/0255554 and 2010/0022455.

The terms “monovalent and multivalent multispecific”, “MMM” and “ELP-MRD fusion protein” are used interchangeably herein. Each of these terms may also be used to refer to a “complex” of the invention; MMM complexes can contain MRDs, ELPs, cytoxic agents, and binding motifs in addition to MRDs that bind to one or more targets. For example, the MMM complex (e.g., ELP-MRD fusion protein) can contain a portion of, or a derivative of, a binding sequence contained in an antibody (e.g., a single binding domain, a ScFv, a CDR region, an FcRN binding sequence, and an Fc gamma receptor binding sequence). The MMM complex (e.g., ELP-MRD fusion protein) can also include a cytotoxic agent or a therapeutic agent.

The term “monovalent and multivalent multispecific complex(es)” or “MMM complex(es) is used herein to refer compositions that are able to bind 2 or more targets and that contain one binding site and/or multiple binding sites for different epitopes. The different epitopes can be on the same or different targets. MMM complexes can be multivalent and multispecific and can therefore bind two or more targets and have two or more binding sites for each of the targets bound by the MMM complex. MMM complexes can also have one (or more) single binding sites for one (or more) target(s) and multiple binding sites for other targets and accordingly, these MMM complexes are monovalent (with respect to the single binding site(s)), multivalent and multispecific. Moreover, MMM complexes can be monovalent and multispecific and thus, only contain single binding sites for two or more different targets. MMM complexes include ELP-MRD fusion proteins.

The term “ ” MMM-Drug complex” or “MMM-cytotoxic agent complex” as used herein, refers to an MMM complex containing one or more cytotoxic agents

The term “cytotoxic agent” as used herein, includes any agent that is detrimental to cells including for example, substance that inhibits or prevents the function of cells and/or causes destruction of cells. The term is intended to include a chemotherapeutic agent, a drug moiety (e.g., a cytokine or prodrug), an antibiotic, a radioactive isotope, a chelating ligand (e.g., DOTA, DOTP, DOTMA, DTPA and TETA), a nucleolytic enzyme, a toxins such as a small molecule toxin or enzymatically active toxin of bacterial, fungal, plant or animal origin, including fragments and/or variants of these toxins. In particular embodiments, the cytotoxic agent is a member selected from: auristatin, dolostantin, MMAE, MMAF, a maytansinoid derivative (e.g., the DM1 (N(2′)-deacetyl-N(2′)-(3-mercapto-1-oxopropyl)-maytansine), DM3 (N(2′)-deacetyl-N2-(4-mercapto-1-oxopentyl)-maytansine) and DM4 (N(2)-deacetyl-N2-(4-mercapto-4-methyl-1-oxopentyl)-maytansine).

The terms “T lymphocyte,” “T cell,” “T cells,” and “T cell population,” are used interchangeably herein to refer to a cell or cells which display on their surface one or more antigens characteristic of T cells, for example, CD3 CD8, and CD4. The term includes progeny of a T cell or T cell population. A “T lymphocyte” or “T cell” includes a cell which expresses CD3 on its cell surface and a T cell antigen receptor (TCR) capable of recognizing antigen when displayed on the surface of autologous cells, or any antigen-presenting matrix, together with one or more MHC molecules or, one or more non-classical MHC molecules. The term “T cells” may refer to any T cells, including for example, lymphocytes that are phenotypically CD3′ (i.e., express CD3 on the cell surface).

As used herein, CD3, is used to refer individually or collectively to a molecule expressed as part of the T cell receptor and having a meaning as typically ascribed to it in the art. In humans, the term CD3 encompasses all known CD3 subunits, for example CD3 delta, CD3 epsilon, CD3 gamma, and CD3 zeta (TCR zeta), as well as CD3 alpha (TCR alpha), and CD3 beta (TCR beta) in individual or independently combined form.

The term “naturally occurring” when used in connection with biological materials such as a nucleic acid molecules, polypeptides, host cells, and the like refers to those which are found in nature and not modified by a human being.

The term “domain” as used herein refers to a part of a molecule or structure that shares common physical or chemical features, for example hydrophobic, polar, globular, helical domains or properties, e.g., a protein binding domain, a DNA binding domain or an ATP binding domain. Domains can be identified by their homology to conserved structural or functional motifs.

The terms “compete,” “ability to compete” and “competes with” are relative terms used to describe an MRD and/or the MMM complex (e.g., ELP-MRD fusion proteins) that produce a 50% inhibition of binding to a target by an MRD, and/or, antibody fragment or domain and/or the MMM complex (e.g., ELP-MRD fusion proteins) as determined in a standard competition assay as described herein or otherwise known in the art, including, but not limited to, competitive assay systems using techniques such as radioimmunoassays (RIA), enzyme immunoassays (EIA), preferably the enzyme linked immunosorbent assay (ELISA), “sandwich” immunoassays, immunoradiometric assays, fluorescent immunoassays, luminescent, electrochemical luminescent, and immunoelectrophoresis assays. Methods for determining binding and affinity of candidate binding molecules are known in the art and include, but are not limited to, affinity chromatography, size exclusion chromatography, equilibrium dialysis, fluorescent probe displacement, and plasma resonance.

An MMM complex (e.g., an ELP-MRD fusion protein), MRD, antibody fragment or domain (e.g., ScFv), other component on an MMM complex (e.g., an ELP-MRD fusion protein), or other molecule, is said to “competitively inhibit” binding of a reference molecule to a given epitope if it binds to that epitope to the extent that it blocks, to some degree, binding of the reference molecule to the epitope. Competitive inhibition can be determined by any method known in the art, for example, competition ELISA assays. As used herein, an MMM complex (e.g., an ELP-MRD fusion protein), MRD, antibody fragment or domain (e.g., ScFv), other component on an MMM complex (e.g., an ELP-MRD fusion protein), or other molecule can be said to competitively inhibit binding of the reference molecule to a given epitope, for example, by at least 90%, at least 80%, at least 70%, at least 60%, or at least 50%.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. It is highly preferred that the addition of conservative substitutions in the sequences of the MMM complexes (e.g., ELP-MRD fusion proteins) of the invention do not abrogate binding of the MMM complex (e.g., ELP-MRD fusion protein) containing the amino acid sequence substitutions to the antigen(s) to which the MMM complex binds. Methods of identifying nucleotide and amino acid conservative substitutions and non-conservative substitutions which do not eliminate polypeptide or antigen binding are well-known in the art (see, e.g., Brummell et al., Biochem. 32:1180-1187 (1993); Kobayashi et al., Protein Eng. 12(10):879-884 (1999); and Burks et al., Proc. Natl. Acad. Sci. USA 94:412-417 (1997)).

A “modular recognition domain” (MRD) or “target binding peptide” is a molecule, such as a protein, that can specifically (non-randomly) bind to a target molecule. The amino acid sequence of a MRD can typically tolerate some degree of variability and still retain a degree of capacity to bind the target molecule. Furthermore, changes in the sequence can result in changes in the binding specificity and in the binding constant between a preselected target molecule and the binding site. In one embodiment, an MRD is an agonist of the target it binds. An MRD agonist refers to a MRD that in some way increases or enhances the biological activity of an Med's target protein or has biological activity comparable to a known agonist of an Med's target protein. In another embodiment, an MRD is an antagonist of the target it binds. An MRD antagonist refers to an MRD that blocks or in some way interferes with the biological activity of an Med's target protein or has biological activity comparable to a known antagonist or inhibitor of an Med's target protein.

“Cell surface receptor” refers to molecules and complexes of molecules capable of receiving a signal and transmitting such a signal across the plasma membrane of a cell. An example of a cell surface receptor is an activated integrin receptor, such as, an activated αvβ3 integrin receptor on a metastatic cell. As used herein, “cell surface receptor” also includes a molecule expressed on a cell surface that is capable of being bound by an MMM complex (e.g., an ELP-MRD fusion protein).

“Target” refers to any molecule or combination of molecules that can be bound by an MMM complex (e.g., an ELP-MRD fusion protein), MRD, antibody variable domain fragment, or other component of the MMM complex (e.g., ELP-MRD fusion protein).

As used herein, a “target-binding site” is any portion of a target that is a known, or yet to be defined, linear or conformational amino acid sequence or other structure that has the ability to be bound by an MMM complex (e.g., an ELP-MRD fusion protein), MRD, antibody variable domain fragment, or other component of the MMM complex (e.g., ELP-MRD fusion protein).

The term “epitope” or “antigenic determinant” are used interchangeably herein and refer to that portion of any molecule capable of being recognized and specifically bound by a particular binding agent (e.g., an MRD or an antibody fragment (e.g., ScFv), or domain). When the recognized molecule is a polypeptide, epitopes can be formed from contiguous amino acids (i.e., a linear epitope), noncontiguous amino acids (i.e., a conformational epitope) and/or other chemically active surface groups of molecules (such as carbohydrates) juxtaposed by tertiary folding of a protein. Epitopes formed from contiguous amino acids are typically retained upon protein denaturation, whereas epitopes formed by tertiary folding are often lost upon protein denaturation. An epitope typically includes at least 3 amino acids, and more usually, at least 5 or 8-10 amino acids in a unique spatial conformation.

The terms “protein” and “polypeptide” are used interchangeably herein to refer to a biological polymer comprising units derived from amino acids (including naturally occurring or synthetic amino acids and both D- and L-amino acids) linked via peptide bonds; a protein can be composed of two or more chains.

A “fusion protein” or fusion polypeptide is a polypeptide comprised of at least two polypeptides and optionally a linking sequence, and that are to operatively linked into one continuous protein. The two polypeptides linked in a fusion protein are typically derived from two independent sources, and therefore a fusion protein comprises two linked polypeptides not normally found linked in nature. The two polypeptides can be operably attached directly by a peptide bond or can be linked indirectly through a linker described herein or otherwise known in the art.

The term “operably linked,” as used herein, indicates that two molecules (e.g., polypeptides) are attached so as to each retain functional activity. Two molecules are “operably linked” whether they are attached directly or indirectly (e.g., via a linker).

The term “linker” refers to a peptide or other moiety that is optionally located between ELPs, MRDs, antibody fragments or domains, therapeutics and other components of the MMM complexes (e.g., ELP-MRD fusion proteins) of the invention. In some embodiments, one or more of the linkers in an MMM complex (e.g., an ELP-MRD fusion proteins) of the invention have from about 1 to 20 amino acids, about 2 to 20 amino acids, or about 4 to 15 amino acids. In one embodiment, the MMM complex (e.g., ELP-MRD fusion proteins) of the invention comprises at least one linker containing 1 to 20 amino acids selected from glycine, alanine, proline, asparagine, glutamine, and lysine.

In another embodiment, a linker is made up of a majority of amino acids that are sterically unhindered, such as glycine and alanine Thus, in some embodiments, the linker is selected from polyglycines (such as (Gly)₅, and (Gly)₈), poly(Gly-Ala), and polyalanines. The linker can also be a non-peptide linker such as an alkyl linker, or a PEG linker. For example, alkyl linkers such as—NH—(CH₂)s-C(O)—, wherein s=2-20 can be used. These alkyl linkers may further be substituted by any non-sterically hindering group such as lower alkyl (e.g., C₁-C₆) lower acyl, halogen (e.g., Cl, Br), CN, NH₂, phenyl, etc. An exemplary non-peptide linker is a PEG linker. In some embodiments, the PEG linker has a molecular weight of about 100 to 5000 kDa, or about 100 to 500 kDa. The peptide linkers can be altered to form derivatives. In some embodiments, the linker is a non-peptide linker such as an alkyl linker, or a PEG linker. In further embodiments, the linker is a “cleavable linker” facilitating release of an MRD or cytotoxic agent within a cell or in the proximity of the cell.

“Target cell” refers to any cell in a subject (e.g., a human, rabbit, mouse, rat, or other animal) that can be targeted by a multispecific and multivalent, MRD, antibody variable domain fragment, or other component of the MMM complex (e.g., ELP-MRD fusion protein) of the invention. The target cell can be a cell expressing or overexpressing a target-binding site that is bound by an MMM complex (e.g., an ELP-MRD fusion protein), such as an activated integrin receptor.

The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells, or tissues infected with pathogens, cancerous cells, or, in cases of autoimmunity or pathological inflammation, normal human cells or tissues.

As used herein, the term “effector cell” refers to an immune cell which is involved in the effector phase of an immune response, as opposed to the cognitive and activation phases of an immune response. Exemplary immune cells include a cell of a myeloid or lymphoid origin, e.g., lymphocytes (e.g., B cells and T cells including cytolytic T cells (CTLs)), killer cells, natural killer cells, macrophages, monocytes, eosinophils, neutrophils, polymorphonuclear cells, granulocytes, mast cells, and basophils). Some effector cells express specific Fc receptors and carry out specific immune functions. In certain embodiments, an effector cell is capable of inducing antibody-dependent cell-mediated cytotoxicity (ADCC), e.g., a neutrophil capable of inducing ADCC. For example, monocytes and macrophages, which express FcR, are involved in specific killing of target cells and presenting antigens to other components of the immune system, or binding to cells that present antigens. In other embodiments, an effector cell can phagocytose a target antigen or target cell. The expression of a particular FcR on an effector cell can be regulated by humoral factors such as cytokines. For example, expression of Fc alpha RI has been found to be up-regulated by G-CSF or GM-CSF. This enhanced expression increases the effector function of Fc alpha RI-bearing cells against targets. Exemplary functions of an effector cell include the phagocytosing or lysing of a target antigen or a target cell.

“Target cell” refers to any cell or pathogen whose elimination would be beneficial in a patient (e.g., a human or animal) and that can be targeted by a composition (e.g., MRD-ELP fusion protein) of the invention.

“Patient,” “subject,” “animal” or “mammal” are used interchangeably and refer to mammals such as human patients and non-human primates, as well as experimental animals such as rabbits, rats, and mice, and other animals. Animals include all vertebrates, e.g., mammals and non-mammals, such as sheep, dogs, cows, chickens, amphibians, and reptiles. In some embodiments, the patient is a human.

“Treating” or “treatment” includes the administration of an MMM complex (e.g., an ELP-MRD fusion protein) to prevent or delay the onset of the symptoms, complications, or biochemical indicia of a disease, condition, or disorder, alleviating the symptoms or arresting or inhibiting further development of the disease, condition, or disorder. Treatment can be prophylactic (to prevent or delay the onset of the disease, or to prevent the manifestation of clinical or subclinical symptoms thereof) or therapeutic suppression or alleviation of symptoms after the manifestation of the disease, condition, or disorder. Treatment can be with an MMM complex (e.g., an ELP-MRD fusion protein) containing composition alone, or in combination with 1, 2, 3 or more additional therapeutic agents.

As used herein, the terms “pharmaceutically acceptable,” or “physiologically tolerable” and grammatical variations thereof, as they refer to compositions, carriers, diluents and reagents, are used interchangeably and represent that the materials are capable of administration to or upon a human without the production of therapeutically prohibitive undesirable physiological effects such as nausea, dizziness, gastric upset and the like.

As used herein, “Modulate,” means adjustment or regulation of amplitude, frequency, degree, or activity. In another related embodiment, such modulation can be positively modulated (e.g., an increase in frequency, degree, or activity) or negatively modulated (e.g., a decrease in frequency, degree, or activity).

“Cancer,” “tumor,” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (metastasize), as well as any of a number of known characteristic structural and/or molecular features. A “cancerous tumor,” or “malignant cell” is understood as a cell having specific structural properties, lacking differentiation and being capable of invasion and metastasis. Examples of cancers that can be treated using the MMM complexes (e.g., ELP-MRD fusion proteins) of the invention include solid tumors and hematologic cancers. Additional, examples of cancers that can be treated using the ELP-MRD fusions of the invention include breast, lung, brain, bone, liver, kidney, colon, head and neck, ovarian, hematopoietic (e.g., leukemia), and prostate cancer. Further examples of cancer that can be treated using the MMM complexes (e.g., ELP-MRD fusion proteins) include, but are not limited to, carcinoma, lymphoma, myeloma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancers.

In additional embodiments, MMM complexes (e.g., ELP, ELP-MRD fusion proteins) are administered to treat a hematologic cancer. In further embodiments, the, MMM complexes (e.g., ELP, ELP-MRD fusion proteins) are administered to treat a cancer selected from: lymphoma, leukemia, myeloma, lymphoid malignancy, cancer of the spleen, and cancer of the lymph nodes. In additional embodiments, the MMM complexes (e.g., ELP-MRD fusion proteins) are administered to treat a lymphoma selected from: Burkitt's lymphoma, diffuse large cell lymphoma, follicular lymphoma, Hodgkin's lymphoma, mantle cell lymphoma, marginal zone lymphoma, mucosa-associated-lymphoid tissue B cell lymphoma, non-Hodgkin's lymphoma, small lymphocytic lymphoma, and a T cell lymphoma. In additional embodiments, the MMM complexes (e.g., ELP-MRD fusion proteins) are administered to treat a leukemia selected from: chronic lymphocytic leukemia, B cell leukemia (CD5+ B lymphocytes), chronic myeloid leukemia, lymphoid leukemia, acute lymphoblastic leukemia, myelodysplasia, myeloid leukemia, acute myeloid leukemia, and secondary leukemia. In additional embodiments, the MMM complexes (e.g., ELP-MRD fusion proteins) are administered to treat multiple myeloma. Other types of cancer and tumors that can be treated using MMM complexes (e.g., ELP-MRD fusion proteins) are described herein or otherwise known in the art.

An “effective amount” of an MMM complex (e.g., an ELP-MRD fusion protein) as disclosed herein is an amount sufficient to carry out a specifically stated purpose such as to bring about an observable change in the level of one or more biological activities related to the target to which the MMM complex (e.g., ELP-MRD fusion protein) binds. In certain embodiments, the change increases the level of target activity. In other embodiments, the change decreases the level of target activity. An “effective amount” can be determined empirically and in a routine manner, in relation to the stated purpose.

The term “therapeutically effective amount” refers to an amount of an MMM complex (e.g., an ELP-MRD fusion protein), MRD, antibody fragment or domain, or therapeutic polypeptide or cytotoxic agent component of an MMM complex (e.g., an ELP-MRD fusion protein) or other drug effective to “treat” a disease or disorder in a subject or mammal. In the case of cancer, therapeutically effective amount of the drug may constitute the amount of drug effective to reduce angiogenesis and neovascularization; reduce the number of cancer cells; reduce the tumor size; inhibit (i.e., slow to some extent or stop) cancer cell infiltration into peripheral organs; inhibit (i.e., slow to some extent or stop) tumor metastasis; inhibit, to some extent, tumor growth or tumor incidence; stimulate immune responses against cancer cells and/or relieve to some extent one or more of the symptoms associated with the cancer. See the definition herein of “treating”. A “therapeutically effective amount” also may refer to an amount effective, at dosages and for periods of time necessary, to achieve a desired therapeutic result. A therapeutically effective amount of a complex of the invention may vary according to factors such as the disease state, age, sex, and weight of the individual, and the ability of the complex to elicit a desired response in the individual. A therapeutically effective amount is also one in which any toxic or detrimental effects of therapeutic complex are outweighed by therapeutically beneficial effects.

To the extent the drug can prevent growth and/or kill existing cancer cells, it can be cytostatic and/or cytotoxic. A “prophylactically effective amount” refers to an amount effective, at dosages and for periods of time necessary, to achieve the desired prophylactic result. Typically, but not necessarily, since a prophylactic dose is used in subjects (patients) prior to or at an earlier stage of disease, the prophylactically effective amount will be less than therapeutically effective amount.

### II. Modular Recognition Domains (MRDs)

The present invention describes an approach that creates monovalent and MMM diagnostics and therapeutics based on the adaptation of modular recognition domains (MRDs), and optionally other modular components, as fusions to one or more ELPs. The interaction between a ligand and its receptor often takes place at a relatively large interface. However, only a few key residues at the interface contribute to most of the binding. In some embodiments, MRDs can mimic ligand binding. In certain embodiments, an MRD can mimic the biological activity of a ligand (an agonist MRD) or inhibit the bioactivity of the ligand (an antagonist MRD), e.g., through competitive binding. MRDs in monovalent and MMM complexes (“MMM”) such as, ELP-MRD fusion proteins, can also affect targets in other ways, e.g., by neutralizing, blocking, stabilizing, aggregating, or crosslinking an MRD target.

MMM complexes (e.g., ELP-MRD fusion proteins) of the invention comprise at least one modular recognition domain (MRD). In one embodiment, the MMM complexes (e.g., ELP-MRD fusion proteins) comprise more than 1 MRD, wherein the MRDs have the same or different specificities. In additional embodiments, the MMM complexes (e.g., ELP-MRD fusion protein) are comprised of a tandem repeat of the same or different MRDs that allow an MMM complex (e.g., an ELP-MRD fusion protein) to bind multiple targets and/or repeating epitopes or different epitopes on the same target.

It is contemplated MMM complexes (e.g., ELP-MRD fusion proteins) comprise 1 or more MRDs that bind to a target site of interest. In some embodiments, MRDs have a length of about 2 to 150 amino acids, about 2 to 125 amino acids, about 2 to 100 amino acids, about 2 to 90 amino acids, about 2 to 80 amino acids, about 2 to 70 amino acids, about 2 to 60 amino acids, about 2 to 50 amino acids, about 2 to 40 amino acids, about 2 to 30 amino acids, or about 2 to 20 amino acids. It is also contemplated in some embodiments, that MRDs have a length of about 10 to 150 amino acids, about 10 to 125 amino acids, about 10 to 100 amino acids, about 10 to 90 amino acids, about 10 to 80 amino acids, about 10 to 70 amino acids, about 10 to 60 amino acids, about 10 to 50 amino acids, about 10 to 40 amino acids, about 10 to 30 amino acids, or about 10 to 20 amino acids. It is further contemplated that MRDs have a length of about 20 to 150 amino acids, about 20 to 125 amino acids, about 20 to 100 amino acids, about 20 to 90 amino acids, about 20 to 80 amino acids, about 20 to 70 amino acids, about 20 to 60 amino acids, about 20 to 50 amino acids, about 20 to 40 amino acids, or about 20 to 30 amino acids. In certain embodiments, the MRDs have a length of about 2 to 60 amino acids. In other embodiments, the MRDs have a length of about 10 to 60 amino acids. In other embodiments, the MRDs have a length of about 10 to 50 amino acids. In additional embodiments, the MRDs have a length of about 10 to 40 amino acids. In additional embodiments, the MRDs have a length of about 10 to 30 amino acids.

In some embodiments, the MRD contains at least one reactive residue. Reactive residues are useful, for example, as sites for the attachment of conjugates such as chemotherapeutic drugs. The reactive residue can be, for example, a cysteine, a lysine, or another reactive residue. Thus, a cysteine can be added to an MRD at either end or within the MRD sequence and/or a cysteine can be substituted for another amino acid in the sequence of an MRD. In addition, a lysine can be added to an MRD at either end or within the MRD sequence and/or a lysine can be substituted for another amino acid in the sequence of an MRD.

In some embodiments, MRDs in the MMM complexes (e.g., ELP-MRD fusion proteins) of the invention are able to bind their respective target in the context of the MMM complex (e.g., an ELP-MRD fusion protein). In some embodiments, an MRD is able to bind its target as a polypeptide that consists of the amino acid sequence of an MRD. In some embodiments, an MRD alone or in the context of the MMM complex (e.g., an ELP-MRD fusion protein) is a target agonist. In other embodiments, an MRD alone or in the context of the MMM complex (e.g., an ELP-MRD fusion protein) is a target antagonist. In certain embodiments, an MRD localizes the MMM complex (e.g., ELP-MRD fusion protein) to an area where an MRD target is located.

In additional embodiments, one or more of the MRD components of the MMM complexes and/or the MMM complex (e.g., ELP-MRD fusion protein) binds to a target with a dissociation constant or Kd of less than 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³M, 5×10⁻¹³ M, 10⁻¹³ M, 5×10⁻¹⁴ M, 10⁻¹⁴ M, 5×10⁻¹⁵ M, or 10⁻¹⁵ M. In one embodiment, one or more of the MRD components of the MMM complexes and/or the MMM complex (e.g., ELP-MRD fusion protein) has a dissociation constant or Kd less than 5×10⁻⁵ M. In another embodiment, one or more of the MRD components of the MMM complexes and/or the MMM complex (e.g., ELP-MRD fusion protein) has a dissociation constant or Kd less than 5×10⁻⁸ M. In another embodiment, one or more of the MRD components of the MMM and/or the MMM complex (e.g., ELP-MRD fusion protein) has a dissociation constant or Kd less than 5×10⁻⁹ M. In an additional embodiment, one or more of the MRD components of the MMM complexes and/or the MMM complex (e.g., ELP-MRD fusion protein) has a dissociation constant or Kd less than 5×10⁻¹⁰ M. In a further embodiment, one or more of the MRD components of the MMM complexes and/or the MMM complex (e.g., ELP-MRD fusion protein) has a dissociation constant or Kd less than 5×10⁻¹¹ M. In another embodiment, one or more of the MRD components of the MMM complexes and/or the MMM complex (e.g., ELP-MRD fusion protein) has a dissociation constant or Kd less than 5×10⁻¹² M.

In specific embodiments, an MRD and/or the MMM complex (e.g., ELP-MRD fusion protein) binds its target with an off rate (k_{off}) of less than 5×10⁻² sec⁻¹, 10⁻² sec⁻¹, 5×10⁻³ sec⁻¹, or 10⁻³ sec⁻¹. In additional embodiments, an MRD and/or the MMM complex (e.g., ELP-MRD fusion protein) binds its target with an off rate (k_{off}) of less than 5×10⁻⁴ sec⁻¹, 10⁻⁴ sec⁻¹, 5×10⁻⁵ sec⁻¹, or 10⁻⁵ sec⁻¹, 5×10⁻⁶ sec⁻¹, 10⁻⁶ sec⁻¹, 5×10⁻⁷ sec⁻¹, or 10⁻⁷ sec⁻¹.

In other specific embodiments, an MRD and/or the MMM complex (e.g., ELP-MRD fusion protein) binds its target with an on rate (kₒₙ) of greater than 10³ M⁻¹ sec⁻¹, 5×10³ M⁻¹ sec⁻¹, 10⁴ M⁻¹ sec⁻¹, or 5×10⁴ M⁻¹ sec⁻¹. In additional embodiments, an MRD and/or the MMM complex (e.g., ELP-MRD fusion protein) can bind its target with an on rate (kₒₙ) of greater than 10⁵ M⁻¹ sec⁻¹, 5×10⁵ M⁻¹ sec⁻¹,10⁶ M⁻¹ sec⁻¹, or 5×10⁶ M⁻¹ sec⁻¹, or 10⁷ M⁻¹ sec⁻¹.

In some embodiments, an MRD has a therapeutic effect when repeatedly administered alone and/or when fused to an Fc in a patient or animal model (e.g., 3 or more times over the course of at least six months).

In some embodiments, an MRD is stable at a desired pH. For example, in some embodiments, an MRD is stable at pH 3-9, pH 3-8, pH 3-7, or pH 4-5.

A. MRD Targets and MRD Sequences

The target of an MRD and/or the MMM complex (e.g., ELP-MRD fusion protein) can be any molecule with which it is desirable for an MRD and/or the MMM complex (e.g., ELP-MRD fusion protein) to interact. A number of exemplary targets are provided, by way of example, herein. An MRD and MMM complex (e.g., ELP-MRD fusion protein) fusion targets described herein are intended to be illustrative and not limiting.

For example, an MRD and/or the MMM complex (e.g., ELP-MRD fusion protein) target can be a soluble factor or a transmembrane protein, such as a cell surface receptor. An MRD and/or the MMM complex (e.g., ELP-MRD fusion protein) can be an extracellular component or an intracellular component. In some embodiments, an MRD and/or the MMM complex (e.g., ELP-MRD fusion protein) target is a factor that regulates cell proliferation, differentiation, or survival. In other embodiments, an MRD and/or the MMM complex (e.g., ELP-MRD fusion protein) target is a cytokine. In additional embodiments, an MRD and/or the MMM complex (e.g., ELP-MRD fusion protein) target is a factor that regulates angiogenesis. In additional embodiments, an MRD and/or the MMM complex (e.g., ELP-MRD fusion protein) target is a factor that regulates one or more immune responses, such as, autoimmunity, inflammation and immune responses against cancer cells. In other embodiments, an MRD and/or the MMM complex (e.g., ELP-MRD fusion protein) target is a factor that regulates cellular adhesion and/or cell-cell interaction. In further embodiments, an MRD and/or the MMM complex (e.g., ELP-MRD fusion protein) target is a cell signaling molecule. In further embodiments, an MRD and/or the MMM complex (e.g., ELP-MRD fusion protein) target is FcRn.

In some embodiments, an MRD and/or the MMM complex (e.g., ELP-MRD fusion protein) target is a disease-related target. The target can be a target characteristic of a particular cancer, and/or of a particular cell type (e.g., a hyperproliferative cell), and/or of a particular pathogen (e.g., a bacterial cell (e.g., tuberculosis, smallpox, anthrax), a virus (e.g., HIV), a parasite (e.g., malaria, leichmaniasis), a fungal infection, a mold, a mycoplasm, or a prion antigen), or an antigen associated with a disorder of the immune system.

In additional embodiments, an MRD and/or the MMM complex (e.g., ELP-MRD fusion protein) target is a cancer target.

In one embodiment, an MRD and/or the MMM complex (e.g., ELP-MRD fusion protein) target is: PDGFRa, PDGFRb, PDGF-A, PDGF-B, PDGF-CC, PDGF-C, PDGF-D, VEGFR1, VEGFR2, VEGFR3, VEGFC, VEGFD, neuropilin 2 (NRP2), betacellulin, PlGF, RET (rearranged during transfection), TIE1, TIE2 (TEK), CA125, CD3, CD4, CD7, CD10, CD13, CD25, CD32, CD32b, CD44, CD49e (integrin alpha 5), CD55, CD64, CD90 (THY1), CD133 (prominin 1), CD147, CD166, CD200, ALDH1, ESA, SHH, DHH, 1HH, patchedl (PTCH1), smoothened (SMO), WNT1, WNT2B, WNT3A, WNT4, WNT4A, WNT5A, WNT5B, WNT7B, WNT8A, WNT10A, WNT10B, WNT16B, LRP5, LRP6, FZD1, FZD2, FZD4, FZD5, FZD6, FZD7, FZD8, Notch, Notch1, Notch3, Notch4, DLL4, Jagged, Jagged1, Jagged2, Jagged3, TNFSF1 (TNFb, LTa), TNFRSF1A (TNFR1, p55, p60), TNFRSF1B (TNFR2), TNFSF6 (Fas Ligand), TNFRSF6 (Fas, CD95), TNFRSF6B (DcR3), TNFSF7 (CD27 Ligand, CD70), TNFRSF7 (CD27), TNFSF8 (CD30 Ligand), TNFRSF8 (CD30), TNFSF11 (RANKL), TNFRSF11A (RANK), TNFSF12 (TWEAK), TNFRSF12 (TWEAKR), TNFSF13 (APRIL), TNFSF13B (BLYS), TNFRSF13B (TACI), TNFRSF13C (BAFFR), TNFSF15 (TL1A), TNFRSF17 (BCMA), TNFRSF19L (RELT), TNFRSF19 (TROY), TNFRSF21 (DR6), TNFRSF25 (DR3), ANG1 (ANGPT1), ANG3 (ANGPTL1), ANG4 (ANGPT4), IL1a, IL1b, IL1R1, IL1R2, IL2, IL2R, IL5, IL5R, IL6R, IL8, IL8R, IL10, IL10R, IL12, IL12R, IL13, IL13R, IL15, IL15R, IL18, IL18R, IL19, IL19R, IL21R, IL23, IL23R, mif, XAG1, XAG3, REGIV, FGF1, FGF2, FGF3, FGF4, FGFR1, FGFR2, FGFR3, ALK, ALK1, ALK7, ALCAM, Artemin, Axl, TGFb, TGFb2, TGFb3, TGFBR1, IGFIIR, BMP2, BMP5, BMP6, BMPR1, GDF3, GDF8, GDF9, N-cadherin, E-cadherin, VE-cadherin, NCAM, L1CAM (CD171), ganglioside GM2, ganglioside GD2, calcitonin, PSGR, DCC, CDCP1, CXCR2, CXCR7, CCR3, CCR5, CCR7, CCR10, CXCL1, CXCL5, CXCL6, CXCL8, CXCL12, CCL3, CCL4, CCL5, CCL11, Claudin1, Claudin2, Claudin3, Claudin4, TMEFF2, neuregulin, MCSF, CSF, CSFR (fms), GCSF, GCSFR, BCAM, HPV, hCG, SR1F, PSA, FOLR2 (folate receptor beta), BRCA1, BRCA2, HLA-DR, ABCC3, ABCB5, HM1.24, LFA1, LYNX, S100A8, S100A9, SCF, Von Willebrand factor, Lewis Y6 receptor, Lewis Y, CA G250 (CA9), integrin avb3 (CNTO95), integrin avb5, activin B1 alpha, leukotriene B4 receptor (LTB4R), neurotensin NT receptor (NTR), 5T4 oncofetal antigen, Tenascin C, MMP, MMP2, MMPI, MMP9, MMP12, MMP14, MMP26, cathepsin G, cathepsin H, cathepsin L, SULF1, SULF2, MET, UPA, MHCl, MN(CA9), TAG-72, TM4SF1, Heparanse (HPSE), syndecan (SDC1), Ephrin B2, Ephrin B4, or relaxin2. MMM complexes (e.g., ELP-MRD fusion proteins) comprising 1, 2, 3, 4, 5, 6, or more MRDs that bind to one of the above targets are also encompassed by the invention. MMM complexes (e.g., ELP-MRD fusion proteins) comprising MRDs that bind to at least 1, 2, 3, 4, 5, 6 or more of the above targets are additionally encompassed by the invention.

In another embodiment, an MRD and/or the MMM complex (e.g., ELP-MRD fusion protein) target is a member selected from: CD19, CD22, CD30, CD33, CD38, CD44v6, TNFSF5 (CD40 Ligand), TNFRSF5 (CD40), CD52, CD54 (ICAM), CD74, CD80, CD200, EPCAM (EGP2), neuropilin 1 (NRP1), TEM1, mesothelin, TGFbeta 1, TGFBR11, phosphatidlyserine, folate receptor alpha (FOLR1), TNFRSF10A (TRAIL R1 DR4), TNFRSF10B (TRAIL R2DR5), CXCR4, CCR4, CCL2, HGF, CRIPTO, VLA5, TNFSF9 (41BB Ligand), TNFRSF9 (41BB, CD137), CTLA4, HLA-DR, IL6, TNFSF4 (OX40 Ligand), TNFRSF4 (OX40), MUC1, MUC18, mucin CanAg, ganglioside GD3, EGFL7, PDGFRa, IL21, IGF1, IGF2, CD117 (cKit), SLAMF7, carcinoembryonic antigen (CEA), FAP, integrin avb3, or integrin α5β3. MMM complex (e.g., ELP-MRD fusion protein) having 1, 2, 3, 4, 5, 6, or more MRDs that bind to one of the above targets are also encompassed by the invention. MMM complex (e.g., ELP-MRD fusion protein) having MRDs that bind to at least 1, 2 or more of the above targets are additionally encompassed by the invention.

In particular embodiments, an MRD and/or the MMM complex (e.g., ELP-MRD fusion protein) of the invention competes for target binding with an antibody selected from: siplizumab CD2 (e.g., MEDI-507, MedImmune), blinatumomab CD19 CD3 (e.g., MT103, Micromet/Medlmmune); XMAB®5574 CD19 (Xencor), SGN-19A CD19 (Seattle Genetics), ASG-5ME (Agenesys and Seattle Genetics), MEDI-551 CD19 (Medlmmune), epratuzumab CD22 (e.g., hLL2, Immunomedics/UCB), inotuzumab ozogamicin CD22 (Pfizer), iratumumab CD30 (e.g., SGN-30 (Seattle Genetics) and MDX-060 (Medarex)), XMAB02513 CD30 (Xencor), brentuximab vedotin CD30 (e.g., SGN-35, Seattle Genetics), gemtuzumab ozogamicin CD33 (e.g., MYLOTARG®, Pfizer), lintuzumab CD33 (e.g., antibody of Seattle Genetics), MOR202, CD38 (MorphoSys), daratumumab CD38 (e.g., Genmab antibody), CP870893 CD40 (Pfizer), dacetuzumab CD40 (e.g., SGN40, Seattle Genetics), ANTOVA® CD40 (Biogen Idec), lucatumumab CD40 (e.g., HCD122, Novartis) XMAB®5485 CD40 (Xencor), teneliximub, ruplizumab CD40L (e.g., ANTOVA®) bivatuzumab mertansine CD44v6, alemtuzumab CD52 (e.g., CAMPATH®/MABCAMPATH®, Genzyme/Bayer), BI505 ICAM1 (Bioinvent), milatuzumab CD74 (e.g., antibody of Immunomedics), galiximab CD80 (Biogen Idec), BMS663513 4-1BB (Bristol-Myers Squibb), Alexion CD200 antibody (Alexion), edrecolomab EPCAM (e.g., MAb17-1A, PANOREX® (GlaxoSmithKline), AT003 EPCAM (Affitech)), adecatumumab EPCAM (e.g., MT201, Micromet), oportuzumab monatox EPCAM, Genentech anti-NRP1 antibody, MORABOO4 TEM1 (Morphotek), MORABOO9 mesothelin (Morphotek), lerdelimumab TGFb1 (e.g., CAT-152, Cambridge Antibody Technology), metelimumab TGFb1 (e.g., CAT-192, Cambridge Antibody Technology), ImClone anti-TGFBR11 antibody, bavituximab phosphatidylserine (e.g., antibody of Peregrine (Peregrine Pharmaceuticals)), AT004 phosphatidylserine (Affitech), AT005 phosphatidylserine (Affitech), MORABO3 folate receptor alpha (Morphotek), farletuzumab folate receptor alpha cancer (e.g., MORAB003, Morphotek), CS 1008 DR4 (Sankyo), mapatumumab DR4 (e.g., HGS-ETR1, Human Genome Sciences), LBY135 DR5 (Novartis), AMG66 DR5 (Amgen), Apomab DR5 (Genentech), PRO95780 (Genentech), lexatumumab DR5, (e.g., HGS-ETR2, Human Genome Sciences), conatumumab DR5, (e.g., AMG655, Amgen), tigatuzumab (e.g., CS-1008), AT009 CXCR4 (Affitech), AT008 CCR4 (Affitech), CNTO-888 CCL2 (Centocor), AMG102 HGF (Amgen), CRIPTO antibody (Biogen Idec), M200 antibody VLA5 (Biogen Idec), ipilimumab CTLA4 (e.g., MDX010, Bristol-Myers Squibb/Medarex), belatacept CTLA4 ECD (e.g., CP-675,206, Pfizer), IMMU114 HLA-DR (Immunomedics), apolizumab HLA-DR, toclizumab IL-6R (e.g., ACTEMR®A/ROACTREMRA®, Hoffman-La Roche), OX86 OX40, pemtumomab PEM/MUC1 (Theragyn), ABX-MA1 MUC-18 (Abgenix), clivatuzumab MUC-18 (e.g., hPAM4, Immunomedics), cantuzumab mertansine mucin CanAg, ecromeximab (Ludwig Institute), Genentech anti-EGFL7 antibody, AMG820 CSFR (Amgen), olaratumab PDGFRa (e.g., antibody of Imclone (Imclone)), IL21 antibody Zymogenetics (Zymogenetics), MEDI-573 IGF1/IGF2 (MedImmune), AMG191 cKit (Amgen), etaracizumab (e.g., MEDI-522, Medlmune), and MLN591 PSMA (Millennium Pharmaceuticals), elotuzumab SLAMF7 (e.g., HuLuc63, PDL), labetuzumab CEA (CEA-CIDE®, Immunomedics), sibrotuzumab FAP, CNTO95 integrin avb3 (Centocor), VITAXIN® integrin avb3 (MedImmune), and voloximab αβ1. MMM complex (e.g., ELP-MRD fusion protein) having 1, 2, 3, 4, 5, 6, or more MRDs that bind to the same epitope as, or competitively inhibit binding of, one of the above antibodies are also encompassed by the invention. MMM complex (e.g., ELP-MRD fusion protein) having MRDs that bind to the same epitope as, or competitively inhibit binding of, at least 1, 2, 3, 4, 5, 6, or more of the above antibodies are additionally encompassed by the invention.

In another embodiment, a target of an MRD and/or the MMM complex (e.g., ELP-MRD fusion protein) is ANG2 (ANGPT2). In one embodiment, an MRD and/or the MMM complex (e.g., ELP-MRD fusion protein) binds to the same epitope as MEDI3617. In another embodiment, an MRD and/or the MMM complex (e.g., ELP-MRD fusion protein) competitively inhibits binding of MEDI3617 to ANG2. MMM complex (e.g., ELP-MRD fusion protein) having 1, 2, 3, 4, 5, 6, or more MRDs that bind to the same epitope as, or competitively inhibit binding of, one of the above antibodies are also encompassed by the invention. MMM complex (e.g., ELP-MRD fusion protein) having MRDs that bind to the same epitope as, or competitively inhibit binding of, at least one or both of the above antibodies are additionally encompassed by the invention.

In certain embodiments, a target of an MRD and/or the MMM complex (e.g., ELP-MRD fusion protein) is EGFR, ErbB2, ErbB3, ErbB4, CD2O, insulin-like growth factor-I receptor, prostate specific membrane antigen, an integrin, or cMet. MMM complex (e.g., ELP-MRD fusion protein) having 1, 2, 3, 4, 5, 6, or more MRDs that bind to one of the above targets are also encompassed by the invention. MMM complex (e.g., ELP-MRD fusion protein) having MRDs that bind to at least 1, 2, 3, 4, 5, 6, or more of the above targets are additionally encompassed by the invention.

In another embodiment, an MRD and/or the MMM complex (e.g., ELP-MRD fusion protein) binds EGFR. In one embodiment, an MRD and/or the MMM complex (e.g., ELP-MRD fusion protein) binds to the same epitope as ERBITUX®. In another embodiment, an MRD and/or the MMM complex (e.g., ELP-MRD fusion protein) competitively inhibits binding of ERBITUX® to EGFR. In another embodiment, an MRD and/or the MMM complex (e.g., ELP-MRD fusion protein) inhibits EGFR dimerization. In another embodiment, an MRD and/or the MMM complex (e.g., ELP-MRD fusion protein) binds to the same epitope as matuzimab or panitumumab. In a further embodiment the MMM complex (e.g., ELP-MRD fusion protein) binds to same epitope as matuzimab and panitumumab. In another embodiment, an MRD and/or the MMM complex (e.g., ELP-MRD fusion protein) competitively inhibits binding of matuzimab or panitumumab to EGFR. In another embodiment, an MRD and/or the MMM complex (e.g., ELP-MRD fusion protein) competitively inhibits binding of matuzimab and panitumumab to EGFR. In another embodiment, an MRD and/or the MMM complex (e.g., ELP-MRD fusion protein) binds to the same epitope as, or competitively inhibits binding to, EGFR by ABX-EGF or MDX-214. In a further embodiment, an MRD and/or the MMM complex (e.g., ELP-MRD fusion protein) binds to the same epitope as, or competitively inhibits binding to, EGFR by ABX-EGF and MDX-214.

In an additional embodiment an MRD and/or the MMM complex (e.g., ELP-MRD fusion protein) binds ErbB2 (Her2). In one embodiment, an MRD and/or the MMM complex (e.g., ELP-MRD fusion protein) binds to the same epitope as trastuzumab (e.g., HERCEPTIN®, Genentech/Roche). In another embodiment, an MRD competitively inhibits binding of trastuzumab to ErbB2. MMM complex (e.g., ELP-MRD fusion protein) having 1, 2, 3, 4, 5, 6, or more MRDs that bind to the same epitope as, or competitively inhibits binding of, trastuzumab are also encompassed by the invention.

In a further embodiment, an MRD and/or the MMM complex (e.g., ELP-MRD fusion protein) inhibits HER2 dimerization. In another embodiment, an MRD and/or the MMM complex (e.g., ELP-MRD fusion protein) inhibits HER2 heterodimerization with HER3 (ErbB3). In a specific embodiment, the antibody is pertuzumab (e.g., OMNITARG® and phrMab2C4, Genentech). In another embodiment, an MRD binds to the same epitope as pertuzumab. In another embodiment, an MRD competitively inhibits binding of ErbB2 by pertuzumab. MMM complexes (e.g., ELP-MRD fusion proteins) having 1, 2, 3, 4, 5, 6, or more MRDs that bind to the same epitope as or competitively inhibit pertuzumab are also encompassed by the invention.

In another embodiment, an MRD and/or the MMM complex (e.g., ELP-MRD fusion protein) binds to the same epitope on ErbB2 as an antibody selected from the group: MDX-210 (Medarex), tgDCC-E1A (Targeted Genetics), MGAH22 (MacroGenics), and pertuzumab (OMNITARG™, 2C4; Genentech). MRDs and/or the MMM complex (e.g., ELP-MRD fusion protein) that compete for target binding with one of the above antibodies are also encompassed by the invention. MMM complex (e.g., ELP-MRD fusion protein) having 1, 2, 3, 4, 5, 6, or more MRDs that bind to the same epitope as one of the above antibodies or competitively inhibit one of the above antibodies are also encompassed by the invention. MMM complex (e.g., ELP-MRD fusion protein) having MRDs that bind to the same epitope as, or competitively inhibit binding of, 1, 2, or 3 of the above antibodies are additionally encompassed by the invention.

In one embodiment an MRD and/or the MMM complex (e.g., ELP-MRD fusion protein) binds ErbB3 (Her3). In one embodiment, an MRD and/or the MMM complex (e.g., ELP-MRD fusion protein) binds to the same epitope as MM121 (Merrimack Pharmaceuticals) or AMG888 (Amgen). In another embodiment, an MRD and/or the MMM complex (e.g., ELP-MRD fusion protein) competitively inhibits binding of MM121 or AMG888 to ErbB3. MMM complex (e.g., ELP-MRD fusion protein) having 1, 2, 3, 4, 5, 6, or more MRDs that bind to the same epitope as, or competitively inhibit binding of, MM121 or AMG888 are also encompassed by the invention. MMM complex (e.g., ELP-MRD fusion protein) having MRDs that bind to the same epitope as, or competitively inhibit binding of, MM121 or AMG888 are additionally encompassed by the invention.

In other embodiments, an MRD and/or the MMM complex (e.g., ELP-MRD fusion protein) binds VEGF. In another specific embodiment, an MRD and/or the MMM complex (e.g., ELP-MRD fusion protein) binds to the same epitope r84 (Peregrine) or 2C3 (Peregrine). In another embodiment, an MRD and/or the MMM complex (e.g., ELP-MRD fusion protein) competitively inhibits VEGF binding by r84 or 2C3. MMM complex (e.g., ELP-MRD fusion protein) having 1, 2, 3, 4, 5, 6, or more MRDs that bind to the same epitope as, or competitively inhibit binding of, one of the above antibodies are also encompassed by the invention. MMM complex (e.g., ELP-MRD fusion protein) having MRDs that bind to the same epitope as, or competitively inhibit binding of, r84 or 2C3 are additionally encompassed by the invention.

In further embodiments, an MRD and/or the MMM complex (e.g., ELP-MRD fusion protein) binds VEGFA. In one embodiment, an MRD and/or the MMM complex (e.g., ELP-MRD fusion protein) binds to the same epitope as bevacizumab (e.g., AVASTIN®, Genentech/Roche) to VEGFA. In an additional embodiment, an MRD and/or the MMM complex (e.g., ELP-MRD fusion protein) binds to the same epitope as AT001 (Affitech). MMM complex (e.g., ELP-MRD fusion protein) having 1, 2, 3, 4, 5, 6, or more MRDs that bind to the same epitope as, or competitively inhibit binding of, bevacizumab or AT001 are also encompassed by the invention. MMM complex (e.g., ELP-MRD fusion protein) having MRDs that bind to the same epitope as, or competitively inhibit binding of, bevacizumab or AT001 are additionally encompassed by the invention.

In other embodiments, an MRD and/or the MMM complex (e.g., ELP-MRD fusion protein) binds VEGFR1. In one embodiment, an MRD and/or the MMM complex (e.g., ELP-MRD fusion protein) competitively inhibits binding of Aflibercept (Regeneron) to VEGFR1. In another embodiment, an MRD and/or the MMM complex (e.g., ELP-MRD fusion protein) inhibits VEGFR1 dimerization. MMM complex (e.g., ELP-MRD fusion protein) having 1, 2, 3, 4, 5, 6, or more MRDs that bind to the same epitope as, or competitively inhibit binding of, Aflibercept are also encompassed by the invention. MMM complex (e.g., ELP-MRD fusion proteins having MRDs that bind to the same epitope as, or competitively inhibit binding of, Aflibercept are additionally encompassed by the invention.

In additional embodiments, an MRD and/or the MMM complex (e.g., ELP-MRD fusion protein) binds VEGFR2. In a specific embodiment, an MRD and/or the MMM complex (e.g., ELP-MRD fusion protein) binds to the same epitope as, ramucirumab (e.g., IMC1121B and IMC1C11, ImClone). In another embodiment, an MRD and/or the MMM complex (e.g., ELP-MRD fusion protein) competitively inhibits binding of ramucirumab to VEGFR2. In an additional embodiment, an MRD and/or the MMM complex (e.g., ELP-MRD fusion protein) inhibits VEGFR2 dimerization. MMM complexes (e.g., ELP-MRD fusion proteins) having 1, 2, 3, 4, 5, 6, or more MRDs that bind to the same epitope as, or competitively inhibit binding of, ramucirumab are also encompassed by the invention. MMM complex (e.g., ELP-MRD fusion protein) having MRDs that bind to the same epitope as, or competitively inhibit binding of, ramucirumab are additionally encompassed by the invention.

In other embodiments, an MRD and/or the MMM complex (e.g., ELP-MRD fusion protein) binds CD20. In one embodiment, an MRD and/or the MMM complex (e.g., ELP-MRD fusion protein) binds to the same epitope as rituximab (e.g., RITUXAN®/MABTHERA®, Genentech/Roche/Biogen Idec). In another embodiment, an MRD and/or the MMM complex (e.g., ELP-MRD fusion protein) competitively inhibits binding of rituximab to CD20. In one embodiment, an MRD and/or the MMM complex (e.g., ELP-MRD fusion protein) binds to the same epitope as GA-101 (Genentech). In another embodiment, an MRD and/or the MMM complex (e.g., ELP-MRD fusion protein) competitively inhibits binding of GA-101 to CD20. In another embodiment, an MRD and/or the MMM complex (e.g., ELP-MRD fusion protein) competitively inhibits binding of rituximab to CD20. In one embodiment, an MRD and/or the MMM complex (e.g., ELP-MRD fusion protein) binds to the same epitope as ocrelizumab (e.g., 2H7; Genentech/Roche/Biogen Idec). In another embodiment, an MRD and/or the MMM complex (e.g., ELP-MRD fusion protein) competitively inhibits binding of ocrelizumab to CD20. In another embodiment, an MRD and/or the MMM complex (e.g., ELP-MRD fusion protein) binds to the same epitope as an antibody selected from: obinutuzumab (e.g., GA101; Biogen Idec/Roche/Glycart), ofatumumab (e.g., ARZERRA® and HuMax-CD20Genmab), veltuzumab (e.g., IMMU-160, Immunomedics), AME-133 (Applied Molecular Evolution), SGN35 (Millennium), TG-20 (GTC Biotherapeutics), afutuzumab (Hoffman-La Roche), and PRO131921 (Genentech). In another embodiment, an MRD and/or the MMM complex (e.g., ELP-MRD fusion protein) competitively inhibits CD20 binding by an antibody selected from: obinutuzumab, ofatumumab, veltuzumab, AME-133, SGN35, TG-20 and PRO131921. MMM complex (e.g., ELP-MRD fusion protein) having 1, 2, 3, 4, 5, 6, or more MRDs that bind to the same epitope as, or competitively inhibit binding of, one of the above antibodies are also encompassed by the invention. MMM complex (e.g., ELP-MRD fusion protein) having MRDs that bind to the same epitope as, or competitively inhibit binding of, at least 1, 2, 3, 4, 5, 6, or more of the above antibodies are additionally encompassed by the invention.

In additional embodiments, an MRD and/or the MMM complex (e.g., ELP-MRD fusion protein) binds IGF1R. In one embodiment, an MRD and/or the MMM complex (e.g., ELP-MRD fusion protein) binds to the same epitope as an antibody selected from: cixutumumab (e.g., IMC-A12, ImClone), figitumumab (e.g., CP-751,871, Pfizer), AMG479 (Amgen), BIIB022 (Biogen Idec), SCH 717454 (Schering-Pough), and R1507 (Hoffman La-Roche). In another embodiment, an MRD and/or the MMM complex (e.g., ELP-MRD fusion protein) competitively inhibits IGF1R binding by an antibody selected from: cixutumumab, figitumumab, AMG479, BIIB022, SCH 717454, and R1507. In another embodiment, an MRD and/or the MMM complex (e.g., ELP-MRD fusion protein) inhibits IGF dimerization. MMM complex (e.g., ELP-MRD fusion protein) having 1, 2, 3, 4, 5, 6, or more MRDs that bind to the same epitope as, or competitively inhibit binding of, one of the above antibodies are also encompassed by the invention. MMM complex (e.g., ELP-MRD fusion protein) having MRDs that bind to the same epitope as, or competitively inhibit binding of, at least 1, 2 or more of the above antibodies are additionally encompassed by the invention.

In further embodiments, an MRD and/or the MMM complex (e.g., ELP-MRD fusion protein) binds integrin. In a specific embodiment, an MRD and/or the MMM complex (e.g., ELP-MRD fusion protein) binds to the same epitope as an antibody selected from: MEDI-522 avb3 (VITAXIN®, MedImmune), CNTO 95 a5b3 (Centocor), JC7U αvβ3, and volociximab a5b1 (e.g., M200, PDL and Biogen Idec). In another embodiment, an MRD and/or the MMM complex (e.g., ELP-MRD fusion protein) binds to the same epitope as an antibody selected from: MEDI-522, CNTO 95, JC7U αvβ3, and volociximab. In another embodiment, an MRD and/or the MMM complex (e.g., ELP-MRD fusion protein) competitively inhibits integrin binding by an antibody selected from: MEDI-522, CNTO 95, JC7U, and M200. In one embodiment, an MRD and/or the MMM complex (e.g., ELP-MRD fusion protein) binds to the same epitope as natalizumab (e.g., TSABR1 ®, Biogen Idec). In another embodiment, an MRD and/or the MMM complex (e.g., ELP-MRD fusion protein) competitively inhibits integrin binding by natalizumab. MMM complex (e.g., ELP-MRD fusion protein) having 1, 2, 3, 4, 5, 6, or more MRDs that bind to the same epitope as, or competitively inhibit binding of, one of the above antibodies are also encompassed by the invention MMM complexes (e.g., ELP-MRD fusion proteins) having MRDs that bind to the same epitope as, or competitively inhibit binding of, at least 1, 2, 3, 4, 5, 6 or more of the above antibodies are additionally encompassed by the invention.

In additional embodiments, an MRD and/or the MMM complex (e.g., ELP-MRD fusion protein) binds cMet. In a specific embodiment, an MRD and/or the MMM complex (e.g., ELP-MRD fusion protein) binds to the same epitope as an antibody selected from: MetMab (OA-5D5, Genentech), AMG-102 (Amgen) and DN30. In another embodiment, an MRD and/or the MMM complex (e.g., ELP-MRD fusion protein) competitively inhibits cMet binding by an antibody selected from: MetMab (OA-5D5), AMG-102 and DN30. MMM complex (e.g., ELP-MRD fusion protein) having 1, 2, 3, 4, 5, 6, or more MRDs that bind to the same epitope as, or competitively inhibit binding of, one of the above antibodies are also encompassed by the invention. MMM complex (e.g., ELP-MRD fusion protein) having MRDs that bind to the same epitope as, or competitively inhibit binding of, at least 1, 2, or more of the above antibodies are additionally encompassed by the invention.

In other embodiments, a target of an MRD and/or the MMM complex (e.g., ELP-MRD fusion protein) is an antigen associated with an autoimmune disorder, inflammatory or other disorder of the immune system or is associated with regulating an immune response.

In another embodiment the MMM complex (e.g., ELP-MRD fusion protein) improves the performance of antigen presenting cells (e.g., dendritic cells). In one embodiment a target of the MMM complex (e.g., ELP-MRD fusion protein) is a member selecting from: CD19, CD20, CD21, CD22, CD23, CD27, CD28, CD30, CD30L, TNFSF14 (LIGHT, HVEM Ligand), CD70, ICOS, ICOSL, CTLA4, PD-1, PDL1 (B7-H1), B7-H4, B7-H3, PDL2 (B7-DC), BTLA, CD46, CD80 (B7-1), CD86 (B7-2), HLA-DR, CD74, PD1, TNFRSF4 (OX40), TNFRSF9 (41BB, CD137), TNFSF4 (OX40 Ligand), TNFSF9 (41BB Ligand), TNFRSF9 (41BB, CD137), TNFRSF1A (TNFR1, p55, p60), TNFRSF1B (TNFR2), TNFRSF13B (TACI), TNFRSF13C (BAFFR), TNFRSF17 (BCMA), BTLA, TNFRSF18 (GITR), MHC-1, TNFRSF5 (CD40), TLR4, TNFRSF14 (HVEM), Fc gamma RIIB, and IL-4R.

In some embodiments, a target of an MRD and/or the MMM complex (e.g., ELP-MRD fusion protein) is an immunoinhibitory target selected from: IL-1, IL-1b, IL-1Ra, L-5, IL6, IL-6R, CD26L, CD28, CD80, FcRn, or Fc Gamma RIIB. MMM complex (e.g., ELP-MRD fusion protein) having 1, 2, 3, 4, 5, 6, or more MRDs that bind to one of the above targets are also encompassed by the invention. MMM complex (e.g., ELP-MRD fusion protein) having MRDs that bind to at least 1, 2, 3, 4, 5, 6, or more of the above targets are additionally encompassed by the invention.

In another embodiment a target of the MMM complex (e.g., ELP-MRD fusion protein) is an immunostimulatory target (e.g., an agonist of a target associated immune cell activation (such as 41BB or CD40) or an antagonist of an inhibitory immune checkpoint (such as CTLA-4)). In other embodiments, a target of an MRD and/or the MMM complex (e.g., ELP-MRD fusion protein) is an immunostimulatory target selected from: CD25, CD28, CTLA-4, PD1, PD11, B7-H1, B7-H4, IL-10, TGFbeta, TNFSF4 (OX40 Ligand), TNFRSF4 (OX40), TNFSF5 (CD40 Ligand), TNFRSF5 (CD40), TNFSF9 (41BB Ligand), TNFRSF9 (41BB, CD137), TNFSF14 (LIGHT, HVEM Ligand), TNFRSF14 (HVEM), TNFSF15 (TL1A), TNFRSF25 (DR3), TNFSF18 (GITR Ligand), and TNFRSF18 (GITR). MMM complexes (e.g., ELP-MRD fusion proteins) having 1, 2, 3, 4, 5, 6, or more MRDs that bind to one of the above targets are also encompassed by the invention. MMM complex (e.g., ELP-MRD fusion protein) having MRDs that bind to at least 1, 2, 3, 4, 5, 6 or more of the above targets are additionally encompassed by the invention.

An MRD that binds to one of the above targets is encompassed by the invention. MMM complexes (e.g., ELP, ELP-MRD fusion proteins) having 1, 2, 3, 4, 5, 6, or more MRDs that bind to 1, 2, 3, 4, 5, 6, or more of the above targets are also encompassed by the invention. In specific embodiments, the MMM complex (e.g., ELP-MRD fusion protein) binds 2, 3 or all 4 targets selected from CTLA-4, TNFRSF18 (GITR), 4-1BB, and CD40. In one embodiment, the MMM complex (e.g., ELP-MRD fusion protein) binds CTLA-4 and 41BB. In another embodiment, the MMM complex (e.g., ELP-MRD fusion protein) binds CTLA-4 and TNFRSF18 (GITR). In another embodiment, the MMM complex (e.g., ELP-MRD fusion protein) binds CTLA-4 and CD40.). In another embodiment, the MMM complex (e.g., ELP-MRD fusion protein) binds CD40 and 41BB. In another embodiment, the MMM complex (e.g., ELP-MRD fusion protein) binds TNFRSF4 (OX40) and 41BB. In another embodiment, the MMM complex (e.g., ELP-MRD fusion protein) binds PD1 and B7-H1. In an additional embodiment the MMM complex (e.g., ELP-MRD fusion protein) enhances an immune response, such as, the immune system's anti-tumor response or an immune response to a vaccine.

In another embodiment a target of the MMM complex (e.g., ELP-MRD fusion protein) is cytokine selected from: IL-1 alpha, IL-1 beta, IL-18, TNFSF2 (TNFa), LTalpha, LT beta, TNFSF11 (RANKL), TNFSF13B (BLYS), TNFSF13 (APRIL), IL-6, IL-7, IL-10, IL-12, IL-15, IL-17A, IL-23, OncoStatinM, TGFbeta, BMP2-15, PDGF (e.g., PDGF-A, PDGF-B, PDGF-CC, PDGF-C, PDGF-D), an FGF family member (e.g., FGF1, FGF2, FGF4, FGF7, FGF8b and FGF19), VEGF (e.g., VEGFA and VEGFB), MIF, and a type I interferon. An MRD that binds to one of the above targets is encompassed by the invention. MMM complexes (e.g., ELP, ELP-MRD fusion proteins) having 1, 2, 3, 4, 5, 6, or more MRDs that bind to 1, 2, 3, 4, 5, 6, or more of the above targets are also encompassed by the invention.

In another embodiment a target of the MMM complex (e.g., ELP-MRD fusion protein) is cytokine selected from: TNF, CD25, CD28, CTLA-4, PD1, PD11, B7-H1, B7-H4, IL-10, TGFbeta, TNFSF4 (OX40 Ligand), TNFRSF4 (OX40), TNFSF5 (CD40 Ligand), TNFRSF5 (CD40), TNFSF9 (41BB Ligand), TNFRSF9 (41BB, CD137), TNFSF14 (LIGHT, HVEM Ligand), TNFRSF14 (HVEM), TNFSF15 (TL1A), TNFRSF25 (DR3), TNFSF18 (GITR Ligand), and TNFRSF18 (GITR). An MRD that binds to one of the above targets is encompassed by the invention. MMM complexes (e.g., ELP, ELP-MRD fusion proteins) having 1, 2, 3, 4, 5, 6, or more MRDs that bind to 1, 2, 3, 4, 5, 6, or more of the above targets are also encompassed by the invention.

In additional embodiments, a target of an MRD and/or the MMM complex (e.g., ELP-MRD fusion protein) is a member selected from: IL1Ra, IL1Rb, IL-2, IL-3, IL-4, IL-7, IL-10, IL-11, IL-15, IL-16, IL-17, IL-17A, IL-17F, IL-18, IL-19, IL-25, IL-32, IL-33, interferon beta, SCF, BCA1/CXCL13, CXCL1, CXCL2, CXCL6, CXCL13, CXCL16, C3AR, CSAR, CXCR1, CXCR2, CCR1, CCR3, CCR7, CCR8, CCR9, CCR10, ChemR23, CCL3, CCL5, CCL11, CCL13, CCL17, CCL18, CCL19, CCL20, CCL21, CCL22, CCL24, CCL25, CCL26, CCL27, MPL, GP130, TLR2, TLR3, TLR4, TLR5, TLR7, TLR8, TLR9, TREM1, TREM2, FcRn, Fc Gamma RIIB, oncostatin M, lymphotoxin alpha (LTa), integrin beta 7 subunit, CD49a (integrin alpha 1), integrin a5b3, MIF, ESM1, WIF1, cathepsin B, cathepsin D, cathepsin K, cathepsin S, TNFSF2 (TNFa), TNFSF3 (LTb), TNFRSF3 (LTBR), TNFSF6 (Fas Ligand), TNFRSF6 (Fas, CD95), TNFRSF6B (DcR3), TNFSF8 (CD30 Ligand), TNFRSF8 (CD30), TNFSF9 (41BB Ligand), TNFRSF9 (41BB, CD137), TNFSF11 (RANKL), TNFRSF11A (RANK), TNFSF14 (LIGHT, HVEM Ligand), TNFRSF14 (HVEM), TNFRSF16 (NGFR), TNFSF18 (GITR Ligand), TNFRSF18 (GITR), TNFRSF19L (RELT), TNFRSF19 (TROY), TNFRSF21 (DR6), CD14, CD23 CD25, CD28, CD36, CD36L, CD39, CD52, CD91, CD137, CD153, CD164, CD200, CD200R, BTLA, B7-1 (CD80), B7-2 (CD86), B7h, ICOS, ICOSL, MHC, CD, B7-H2, B7-H3, B7-H4, B7x, SLAM, KIM-1, SLAMF2, SLAMF3, SLAMF4, SLAMF5, SLAMF6, and SLAMF7. MMM complex (e.g., ELP-MRD fusion protein) having 1, 2, 3, 4, 5, 6, or more MRDs that bind to one of the above targets are also encompassed by the invention. MMM complex (e.g., ELP-MRD fusion protein) having MRDs that bind to at least 1, 2, 3, 4, 5, 6, or more of the above targets are additionally encompassed by the invention.

In other embodiments, a target of an MRD and/or the MMM complex (e.g., ELP-MRD fusion protein) is a member selected from: TNFSF1A (TNF-alpha), TNFRSF1A (TNFR1, p55, p60), TNFRSF1B (TNFR2), TNFSF7 (CD27 Ligand, CD70), TNFRSF7 (CD27), TNFSF13B (BLYS), TNFSF13 (APRIL), TNFRSF13B (TACI), TNFRSF13C (BAFFR), TNFRSF17 (BCMA), TNFSF15 (TL1A), TNFRSF25 (DR3), TNFSF12 (TWEAK), TNFRSF12 (TWEAKR), TNFSF4 (OX40 Ligand), TNFRSF4 (OX40), TNFSF5 (CD40 Ligand), TNFRSF5 (CD40), IL-1, IL-1b, IL1R, IL-2R, IL4-Ra, IL-5, IL-5R, IL-6, IL6R, IL9, IL12, IL-13, IL-14, IL-15, IL-15R, IL-17f, IL-17R, Il-17Rb, IL-17RC, IL-20, IL-21, IL-22RA, IL-23, IL-23R, IL-31, TSLP, TSLPR, interferon alpha, interferon gamma, B7RP-1, cKit, GMCSF, GMCSFR, CTLA-4, CD2, CD3, CD4, CD11a, CD18, CD20, CD22, CD26L, CD30, CD40, CD80, CD86, CXCR3, CXCR4, CCR2, CCR4, CCR5, CCR8, CCL2, CXCL10, P1GF, PD1, B7-DC(PDL2), B7-H1 (PDLL), alpha4 integrin subunit, A4B7 integrin, C5, RhD, IgE, and Rh. MMM complex (e.g., ELP-MRD fusion protein) having 1, 2, 3, 4, 5, 6, or more MRDs that bind to one of the above targets are also encompassed by the invention. MMM complex (e.g., ELP-MRD fusion protein) having MRDs that bind to at least 1, 2, 3, 4, 5, 6, or more of the above targets are additionally encompassed by the invention.

In particular embodiments, an MRD and/or the MMM complex (e.g., ELP-MRD fusion protein) binds to the same epitope as, or competitively inhibits binding of, an antibody selected from: SGN-70 CD70 (Seattle Genetics), SGN-75 CD70 (Seattle Genetics), Belimumab BLYS (e.g., BENLYSTA®, Human Genome Sciences/GlaxoSmithKline), Atacicept BLYS/APRIL (Merck/Serono), TWEAK (e.g., Biogen mAb), TL1A antibodies of CoGenesys/Teva (e.g., huml1D8, hum25B9, and humlB4 (U.S. Patent Application Publication 2009/0280116), OX40 mAb, humAb OX40L (Genentech), rilonacept IL1 trap (e.g., ARCALYST®, Regeneron), catumaxomab IL1b (e.g., REMOVAB®, Fresenius Biotech GmbH), Xoma052 IL1b (Lilly), canakinumab IL1beta (e.g., ILARIS® (Novartis) and ACZ885 (Novartis)), AMG108 IL1R (Amgen), daclizumab IL2Ra (e.g., ZENAPAX®, Hoffman-La Roche), basiliximab IL2Ra (e.g., SIMULECT®, Novartis), AMGN-317 IL-4-a (Amgen), pascolizumab IL-4 (PDL), mepolizumab IL5 (e.g., BOSATRIA®, GlaxoSmithKline), reslizumab IL5 (e.g., SCH55700, Ception Therapeutics), MEDI-563 IL-5R (MedImmune), BIW-8405, IL-5R (BioWa), etanercept TNFR2-fc (e.g., ENBREL®, Amgen), siltuximab IL6 (e.g., CNT0328, Centocor), CNTO-136 IL6 (Centocor), CDP-6038 IL6 (UCB), AMGN-220 IL6 (Amgen), REGN-88 IL6R (Regeneron), tocilizumab IL6R (e.g., ACTEMRA™/ROACTEMRA™, Chugai/Roche), MEDI-528 IL9 (MedImmune), briakinumab IL-12/13 (e.g., ABT-874, Abbott), ustekinumab IL-12, IL-23 (e.g., STELARA® and CNTO 1275, Centocor), TNX-650 IL-13 (Tanox), lebrikizumab IL-13 (Genentech), CAT354 IL-13 (Cambridge Antibody Technology), AMG714 IL-15 (Amgen), CRB-15 IL-15R (Hoffman La-Roche), AMG827 IL-17R (Amgen), IL-17RC antibody of Zymogenetics/Merck Serono, IL-20 antibody of Zymogenetics, IL-20 antibody of Novo Nordisk, IL-21 antibody of Novo Nordisk (e.g., NCT01038674), IL-21 antibody Zymogenetics (Zymogenetics), IL-22RA antibody of Zymogenetics, IL-31 antibody of Zymogenetics, AMG157 TSLP (Amgen), MEDI-545 interferon alpha (Medlmmune), MEDI-546 interferon alpha pathway component (Medlmmune), AMG811 interferon gamma (Amgen), INNO202 interferon gamma (Innogenetics/Advanced Biotherapy), HuZAF interferon-gamma (PDL), AMG557 B7RP1 (Amgen), AMG191 cKit (Amgen), MOR103GMCSF (MorphoSys), CAM-3001 GMCSFR (Medlmmune), tremelimumab CTLA4 (e.g., CP-675,206, Pfizer), iplimumab CTLA4 (e.g., MDX-010, BMS/Medarex), alefacept CD2 (e.g., AMEVIVE®, Astellas), siplizumab CD2 (e.g., MEDI-507, MedImmune), otelixizumab CD3 (e.g., TRX4, Tolerx/GlaxoSmithKline), teplizumab CD3 (e.g., MGA031, MacroGenics/Eli Lilly), visilizumab CD3 (e.g., NUVION®, PDL), muromonab-CD3 CD3 (Ortho), ibalizumab (e.g., TMB-355 and TNX-355, TaiMed Biologics), zanolimumab CD4 (e.g., HUMAX-CD4®, Genmab), cedelizumab CD4 (Euroasian Chemicals), keliximab CD4, priliximab CD4 (e.g., cMT412, Centocor), BT-061 CD4 (BioTest AG), efalizumab CD11a (e.g., RAPTIVA®/XANELIM™, Genentech/Roche/Merck-Serono), MLN01 CD18 (Millennium Pharmaceuticals), epratuzumab CD22 (e.g., Amgen antibody) and hLL2; (Immunomedics/UCB)), aselizumab CD26L, iratumumab CD30 (e.g., SGN30 (Seattle Genetics) and MDX-060 (Medarex), SGN40 CD40 (Seattle Genetics), ANTOVA® CD40 ligand (Biogen Idec), abatacept CD80 CD86 (e.g., ORENCIA®, Bristol-Myers Squibb), CT-011 PD1 (Cure Tech), AT010 CXCR3 (Affitech), MLN₁₂O₂ CCR2 (Millennium Pharmaceuticals), AMG-761 CCR4 (Amgen), HGS004 CCR5 (Human Genome Sciences), PRO140 (Progenics), MDX-1338 CXCR4 (Medarex), CNTO-888 CCL2 (Centocor), ABN912 CCL2 (Novartis), MDX-1100 CXCL10 (Medarex), TB-403 PlGF (Bioinvent), natalizumab integrin Alpha4 subunit (e.g., TYSABR1 ®, Biogen Idec/Elan), vedolizumab integrin A4B7 (e.g., MLN2, Millennium Pharmaceuticals/Takeda), eculizumab C5 Compliment (e.g., SOLIRIS®, Alexion), pexelizumab C5 Compliment (Alexion), omalizumab IgE (e.g., XOLAIR®, Genentech/Roche/Novartis), talizumab (e.g., TNX-901, Tanox), toralizumab (IDEC 131, IDEC), bertilimumab eotaxin (e.g., iCo-008, iCo Therapeutics Inc.), ozrolimupab RhD (e.g., Sym001, Symphogen A/S), atorolimumab and morolimumab (Rh factor). MMM complex (e.g., ELP-MRD fusion protein) having 1, 2, 3, 4, 5, 6, or more MRDs that bind to the same epitope as, or competitively inhibit binding of one, of the above antibodies are also encompassed by the invention. MMM complex (e.g., ELP-MRD fusion protein) having MRDs that bind to the same epitope as, or competitively inhibit binding of, at least 1, 2, 3, 4, 5, 6 or more of the above antibodies are additionally encompassed by the invention.

In some embodiments, an MRD and/or the MMM complex (e.g., ELP-MRD fusion protein) binds TNF. In another embodiment, an MRD and/or the MMM complex (e.g., ELP-MRD fusion protein) binds to the same epitope as adalimumab (e.g., HUMIRA®/TRUDEXA®, Abbott). In another embodiment, an MRD and/or the MMM complex (e.g., ELP-MRD fusion protein) competitively inhibits binding of adalimumab to TNF. In an additional embodiment, an MRD and/or the MMM complex (e.g., ELP-MRD fusion protein) binds to the same epitope as infliximab. In another embodiment, an MRD and/or the MMM complex (e.g., ELP-MRD fusion protein) competitively inhibits binding of infliximab to TNF. In a further embodiment, an MRD and/or the MMM complex (e.g., ELP-MRD fusion protein) competitively inhibits binding of: certolizumab (e.g., CIMZIA®, UCB), golimumab (e.g., SIMPONI™, Centocor), or AME-527 (Applied Molecular Evolution) to TNF. In an additional embodiment, an MRD binds to the same epitope as certolizumab (e.g., CIMZIA®, UCB), golimumab (e.g., SIMPONI™, Centocor), or AME-527 (Applied Molecular Evolution). In another embodiment, an MRD and/or the MMM complex (e.g., ELP-MRD fusion protein) competitively inhibits binding of certolizumab, golimumab, or AME-527, to TNF. MMM complex (e.g., ELP-MRD fusion protein) having 1, 2, 3, 4, 5, 6, or more MRDs that bind to the same epitope as, or competitively inhibit binding of, one of the above antibodies are also encompassed by the invention. MMM complex (e.g., ELP-MRD fusion protein) having MRDs that bind to the same epitope as, or competitively inhibit binding of, at least 1, 2, 3, 4, 5, 6 or more of the above antibodies are additionally encompassed by the invention.

In particular embodiments, an MRD and/or the MMM complex (e.g., ELP-MRD fusion protein) binds the target: amyloid beta (Abeta), beta amyloid, complement factor D, PLP, ROBO4, ROBO, GDNF, NGF, LINGO, or myostatin. MMM complex (e.g., ELP-MRD fusion protein) having 1, 2, 3, 4, 5, 6, or more MRDs that bind to one of the above targets are also encompassed by the invention. MMM complex (e.g., ELP-MRD fusion protein) having MRDs that bind to at least 1, 2, 3, 4, 5, 6 or more of the above targets are additionally encompassed by the invention.

In some embodiments, an MRD and/or the MMM complex (e.g., ELP-MRD fusion protein) binds to the same epitope as gantenerumab (e.g., R1450, Hoffman La-Roche), bapineuzumab beta amyloid 9 (Elan and Wyeth), solanezumab beta amyloid 9 (Lilly), tanezumab NGF (e.g., RN624, Pfizer), BIIB033 LINGO (Biogen Idec), or stamulumab myostatin (Wyeth). In another embodiment, an MRD and/or the MMM complex (e.g., ELP-MRD fusion protein) competitively inhibits target binding by gantenerumab, bapineuzumab, solarezumab, tanezumab, BIIB033, or stamulumab. MRDs and/or the MMM complex (e.g., ELP-MRD fusion protein) that compete for target binding with one of the above antibodies is also encompassed by the invention. MMM complex (e.g., ELP-MRD fusion protein) having 1, 2, 3, 4, 5, 6, or more MRDs that bind to the same epitope as, or competitively inhibit binding of, one of the above antibodies are also encompassed by the invention. MMM complex (e.g., ELP-MRD fusion protein) having MRDs that bind to the same epitope as, or competitively inhibit binding of, at least 1, 2, 3, 4, 5, 6 or more of the above antibodies are additionally encompassed by the invention.

In another embodiment, the target of an MRD and/or the MMM complex (e.g., ELP-MRD fusion protein) is: oxidized LDL, gpIIB, gpIIIa, PCSK9, Factor VIII, integrin a2bB3, AOC3, or mesothelin. In specific embodiments, the antibody in the MMM complex (e.g., ELP-MRD fusion protein) is BI-204 oxidized LDL (BioInvent), abciximab gpIIB, gpIIIa (e.g., REOPRO, Eli Lilly), AMG-145 PCSK9 (Amgen), TB-402 Factor VIII (BioInvent), vapaliximab, or tadocizumab integrin a2bB3 (Yamonochi Pharma). In another embodiment, an MRD and/or the MMM complex (e.g., ELP-MRD fusion protein) binds to the same epitope as BI-204, abciximab, AMG-145, TB-402, or tadocizumab. In another embodiment, the antibody an MRD and/or the MMM complex (e.g., ELP-MRD fusion protein) competitively inhibits binding of BI-204, abciximab, AMG-145, TB-402, vapaliximab, or tadocizumab. MRDs and/or the MMM complex (e.g., ELP-MRD fusion protein) that compete for target binding with one of the above antibodies is also encompassed by the invention. MMM complex (e.g., ELP-MRD fusion protein) having 1, 2, 3, 4, 5, 6, or more MRDs that bind to one of the above targets are also encompassed by the invention. MMM complex (e.g., ELP-MRD fusion protein) having MRDs that bind to at least 1, 2, 3, 4, 5, 6, or more of the above targets are additionally encompassed by the invention.

In other embodiments, a target of an MRD and/or the MMM complex (e.g., ELP-MRD fusion protein) is associated with bone growth and/or metabolism. In certain embodiments, a target of an MRD and/or the MMM complex (e.g., ELP-MRD fusion protein) is TNFSF11 (RANKL). In a specific embodiment, an MRD and/or the MMM complex (e.g., ELP-MRD fusion protein) binds to the same epitope as denosumab (e.g., AMG-162, Amgen). In other embodiments, the target of an MRD and/or the MMM complex (e.g., ELP-MRD fusion protein) is: DKK1, osteopontin, cathepsin K, TNFRSF19L (RELT), TNFRSF19 (TROY), or sclerostin (CDP-7851 UCB Celltech). In other embodiments, an MRD and/or the MMM complex (e.g., ELP-MRD fusion protein) binds to the same epitope as AMG617 or AMG785 (e.g., CDP7851, Amgen). In another embodiment, an MRD and/or the MMM complex (e.g., ELP-MRD fusion protein) competitively inhibits target binding of AMG617 or AMG785 (e.g., CDP7851, Amgen). In another embodiment, an MRD and/or the MMM complex (e.g., ELP-MRD fusion protein) competitively inhibits binding of sclerostin by AMG617 or AMG785. MRDs and/or the MMM complex (e.g., ELP-MRD fusion protein) that compete for target binding with one of the above antibodies is also encompassed by the invention. MMM complex (e.g., ELP-MRD fusion protein) having 1, 2, 3, 4, 5, 6, or more MRDs that bind to one of the above targets are also encompassed by the invention. MMM complex (e.g., ELP-MRD fusion protein) having MRDs that bind to at least 1, 2, 3, 4, 5, 6, or more of the above targets are additionally encompassed by the invention. MMM complex (e.g., ELP-MRD fusion protein) having 1, 2, 3, 4, 5, 6, or more MRDs that bind to the same epitope as, or competitively inhibit binding of one of the above antibodies are also encompassed by the invention. MMM complex (e.g., ELP-MRD fusion protein) having MRDs that bind to the same epitope as, or competitively inhibit binding of, at least 1, 2, 3, 4, 5, 6, or more of the above antibodies are additionally encompassed by the invention.

In additional embodiments, a target of an MRD and/or the MMM complex (e.g., ELP-MRD fusion protein) is a bacterial antigen, a viral antigen, a mycoplasm antigen, a prion antigen, or a parasite antigen (e.g., one infecting a mammal).

In other embodiments, a target of an MRD and/or the MMM complex (e.g., ELP-MRD fusion protein) is a viral antigen. In one embodiment, the target of an MRD and/or the MMM complex (e.g., ELP-MRD fusion protein) is anthrax, hepatitis b, rabies, Nipah virus, west nile virus, a mengititis virus, or CMV. In other embodiments, an MRD and/or the MMM complex (e.g., ELP-MRD fusion protein) competes with antigen binding with ABTHRAX® (Human Genome Sciences), exbivirumab, foravirumab, libivirumab, rafivirumab, regavirumab, sevirumab (e.g., MSL-109, Protovir), tuvirumab, raxibacumab, Nipah virus M102.4, or MGAWN1® (MacroGenics) for target binding. MMM complex (e.g., ELP-MRD fusion protein) having 1, 2, 3, 4, 5, 6, or more MRDs that bind to one of the above targets are also encompassed by the invention. MMM complex (e.g., ELP-MRD fusion protein) having MRDs that bind to at least 1, 2, 3, 4, 5, 6, or more of the above targets are additionally encompassed by the invention. MMM complex (e.g., ELP-MRD fusion protein) having 1, 2, 3, 4, 5, 6, or more MRDs that bind to the same epitope as, or competitively inhibit binding of, one of the above antibodies are also encompassed by the invention. MMM complex (e.g., ELP-MRD fusion protein) having MRDs that bind to the same epitope as, or competitively inhibit binding of, at least 1, 2 or more of the above antibodies are additionally encompassed by the invention.

In further embodiments, a target of an MRD and/or the MMM complex (e.g., ELP-MRD fusion protein) is RSV. In other embodiments, an MRD and/or ELP-MRD binds to the same epitope as, motavizumab (e.g., NUMAX®, MEDI-577; Medlmmune) or palivizumab RSV fusion f protein (e.g., SYNAGIS®, Medlmmune). In other embodiments, an MRD and/or the MMM complex (e.g., ELP-MRD fusion protein) competes for target binding with motavizumab (e.g., NUMAX®, MEDI-577; Medlmmune) or palivizumab RSV fusion f protein (e.g., SYNAGIS®, Medlmmune). In other embodiments, an MRD and/or the MMM complex (e.g., ELP-MRD fusion protein) competes for target binding with felvizumab. In other embodiments, an MRD and/or the MMM complex (e.g., ELP-MRD fusion protein) competitively inhibits target binding by felvizumab. MRDs and/or the MMM complex (e.g., ELP-MRD fusion protein) that compete for target binding with one of the above antibodies is also encompassed by the invention. MMM complex (e.g., ELP-MRD fusion protein) having 1, 2, 3, 4, 5, 6, or more MRDs that bind to the same epitope as, or competitively inhibit binding of, one of the above antibodies are also encompassed by the invention. MMM complex (e.g., ELP-MRD fusion protein) having MRDs that bind to the same epitope as, or competitively inhibit binding of, at least 1, 2, 3, 4, 5, 6, or more of the above antibodies are additionally encompassed by the invention.

In other embodiments, a target of an MRD and/or the MMM complex (e.g., ELP-MRD fusion protein) is a bacterial or fungal antigen. In other embodiments, an MRD and/or the MMM complex (e.g., ELP-MRD fusion protein) binds to the same epitope as nebacumab, edobacomab (e.g., E5), tefibazumab (Inhibitex), panobacumab (e.g., KBPA101, Kenta), pagibaximab (e.g., BSYX-A110, Biosynexus), urtoxazumab, or efungumab (e.g., MYCOGRAB®, Novartis). In other embodiments, an MRD and/or the MMM complex (e.g., ELP-MRD fusion protein) competitively inhibits antigen binding by nebacumab, edobacomab, tefibazumab, panobacumab, pagibaximab, urtoxazumab, or efungumab. MRDs and/or the MMM complex (e.g., ELP-MRD fusion protein) that compete for target binding with one of the above antibodies is also encompassed by the invention. MMM complex (e.g., ELP-MRD fusion protein) having 1, 2, 3, 4, 5, 6, or more MRDs that bind to the same epitope as, or competitively inhibit binding of, one of the above antibodies are also encompassed by the invention. MMM complex (e.g., ELP-MRD fusion protein) having MRDs that bind to the same epitope as, or competitively inhibit binding of, at least 1, 2, 3, 4, 5, 6, or more of the above antibodies are additionally encompassed by the invention.

In another embodiment, an MRD and/or the MMM complex (e.g., ELP-MRD fusion protein) binds to the same epitope as 38C2. In a further embodiment, an MRD and/or the MMM complex (e.g., ELP-MRD fusion protein) competitively inhibits 38C2 binding.

In an additional embodiment, an MRD and/or the MMM complex (e.g., ELP-MRD fusion protein) binds to A33 antigen. Human A33 antigen is a transmembrane glycoprotein of the Ig superfamily. Several properties of the A33 antigen suggest that it is a promising target for immunotherapy of colon cancer. These properties include (i) the highly restricted expression pattern of the A33 antigen, (ii) the expression of large amounts of the A33 antigen on colon cancer cells, (iii) the absence of secreted or shed A33 antigen, (iv) the fact that upon binding of antibody A33 to the A33 antigen, antibody A33 is internalized and sequestered in vesicles, and (v) the targeting of antibody A33 to A33 antigen expressing colon cancer in preliminary clinical studies.

Numerous target-binding sites are contemplated as a target of an ELP-MRD fusions of the present invention, including for example, epidermal growth factor receptor (EGFR), CD20, tumor antigens, ErbB2, ErbB3, ErbB4, insulin-like growth factor-I receptor, nerve growth factor (NGR), hepatocyte growth factor receptor, and tumor-associated surface antigen epithelial cell adhesion molecule (Ep-CAM). MRDs can be directed towards these target-binding sites or the corresponding ligands.

In some embodiments, an MRD and/or the MMM complex (e.g., ELP-MRD fusion protein) binds to a human protein. In some embodiments, an MRD and/or the MMM complex (e.g., ELP-MRD fusion protein) binds to both a human protein and its ortholog in mouse, rabbit, hamster or rabbit ortholog.

In some embodiments, an MRD and/or the MMM complex (e.g., ELP-MRD fusion protein) binds to a human target protein. In some embodiments, an MRD and/or the MMM complex (e.g., ELP-MRD fusion protein) binds to both a human protein and its monkey, mouse, rabbit, and/or hamster ortholog.

In additional embodiments, an MRD and/or the MMM complex (e.g., ELP-MRD fusion protein) target is a target that has been validated in an animal model or clinical setting.

In some embodiments, described herein, an MRD and/or the MMM complex (e.g., ELP-MRD fusion protein) bindsan integrin. The role of integrins such as αvβ3 and αvβ5 as tumor-associated markers has been well documented. A recent study of 25 permanent human cell lines established from advanced ovarian cancer demonstrated that all lines were positive for αvβ5 expression and many were positive for αvβ3 expression. Studies have also shown that αvβ3 and αvβ5 is highly expressed on malignant human cervical tumor tissues. Integrins have also demonstrated therapeutic effects in animal models of Kaposi's sarcoma, melanoma, and breast cancer.

A number of integrin αvβ3 and αvβ5 antagonists are in clinical development.

These include cyclic RGD peptides and synthetic small molecule RGD mimetics. Two antibody-based integrin antagonists are currently in clinical trials for the treatment of cancer. The first is VITAXIN® (MEDI-522, Abegrein), the humanized form of the murine anti-human αvβ3 antibody LM609. A dose-escalating phase I study in cancer patients demonstrated that VITAXIN® is safe for use in humans. Another antibody in clinical trials is CNT095, a fully human Ab that recognizes αv integrins. A Phase I study of CNT095 in patients with a variety of solid tumors has shown that it is well tolerated. Cilengitide (EMD 121974), a peptide antagonist of αvβ3 and αvβ5, has also proven safe in phase I trials. Furthermore, there have been numerous drug targeting and imaging studies based on the use of ligands for these receptors. These preclinical and clinical observations demonstrate the importance of targeting αvβ3 and αvβ5 and studies have consistently reported that targeting through these integrins is safe.

Clinical trials are also ongoing for antagonists targeting α5vβ1 for treating metastatic melanoma, renal cell carcinoma, and non-small cell lung cancer (M200 (volociximab) and malignant glioma (ATN-161).

Integrin-binding MRD and/or the MMM complex (e.g., ELP-MRD fusion protein) containing one more RGD tripeptide sequence motifs represent an example of MRDs of the invention. Ligands having the RGD motif as a minimum recognition domain and from which MRDs of the invention can be derived are well known, a partial list of which includes, with the corresponding integrin target in parenthesis, fibronectin (α3β1, α5β1, αvβ1, αllbβ3, αvβ3, and α3β1) fibrinogen (αMβ2 and αllbβ1) von Willebrand factor (αllbβ3 and αvβ3), and vitronectin (αllbβ3, αvβ3 and αvβ5).

In one embodiment, the MMM complex (e.g., ELP-MRD fusion protein) comprises and RGD binding MRD having a sequence selected from the group consisting of: YCRGDCT (SEQ ID NO:50); PCRGDCL (SEQ ID NO:51); TCRGDCY (SEQ ID NO:52); and LCRGDCF (SEQ ID NO:53).

An MMM complex (e.g., an ELP-MRD fusion protein) comprising an MRD that mimics a non-RGD-dependent binding site on an integrin receptor and having the target binding specificity of a high affinity ligand that recognizes the selected integrin is also contemplated in the present invention. MRDs that bind to an integrin receptor and disrupt binding and/or signaling activity of the integrin are also contemplated.

In some embodiments, an MRD and/or the MMM complex (e.g., ELP-MRD fusion protein) binds an angiogenic molecule. Angiogenesis is essential to many physiological and pathological processes. Ang2 has been shown to act as a proangiogenic molecule. Administration of Ang2-selective inhibitors is sufficient to suppress both tumor angiogenesis and corneal angiogenesis. Therefore, Ang2 inhibition alone or in combination with inhibition of other angiogenic factors, such as VEGF, can represent an effective antiangiogenic strategy for treating patients with solid tumors.

MRDs and/or the MMM complex (e.g., ELP-MRD fusion protein) that bind to angiogenic receptors, angiogenic factors, and/or Ang2 are also encompassed by the invention. In a specific embodiment, the MRD and/or the MMM complex (e.g., ELP-MRD fusion protein) binds Ang2. In one embodiment, an MRD and/or the MMM complex (e.g., ELP-MRD fusion protein) contains a sequence selected from the group: MGAQTNFMPMDDLEQRLYEQFILQQGLE (SEQ ID NO:7); MGAQTNFMPMDNDELLLYEQFILQQGLE (SEQ ID NO:8); MGAQTNFMPMDATETRLYEQFILQQGLE (SEQ ID NO:9); AQQEECEWDPWTCEHMGSGSATGGSGSTASSGSGSATHQEECEWDPWTCEHMLE (SEQ ID NO:10) (2xCon4); MGAQTNFMPMDNDELLNYEQFILQQGLE (SEQ ID NO:11); and PXDNDXLLNY (SEQ ID NO:12) where X is one of the 20 naturally-occurring amino acids.

In other embodiments, an MRD and/or the MMM complex (e.g., ELP-MRD fusion protein) binds an angiogenic cytokine and contains a sequence selected from the group: MGAQTNFMPMDNDELLLYEQFILQQGLEGGSGSTASSGSGSSLGAQTNFMPMDNDELLLY (SEQ ID NO:20); AQQEECEWDPWTCEHMGSGSATGGSGSTASSGSGSATHQEECEWDPWTCEHMLE (SEQ ID NO:10); AQQEECEFAPWTCEHM (SEQ ID NO:21) (ConFA); core nEFAPWTn (SEQ ID NO:22) where n is from about 0 to 50 amino acid residues; AQQEECEFAPWTCEHMGSGSATGGSGSTASSGSGSATHQEECEFAPWTCEHMLE (SEQ ID NO:23) (2xConFA); and AQQEECELAPWTCEHM (SEQ ID NO:24) (ConLA).

In additional embodiments, an MRD and/or the MMM complex (e.g., ELP-MRD fusion protein) binds an angiogenic cytokine and contains a sequence selected from the group: XnELAPWTXn where n is from about 0 to 50 amino acid residues and X is any amino acid (SEQ ID NO:25); AQQEECELAPWTCEHMGSGSATGGSGSTASSGSGSATHQEECELAPWTCEHMLE (SEQ ID NO:26) (2xConLA); AQQEECEFSPWTCEHM (SEQ ID NO:27) (ConFS); XnEFSPWTXn where n is from about 0 to 50 amino acid residues and X is any amino acid (SEQ ID NO:28); AQQEECEFSPWTCEHMGSGSATGGSGSTASSGSGSATHQEECEFSPWTCEHMLE (SEQ ID NO:29) (2xConFS); AQQEECELEPWTCEHM (SEQ ID NO:30) (ConLE); XnELEPWTXn where n is from about 0 to 50 amino acid residues (SEQ ID NO:31) and wherein X is any amino acid; and AQQEECELEPWTCEHMGSGSATGGSGSTASSGSGSATHQEECELEPWTCEHMLE (SEQ ID NO:32) (2xConLE).

In one embodiment, an MRD and/or the MMM complex (e.g., ELP-MRD fusion protein) binds Ang2 and contains a sequence selected from the group consisting of: GAQTNFMPMDDLEQRLYEQFILQQGLE (SEQ ID NO:144) (ANGa); LWDDCYFFPNPPHCYNSP (SEQ ID NO:148) (ANGb); LWDDCYSYPNPPHCYNSP (SEQ ID NO:149) (ANGc); LWDDCYSFPNPPHCYNSP (SEQ ID NO:150) (ANGd); DCAVYPNPPWCYKMEFGK (SEQ ID NO:151) (ANGe); PHEECYFYPNPPHCYTMS (SEQ ID NO:152) (ANGf); and PHEECYSYPNPPHCYTMS (SEQ ID NO:153) (ANGg).

It should be understood that MRDs in the MMM complex (e.g., ELP-MRD fusion protein) can be present in tandem dimers, trimers or other multimers either homologous or heterologous in nature. For example, one can dimerize identical Con-based sequences such as in 2xConFA to provide a homologous dimer, or the Con peptides can be mixed such that ConFA is combined with ConLA to create ConFA-LA heterodimer with the sequence:

(SEQ ID NO: 33)
AQQEECEFAPWTCEHMGSGSATGGSGSTASSGSGSATHQEECELAPWTCE
HMLE.

Another heterodimer of the invention is ConFA combined with ConFS to create

ConFA-FS with the sequence: AQQEECEFAPWTCEHMGSGSATGGSGSTASSGSGSATHQEECEFSPWTCEHMLE (SEQ ID NO:34).

Other such peptide combinations that create functional Ang2 binding MRDs are encompassed by the invention.

The invention also includes a human Ang2 binding MRD and/or the MMM complex (e.g., ELP-MRD fusion protein) having a core sequence selected from: XnEFAPWTXn where n is from about 0 to 50 amino acid residues (SEQ ID NO:22); XnELAPWTXn where n is from about 0 to 50 amino acid residues (SEQ ID NO:25); XnEFSPWTXn where n is from about 0 to 50 amino acid residues (SEQ ID NO:28); XnELEPWTXn where n is from about 0 to 50 amino acid residues (SEQ ID NO:31); and XnAQQEECEX₁X₂PWTCEHMXn where n is from about 0 to 50 amino acid residues and X represents any natural amino acid (SEQ ID NO:57).

In some embodiments, an MRD and/or the MMM complex (e.g., ELP-MRD fusion protein) binds vascular endothelial growth factor (VEGF). Phage display selections and structural studies of VEGF neutralizing peptides in complex with VEGF have been reported. These studies have revealed that peptide vl 14 (VEPNCDIHVMWEWECFERL) (SEQ ID NO:13) is VEGF specific, binds VEGF with 0.2 μM affinity, and neutralizes VEGF-induced proliferation of Human Umbilical Vein Endothelial Cells (HUVEC). Since VEGF is a homodimer, the peptide occupies two identical sites at either end of the VEGF homodimer. In a specific embodiment, the ELP-MRD fusion of the invention comprises v114. In other embodiments, the ELP-MRD fusion comprises a V114 variant/derivative that competitively inhibit the ability of the antibody-vl14 fusion to bind to VEGF. In another embodiment, the ELP-MRD fusion comprises an MRD with the sequence ATWLPPP (SEQ ID NO:71), which inhibits VEGF-mediated angiogenesis. Binetruy-Tournaire et al., EMBO 19:1525-1533 (2000), which is herein incorporated by reference.

Insulin-like growth factor-I receptor-specific MRDs can also be used in the present invention. In one embodiment, an MRD sequence that targets the insulin-like growth factor-I receptor is SFYSCLESLVNGPAEKSRGQWDGCRKK (SEQ ID NO:14).

In one embodiment, the invention includes an MRD and/or the MMM complex (e.g., ELP-MRD fusion protein) that binds IGF and has the sequence: NFYQCIX₁X₂LX₃X₄X₅P AEKSRGQWQECRTGG (SEQ ID NO:58), wherein X₁ is E or D; X₂ is any amino acid; X₃ is any amino acid; X₄ is any amino acid and X₅ is any amino acid.

In another embodiment, an MRD and/or the MMM complex (e.g., ELP-MRD fusion protein) binds IGF1R and contains a sequence selected from the group: NFYQCIEMLASHPAEKSRGQWQECRTGG (SEQ ID NO:35); NFYQCIEQLALRPAEKSRGQWQECRTGG (SEQ ID NO:36); NFYQCIDLLMAYPAEKSRGQWQECRTGG (SEQ ID NO:37); NFYQCIERLVTGPAEKSRGQWQECRTGG (SEQ ID NO:38); NFYQCIEYLAMKPAEKSRGQWQECRTGG (SEQ ID NO:39); and NFYQCIEALQSRPAEKSRGQWQECRTGG (SEQ ID NO:40).

In another embodiment, an MRD and/or the MMM complex (e.g., ELP-MRD fusion protein) binds IGF1R and contains a sequence selected from the group: NFYQCIEALSRSPAEKSRGQWQECRTGG (SEQ ID NO:41); NFYQCIEHLSGSPAEKSRGQWQECRTG (SEQ ID NO:42); NFYQCIESLAGGPAEKSRGQWQECRTG (SEQ ID NO:43); NFYQCIEALVGVPAEKSRGQWQECRTG (SEQ ID NO:44); and NFYQCIEMLSLPPAEKSRGQWQECRTG (SEQ ID NO:45).

In another embodiment, the IGF1R binding MRD and/or the MMM complex (e.g., ELP-MRD fusion protein) contains a sequence selected from the group: NFYQCIEVFWGRPAEKSRGQWQECRTG (SEQ ID NO:46); NFYQCIEQLSSGPAEKSRGQWQECRTG (SEQ ID NO:47); NFYQCIELLSARPAEKSRGQWAECRAG (SEQ ID NO:48); and NFYQCIEALARTPAEKSRGQWVECRAP (SEQ ID NO:49).

Vascular homing-specific MRDs and/or the MMM complex (e.g., ELP-MRD fusion protein) are also contemplated for use in the present invention. A number of studies have characterized the efficacy of linking the vascular homing peptide to other proteins like IL-12 or drugs to direct their delivery in live animals. One example of an MRD sequence that is a vascular homing peptide that is envisioned to be included within an ELP-MRD fusion of the invention is ACDCRGDCFCG (SEQ ID NO:15).

In one embodiment, an MRD and/or the MMM complex (e.g., ELP-MRD fusion protein) binds to EGFR and has a sequence selected from the group:

(SEQ ID NO: 16)
VDNKFNKELEKAYNEIRNLPNLNGWQ
MTAFIASLVDDPSQSANLLAEAKKLNDAQAPK;
and
(SEQ ID NO: 17)
VDNKFNK
EMWIAWEEIRNLPNLNGWQMTAFIASLVDDPSQSANLLAEAKKLNDAQAP
K.

In another embodiment, an MRD and/or the MMM complex (e.g., ELP-MRD fusion protein) binds ErbB2 and has the sequence:

(SEQ ID NO: 18)
VDNKFNKEMRNAYWEIALLPNLNNQQKRAFIRSLYDDPS
QSANLLAEAKKLNDAQAPK.

In some embodiments, an MRD and/or the MMM complex (e.g., ELP-MRD fusion protein) binds a target selected from the group consisting of an angiogenic cytokine and an integrin. In a specific embodiment, an MRD comprises the sequence of SEQ ID NO:8. In another specific embodiment, an MRD comprises the sequence of SEQ ID NO:14. In another specific embodiment, an MRD comprises the sequence of SEQ ID NO:69.

In one embodiment, an MRD is about 2 to 150 amino acids. In another embodiment, an MRD is about 2 to 60 amino acids.

In an additional embodiment, the MMM complex (e.g., ELP-MRD fusion protein) comprises an MRD containing a sequence selected from the group consisting of SEQ ID NO:8, SEQ ID NO:14, and SEQ ID NO:70.

In one embodiment, an MRD and/or the MMM complex (e.g., ELP-MRD fusion protein) binds a cellular antigen. In a specific embodiment an MRD and/or the MMM complex (e.g., ELP-MRD fusion protein) binds CD20.

In another embodiment, an MRD and/or the MMM complex (e.g., ELP-MRD fusion protein) binds an integrin. In one embodiment, the peptide sequence of the integrin targeting MRD is YCRGDCT (SEQ ID NO:3). In an additional embodiment, the peptide sequence of the integrin targeting MRD is PCRGDCL (SEQ ID NO:4). In yet another embodiment, the peptide sequence of the integrin targeting MRD is TCRGDCY (SEQ ID NO:5). In another embodiment, the peptide sequence of the integrin targeting MRD is LCRGDCF (SEQ ID NO:6).

In an additional embodiment, an MRD and/or the MMM complex (e.g., ELP-MRD fusion protein) binds an angiogenic cytokine. In one embodiment, the peptide sequence of an angiogenic cytokine targeting (i.e. binding) MRD is MGAQTNFMPMDDLEQRLYEQFILQQGLE (SEQ ID NO:7). In another embodiment, the peptide sequence of an angiogenic cytokine targeting MRD is MGAQTNFMPMDNDELLLYEQFILQQGLE (SEQ ID NO:8). In yet another embodiment, the amino acid sequence of an angiogenic cytokine targeting MRD is MGAQTNFMPMDATETRLYEQFILQQGLE (SEQ ID NO:9). In another embodiment, the amino acid sequence of an angiogenic cytokine targeting MRD is AQQEECEWDPWTCEHMGSGSATGGSGSTASSGSGSATHQEECEWDPWTCEHMLE (SEQ ID NO:10). In an additional embodiment, the amino acid sequence of an angiogenic cytokine targeting MRD is MGAQTNFMPMDNDELLNYEQFILQQGLE (SEQ ID NO:11). In another embodiment, the amino acid sequence of an angiogenic cytokine targeting MRD is PXDNDXLLNY (SEQ ID NO:12), wherein X is one of the 20 naturally-occurring amino acids. In a further embodiment, the targeting MRD peptide has the core sequence MGAQTNFMPMDXn (SEQ ID NO:56), wherein X is any amino acid and n is from about 0 to 15.

In a further embodiment, the targeting MRD peptide contains a core sequence selected from:

XnEFAPWTXn where n is from about 0 to 50 amino acid residues (SEQ ID NO:22);
XnELAPWTXn where n is from about 0 to 50 amino acid residues (SEQ ID NO:25);
XnEFSPWTXn where n is from about 0 to 50 amino acid residues (SEQ ID NO:28);
XnELEPWTXn where n is from about 0 to 50 amino acid residues (SEQ ID NO:31); and
XnAQQEECEX₁X₂PWTCEHMXn where n is from about 0 to 50 amino acid residues and X, X₁ and X₂ are any amino acid (SEQ ID NO:57).

Exemplary peptides containing such core peptides encompassed by the invention include for example:

(SEQ ID NO: 21)
AQQEECEFAPWTCEHM;
(SEQ ID NO: 23)
AQQEECEFAPWTCEHMGSGSATGGSGSTASSGSGSATHQEECEFAPWTCE
HMLE;
(SEQ ID NO: 24)
AQQEECELAPWTCEHM;
(SEQ ID NO: 26)
AQQEECELAPWTCEHMGSGSATGGSGSTASSGSGSATHQEECELAPWTCE
HMLE;
(SEQ ID NO: 27)
AQQEECEFSPWTCEHM;
(SEQ ID NO: 29)
AQQEECEFSPWTCEHMGSGSATGGSGSTASSGSGSATHQEECEFSPWTCE
HMLE 2xConFS;
(SEQ ID NO: 30)
AQQEECELEPWTCEHM;
(SEQ ID NO: 32)
AQQEECELEPWTCEHMGSGSATGGSGSTASSGSGSATHQEECELEPWTCE
HMLE;
(SEQ ID NO: 33)
AQQEECEFAPWTCEHMGSGSATGGSGSTASSGSGSATHQEECELAPWTCE
HMLE;
(SEQ ID NO: 34)
AQQEECEFAPWTCEHMGSGSATGGSGSTASSGSGSATHQEECEFSPWTCE
HMLE;
and
(SEQ ID NO: 10)
AQQEECEWDPWTCEHMGSGSATGGSGSTASSGSGSATHQEECEWDPWTCE
HMLE.

In one embodiment, a target of an MRD and/or the MMM complex (e.g., ELP-MRD fusion protein) is ErbB2. In a further embodiment, the MRD and/or the MMM complex (e.g., ELP-MRD fusion protein) binds ErbB3. In an additional embodiment, the MRD and/or the MMM complex (e.g., ELP-MRD fusion protein) binds a tumor-associated surface antigen epithelial cell adhesion molecule (Ep-CAM).

In one embodiment, the target to which an MRD binds is VEGF. In one embodiment, the peptide sequence of the VEGF targeting MRD is VEPNCDIHVMWEWECFERL (SEQ ID NO:13).

In one embodiment, the target to which an MRD and/or the MMM complex (e.g., ELP-MRD fusion protein) binds is an insulin-like growth factor-I receptor (IGF1R). In another embodiment, the peptide sequence of an insulin-like growth factor-I receptor targeting MRD comprises SFYSCLESLVNGPAEKSRGQWDGCRKK (SEQ ID NO:14). Other illustrative IGF1R targeting MRDs include, for example, a peptide having an amino acid sequence that has the formula NFYQCIX₁X₂LX₃X₄X₅PAEKSRGQWQECRTGG (SEQ ID NO:58), wherein X₁ is E or D; X₂ is any amino acid; X₃ is any amino acid; X₄ is any amino acid; and X₅ is any amino acid. Other illustrative IGF1R targeting MRDs include, for example, a peptide sequence having the formula of XXXXCXEXXXXXPAEKSRGQWXXCXXX (SEQ ID NO: 101).

Illustrative peptides that contain such formula include:

(SEQ ID NO: 35)
NFYQCIEMLASHPAEKSRGQWQECRTGG;
(SEQ ID NO: 36)
NFYQCIEQLALRPAEKSRGQWQECRTGG;
(SEQ ID NO: 38)
NFYQCIERLVTGPAEKSRGQWQECRTGG;
(SEQ ID NO: 39)
NFYQCIEYLAMKPAEKSRGQWQECRTGG;
(SEQ ID NO: 40)
NFYQCIEALQSRPAEKSRGQWQECRTGG;
(SEQ ID NO: 41)
NFYQCIEALSRSPAEKSRGQWQECRTGG;
(SEQ ID NO: 42)
NFYQCIEHLSGSPAEKSRGQWQECRTG;
(SEQ ID NO: 43)
NFYQCIESLAGGPAEKSRGQWQECRTG;
(SEQ ID NO: 44)
NFYQCIEALVGVPAEKSRGQWQECRTG;
(SEQ ID NO: 45)
NFYQCIEMLSLPPAEKSRGQWQECRTG;
(SEQ ID NO: 46)
NFYQCIEVFWGRPAEKSRGQWQECRTG;
(SEQ ID NO: 47)
NFYQCIEQLSSGPAEKSRGQWQECRTG;
(SEQ ID NO: 48)
NFYQCIELLSARPAEKSRGQWAECRAG;
(SEQ ID NO: 49)
NFYQCIEALARTPAEKSRGQWVECRAP;
(SEQ ID NO: 67)
NFYQCIESLVNGPAEKSRGQWDGCRKK
(Rm1-67);
(SEQ ID NO: 68)
NFYQCIESLVNGPAEKSRGQWVECRAP
(Rm2-2-218);
(SEQ ID NO: 69)
NFYQCIESLVNGPAEKSRGQWAECRAG
(Rm2-2-316);
and
(SEQ ID NO: 70)
NFYQCIESLVNGPAEKSRGQWQECRTG
(Rm2-2-319).

Other illustrative IGF1R targeting MRDs include, for example, a peptide sequence having the formula: NFYQCIDLLMAYPAEKSRGQWQECRTGG (SEQ ID NO:37).

In one embodiment, a target of an MRD and/or the MMM complex (e.g., ELP-MRD fusion protein) is a tumor antigen.

In an additional embodiment, a target of an MRD and/or the MMM complex (e.g., ELP-MRD fusion protein) is an epidermal growth factor receptor (EGFR). In another embodiment a target of an MRD and/or the MMM complex (e.g., ELP-MRD fusion protein) is an angiogenic factor. In an additional embodiment, a target of an MRD and/or the MMM complex (e.g., ELP-MRD fusion protein) is an angiogenic receptor.

In additional embodiments, the invention encompasses MRD and/or the MMM complex (e.g., ELP-MRD fusion protein) that is a vascular homing peptide. In one embodiment, the peptide sequence of a vascular homing peptide MRD comprises the sequence ACDCRGDCFCG (SEQ ID NO:15).

In a further embodiment, a target of an MRD and/or the MMM complex (e.g., ELP-MRD fusion protein) is a nerve growth factor.

In another embodiment, the MRD and/or the MMM complex (e.g., ELP-MRD fusion protein) binds to EGFR, ErbB2, ErbB3, ErbB4, CD20, insulin-like growth factor-I receptor, or prostate specific membrane antigen.

In one embodiment, the peptide sequence of the EGFR targeting (binding) MRD is VDNKFNKELEKAYNEIRNLPNLNGWQMTAFIASLVDDPSQSANLLAEAKKLNDAQAPK (SEQ ID NO:16). In one embodiment, the peptide sequence of the EGFR targeting MRD is VDNKFNKEMWIAWEEIRNLPNLNGWQMTAFIASLVDDPSQSANLLAEA KKLNDAQAPK (SEQ ID NO:17). In another embodiment, the peptide sequence of the ErbB2 targeting MRD is VDNKFNKEmRNAYWEIALLPNLNNQQKRAFIRSLYDDPSQSANLLAEAKKLNDAQAPK (SEQ ID NO:18).

In an additional embodiment an MRD and/or the MMM complex (e.g., ELP-MRD fusion protein) binds a serum protein. Examples of serum proteins bound by an MRD and/or the MMM complex (e.g., ELP-MRD fusion protein) according to this embodiment include, but are not limited to, serum albumin (e.g., human serum albumin (HSA)), thyroxin-binding protein, transferrin, fibrinogen, an immunoglobulin (e.g., IgG, IgE or IgM). Additional, serum proteins bound by an MRD and/or the MMM complex (e.g., ELP-MRD fusion protein) according to certain embodiments, of the invention include, but are not limited to, one or more of the serum proteins listed in WO 04/003019, EP 0368684, WO 91/01743, WO 01/45746 and WO 04/003019, WO 06/040153, and Harmsen et al., Vaccine, 23 (41); 4926-42 (2005), each of which is herein incorporated by reference.

Human serum albumin is a 585 amino acid polypeptide that functions as a carrier of endogenous and exogenous ligands. In one embodiment an MRD and/or the MMM complex (e.g., ELP-MRD fusion protein) comprises one or more amino acid sequences that confer an increased half-life in vivo to the MMM complex (e.g., ELP-MRD fusion protein). In particular embodiments, the amino acid sequences bind a human serum protein such as, human serum albumin. In particular embodiments, the binding of the MMM complex (e.g., ELP-MRD fusion protein) does not displace thyroxine from albumin. In additional embodiments, the binding of the MMM complex (e.g., ELP-MRD fusion protein) does not displace warfarin or digoxin from albumin.

Without being bound by theory, the binding of an MMM complex (e.g., an ELP-MRD fusion protein) to a carrier protein is believed to confer upon the MMM complex (e.g., ELP-MRD fusion protein) an improved pharmacodynamic profile that includes, but is not limited to, improved tumor targeting, tumor penetration, diffusion within the tumor, and enhanced therapeutic activity compared to the MMM complex (e.g., ELP-MRD fusion protein) in which the carrier protein binding sequence is missing (see, e.g., WO 01/45746, the contents of which are herein incorporated by reference).

In some embodiments, an MRD and/or the MMM complex (e.g., ELP-MRD fusion protein) has an affinity for serum albumin of greater than 50% bound under physiological conditions. In additional embodiments, an MRD and/or the MMM complex (e.g., ELP-MRD fusion protein) has an affinity for serum albumin of greater than 60%, 70%, 80%, 90%, or 95%) bound under physiological conditions are suitable. Generally, a MRD and/or the MMM complex (e.g., ELP-MRD fusion protein) that binds to a plasma protein (e.g., albumin) to a higher degree will have a longer half-life in the blood. It is envisioned however, that a range of plasma protein binding properties can be used to tailor the half-life of an MRD and/or the MMM complex (e.g., ELP-MRD fusion protein) to a suitable level. For example, binding should be strong enough to extend the half life, but not so strong that there is no free fraction of an MRD and/or the MMM complex (e.g., ELP-MRD fusion protein) available to exert a beneficial physiological effect. Additionally, it is envisioned that an MRD and/or the MMM complex (e.g., ELP-MRD fusion protein) having an intermediate affinity for a plasma protein such as albumin, is optionally chosen in the instances in which some degree of extravasation of an MRD and/or the MMM complex (e.g., ELP-MRD fusion protein) is desired.

In some embodiments, MRDs and/or the MMM complex (e.g., ELP-MRD fusion protein) comprise naturally occurring amino acid sequences that bind a plasma protein, such as serum albumin. In other embodiments, MRDs and/or the MMM complex (e.g., ELP-MRD fusion protein) comprise one or more non-naturally occurring amino acid sequences that bind a plasma protein, such as serum albumin. In some embodiments, the plasma protein binding MRD sequence is between about 10 and 30 or between about 10 and 20 amino acid residues. Examples of binding sequences include both linear and cyclic peptides, and combinations thereof.

In some embodiments, an MRD and/or the MMM complex (e.g., ELP-MRD fusion protein) binds serum albumin and comprises an amino acid sequence selected from: XXCCXXFCXAspWPXXXSC (SEQ ID NO:54), VCYXXXICF (SEQ ID NO:55) CYX1PGXC (SEQ ID NO:88) and AspXCLPXWGCLW (SEQ ID NO:89), where X is any amino acid and X1 is an amino acid residue selected from the group consisting of I, F, Y, and V. In one embodiment, an MRD and/or the MMM complex (e.g., ELP-MRD fusion protein) comprises the sequence: XCLPRXWGCLW (SEQ ID NO:90), where X is any amino acid.

In additional embodiments, an MRD and/or the MMM complex (e.g., ELP-MRD fusion protein) binds serum albumin and comprises an amino acid sequence selected from: DLCLRDWGCLW (SEQ ID NO:91); DICLPRWGCLW (SEQ ID NO:92); MEDICLPRWGCLWGD (SEQ ID NO: 114); QRLMEDICLPRWGCLWEDDE (SEQ ID NO:93) QGLIGDICLPRWGCLWGRSV (SEQ ID NO:94); QGLIGDICLPRWGCLWGRSVK (SEQ ID NO:95) EDICLPRWGCLWEDD (SEQ ID NO:96) RLMEDICLPRWGCLWEDD (SEQ ID NO:97); MEDICLPRWGCLWEDD (SEQ ID NO:98) MEDICLPRWGCLWED (SEQ ID NO:99) RLMEDICLARWGCLWEDD (SEQ ID NO:100) EVRSFCTRWPAEKSCKPLRG (SEQ ID NO:106) RAPESFVCYWETICFERSEQ (SEQ ID NO:107), and EMCYFPGICWM (SEQ ID NO:108). Members of this group are believed to confer binding ability of an MRD and/or the MMM complex (e.g., ELP-MRD fusion protein) across several species of serum albumin (e.g., human and rat).

In another embodiment, an MRD and/or the MMM complex (e.g., ELP-MRD fusion protein) binds human serum albumin and competes for binding of human serum albumin in an in vitro assay with peptide ligands having the general formulae: DXCLPXWGCLW (SEQ ID NO:109); FCXDWPXXXSC (SEQ ID NO:110); VCYXXXICF (SEQ ID NO:111); or CYX.₁PGXCX (SEQ ID NO:112) where Xaa is an amino acid, x and z are preferably 4 or 5, and X1 is a member selected from: the group consisting of: I, F, Y, and V Xaa1 is a member selected from the group consisting of: I, F, Y, and V. In an additional embodiment, an MRD and/or the MMM complex (e.g., ELP-MRD fusion protein) of the present invention bind human serum albumin and contains a sequence selected from QRLMEDICLPRWGCLWEDDF (SEQ ID NO:113) DICLPRWGCLWED (SEQ ID NO:115); IEDICLPRWGCLWE (SEQ ID NO:116); DICLPRWGCLW (SEQ ID NO:117); DICLPRWGCL (SEQ ID NO:118). Additional albumin binding MRD sequences that can be included in the MMM complex (e.g., ELP-MRD fusion protein) of the invention include one or more sequences corresponding to SEQ ID NOS: 2-491 of U.S. application publication number 2007/0202045, which is herein incorporated by reference. In additional embodiments, an MRD, antibody variable domain fragment and/or the MMM complex (e.g., ELP-MRD fusion protein) of the invention competes with any of the peptide ligands described or referred to above for binding with human serum albumin.

Methods for determining the affinity of candidate binding molecules (e.g., candidate albumin binding molecules) to a target molecule of interest (e.g., albumin) are known in the art and include, but are not limited to, affinity chromatography, size exclusion chromatography, equilibrium dialysis, and fluorescent probe displacement cases where the solubility of the binding groups alone is limited, it may be desirable to assess the relative affinity by derivatizing the binding group with a solubilizing fragment (e.g., Gd-DTPA). For example, HSA binding can be assessed by equilibrium dialysis or ultrafiltration using 4.5% weight/volume HSA in a buffer (pH 7.4) or by fluorescent probe displacement in which a fluorescent probe that fluoresces when bound to HSA is used. Affinity can be assessed by determining if the fluorescent probe is displaced from the binding site on HSA by the albumin binding moiety. A decrease in fluorescence indicates that the albumin binding moiety displaced the probe and the resulting data can be fit to obtain an inhibition equilibrium constant, Ki which reflects the affinity of the binding group for a given probe's binding site.

In some embodiments, MRDs are affibodies. Affibodies represent a class of affinity proteins based on a 58-amino acid residue protein domain derived from one of the IgG-binding domains of staphylococcal protein A. This three helix bundle domain has been used as a scaffold for the construction of combinatorial phagemid libraries, from which affibody variants that bind a desired target molecule, such as one or more of the targets disclosed herein, can routinely be selected using phage display technology (see, e.g., Nord et al., Nat. Biotechnol. 15:772-7 (1997), and Ronmark et al., Eur. J. Biochem., 269:2647-2655 (2002)). Further details of affibodies and methods of producing affibodies are provided by reference to U.S. Pat. No. 5,831,012, which is herein incorporated by reference.

In other embodiments, an MRD of the invention (e.g., an MRD on an MRD-ELP fusion protein) contains one or more amino acid residues or sequences of amino acid residues (including derivatives, analogs, and mimetics thereof) that are preferentially targeted by chemistries or other processes that covalently or non-covalently link a molecular entity to the MRD, as compared to, the MRD without the preferentially targeted sequences or the ELP component of the MRD-ELP fusion protein. For example, in some embodiments, the amino acid sequence of the MRD contains one or more residues having a reactive side chain (e.g., cysteine or lysine) that allows for selective or preferential linkage of the MRD to cytotoxic agents (e.g., drug and prodrug conjugates, toxins, and bioactive ligands) or imaging agents. MMM complexes containing such cytotoxic agent conjugates are referred to herein as MMM-Drug complexes or MMM-cytotoxic agent conjugates.

In other embodiments, the MRDs comprise one or more amino acid residues or sequences of amino acid residues (including derivatives, analogs, and mimetics thereof), that are preferentially targeted by chemistries or other processes that covalently or non-covalently link a molecular entity to the MRD, as compared to the ELP component of the MRD-ELP fusion protein. For example, in some embodiments, the amino acid sequence of the MRD contains one or more residues having reactive side chains (e.g., cysteine or lysine) that allow for selective or preferential linkage of the MRD to drug conjugates, imaging agents or bioactive ligands. The use of these “linking” MRDs to arm an MRD-comprising ELP with a “payload” overcomes many of the issues associated with antibody destabilization and reduction in antibody activity that have frequently been observed using conventional methods for generating immunotoxins. The “payload” component of an MRD-ELP fusion protein complex of the invention can be any composition that confers a beneficial therapeutic, diagnostic, or prognostic effect, or that provide an advantage in manufacturing, purifying or formulating an MRD-ELP fusion protein. In some embodiments, the payload is a chemotherapeutic drug, or a prodrug, such as, doxorubicin or a maytansinoid-like drug. In additional embodiments, the payload is another MRD, a toxin, a chemotherapeutic drug, a catalytic enzyme, a prodrug, a radioactive nuclide, a chelator (e.g., for the attachment of lanthanides).

In some embodiments, the MRD is conformationally constrained. In other embodiments, the MRD is not conformationally constrained. In some embodiments, the MRD contains one cysteine residue. The cysteine residue in the MRD can form an interchain bond (e.g., between cysteines within the same MRD, different peptide linked MRDs, and an MRD and a peptide linked ELP). In some embodiments, the MRD(s) participating in the interchain bond is/are associated with a single core target-binding domain. In other embodiments, the MRD(s) participating in the interchain bond is/are associated with multiple core target-binding domains. In an alternative embodiment, the cysteine residue in the MRD can form an interchain bond (e.g., between cysteines of non-peptide linked MRDs or an MRD and an ELP that are not linked by a peptide bind). In some embodiments, the MRD(s) associated with the interchain bond is/are associated with a single core target-binding domain (i.e., 2 MRDs located on different polypeptide chains form one or more interchain bonds and collectively form one target binding site). Thus, for example, the invention encompasses MMM complexes (e.g., ELP-MRD fusion proteins) wherein MRDs located on the carboxyl terminus of the heavy chain interact (e.g., via a disulfide bond) so as to form a single target binding site. In other embodiments, the MRD(s) associated with the interchain bond is/are associated with multiple core target-binding domains. Alternatively, as discussed herein, the MRD can contain one or more cysteine residues (or other residue having a reactive side chain (e.g., lysine)) that allows for selective or preferential linkage of the MRD to a cytotoxic agent.

In some embodiments, the MRD contains two cysteine residues outside the core target-binding domain. In some embodiments, the MRD contains two cysteine residues located within the core target-binding domain at each end of the target-binding domain. In some embodiments, a first cysteine is located near the terminus of the molecule (i.e. at the C-terminus of an MRD on the C-terminus of a linker or antibody chain or at the N-terminus of an MRD on the N-terminus of a linker or antibody chain). Thus, in some embodiments, a first cysteine is located within 1 amino acid, within 2 amino acids, within 3 amino acids, within 4 amino acids, within 5 amino acids, or within 6 amino acids of the terminus of the molecule. In some embodiments, a second cysteine is located near the MRD fusion location (i.e. at the N-terminus of an MRD on the C-terminus of a linker or ELP or at the C-terminus of an MRD on the N-terminus of a linker or ELP chain). Thus, in some embodiments, a second cysteine is located within 1 amino acid, within 2 amino acids, within 3 amino acids, within 4 amino acids, within 5 amino acids, within 10 amino acids, or within 15 amino acids from the MRD fusion.

In some embodiments, the MRD is capped with stable residues. In some embodiments, the MRD is disulfide capped. In some embodiments, the MRD does not contain cleavage sites.

In some embodiments, the MRD has been selected to not contain known potential human T-cell epitopes.

In some particular embodiments, the MRD has a particular hydrophobicity. For example, the hydrophobicity of MRDs can be compared on the basis of retention times determined using hydrophobic interaction chromatography or reverse phase liquid chromatography.

The MRD target can be any molecule that it is desirable for an MRD-ELP fusion protein to interact with. For example, the MRD target can be a soluble factor or a transmembrane protein, such as a cell surface receptor. The MRD target can also be an extracellular component or an intracellular component. In certain non-exclusive embodiments, the MRD target is a factor that regulates cell proliferation, differentiation, or survival. In other nonexclusive embodiments, the MRD target is a cytokine. In another nonexclusive embodiment, the MRD target is a factor that regulates angiogenesis. In another nonexclusive embodiment, the MRD target is a factor that regulates cellular adhesion and/or cell-cell interaction. In certain non-exclusive embodiments, the MRD target is a cell signaling molecule. In another nonexclusive embodiment, the MRD target is a factor that regulates one or more immune responses, such as, autoimmunity, inflammation and immune responses against cancer cells. In another nonexclusive embodiment, the MRD target is a factor that regulates cellular adhesion and/or cell-cell interaction. In an additional nonexclusive embodiment, the MRD target is a cell signaling molecule. In another embodiment, an MRD can bind a target that is itself an MRD. The ability of MRDs to bind a target and block, increase, or interfere with the biological activity of the MRD target can be determined using or routinely modifying assays, bioassays, and/or animal models known in the art for evaluating such activity.

The MRDs are able to bind their respective target when the MRDs are attached to an ELP. In some embodiments, the MRD is able to bind its target when not attached to an ELP. In some embodiments, the MRD is a target agonist. In other embodiments, the MRD is a target antagonist. In certain embodiments, the MRD can be used to localize an MMM complex (e.g., an ELP-MRD fusion protein) to an area where the MRD target is located.

B. Methods of Identifying, Making, and Testing MRDs and/or the MMM Complex (e.g., ELP-MRD Fusion Protein) MMM Complexes that Bind a Target of Interest

The sequence of an MRD can be determined several ways. For example, MRD sequences can be derived from natural ligands or known sequences that bind to a specific target-binding site. Additionally, phage display technologies have emerged as a powerful method in identifying peptides which bind to target receptors and ligands. In peptide phage display libraries, naturally occurring and non-naturally occurring (e.g., random peptide) sequences can be displayed by fusion with coat proteins of filamentous phage. The methods for elucidating binding sites on polypeptides using phage display vectors has been previously described, in particular in WO 94/18221, which is herein incorporated by reference. The methods generally involve the use of a filamentous phage (phagemid) surface expression vector system for cloning and expressing polypeptides that bind to the pre-selected target site of interest.

Methods for preparing MRDs include the use of phage display vectors for their particular advantage of providing a means to screen a very large population of expressed display proteins and thereby locate one or more specific clones that code for a desired target binding reactivity. The ability of the polypeptides encoded by the clones to bind a target and/or alter the biological activity of the target can be determined using or routinely modifying assays and other methodologies described herein or otherwise known in the art.

For example, phage display technology can be used to identify and improve the binding properties of MRDs. See, for example, Scott et al., Science 249: 386 (1990); Devlin et al., Science 249: 404 (1990); U.S. Pat. Nos. 5,223,409, 5,733,731, 5,498,530, 5,432,018, 5,338,665, 5,922,545; WO 96/40987, and WO 98/15833, which are herein incorporated by reference. In peptide phage display libraries, natural and/or non-naturally occurring peptide sequences can be displayed by fusion with coat proteins of filamentous phage. The displayed peptides can be affinity-eluted against a target of interest if desired. The retained phage can be enriched by successive rounds of affinity purification and repropagation. The best binding peptides can be sequenced to identify key residues within one or more structurally related families of peptides. See, e.g., Cwirla et al., Science 276: 1696-9 (1997), in which two distinct families were identified. The peptide sequences may also suggest which residues can be safely replaced by alanine scanning or by mutagenesis at the DNA level. Mutagenesis libraries can be created and screened to further optimize the sequence of the best binders. Lowman, Ann. Rev. Biophys. Biomol. Struct. 26: 401 24 (1997).

Structural analysis of protein-protein interaction may also be used to suggest peptides that mimic the binding activity of large protein ligands. In such an analysis, the crystal structure may suggest the identity and relative orientation of critical residues of the large protein ligand, from which a peptide such as an MRD can be designed. See, e.g., Takasaki et al., Nature Biotech 15: 1266 70 (1997). These analytical methods may also be used to investigate the interaction between a target and an MRD selected by phage display, which can suggest further modification of MRDs to increase binding affinity.

Other methods known in the art can be used to identify MRDs. For example, a peptide library can be fused to the carboxyl terminus of the lac repressor and expressed in E. coli. Another E. coli-based method allows display on the cell's outer membrane by fusion with a peptidoglycan-associated lipoprotein (PAL). These and related methods are collectively referred to as “E. coli display.” In another method, translation of random RNA is halted prior to ribosome release, resulting in a library of polypeptides with their associated RNA still attached. This and related methods are collectively referred to as “ribosome display.” Other known methods employ chemical linkage of peptides to RNA. See, for example, Roberts and Szostak, Proc. Natl. Acad. Sci. USA, 94: 12297 303 (1997). This and related methods are collectively referred to as “RNA-peptide screening, RNA display and mRNA display.” Chemically derived peptide libraries have been developed in which peptides are immobilized on stable, non-biological materials, such as polyethylene rods or solvent-permeable resins. Another chemically derived peptide library uses photolithography to scan peptides immobilized on glass slides. These and related methods are collectively referred to as “chemical-peptide screening.” Chemical-peptide screening can be advantageous in that it allows use of D-amino acids and other unnatural analogues, as well as non-peptide elements. Both biological and chemical methods are reviewed in Wells and Lowman, Curr. Opin. Biotechnol., 3: 355 62 (1992). Furthermore, constrained libraries, linear libraries, and/or focused libraries (comprised of structurally related domains that share significant primary sequence homology) can be used to identify, characterize, and modify MRDs.

An improved MRD that binds a desired target can also be prepared based on a known MRD sequence. For example, at least 1, 2, 3, 4, 5, or more amino acid mutations (e.g., conservative or non-conservative substitutions), deletions or insertions can be introduced into a known MRD sequence and the resulting MRD can be screened for binding to the desired target and biological activity, such as the ability to antagonize target biological activity or to agonize target biological activity. In another embodiment, the sites selected for modification are affinity matured using phage display techniques known in the art. See, e.g., Lowman, Ann. Rev. Biophys. Biomol. Struct. 26:401-4 24 (1997). Any technique for mutagenesis known in the art can be used to modify nucleotide(s) in a DNA sequence, for purposes of making amino acid addition(s), substitution(s) or deletion(s) in the MRD and or MMM complex (e.g., ELP-MRD fusion protein) sequence, or for creating/deleting restriction sites and sequences coding for desired amino acids (e.g., cysteine) or sequence of amino acids, to facilitate further manipulations of the MMM complexes of the invention. Such techniques include, but are not limited to, chemical mutagenesis, in vitro site-directed mutagenesis (Kunkel, Proc. Natl. Acad. Sci. USA 82:488 (1985); Hutchinson et al., J. Biol. Chem. 253:6551 (1978)), oligonucleotide-directed mutagenesis (Smith, Ann. Rev. Genet. 19:423-463 (1985); Hill et al., Methods Enzymol. 155:558-568 (1987)), PCR-based overlap extension (Ho et al., Gene 77:51-59 (1989)), PCR-based megaprimer mutagenesis (Sarkar et al., Biotechniques 8:404-407 (1990)), etc. Affinity maturation strategies can be used to generate high affinity MRDs that can be used in the MMM complex (e.g., ELP-MRD fusion protein) described herein.

Additionally, MRDs can be identified based on their effects in assays that measure particular pathways or activities. For example, assays that measure signaling pathways (e.g., phosphorylation studies or multimerization), ion channel fluxes, intracellular cAMP levels, cellular activities such as migration, adherence, proliferation, or apoptosis, and viral entry, replication, budding, or integration can be used to identify, characterize, and improve MRDs.

The ability of an MRD and/or the MMM complex (e.g., ELP-MRD fusion protein) to bind to a target and to block, increase, or interfere with the biological activity of an MRD and/or the MMM complex (e.g., ELP-MRD fusion protein) target can be determined using or routinely modifying assays, bioassays, and/or animal models known in the art for evaluating such activity.

Variants and derivatives of MRDs that retain the ability to bind the target are included within the scope of the present invention. Included within variants are insertional, deletional, and substitutional variants, as well as variants that include MRDs presented herein with additional amino acids at the N- and/or C-terminus, including from about 0 to 50, 0 to 40, 0 to 30, 0 to 20 amino acids and the like. It is understood that a particular MRD of the present invention can be modified to contain 1, 2, or all 3 types of variants. Insertional and substitutional variants may contain natural amino acids, unconventional amino acids, or both. In some embodiments, an MRD contains a sequence with no more than 1, 2, 3, 4, 5, 6, 7, 8, 9, 10, 15, or 20 amino acid differences when compared to an MRD sequence described herein. In some embodiments, the amino acid differences are substitutions. These substitutions can be conservative or non-conservative in nature and can include unconventional or non-natural amino acids. In other embodiments, an MRD contains a sequence that competitively inhibits the ability of an MRD-containing sequence described herein to bind with a target molecule. The ability of an MRD to competitively inhibit another MRD-containing sequence can be determined using techniques known in the art, including ELISA and BIAcore analysis.

The MMM complexes, such as ELP-MRD fusion proteins, used according to the methods of the invention also include derivatives of MMM complexes described herein that are modified, e.g., by the covalent attachment of any type of molecule to an MRD such that covalent attachment does not prevent an MRD from specifically binding to its target. For example, MRD derivatives include MRDs that have been modified, e.g., by glycosylation, acetylation, pegylation, phosphorylation, amidation, or derivatization by known protecting/blocking groups. Any of numerous chemical modifications can be carried out by known techniques, including, but not limited to acetylation, formylation, etc. Additionally, the derivative may contain one or more non-classical amino acids.

MRDs can be synthesized with covalently attached molecules that are not amino acids but aid in the purification, identification, and/or tracking of an MRD in vitro or in vivo. (e.g., biotin for reacting with avidin or avidin-labeled molecules).

The ability of an MRD to bind its target can be assessed using any technique that assesses molecular interaction. For example, MRD-target interaction can be assayed as described in the Examples below or alternatively, using in vitro or in vivo binding assays such as western blots, radioimmunoassays, ELISA (enzyme linked immunosorbent assay), “sandwich” immunoassays, immunoprecipitation assays, fluorescent immunoassays, protein A immunoassays, and immunohistochemistry (IHC). Assays evaluating the ability of an MRD to functionally affect it's target (e.g., assays to measure signaling, proliferation, migration etc.) can also be used to indirectly assess MRD-target interaction.

An improved MRD that has a particular half-life in vivo can also be prepared based on a known MRD sequence. For example, at least 1, 2, 3, 4, 5, or more amino acid mutations (e.g., conservative or non-conservative substitutions), deletions or insertions can be introduced into a known MRD sequence and the resulting MRD can be screened for increased half-life. Thus, variants and derivatives of the MRDs that retain the ability to bind the target and have an increased half-life can be included in MMM complexes (e.g., ELP-MRD fusion proteins). Thus, in some embodiments, an MRD in an MMM complex (e.g., an ELP-MRD fusion protein) ELP-MRD fusion protein has a half-life of at least about 5, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 110, at least about 120, at least about 130, at least about 140, or at least about 150 hours. In some embodiments, an MRD in an MMM complex (e.g., an ELP-MRD fusion protein) ELP-MRD fusion protein has a half-life of at least about 5, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 110, at least about 120, at least about 130, at least about 140, or at least about 150 hours.

Once the sequence of an MRD has been elucidated, the peptides can be prepared by any of the methods known in the art. For example, an MRD peptides can be chemically synthesized and operably attached to the ELP or can be synthesized using recombinant technology. For example, MRDs can be synthesized in solution or on a solid support using known techniques. Various automatic synthesizers are commercially available and can be used in accordance with known protocols. See, for example, Tam et al., J. Am. Chem. Soc., 105:6442 (1983); Merrifield, Science 232:341-347 (1986); Barany and Merrifield, The Peptides, Gross and Meienhofer, eds, Academic Press, New York, 1-284; Barany et al., Int. J. Pep. Protein Res., 30:705 739 (1987); and U.S. Pat. No. 5,424,398, which are herein incorporated by reference.

### II. Elastin-Like Peptides (ELPs)

The ELP component of the MMM complex (e.g., ELP-MRD fusion protein) of the invention generally contain repeats of structural units of from about three to about twenty amino acids. The length of the individual structural units, in a particular ELP component, can vary or can be uniform. In some embodiments, the ELP component is constructed of a polytetra-, polypenta-, polyhexa-, polyhepta-, polyocta, and/or polynonapeptide motif of repeating structural units.

In some embodiments, one or more ELP component(s) of an MMM complex (e.g., an ELP-MRD fusion protein) comprises tandem repeating units of the pentapeptide sequence VPGXG (SEQ ID NO:119), where X (i.e. the “guest residue”) is any natural or non-natural amino acid residue, and wherein X optionally varies among repeats units. In some embodiments, X is a member selected from: A, R, N, D, C, E, Q, G, H, I, L, K, M, F, S, T, W, Y and V. In additional embodiments, X is a natural amino acid other than proline or cysteine. In further embodiments, at least one of the guest residues is an amino acid selected from the group consisting of: V, I, L, A, G, and W.

In additional embodiments, one or more guest residues in an ELP MRD component is a non-classical (non-genetically encoded) amino acid. Examples of non-classical amino acids include, but are not limited to: D-isomers of the common amino acids, 2,4-diaminobutyric acid, alpha-amino isobutyric acid, A-aminobutyric acid, Abu, 2-amino butyric acid, gamma-Abu, epsilon-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, beta-alanine, fluoro-amino acids, designer amino acids such as beta-methyl amino acids, C alpha-methyl amino acids, and N alpha-methyl amino acids.

In additional embodiments, the percentage of VPGXG (SEQ ID NO:119) pentapeptide units in an ELP component of the MMM complex (e.g., ELP-MRD fusion protein) (which can comprise structural units other than VPGXG (SEQ ID NO:119)) is greater than about 75%, about 85%, or about 95% of the ELP component. In further embodiments, the percentage of VPGXG (SEQ ID NO:119) pentapeptide units in at least 2 ELP components of an MMM complex (e.g., an ELP-MRD fusion protein) is greater than about 75%, about 85%, or about 95%. In some embodiments, ELP components of an MMM complex (e.g., an ELP-MRD fusion protein) contain motifs having a 5 to 15-unit repeat (e.g., about 10-unit repeat) of the VPGXG (SEQ ID NO:119)) pentapeptide, with the guest residue X varying among at least 2 or at least 3 of the units. This repeat motif may itself be repeated, for example, from about 5 to about 12 times, such as about 8 to 10 times, to create an exemplary ELP component of the MMM complex (e.g., ELP-MRD fusion protein).

In particular embodiments, the guest residue composition of an ELP component of the MMM complex (e.g., ELP-MRD fusion protein) is selected in order to retain or achieve a desired inverse phase transition property. In some embodiments, an MRD comprises 3, 5, 7, or 9 or 10 pentapeptide VPGXG (SEQ ID NO:119) repeats where the guest residues are V, G, or A. In some embodiments, an MRD contains 3-5, 3-10, or 3-15 pentapeptide VPGXG (SEQ ID NO:119) repeats where the guest residues are predominantly V, G, or A. In particular embodiments, the guest residues of the ELP contents of an MMM complex (e.g., an ELP-MRD fusion protein) are V, G, and A in a 5:3:2 molar ratio. In some embodiments, an MRD comprises 3, 5, 7, or 9 or 10 pentapeptide VPGXG (SEQ ID NO:119) repeats where the guest residues are V, G, A or C. In additional embodiments, the guest residues of the ELP contents of an MMM complex (e.g., an ELP-MRD fusion protein) are V, G, A and C in a 4:3:2:1 molar ratio.

In additional embodiments, an MMM complex (e.g., an ELP-MRD fusion protein) comprises tandem repeating units of a pentapeptide sequence selected from the group consisting of VPGXG (SEQ ID NO:119), IPGXG (SEQ ID NO:120), and LPGXG (SEQ ID NO:121), or a combination thereof, where X is as defined above.

In additional embodiments, one or more ELP components of an MMM complex (e.g., an ELP-MRD fusion protein) contains one or more structural units selected from the group consisting of: VPGG (SEQ ID NO:122); IPGG (SEQ ID NO:123); AVGVP (SEQ ID NO:124); IPGXG (SEQ ID NO:125), IPGVG (SEQ ID NO:126); LPGXG (SEQ ID NO:127), LPGVG (SEQ ID NO:128); VAPGVG (SEQ ID NO:129); GVGVPGVG (SEQ ID NO:130); VPGFGVGAG (SEQ ID NO:131); and VPGVGVPGG (SEQ ID NO:132), wherein X is any natural or non-natural amino acid residue, and wherein X optionally varies among repeats units. In some embodiments, and ELP component of an MMM complex (e.g., an ELP-MRD fusion protein) is made up of one of the above structural repeats. In additional embodiments, 2 or more of the above structural repeats are used in combination to form an ELP component of an MMM complex (e.g., an ELP-MRD fusion protein). In another embodiment, an ELP component is formed entirely (or almost entirely) of one or a combination of at least 2, 3 or 4 of the above structural units. In other embodiments, at least 75%, or at least 80%, or at least 90% of an ELP component is formed from one or a combination of the above structural units. In additional embodiments, 2 or more ELP components of an MMM complex (e.g., an ELP-MRD fusion protein) contain the same sequence.

ELP components of the invention may occur as repeating structural units, including tandem-repeating units, and/or in any combination with other components of the MMM complex (e.g., ELP-MRD fusion protein) that confer desirable properties to the MMM complex (e.g., ELP-MRD fusion protein). The structural units of the ELP components of the MMM complex (e.g., ELP-MRD fusion protein) can vary in size. In some embodiments, the MMM complex (e.g., ELP-MRD fusion protein) contains at least one ELP component that comprises from about 5 to about 15, or from about 10 to about 20 structural units. In additional embodiments, the MMM complex (e.g., ELP-MRD fusion protein) contains at least one ELP component that comprises from about 15 to about 150 structural units, from about 20 to about 100 structural units or from about 50 to about 500 structural units.

The number of ELP components of the MMM complex (e.g., ELP-MRD fusion protein) and their respective positions within the fusion protein, can vary among embodiments, of the invention. For example, ELP component can be placed at either or both termini of an MRD or other component of an MMM complex and/or interspersed within the MMM complex (e.g., ELP-MRD fusion protein).

In some embodiments, the ELP component, or in some cases therapeutic agent, has a size of less than about 65 kDa, or less than about 60 kDa, or less than about 55 kDa, or less than about 50 kDa, or less than about 40 kDa, or less than about 30 kDa, or less than about 25 kDa.

In additional embodiments, the ELP component(s) of the MMM complex (e.g., ELP-MRD fusion protein) has a molecular weight of less than 9 kDa. In further embodiment ELP component(s) of the MMM complex (e.g., ELP-MRD fusion protein) has a molecular weight of less than less than 8 kDa, 7 kDa, 6 kDa, 5 kDa, or 4 kDa. In further embodiments, the ELP component(s) of the MMM complex (e.g., ELP-MRD fusion protein) has a molecular weight of between 2-8 kDa, between 4-8 kDa, or between 4-7 kDa. In additional embodiments, the ELP component(s) of the MMM complex (e.g., ELP-MRD fusion protein) has a molecular weight of less than 9 kDa and the phase transition behavior of the MMM complex (e.g., ELP-MRD fusion protein) is distinct from the phase transition behavior of the phase transition protein(s). In further embodiments, the phase transition behavior of the MMM complex (e.g., ELP-MRD fusion protein) is distinct from the phase transition behavior of the ELP component(s) (i.e., phase transition protein(s)) of the MMM complex (e.g., ELP-MRD fusion protein) and the MMM complex (e.g., ELP-MRD fusion protein) has a molecular weight of less than less than 8 kDa, 7 kDa, 6 kDa, 5 kDa, or 4 kDa. In further embodiments, the MMM complex (e.g., ELP-MRD fusion protein) does not comprise oligomeric repeats of the pentapeptide Val-Pro-Gly-X-Gly, wherein X is any natural or non-natural amino acid residue.

In further embodiments, the ELP component(s) of the MMM complex (e.g., ELP-MRD fusion protein) has a molecular weight of at least 8 kDa, 9 kDa, 10 kDa, 15 kDa, 20 kDa, 30 kDa, 40 kDa, 50 kDa, 60 kDa, 70 kDa, or 750 kDa. In further embodiments, the ELP component(s) of the MMM complex (e.g., ELP-MRD fusion protein) has a molecular weight of between 9-75 kDa, between 9-60 kDa or 9-50 kDa. In additional embodiments, the ELP component(s) of the MMM complex (e.g., ELP-MRD fusion protein) has a molecular weight of at least 9 kDa and the phase transition behavior of the MMM complex (e.g., ELP-MRD fusion protein) is distinct from the Tt of the phase ELP(s) in the MMM complex (e.g., ELP-MRD fusion protein). In further embodiments, the phase transition behavior of the MMM complex (e.g., ELP-MRD fusion protein) is distinct from the phase transition behavior of the ELP component(s) of the MMM complex (e.g., ELP-MRD fusion protein) and said ELP component(s) has as a molecular weight of at least 8 kDa, 9 kDa, 10 kDa, 15 kDa, 20 kDa, 30 kDa, 40 kDa, 50 kDa, 60 kDa, 70 kDa, or 100 kDa.

In some embodiments, one or more ELP components and/or the MMM complex (e.g., ELP-MRD fusion protein) is capable of undergoing a reversible inverse phase transition. According to these embodiments, the ELP component and/or the MMM complex (e.g., ELP-MRD fusion protein) is structurally disordered, and soluble in water below Tt, but exhibits a sharp phase transition when the temperature is raised above the Tt, leading to aggregation and desolvation of the ELP component and/or the MMM complex (e.g., ELP-MRD fusion protein) displaying this phase transition profile are believed to have distinct advantages in protein recovery and purification compared to conventional protein-based therapeutics. In some embodiments, the ELP component and/or the MMM complex (e.g., ELP-MRD fusion protein) does not undergo a reversible inverse phase transition, at a biologically relevant Tt. In some embodiments, the ELP component and/or the MMM complex (e.g., ELP-MRD fusion protein) does not undergo a reversible inverse phase transition.

The structural units making up ELP-MRD components of the invention can be separated by one or more amino acid residues that do not eliminate the overall effect of the molecule MRD and/or the MMM complex (e.g., ELP-MRD fusion protein) (e.g., the ability to undergo phase transition or therapeutic activity).

According to some embodiments, the Tt of the ELPs and/or the MMM complex (e.g., ELP-MRD fusion protein) of the invention are tunable to achieve the phase transition properties desired for the corresponding MRD and/or MMM complex (e.g., ELP-MRD fusion protein). For example, in those situations where the ELP is composed of pentameric units corresponding to VPGXG (SEQ ID NO:119), IPGXG (SEQ ID NO:125), or LPGXG (SEQ ID NO:127), the Tt of MRDs and/or the MMM complex (e.g., ELP-MRD fusion protein) of the invention is a function of the hydrophobicity of the guest residues of the ELP. Thus, by varying the identity of the guest residue(s) and their mole fraction(s), ELPs can be synthesized that exhibit an inverse transition over a 0-100° C. range. Accordingly, the Tt at a given ELP length can be decreased by incorporating a larger fraction of hydrophobic guest residues in the ELP sequence. Examples of hydrophobic guest residues that can be incorporated at the guest residues to lower Tt include valine, leucine, isoleucine, phenylalanine, tryptophan tyrosine, and methionine. In contrast, Tt can be increased by incorporating residues, such as glutamic acid, cysteine, lysine, aspartate, alanine, asparagine, serine, threonine, glycine, arginine, glutamine, alanine, serine, threonine and glutamic acid.

According to some embodiments, the ELP components of an MMM complex (e.g., an ELP-MRD fusion protein) are selected or designed to provide a Tt ranging from about 10° to about 80° C., from about 35° to about 60° C., from about 38° to about 45° C. or from about 50° to about 65° C. In some embodiments, the Tt is greater than about 30° C., greater than about 40° C., greater than about 42° C., greater than about 45° C., greater than about 50° C. or greater than about 55° C. In some embodiments, the Tt of the MMM complex (e.g., ELP-MRD fusion protein) is above the body temperature of the subject or patient (e.g., >37° C.) thereby remaining soluble in vivo. In other embodiments, Tt is below the body temperature (e.g., <37° C.) to provide alternative advantages, such as in vivo formation of a drug depot for sustained release of therapeutic agent.

Additionally, the Tt of the MMM complex (e.g., ELP-MRD fusion protein) of the invention can be regulated by varying ELP length, as the Tt generally increases with decreasing MW. For polypeptides having a molecular weight >100 kDa the hydrophobicity scale developed by Urry et al. (WO 08/030,968, which is hereby incorporated by reference) is preferred for predicting the approximate Tt of a specific ELP sequence. However, in some embodiments, ELP component length can be kept relatively small, while maintaining a target Tt, by incorporating a larger fraction of hydrophobic guest residues (e.g., amino acid residues having hydrophobic side chains) in the ELP sequence.

As described in WO 08/030,968 and WO 09/158,704, which are hereby incorporated by reference, ELP-therapeutic fusion proteins have been demonstrated to exhibit significant half lives and to retain the biological activity of therapeutic component of the fusion protein. Therefore, according to some embodiments, MMM complexes (e.g., ELP-MRD fusion proteins) of the invention increase the half-life of an MRD component, antibody component, and/or therapeutic component of an MMM complex (e.g., an ELP-MRD fusion protein) (e.g., by greater than 10%, 20%, 30%, or 50%) compared to the half-life of the free (unconjugated or unfused) form of MRD component, antibody component, and/or therapeutic component.

ELP-MRD fusions comprise one or more ELP components that comprise or consist of structural peptide units or sequences that are related to, or derived from, the elastin precursor, and can collectively confer improvement in one or more of the following properties of an MRD compared to MRD alone: bioavailability, therapeutically effective dose, biological action, formulation compatibility, resistance to proteolysis, solubility, half-life, or other measure of persistence in the body subsequent to administration and/or rate of clearance from the body.

In some embodiments, an MMM complex (e.g., an ELP-MRD fusion protein) contains 2 or more ELPs having different Tt profiles over a range of different temperatures. For example, in one embodiment an ELP has a Tt below physiological or nearly physiological temperatures and conditions (e.g., 32°-42° C.) and another ELP in the MMM complex (e.g., ELP-MRD fusion protein) has a Tt that is above normal physiological conditions (e.g., greater than 47° C.). In another example, an ELP has a Tt below physiological or nearly physiological temperatures and conditions (e.g., 32°-42° C.) and another ELP in the MMM complex (e.g., ELP-MRD fusion protein) has a Tt at slightly above normal physiological conditions (e.g., 43°-47° C.). Thus in some embodiments, at least one ELP in an MMM complex (e.g., an ELP-MRD fusion protein) constitutes a hydrophobic component of the fusion protein and at least one ELP constitutes a hydrophilic component of the fusion protein under physiological or near physiological conditions. According to these embodiments, at temperatures below the Tt of both ELP components, the MMM complex (e.g., ELP-MRD fusion protein) and its respective hydrophilic and hydrophobic ELPs exist as soluble monomers, but raising the temperature above the Tt of hydrophobic MRD component causes a collapse of the hydrophobic MRD. The collapse of this hydrophobic ELP results in the formation of multimeric stellate micelles that contain a core composed of the desolvated, hydrophobic ELP and a astrals containing solvated hydrophilic ELP and other components of the MMM complex (e.g., ELP-MRD fusion protein). Further increases in temperatures that exceed the Tt of the hydrophilic ELP lead to desolvation of the hydrophilic block, and the collapse of the stellate micells to form polydisperse micron size aggregates.

Elastin repeat containing fusion proteins containing copolymeric blocks that exhibit distinct Tts have been constructed and have been shown to exhibit numerous potential applications in drug delivery and other applications. See, e.g., Meyer et al., Biomacromolecules 3:357-367 (2002); Dreher et al., J. Am. Chem. Soc. 130:687-694 (2008); Lee et al., Adv. Mater. 12:1105-1110 (2000); and Simnick et al., J. Am. Chem. Soc. 4(4):2217-2227 (2010), each of which is herein incorporated by reference.

### III. Additional Modular Components of MMM Complexes

A. Antibody Fragments and Domains

In some embodiments, ELP-MRD fusions of the invention comprise an antibody fragment or domain (e.g., ScFv, diabody, EP 404,097; WO 93/111161; and Holliger et al., Proc. Natl. Acad. Sci. USA, 90: 6444-6448 (1993)).

The antibody fragment or domain can be any fragment or domain of antibody. For example, an ELP-MRD fusion can contain an antibody fragment or domain that is an effector domain. An ELP-MRD fusion can also contain an antibody fragment or domain that is an antigen-binding fragment or domain.

i. Antibody Effector Fragments and Domains

Thus, in some embodiments, an MMM complex (e.g., an ELP-MRD fusion protein) MMM complex (e.g., an ELP-MRD fusion protein) comprises an immunoglobulin effector domain (e.g., an immunoglobulin Fc effector domain), or derivative of an immunoglobulin effector domain that confers one or more effector functions to the MMM complex (e.g., ELP-MRD fusion protein) and/or confers upon the MMM complex (e.g., ELP-MRD fusion protein) the ability to bind to one or more Fc receptors. For example, in some embodiments, an MRD contains an immunoglobulin effector domain that comprises one or more CH2 and/or CH3 domains of an antibody having effector function provided by the CH2 and CH3 domains. Other sequences in the MMM complex (e.g., ELP-MRD fusion protein) that provide an effector function and are encompassed by the invention will be clear to those skilled in the art and can routinely be chosen and designed into an MMM complex (e.g., an ELP-MRD fusion protein) of the invention on the basis of the desired effector function(s). See for example, WO 04/058820, WO 99/42077 and WO 05/017148, each of which is herein incorporated by reference.

In some embodiments, MMM complexes (e.g., ELP-MRD fusion proteins) are able to participate in immunological effector activities including, for example, antibody dependent cell mediated cytotoxicity (ADCC; e.g., via target binding on a cell surface and the engagement and induction of cytotoxic effector cells bearing appropriate Fc receptors, such as Natural Killer cells bearing FcR gamma III, under appropriate conditions) and/or complement fixation in complement dependent cytotoxicity (CDC; e.g., via target binding on a cell surface and the recruitment and activation of cytolytic proteins that are components of the blood complement cascade). For reviews of ADCC and CDC see, e.g., Carter, Nat. Rev. Canc. 1:118 (2001); Sulica et al., Int. Rev. Immunol. 20:371 (2001); Maloney et al., Semin. Oncol. 29:2 (2002); Sondel et al., Hematol Oncol. Clin. North Am 15(4):703-21 (2001); Maloney et al., Antican. Drugs 12 Supp1.2:1-4 (2001). Hence, in some embodiments, an MMM complex (e.g., an ELP-MRD fusion protein) is capable of binding or specifically binding at least one target (e.g., a target present on an immune effector cell). Such MMM complexes (e.g., ELP-MRD fusion proteins) are believed to advantageously recruit desired immune effector cell function(s) to thereby elicit a desired therapeutic effect. It is well known that immune effector cells having different specialized immune functions. These cells can be identified or distinguished from one another on the basis of their differential expression of a wide variety of cell surface antigens, including many of the antigens described herein to which, in some embodiments, an MRD and/or the MMM complex (e.g., ELP-MRD fusion protein) of the invention can specifically bind. As noted herein, immune effector cells include any cell that is capable of directly mediating an activity that is a component of immune system function.

MRDs containing immunoglobulin sequences and derivatives of immunoglobulin sequences that confer the MMM complex (e.g., ELP-MRD fusion protein) with an increased half life are also encompassed by the invention. In some embodiments, an MRD of the MMM complex (e.g., ELP-MRD fusion protein) contains amino acid sequences corresponding to a constant domain (i.e., CH2 and/or CH3 domains) that confers increased half-life without any biologically significant effector function. Fusion proteins or derivatives with increased half-life can have a molecular weight of more than 50 kDa, the cut-off value for renal absorption

Immunoglobulin heavy chain constant region polypeptides include, by way of example, CH2/CH3 constant region polypeptides. The CH2/CH3 constant region polypeptides can be derived, separately or together, from, for example, human IgGs, human IgAs, and/or human IgE. The CH2/CH3 constant region polypeptides can be derived from naturally and/or non-naturally occurring immunoglobulin heavy chain constant region polypeptides.

Accordingly, in some embodiments, one or more MRDs of an ELP-MRD fusion of the invention confers upon the MMM complex (e.g., ELP-MRD fusion protein) a biochemical characteristic of an immunoglobulin that includes but is not limited to an activity selected from: the ability to confer one or more effector functions, the ability to non-covalently dimerize, the ability to localize at the site of a tumor, and an increased serum half-life when compared to the MMM complex (e.g., ELP-MRD fusion protein) in which said one or more MRDS have been deleted. In some embodiments, an ELP-MRD fusion contains an immunoglobulin effector domain or half-life influencing domain that corresponds to an immunoglobulin domain or fragment in which at least a fraction of one or more of the constant region domains has been altered so as to provide desired biochemical characteristics such as reduced or increased effector functions, the ability to non-covalently dimerize, increased ability to localize at the site of a tumor, reduced serum half-life, or increased serum half-life when compared with an immunoglobulin fragment having the corresponding unaltered immunoglobulin sequence. These alterations of the constant region domains can be amino acid substitutions, insertions, or deletions.

“Antibody-dependent cell-mediated cytotoxicity” or “ADCC” refers to a form of cytotoxicity in which secreted Ig bound onto Fc receptors (FcRs) expressed on certain cytotoxic cells (e.g., Natural Killer (NK) cells, neutrophils, and macrophages) enables these cytotoxic effector cells to bind specifically to an antigen-bearing target cell and subsequently kill the target cell with cytotoxins. Specific high-affinity IgG antibodies directed to the surface of target cells “arm” the cytotoxic cells and are required for such killing. Lysis of the target cell is extracellular, requires direct cell-to-cell contact or localization of the cytotoxic cells to the target cells or tissue, and does not involve complement.

As used herein, the term “enhances ADCC” (e.g., referring to cells) is intended to include any measurable increase in cell lysis when contacted with a variant MRD-ELP fusion protein as compared to the cell killing of the same cell in contact with a MRD-ELP fusion protein that has not been so modified in a way that alters ADCC in the presence of effector cells (for example, at a ratio of target cells:effector cells of 1:50), e.g., an increase in cell lysis by at least about 10%, 20%, 30%, 40%, 50%, 60%, 70%, 80%, 90%, 100%, 150%, 200%, 250%, 300%, or 325%.

In some embodiments, an MMM complex (e.g., an ELP-MRD fusion protein) contains an amino acid sequence of an immunoglobulin effector domain or a derivative of an immunoglobulin effector domain that confers antibody dependent cellular cytotoxicity (ADCC) to the MMM complex (e.g., ELP-MRD fusion protein). In additional embodiments, an MMM complex (e.g., an ELP-MRD fusion protein) contains a sequence of an immunoglobulin effector domain that has been modified to increase ADCC (see, e.g., Bruhns et al., Blood 113:3716-3725 (2009); Shields et al., J. Biol. Chem. 276:6591-6604 (2001); Lazar et al., Proc. Natl. Acad. Sci. USA 103:4005-4010 (2006); Stavenhagen et al., Cancer Res., 67:8882-8890 (2007); Horton et al., Cancer Res. 68:8049-8057 (2008); Zalevsky et al., Blood 113:3735-3743 (2009); Bruckheimer et al., Neoplasia 11:509-517 (2009); Allan et al., WO 06/020114; Strohl, Curr. Op. Biotechnol. 20:685-691 (2009); and Watkins et al., WO 04/074455, each of which is herein incorporated by reference). Examples of immunoglobulin fragment engineering modifications contained in an amino acid sequence in an MMM complex (e.g., an ELP-MRD fusion protein) that increases ADCC include immunoglobulin effector domain sequences having one or more modifications corresponding to: IgG1-S298A, E333A, K334A; IgG1-S239D, 1332E; IgG1-S239D, A330L, 1332E; IgG1-P2471, A339D or Q; IgG1-D280H, K2905 with or without S298D or V; IgG1-F243L, R292P, Y300L; IgG1-F243L, R292P, Y300L, P396L; and IgG1-F243L, R292P, Y300L, V305I, P396L; wherein the numbering of the residues in the Fc region is that of the EU index as in Kabat.

In other embodiments, an MMM complex (e.g., an ELP-MRD fusion protein) contains a sequence of an immunoglobulin effector domain that has been modified to decrease ADCC (see, e.g., Idusogie et al., J. Immunol. 166:2571-2575 (2001); Sazinsky et al., Proc. Natl. Acad. Sci. USA 105:20167-20172 (2008); Davis et al., J. Rheumatol. 34:2204-2210 (2007); Bolt et al., Eur. J. Immunol. 23:403-411 (1993); Alegre et al., Transplantation 57:1537-1543 (1994); Xu et al., Cell Immunol. 200:16-26 (2000); Cole et al., Transplantation 68:563-571 (1999); Hutchins et al., Proc. Natl. Acad. Sci. USA 92:11980-11984 (1995); Reddy et al., J. Immunol. 164:1925-1933 (2000); Mueller et al., WO 97/11971; Bell et al., WO 07/106,585; Strohl, US 2007/0148167A1; McEarchern et al., Blood 109:1185-1192 (2007); Strohl, Curr. Op. Biotechnol. 20:685-691 (2009); and Kumagai et al., J. Clin. Pharmacol. 47:1489-1497 (2007), each of which is herein incorporated by reference). Examples of immunoglobulin fragment sequence engineering modifications contained in an amino acid sequence in an MMM complex (e.g., an ELP-MRD fusion protein) that decreases ADCC include immunoglobulin effector domain sequences having one or more modifications corresponding to: IgG1-K326W, E333S; IgG2-E333S; IgG1-N297A; IgG1-L234A, L235A; IgG2-V234A, G237A; IgG4-L235A, G237A, E318A; IgG4-S228P, L236E; IgG2-EU sequence 118-260; IgG4-EU sequence 261-447; IgG2-H268Q, V309L, A330S, A331S; IgG1-C220S, C226S, C229S, P238S; IgG1-C226S, C229S, E233P, L234V, L235A; or IgG1-L234F, L235E, P331S.

In additional embodiments, an MMM complex (e.g., an ELP-MRD fusion protein) contains an amino acid sequence of an immunoglobulin effector domain, or a derivative of an immunoglobulin effector domain, that confers antibody-dependent cell phagocytosis (ADCP) to the MMM complex (e.g., ELP-MRD fusion protein). In additional embodiments, an MMM complex (e.g., an ELP-MRD fusion protein) contains a sequence of an immunoglobulin effector domain that has been modified to increase antibody-dependent cell phagocytosis (ADCP); (see, e.g., Shields et al., J. Biol. Chem. 276:6591-6604 (2001); Lazar et al., Proc. Natl. Acad. Sci. USA 103:4005-4010 (2006); Stavenhagen et al., Cancer Res., 67:8882-8890 (2007); Richards et al., Mol. Cancer. Ther. 7:2517-2527 (2008); Horton et al., Cancer Res. 68:8049-8057 (2008), Zalevsky et al., Blood 113:3735-3743 (2009); Bruckheimer et al., Neoplasia 11:509-517 (2009); Allan et al., WO 06/020114; Strohl, Curr. Op. Biotechnol. 20:685-691 (2009); and Watkins et al., WO 04/074455, each of which is herein incorporated by reference). Examples of immunoglobulin fragment engineering modifications contained in an amino acid sequence in an MMM complex (e.g., an ELP-MRD fusion protein) that increases ADCP include immunoglobulin effector domain sequences having one or more modifications corresponding to: IgG1-S298A, E333A, K334A; IgG1-S239D, 1332E; IgG1-S239D, A330L, 1332E; IgG1-P2471, A339D or Q; IgG1-D280H, K290S with or without S298D or V; IgG1-F243L, R292P, Y300L; IgG1-F243L, R292P, Y300L, P396L; IgG1-F243L, R292P, Y300L, V3051, P396L; and IgG1-G236A, 5239D, 1332E.

In other embodiments, an MMM complex (e.g., an ELP-MRD fusion protein) contains a sequence of an immunoglobulin effector domain that has been modified to decrease ADCP (see, e.g., Sazinsky et al., Proc. Natl. Acad. Sci. USA 105:20167-20172 (2008); Davis et al., J. Rheumatol. 34:2204-2210 (2007); Bolt et al., Eur. J. Immunol. 23:403-411 (1993); Alegre et al., Transplantation 57:1537-1543 (1994); Xu et al., Cell Immunol. 200:16-20 (2000); Cole et al., Transplantation 68:563-571 (1999); Hutchins et al., Proc. Natl. Acad. Sci. USA 92:11980-11984 (1995); Reddy et al., J. Immunol. 164:1925-1933 (2000); Mueller et al., WO 97/11971; Bell et al., WO 07/106,585; Strohl et al., US 2007/0148167A1; McEarchern et al., Blood 109:1185-1192 (2007); Strohl, Curr. Op. Biotechnol. 20:685-691 (2009); and Kumagai et al., J. Clin. Pharmacol. 47:1489-1497 (2007), each of which is herein incorporated by reference). By way of example, MMM complexes (e.g., ELP-MRD fusion proteins) can contain an antibody fragment or domain that contains one or more of the following modifications that decrease ADCC: IgG1-N297A; IgG1-L234A, L235A; IgG2-V234A, G237A; IgG4-L235A, G237A, E318A; IgG4-S228P, L236E; IgG2 EU sequence 118-260; IgG4-EU sequence 261-447; IgG2-H268Q, V309L, A330S, A331S; IgG1-C220S, C226S, C229S, P238S; IgG1-C226S, C229S, E233P, L234V, L235A; and IgG1-L234F, L235E, P331S.

“Complement dependent cytotoxicity” and “CDC” refer to the lysing of a target cell in the presence of complement. The complement activation pathway is initiated by the binding of the first component of the complement system (C1q) to a molecule, an antibody for example, complexed with a cognate antigen. To assess complement activation, a CDC assay, e.g., as described in Gazzano-Santoro et al., J. Immunol. Methods 202:163 (1996), can be performed. In one embodiment, an Fc variant protein has enhanced CDC activity relative to a comparable molecule. In a specific embodiment, an Fc variant protein has CDC activity that is at least 2 fold, or at least 3 fold, or at least 5 fold, or at least 10 fold, or at least 50 fold, or at least 100 fold greater than that of a comparable molecule. In other embodiments, the Fc variant protein has both enhanced CDC activity and enhanced serum half-life relative to a comparable molecule.

In additional embodiments, an MMM complex (e.g., an ELP-MRD fusion protein) contains an amino acid sequence of an immunoglobulin effector domain, or a derivative of an immunoglobulin effector domain, that confers complement-dependent cytotoxicity (CDC) to the MMM complex (e.g., ELP-MRD fusion protein). In further embodiments, the MMM complex (e.g., ELP-MRD fusion protein) contains a sequence of an immunoglobulin effector domain that has been modified to increase complement-dependent cytotoxicity (CDC) (see, e.g., (see, e.g., Idusogie et al., J. Immunol. 166:2571-2575 (2001); Strohl, Curr. Op. Biotechnol. 20:685-691 (2009); and Natsume et al., Cancer Res. 68:3863-3872 (2008), each of which is herein incorporated by reference in its entirety). By way of example, MMM complexes (e.g., ELP-MRD fusion proteins) can contain a derivative amino acid sequence of an immunoglobulin effector domain that confers complement-dependent cytotoxicity (CDC) to the MMM complex (e.g., ELP-MRD fusion protein) and that contains one or more of the following modifications that increase CDC: IgG1-K326A, E333A; and IgG1-K326W, E333S, IgG2-E333S′.

In additional embodiments, the MMM complex (e.g., ELP-MRD fusion protein), contains an amino acid sequence of an immunoglobulin effector domain, or a derivative of an immunoglobulin effector domain, that confers the ability to bind Fc gamma RIIbFc Gamma receptor to the ELP-MRD fusion. In additional embodiments, an MMM complex (e.g., an ELP-MRD fusion protein) contains a sequence of an immunoglobulin effector domain that confers the ability to bind Fc gamma RIIbFc Gamma receptor to the MMM complex and that has been modified to increase inhibitory binding to Fc gamma RIIb Fc Gamma receptor (see, e.g., Chu et al., Mol. Immunol. 45:3926-3933 (2008)). By way of example, MMM complexes (e.g., ELP-MRD fusion proteins) can contain a derivative amino acid sequence of an immunoglobulin effector domain that confers the ability to bind Fc gamma RIIbFc Gamma receptor to the MMM complex (e.g., ELP-MRD fusion protein) and that contains one or both of the following modifications IgG1-S267E, L328F that increase binding to inhibitory Fc gamma RIIb Fc Gamma receptor. In other embodiments, an MMM complex (e.g., an ELP-MRD fusion protein) contains a sequence of an immunoglobulin effector domain that contains an amino acid sequence of an immunoglobulin effector domain, or a derivative of an immunoglobulin effector domain, that confers complement-dependent cytotoxicity (CDC) to the MMM complex, but that has been modified to decrease CDC (see, e.g., Int. Appl. publications WO 97/11971 and WO 07/106,585; U.S. Appl. Publication No. 2007/0148167A1; McEarchern et al., Blood 109:1185-1192 (2007); Hayden-Ledbetter et al., Clin. Cancer 15:2739-2746 (2009); Lazar et al., Proc. Natl. Acad. Sci. USA 103:4005-4010 (2006); Bruckheimer et al., Neoplasia 11:509-517 (2009); Strohl, Curr. Op. Biotechnol. 20:685-691 (2009); and Sazinsky et al., Proc. Natl. Acad. Sci. USA 105:20167-20172 (2008); each of which is herein incorporated by reference in its entirety). By way of example, MMM complexes (e.g., ELP-MRD fusion proteins) can contain an antibody fragment or domain that that confers complement-dependent cytotoxicity (CDC) to the MMM complex, but that contains one or more of the following modifications that decrease CDC: IgG1-S239D, A330L, 1332E; IgG2 EU sequence 118-260; IgG4-EU sequence 261-447; IgG2-H268Q, V309L, A330S, A331S; IgG1-C226S, C229S, E233P, L234V, L235A; IgG1-L234F, L235E, P331S; and IgG1-C226S, P230S.

The half-life of an IgG is mediated by its pH-dependent binding to the neonatal receptor FcRn. In certain embodiments, the MMM complex is an ELP-MRD fusion protein, contains an amino acid sequence of an immunoglobulin effector domain, or a derivative of an immunoglobulin effector domain, that confers the ability to bind neonatal receptor FcRn to the to the MMM complex (ELP-MRD fusion protein). In certain embodiments, an MMM complex (e.g., an ELP-MRD fusion protein) contains a sequence of an immunoglobulin FcRn binding domain that confers the ability to bind neonatal receptor FcRn and that has been modified to enhance binding to FcRn (see, e.g., Petkova et al., Int. Immunol. 18:1759-1769 (2006); Dall'Acqua et al., J. Immunol. 169:5171-5180 (2002); Oganesyan et al., Mol. Immunol. 46:1750-1755 (2009); Dall'Acqua et al., J. Biol. Chem. 281:23514-23524 (2006), Hinton et al., J. Immunol. 176:346-356 (2006); Datta-Mannan et al., Drug Metab. Dispos. 35:86-94 (2007); Datta-Mannan et al., J. Biol. Chem. 282:1709-1717 (2007); Int. Appl. publication WO 06/130834; Strohl, Curr. Op. Biotechnol. 20:685-691 (2009); and Yeung et al., J. Immunol. 182:7663-7671 (2009) each of which is herein incorporated by reference in its entirety).

In additional embodiments, the MMM complex (e.g., ELP-MRD fusion protein), contains a sequence of an immunoglobulin effector domain that confers the ability to bind neonatal receptor FcRn to the MMM complex and that has been modified to selectively bind FcRn at pH 6.0, but not pH 7.4. By way of example, MMM complexes (e.g., ELP-MRD fusion proteins) can contain an antibody fragment or domain that contains one or more of the following immunoglobulin effector domain modifications that increase half-life: IgG1-M252Y, S254T, T256E; IgG1-T250Q, M428L; IgG1-H433K, N434Y; IgG1-N434A; and IgG1-T307A, E380A, N434A.

In other embodiments, the MMM complex (e.g., ELP-MRD fusion protein), contains a sequence of an immunoglobulin effector domain onfers the ability to bind neonatal receptor FcRn to the MMM complex and that has been modified to decrease binding to FcRn (see, e.g., Petkova et al., Int. Immunol. 18:1759-1769 (2006); Datta-Mannan et al., Drug Metab. Dispos. 35:86-94 (2007); Datta-Mannan et al., J. Biol. Chem. 282:1709-1717 (2007); Strohl, Curr. Op. Biotechnol. 20:685-691 (2009); and Vaccaro et al., Nat. Biotechnol. 23:1283-1288 (2005), each of which is herein incorporated by reference in its entirety). By way of example, MMM complexes (e.g., ELP-MRD fusion proteins) can contain an antibody fragment or domain that confers the ability to bind neonatal receptor FcRn and that has been modified to contain one or more of the following modifications that decrease half-life: IgG1-M252Y, S254T, T256E; H433K, N434F, 436H; IgG1-I253A; and IgG1-P2571, N434H and D376V, N434H.

According to another embodiment, the MMM complex (e.g., ELP-MRD fusion protein), contains an amino acid sequence that confers an immunoglobulin effector function to the MMM complex and wherein the amino acid sequence has been modified to contain at least one substitution in its sequence corresponding to the Fc region position selected from the group consisting of: 238, 239, 246, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 332, 333, 334, 335, 337, 338, 340, 360, 373, 376, 378, 382, 388, 389, 398, 414, 416, 419, 430, 434, 435, 437, 438 and 439, wherein the numbering of the residues in the Fc region is according to the EU numbering system. In a specific embodiment, the MMM complex (e.g., ELP-MRD fusion protein) contains a sequence of an immunoglobulin effector domain wherein at least one residue corresponding to position 434 is a residue selected from the group consisting of A, W, Y, F and H. According to another embodiment, the MMM complex (e.g., ELP-MRD fusion protein), wherein the ELP contains a sequence of an immunoglobulin effector fragment derivative having the following respective substitutions S298A/E333A/K334A. In an additional embodiment, the MMM complex (e.g., ELP-MRD fusion protein) contains an immunoglobulin effector domain derivative having a substitution corresponding to K322A. In another embodiment, the MMM complex (e.g., ELP-MRD fusion protein), wherein the ELP contains a sequence of an immunoglobulin effector domain derivative having one or any combination of the following substitutions K246H, H268D, E283L, S324G, S239D and 1332E. According to yet another embodiment, the MMM complex is (e.g., ELP-MRD fusion protein), wherein the ELP contains a sequence of an immunoglobulin effector domain derivative having substitutions corresponding to D265A/N297A.

In certain embodiments, the MMM complex (e.g., ELP-MRD fusion protein), contains a sequence of an immunoglobulin effector domain that confers an immunoglobulin effector function to the MMM complex and that has been glyocoengineered or mutated to increase effector function using techniques known in the art. For example, the inactivation (through point mutations or other means) of a constant region domain sequence contained in an ELP-MRD of the invention may reduce Fc receptor binding of the circulating MMM complex (e.g., ELP-MRD fusion protein) thereby increasing tumor localization. In other cases it may be that constant region modifications consistent with the instant invention moderate complement binding and thus reduce the serum half life and nonspecific association of a conjugated cytotoxin. Yet other modifications of the constant region can be used to modify disulfide linkages or oligosaccharide moieties that allow for enhanced localization due to increased antigen specificity or antibody flexibility. The resulting physiological profile, bioavailability and other biochemical effects of the modifications, such as tumor localization, biodistribution and serum half-life, can easily be measured and quantified using well know immunological techniques without undue experimentation.

In certain embodiments, an immune effector cell comprises a cell surface receptor for an immunoglobulin or other peptide binding molecule, such as a receptor for an immunoglobulin constant region and including the class of receptors commonly referred to as “Fc receptors” (“FcR”s). A number of FcRs have been structurally and/or functionally characterized and are well known in the art, including FcR having specific abilities to interact with a restricted subset of immunoglobulin heavy chain isotypes, or that interact with Fc domains with varying affinities, and/or which can be expressed on restricted subsets of immune effector cells under certain conditions (e.g., Kijimoto-Ochichai et al., Cell Mol. Life. Sci. 59:648 (2002); Davis et al., Curr. Top. Microbiol. Immunol. 266:85 (2002); Pawankar, Curr. Opin. Allerg. Clinmmunol. 1:3 (2001); Radaev et al., Mol. Immunol. 38:1073 (2002); Wurzburg et al., Mol. Immunol. 38:1063 (2002); Sulica et al., Int. Rev. Immunol. 20:371 (2001); Underhill et al., Ann. Rev. Immunol. 20:825 (2002); Coggeshall, Curr. Dir. Autoimm. 5:1 (2002); Mimura et al., Adv. Exp. Med. Biol. 495:49 (2001); Baumann et al., Adv. Exp. Med. Biol. 495:219 (2001); Santoso et al., Ital. Heart J. 2:811 (2001); Novak et al., Curr. Opin. Immunol. 13:721 (2001); Fossati et al., Eur. J. Clin. Invest. 31:821 (2001)).

Cells that are capable of mediating ADCC are examples of immune effector cells. Other immune effector cells include Natural Killer cells, tumor-infiltrating T lymphocytes (TILs), cytotoxic T lymphocytes, and granulocytic cells such as cells that comprise allergic response mechanisms. Immune effector cells thus include, but are not limited to, cells of hematopoietic origin including cells at various stages of differentiation within myeloid and lymphoid lineages and which may (but need not) express one or more types of functional cell surface FcR, such as T lymphocytes, B lymphocytes, NK cells, monocytes, macrophages, dendritic cells, neutrophils, basophils, eosinophils, mast cells, platelets, erythrocytes, and precursors, progenitors (e.g., hematopoietic stem cells), as well as quiescent, activated, and mature forms of such cells. Other immune effector cells may include cells of non-hematopoietic origin that are capable of mediating immune functions, for example, endothelial cells, keratinocytes, fibroblasts, osteoclasts, epithelial cells, and other cells. Immune effector cells can also include cells that mediate cytotoxic or cytostatic events, or endocytic, phagocytic, or pinocytotic events, or that effect induction of apoptosis, or that effect microbial immunity or neutralization of microbial infection, or cells that mediate allergic, inflammatory, hypersensitivity and/or autoimmune reactions.

IL-12 is known to enhance cytolytic T-cell responses, promote the development of helper T cells, enhance the activity of natural killer (NK) cells, and induces the secretion of IFN-gamma in T and NK cells. IL-12 also increases many helper and effector cells that mediate apoptosis. Accordingly, in one embodiment, an MMM complex (e.g., an ELP-MRD fusion protein) that binds an effector component also contains an MRD or antibody fragment or domain that binds IL-12. In another embodiment, an MMM complex (e.g., an ELP-MRD fusion protein) contains IL-12 or a therapeutically active fragment or derivative thereof. In additional embodiments, the MMM complexes (e.g., ELP-MRD fusion proteins) that bind effector components are administered or co-administered with Interleukin-12 (IL-12). In another aspect of the invention, one or more of the above MMM complexes (e.g., ELP-MRD fusion proteins) that bind an effector component and IL-12 also contains an MRD(s) or antibody fragment or domain that binds CD20 and/or CD19. In further embodiments, a therapeutically effective amount of one of the above MMM complexes (e.g., ELP-MRD fusion proteins) is administered to a patient to treat a B-cell malignancy (e.g., B-cell non-Hodgkin's lymphoma (NHL)). In further embodiments, a therapeutically effective amount of one of the above MMM complexes (e.g., ELP-MRD fusion proteins) is administered to a patient to treat an autoimmune disease (e.g., rheumatoid arthritis or SLE).

ii. Antibody Binding Region Fragments and Domains

ELP-MRD fusion can also contain an antibody fragment or domain that is an antigen-binding fragment or domain that binds a specific antigen. In a specific embodiment, the antibody fragment is an ScFv. In another specific embodiment, the antibody fragment is a single domain antibody or a derivative of a single domain antibody. In another embodiment, the “antibody fragment” is an antibody target binding mimetic. Examples of a antibody target binding mimetics that may be contained in the MMM complexes of the invention include, but are not limited to, affibodies, affilins, affitins, anticalins, avimers, DARPins, Kunitz domain derived peptides, knottins and monobodies.

The antibody target of the MRD-ELP fusion protein (e.g., the target of the antigenic binding domain, ScFv, or single domain antibody) can be any molecule that it is desirable for a MRD-ELP fusion protein to interact with. For example, the antibody target can be a soluble factor or the antibody target can be a transmembrane protein, such as a cell surface receptor. The antibody target can also be an extracellular component or an intracellular component. In certain embodiments, the antibody target is a factor that regulates cell proliferation, differentiation, or survival. In other embodiments, the antibody target is a cytokine. In another nonexclusive embodiment, the antibody target is a factor that regulates angiogenesis. In another nonexclusive embodiment, the antibody target is a factor that regulates one or more immune responses, such as, autoimmunity, inflammation and immune responses against cancer cells. In another nonexclusive embodiment, the antibody target is a factor that regulates cellular adhesion and/or cell-cell interaction. In certain nonexclusive embodiments, the antibody target is a cell signaling molecule. The ability of an antibody to bind to a target and to block, increase, or interfere with the biological activity of the antibody target can be determined using or routinely modifying assays, bioassays, and/or animal models known in the art for evaluating such activity.

Thus, in some embodiments, an antibody fragment or domain in an MMM complex (e.g., an ELP-MRD fusion protein) binds a disease-related antigen. Antigens bound by MMM complexes of the invention can be an antigen characteristic of a particular cancer, and/or of a particular cell type (e.g., a hyperproliferative cell), and/or of a particular pathogen (e.g., a bacterial cell (e.g., tuberculosis, smallpox, anthrax)), a virus (e.g., HIV), a parasite (e.g., malaria, leichmaniasis), a fungal infection, a mold, a mycoplasm, a prion antigen, or an antigen associated with a disorder of the immune system.

In some embodiments, an MMM complex (e.g., an ELP-MRD fusion protein) contains an antibody fragment or domain that binds to an antigen or epitope that has been validated in an animal model or clinical setting.

In other embodiments, an MMM complex (e.g., an ELP-MRD fusion protein) contains an antibody fragment or domain that binds a cancer antigen.

An antibody fragment or domain in an MMM complex (e.g., an ELP-MRD fusion protein) and an MRD in the MMM complex (e.g., ELP-MRD fusion protein) can bind to the same or different targets or substrates.

In one embodiment, an antibody fragment or domain, or an MMM complex (e.g., an ELP-MRD fusion protein) comprising an antibody fragment or domain binds to a member selected from: PDGFRa, PDGFRb, PDGF-A, PDGF-B, PDGF-CC, PDGF-C, PDGF-D, VEGFR1, VEGFR2, VEGFR3, VEGFC, VEGFD, neuropilin 2 (NRP2), betacellulin, P1GF, RET (rearranged during transfection), TIE1, TIE2 (TEK), CAl25, CD3, CD4, CD7, CD10, CD13, CD25, CD32, CD32b, CD44, CD49e (integrin alpha 5), CD55, CD64, CD90 (THY1), CD133 (prominin 1), CD147, CD166, CD200, ALDH1, ESA, SHH, DHH, 1HH, patchedl (PTCH1), smoothened (SMO), WNT1, WNT2B, WNT3A, WNT4, WNT4A, WNT5A, WNT5B, WNT7B, WNT8A, WNT10A, WNT10B, WNT16B, LRP5, LRP6, FZD1, FZD2, FZD4, FZD5, FZD6, FZD7, FZD8, Notch, Notch1, Notch3, Notch4, DLL4, Jagged, Jagged1, Jagged2, Jagged3, TNFSF1 (TNFb, LTa), TNFRSF1A (TNFR1, p55, p60), TNFRSF1B (TNFR2), TNFSF6 (Fas Ligand), TNFRSF6 (Fas, CD95), TNFRSF6B (DcR3), TNFSF7 (CD27 Ligand, CD70), TNFRSF7 (CD27), TNFSF8 (CD30 Ligand), TNFRSF8 (CD30), TNFSF11 (RANKL), TNFRSF11A (RANK), TNFSF12 (TWEAK), TNFRSF12 (TWEAKR), TNFSF13 (APRIL), TNFSF13B (BLYS), TNFRSF13B (TACI), TNFRSF13C (BAFFR), TNFSF15 (TL1A), TNFRSF17 (BCMA), TNFRSF19L (RELT), TNFRSF19 (TROY), TNFRSF21 (DR6), TNFRSF25 (DR3), ANG1 (ANGPT1), ANG3 (ANGPTL1), ANG4 (ANGPT4), IL1a, IL1b, IL1R1, IL1R2, IL2, IL2R, IL5, IL5R, IL6R, IL8, IL8R, IL10, IL10R, IL12, IL12R, IL13, IL13R, IL15, IL15R, IL18, IL18R, IL19, IL19R, IL21R, IL23, IL23R, mif, XAG1, XAG3, REGIV, FGF1, FGF2, FGF3, FGF4, FGFR1, FGFR2, FGFR3, ALK, ALK1, ALK7, ALCAM, Artemin, Axl, TGFb, TGFb2, TGFb3, TGFBR1, IGFIIR, BMP2, BMP5, BMP6, BMPR1, GDF3, GDF8, GDF9, N-cadherin, E-cadherin, VE-cadherin, NCAM, L1CAM (CD171), ganglioside GM2, ganglioside GD2, calcitonin, PSGR, DCC, CDCP1, CXCR2, CXCR7, CCR3, CCR5, CCR7, CCR10, CXCL1, CXCL5, CXCL6, CXCL8, CXCL12, CCL3, CCL4, CCL5, CCL11, Claudin1, Claudin2, Claudin3, Claudin4, TMEFF2, neuregulin, MCSF, CSF, CSFR (fms), GCSF, GCSFR, BCAM, HPV, hCG, SR1F, PSA, FOLR2 (folate receptor beta), BRCA1, BRCA2, HLA-DR, ABCC3, ABCB5, HM1.24, LFA1, LYNX, S100A8, S100A9, SCF, Von Willebrand factor, Lewis Y6 receptor, Lewis Y, CA G250 (CA9), integrin avb3 (CNTO95), integrin avb5, activin B1 alpha, leukotriene B4 receptor (LTB4R), neurotensin NT receptor (NTR), 5T4 oncofetal antigen, Tenascin C, MMP, MMP2, MMPI, MMP9, MMP12, MMP14, MMP26, cathepsin G, cathepsin H, cathepsin L, SULF1, SULF2, MET, UPA, MHCl, MN(CA9), TAG-72, TM4SF1, Heparanse (HPSE), syndecan (SDC1), Ephrin B2, Ephrin B4, or relaxin2. MMM complexes (e.g., ELP-MRD fusion proteins) that bind 1, 2, 3, 4, 5, 6, or more of the same antigens as the above antibodies are also encompassed by the invention. Additionally, MMM complexes (e.g., ELP-MRD fusion proteins) that bind 1, 2, 3, 4, 5, 6, or more of the same epitopes as, or competitively inhibit binding of, one of the above antibodies are also encompassed by the invention. Moreover, MMM complexes (e.g., ELP-MRD fusion proteins) having 1, 2, 3, 4, 5, 6, or more antibody fragments or domains (e.g., ScFvs) that bind to one or more of the above antigens are also encompassed by the invention. MMM complexes (e.g., ELP-MRD fusion proteins) having antibody fragments or domains that bind to 1, 2, 3, 4, 5, 6, or more of the above antigens are also encompassed by the invention.

In another embodiment, an antibody fragment or domain, or an MMM complex (e.g., an ELP-MRD fusion protein) comprising an antibody fragment or domain binds to a member selected from: CD19, CD22, CD30, CD33, CD38, CD44v6, TNFSF5 (CD40 Ligand), TNFRSF5 (CD40), CD52, CD54 (ICAM), CD74, CD80, CD200, EPCAM (EGP2), neuropilin 1 (NRP1), TEM1, mesothelin, TGFbeta 1, TGFBR11, phosphatidlyserine, folate receptor alpha (FOLR1), TNFRSF10A (TRAIL R1DR4), TNFRSF10B (TRAIL R2DR5), CXCR4, CCR4, CCL2, HGF, CRIPTO, VLA5, TNFSF9 (41BB Ligand), TNFRSF9 (41BB, CD137), CTLA4, HLA-DR, IL6, TNFSF4 (OX40 Ligand), TNFRSF4 (OX40), MUC1, MUC18, mucin CanAg, ganglioside GD3, EGFL7, PDGFRa, IL21, IGF1, IGF2, CD117 (cKit), PSMA, SLAMF7, carcinoembryonic antigen (CEA), FAP, integrin avb3, or integrin α5β3. MMM complexes (e.g., ELP-MRD fusion proteins) that bind 1, 2, 3, 4, 5, 6, or more of the same antigens as the above antibodies are also encompassed by the invention. Additionally, MMM complexes (e.g., ELP-MRD fusion proteins) that bind 1, 2, 3, 4, 5, 6, or more of the same epitopes as, or competitively inhibit binding of, one of the above antibodies are also encompassed by the invention. Moreover, MMM complexes (e.g., ELP-MRD fusion proteins) having 1, 2, 3, 4, 5, 6, or more antibody fragments or domains (e.g., ScFvs) that bind to one of the above antigens are also encompassed by the invention. MMM complexes (e.g., ELP-MRD fusion proteins) having 1, 2, 3, 4, 5, 6, or more antibody fragments or domains (e.g., ScFv) that bind to one or more of the above antigens are also encompassed by the invention. MMM complexes (e.g., ELP-MRD fusion proteins) having antibody fragments or domains that bind to 1, 2, 3, 4, 5, 6, or more of the above antigens are also encompassed.

In particular embodiments, an antibody fragment or domain, or an MMM complex (e.g., an ELP-MRD fusion protein) comprising an antibody fragment or domain competes for epitope binding with an antibody selected from: siplizumab CD2 (e.g., MEDI-507, Medlmmune), blinatumomab CD19 CD3 (e.g., MT103, Micromet/Medlmmune); XMAB®5574 CD19 (Xencor), SGN-19A CD19 (Seattle Genetics), ASG-5ME (Agenesys and Seattle Genetics), MEDI-551 CD19 (Medlmmune), epratuzumab CD22 (e.g., hLL2, Immunomedics/UCB), inotuzumab ozogamicin CD22 (Pfizer), iratumumab CD30 (e.g., SGN-30 (Seattle Genetics) and MDX-060 (Medarex)), XMAB®2513 CD30 (Xencor), brentuximab vedotin CD30 (e.g., SGN-35, Seattle Genetics), gemtuzumab ozogamicin CD33 (e.g., MYLOTARG®, Pfizer), lintuzumab CD33 (e.g., antibody of Seattle Genetics), MOR202, CD38 (MorphoSys), daratumumab CD38 (e.g., Genmab antibody), CP870893 CD40 (Pfizer), dacetuzumab CD40 (e.g., SGN40, Seattle Genetics), ANTOVA® CD40 (Biogen Idec), lucatumumab CD40 (e.g., HCD122, Novartis) XMAB®5485 CD40 (Xencor), teneliximub, ruplizumab CD40L (e.g., ANTOVA®) bivatuzumab mertansine CD44v6, alemtuzumab CD52 (e.g., CAMPATH®/MABCAMPATH®, Genzyme/Bayer), BI505 ICAM1 (Bioinvent), milatuzumab CD74 (e.g., antibody of Immunomedics), galiximab CD80 (Biogen Idec), BMS663513 4-1BB (Bristol-Myers Squibb), Alexion CD200 antibody (Alexion), edrecolomab EPCAM (e.g., MAb17-1A, PANOREX® (GlaxoSmithKline), AT003 EPCAM (Affitech)), adecatumumab EPCAM (e.g., MT201, Micromet), oportuzumab monatox EPCAM, Genentech anti-NRP1 antibody, MORAB004 TEM1 (Morphotek), MORAB009 mesothelin (Morphotek), lerdelimumab TGFb1 (e.g., CAT-152, Cambridge Antibody Technology), metelimumab TGFb1 (e.g., CAT-192, Cambridge Antibody Technology), ImClone anti-TGFBR11 antibody, bavituximab phosphatidylserine (e.g., antibody of Peregrine (Peregrine Pharmaceuticals)), AT004 phosphatidylserine (Affitech), AT005 phosphatidylserine (Affitech), MORABO3 folate receptor alpha (Morphotek), farletuzumab folate receptor alpha cancer (e.g., MORAB003, Morphotek), CS1008 DR4 (Sankyo), mapatumumab DR4 (e.g., HGS-ETR1, Human Genome Sciences), LBY135 DR5 (Novartis), AMG66 DR5 (Amgen), Apomab DR5 (Genentech), PRO95780 (Genentech), lexatumumab DR5, (e.g., HGS-ETR2, Human Genome Sciences), conatumumab DR5, (e.g., AMG655, Amgen), tigatuzumab (e.g., CS-1008), AT009 CXCR4 (Affitech), AT008 CCR4 (Affitech), CNTO-888 CCL2 (Centocor), AMG102 HGF (Amgen), CRIPTO antibody (Biogen Idec), M200 antibody VLA5 (Biogen Idec), ipilimumab CTLA4 (e.g., MDX010, Bristol-Myers Squibb/Medarex), belatacept CTLA4 ECD (e.g., CP-675,206, Pfizer), IMMU114 HLA-DR (Immunomedics), apolizumab HLA-DR, toclizumab IL-6R (e.g., ACTEMR®A/ROACTREMRA®, Hoffman-La Roche), OX86 OX40, pemtumomab PEM/MUC1 (Theragyn), ABX-MA1 MUC-18 (Abgenix), clivatuzumab MUC-18 (e.g., hPAM4, Immunomedics), cantuzumab mertansine mucin CanAg, ecromeximab (Ludwig Institute), Genentech anti-EGFL7 antibody, AMG820 CSFR (Amgen), olaratumab PDGFRa (e.g., antibody of ImClone (ImClone)), IL21 antibody Zymogenetics (Zymogenetics), MEDI-573 IGF1/IGF2 (Medlmmune), AMG191 cKit (Amgen), etaracizumab (e.g., MEDI-522, Medlmune), and MLN591 PSMA (Millennium Pharmaceuticals), elotuzumab SLAMF7 (e.g., HuLuc63, PDL), labetuzumab CEA (CEA-CIDE®, Immunomedics), sibrotuzumab FAP, CNTO95 integrin avb3 (Centocor), VITAXIN® integrin avb3 (Medlmmune), and voloximab αβ1. MMM complexes (e.g., ELP-MRD fusion proteins) that bind 1, 2, 3, 4, 5, 6, or more of the same antigens as the above antibodies are also encompassed by the invention. Additionally, MMM complexes (e.g., ELP-MRD fusion proteins) that bind 1, 2, 3, 4, 5, 6, or more of the same epitopes as, or competitively inhibit binding of one of the above antibodies are also encompassed by the invention. MMM complexes (e.g., ELP-MRD fusion proteins) having 1, 2, 3, 4, 5, 6, or more antibody fragments or domains (e.g., ScFv) that bind to the same antigen as the above antibodies are also encompassed by the invention. MMM complexes (e.g., ELP-MRD fusion proteins) having 1, 2, 3, 4, 5, 6, or more antibody fragments or domains that bind to the same epitope as, or competitively inhibit binding of, one of the above antibodies are also encompassed by the invention. MMM complexes (e.g., ELP-MRD fusion proteins) having antibody fragments or domains that bind to the same epitope as, or competitively inhibit binding of, at least 1, 2 or more of the above antibodies are additionally encompassed by the invention. MMM complexes (e.g., ELP-MRD fusion proteins) having 1, 2, 3, 4, 5, 6, or more antibody fragments or domains that are fragments or domains of the above antibodies are also encompassed.

In another embodiment, an antibody fragment or domain, or an MMM complex (e.g., an ELP-MRD fusion protein) comprising an antibody fragment or domain binds to ANG2 (ANGPT2). In one embodiment, the antibody fragment or domain, or an MMM complex (e.g., an ELP-MRD fusion protein) comprising an antibody fragment or domain binds to the same epitope as MEDI3617. In another embodiment, the antibody fragment or domain, or an MMM complex (e.g., an ELP-MRD fusion protein) comprising an antibody fragment or domain competitively inhibits binding of MEDI3617 to ANG2.

In certain embodiments, an antibody fragment or domain, or an MMM complex (e.g., an ELP-MRD fusion protein) comprising an antibody fragment or domain binds to EGFR, ErbB2, ErbB3, ErbB4, CD20, insulin-like growth factor-I receptor, prostate specific membrane antigen, an integrin, or cMet. MMM complexes (e.g., ELP-MRD fusion proteins) having 1, 2, 3, 4, 5, 6, or more antibody fragments or domains (e.g., ScFvs) that bind to one of the above antigens are also encompassed by the invention. MMM complexes (e.g., ELP-MRD fusion proteins) that bind to one or more of the above antigens are also encompassed by the invention. MMM complexes (e.g., ELP-MRD fusion proteins) having antibody fragments or domains that bind to 1, 2, 3, 4, 5, 6, or more of the above antigens are also encompassed by the invention. MMM complexes (e.g., ELP-MRD fusion proteins) that bind to 1, 2, 3, 4, 5, 6, or more of the above antigens are also encompassed by the invention.

In another embodiment, an antibody fragment or domain and/or the MMM complex (e.g., ELP-MRD fusion protein) comprising an antibody fragment or domain binds EGFR. In one embodiment, the antibody fragment or domain and/or the MMM complex (e.g., ELP-MRD fusion protein) comprising an antibody fragment or domain binds to the same epitope as ERBITUX®. In an additional embodiment, the antibody fragment or domain and/or the MMM complex (e.g., ELP-MRD fusion protein) comprising an antibody fragment or domain competitively inhibits binding of ERBITUX® to EGFR. In a further embodiment, the antibody fragment or domain and/or the MMM complex (e.g., ELP-MRD fusion protein) comprising an antibody fragment or domain inhibits EGFR dimerization. In another embodiment, the antibody fragment or domain and/or the MMM complex (e.g., ELP-MRD fusion protein) comprising an antibody fragment or domain binds to the same epitope as matuzimab or panitumumab. In an additional embodiment, the antibody fragment or domain and/or the MMM complex (e.g., ELP-MRD fusion protein) comprising an antibody fragment or domain competitively inhibits binding of matuzimab or panitumumab to EGFR. In a further embodiment, the antibody fragment or domain and/or the MMM complex (e.g., ELP-MRD fusion protein) comprising an antibody fragment or domain binds to the same epitope as ABX-EGF or MDX-214. In another embodiment, an antibody fragment or domain and/or the MMM complex (e.g., ELP-MRD fusion protein) comprising an antibody fragment or domain competitively inhibits binding of ABX-EGF or MDX-214 to EGFR.

In another embodiment the antibody fragment or domain and/or the MMM complex (e.g., ELP-MRD fusion protein) comprising an antibody fragment or domain binds ErbB2 (Her2). In one embodiment, the antibody fragment or domain and/or the MMM complex (e.g., ELP-MRD fusion protein) comprising an antibody fragment or domain binds to the same epitope as trastuzumab (e.g., HERCEPTIN®, Genentech/Roche). In another embodiment, the antibody fragment or domain and/or the MMM complex (e.g., ELP-MRD fusion protein) comprising an antibody fragment or domain competitively inhibits binding of trastuzumab to ErbB2. MMM complexes (e.g., ELP-MRD fusion proteins) having 1, 2, 3, 4, 5, 6, or more antibody fragments or domains that bind to the same epitope as, or competitively inhibit binding of trastuzumab are also encompassed by the invention. MMM complexes (e.g., ELP-MRD fusion proteins) having 1, 2, 3, 4, 5, 6, or more antibody fragments or domains that are fragments or domains of trastuzumab are also encompassed.

In another embodiment, the antibody fragment or domain and/or the MMM complex (e.g., ELP-MRD fusion protein) comprising an antibody fragment or domain inhibits HER2 dimerization. In another embodiment, the antibody fragment or domain and/or the MMM complex (e.g., ELP-MRD fusion protein) comprising an antibody fragment or domain inhibits HER2 heterodimerization with HER3 (ErbB3). In a specific embodiment, the antibody fragment or domain is a fragment or domain of pertuzumab (e.g., OMNITARG® and phrMab2C4, Genentech). In another embodiment, the antibody fragment or domain and/or the MMM complex (e.g., ELP-MRD fusion protein) comprising an antibody fragment or domain binds to the same epitope as pertuzumab. In another embodiment, the antibody fragment or domain and/or the MMM complex (e.g., ELP-MRD fusion protein) comprising an antibody fragment or domain competitively inhibits binding of ErbB2 by pertuzumab. MMM complexes (e.g., ELP-MRD fusion proteins) having 1, 2, 3, 4, 5, 6, or more antibody fragments or domains that bind to the same epitope as, or competitively inhibit binding of, pertuzumab are also encompassed by the invention. MMM complexes (e.g., ELP-MRD fusion proteins) having 1, 2, 3, 4, 5, 6, or more antibody fragments or domains that are fragments or domains of pertuzumab are also encompassed.

In another embodiment, the antibody fragment or domain and/or the MMM complex (e.g., ELP-MRD fusion protein) comprising an antibody fragment or domain binds to the same epitope on ErbB2 as an antibody selected from the group: MDX-210 (Medarex), tgDCC-E1A (Targeted Genetics), MGAH22 (MacroGenics), and pertuzumab (OMNITARG™, 2C4; Genentech). MMM complexes (e.g., ELP-MRD fusion proteins) having 1, 2, 3, 4, 5, 6, or more antibody fragments or domains (e.g., ScFv) that bind to the same antigen as the above antibodies are also encompassed by the invention. MMM complexes (e.g., ELP-MRD fusion proteins) having 1, 2, 3, 4, 5, 6, or more antibody fragments or domains that bind to the same epitope as, or competitively inhibit binding of, one of the above antibodies are also encompassed by the invention. MMM complexes (e.g., ELP-MRD fusion proteins) having antibody fragments or domains that bind to the same epitope as, or competitively inhibit binding of, at least 1, 2 or more of the above antibodies are additionally encompassed by the invention. MMM complexes (e.g., ELP-MRD fusion proteins) having 1, 2, 3, 4, 5, 6, or more antibody fragments or domains that are fragments or domains of the above antibodies are also encompassed.

Thus, in some embodiments, an antibody fragment or domain and/or the MMM complex (e.g., ELP-MRD fusion protein) comprising an antibody fragment or domain comprises one or more of the CDRs of the anti-ErbB2 antibody trastuzumab. The CDR, VH, and VL sequences of trastuzumab are provided in Table 1.

TABLE 1

| CDR | Sequence |
|---|---|
| VL- | RASQDVNTAVAW |
| CDR1 | (SEQ ID NO: 59) |
| VL- | SASFLYS |
| CDR2 | (SEQ ID NO: 60) |
| VL- | QQHYTTPPT |
| CDR3 | (SEQ ID NO: 61) |
| VH- | GRNIKDTYIH |
| CDR1 | (SEQ ID NO: 62) |
| VH- | RIYPTNGYTRYADSVKG |
| CDR2 | (SEQ ID NO: 63) |
| VH- | WGGDGFYAMDY |
| CDR3 | (SEQ ID NO: 64) |
| VL | DIQMTQSPSSLSASVGDRVTITCRASQDVNTAVAWYQQKPGKAPK |
| | LLIYSASFLYSGVPSRFSGSRSGTDFTLTISSLQPEDFATYYCQQH |
| | YTTPPTFGQGTKVEIKRT |
| | (SEQ ID NO: 65) |
| VH | EVQLVESGGGLVQPGGSLRLSCAASGFNIKDTYIHWVRQAPGKGL |
| | EWVARIYPTNGYTRYADSVKGRFTISADTSKNTAYLQMNSLRAED |
| | TAVYYCSRWGGDGFYAMDYWGQGTLVTVSS |
| | (SEQ ID NO: 66) |

In one embodiment the antibody fragment or domain and/or the MMM complex (e.g., ELP-MRD fusion protein) comprising an antibody fragment or domain binds ErbB3 (Her3). In one embodiment, the antibody fragment or domain and/or the MMM complex (e.g., ELP-MRD fusion protein) comprising an antibody fragment or domain binds to the same epitope as MM121 (Merrimack Pharmaceuticals) or AMG888 (Amgen). MMM complexes (e.g., ELP-MRD fusion proteins) having 1, 2, 3, 4, 5, 6, or more antibody fragments or domains (e.g., ScFv) that bind to the same antigen as the above antibodies are also encompassed by the invention. MMM complexes (e.g., ELP-MRD fusion proteins) having 1, 2, 3, 4, 5, 6, or more antibody fragments or domains that bind to the same epitope as, or competitively inhibit binding of, one of the above antibodies are also encompassed by the invention. MMM complexes (e.g., ELP-MRD fusion proteins) having antibody fragments or domains that bind to the same epitope as, or competitively inhibit binding of, 1 or 2 of the above antibodies are additionally encompassed by the invention. MMM complexes (e.g., ELP-MRD fusion proteins) having 1, 2, 3, 4, 5, 6, or more antibody fragments or domains that are fragments or domains of the above antibodies are also encompassed.

In another embodiment the antibody fragment or domain and/or the MMM complex (e.g., ELP-MRD fusion protein) comprising an antibody fragment or domain binds VEGFA. In one embodiment, the antibody fragment or domain and/or the MMM complex (e.g., ELP-MRD fusion protein) comprising an antibody fragment or domain binds to the same epitope as bevacizumab (AVASTIN®, Genentech/Roche) to VEGFA. In an additional embodiment, the antibody fragment or domain and/or the MMM complex (e.g., ELP-MRD fusion protein) comprising an antibody fragment or domain binds to the same epitope as AT001 (Affitech). In another embodiment, the antibody fragment or domain and/or the MMM and/or the MMM complex (e.g., ELP-MRD fusion protein) comprising an antibody fragment or domain competitively inhibits binding of AT001 to VEGFA. MMM complexes (e.g., ELP-MRD fusion proteins) containing antibody fragment or domain that binds to the same epitope as one or more of the above antibodies are also encompassed by the invention. MMM complexes (e.g., ELP-MRD fusion proteins) having 1, 2, 3, 4, 5, 6, or more antibody fragments or domains (e.g., ScFv) that bind to the same antigen as the above antibodies are also encompassed by the invention. MMM complexes (e.g., ELP-MRD fusion proteins) having 1, 2, 3, 4, 5, 6, or more antibody fragments or domains that bind to the same epitope as, or competitively inhibit binding of, one of the above antibodies are also encompassed by the invention. MMM complexes (e.g., ELP-MRD fusion proteins) having antibody fragments or domains that bind to the same epitope as, or competitively inhibit binding of, 1 or 2 of the above antibodies are additionally encompassed by the invention. MMM complexes (e.g., ELP-MRD fusion proteins) having 1, 2, 3, 4, 5, 6, or more antibody fragments or domains that are fragments or domains of the above antibodies are also encompassed.

Thus, in some embodiments, the antibody fragments or domains in the MMM complex (e.g., ELP-MRD fusion protein) comprise one or more of the CDRs of the anti-VEGF antibody bevacizumab. The CDR, VH, and VL sequences of bevacizumab are provided in Table 2.

TABLE 2

| CDR | Sequence |
|---|---|
| VL- | SASQDISNYLN |
| CDR1 | (SEQ ID NO: 72) |
| VL- | FTSSLHS |
| CDR2 | (SEQ ID NO: 73) |
| VL- | QQYSTVPWT |
| CDR3 | (SEQ ID NO: 74) |
| VH- | GYTFTNYGMN |
| CDR1 | (SEQ ID NO: 75) |
| VH- | WINTYTGEPTYAADFKR |
| CDR2 | (SEQ ID NO: 76) |
| VH- | YPHYYGSSHWYFDV |
| CDR3 | (SEQ ID NO: 77) |
| VL | DIQMTQSPSSLSASVGDRVTITCSASQDISNYLNWYQQKPGKAPKV |
| | LIYFTSSLHSGVPSRFSGSGSGTDFTLTISSLQPEDFATYYCQQYS |
| | TVPWTFGQGTKVEIKR |
| | (SEQ ID NO: 78) |
| VH | EVQLVESGGGLVQPGGSLRLSCAASGYTFTNYGMNWVRQAPGKG |
| | LEWVGWINTYTGEPTYAADFKRRFTFSLDTSKSTAYLQMNSLRAE |
| | DTAVYYCAKYPHYYGSSHWYFDVWGQGTLVTVSS |
| | (SEQ ID NO: 79) |

In one embodiment, the antibody fragment or domain and/or the MMM and/or the MMM complex (e.g., ELP-MRD fusion protein) comprising an antibody fragment or domain binds VEGFR1. In one embodiment, the antibody fragment or domain and/or the MMM and/or the MMM complex (e.g., ELP-MRD fusion protein) comprising an antibody fragment or domain competitively inhibits binding of Aflibercept (Regeneron) to VEGFR1. In another embodiment, antibody fragment or domain and/or the MMM and/or the MMM complex (e.g., ELP-MRD fusion protein) comprising an antibody fragment or domain inhibits VEGFR1 dimerization. MMM complexes (e.g., ELP-MRD fusion proteins) having 1, 2, 3, 4, 5, 6, or more antibody fragments or domains (e.g., ScFv) that bind to the same antigen as Aflibercept are also encompassed by the invention. MMM complexes (e.g., ELP-MRD fusion proteins) having 1, 2, 3, 4, 5, 6, or more antibody fragments or domains that bind to the same epitope as, or competitively inhibit binding of, Aflibercept are also encompassed by the invention. MMM complexes (e.g., ELP-MRD fusion proteins) having 1, 2, 3, 4, 5, 6, or more antibody fragments or domains that are fragments or domains of Aflibercept are also encompassed by the invention.

In other embodiments, the antibody fragment or domain in the MRD-ELP fusion protein specifically binds to an FGF receptor (e.g., FGFR1, FGFR2, FGFR3, or FGFR4). In one embodiment, the antibody fragment or domain in the MRD-ELP fusion protein is an antibody fragment or domain that specifically binds to FGFR1 (e.g., FGFR1-IIIC). In a specific embodiment, the antibody is IMC-A1 (Imclone). In one embodiment, the antibody fragment or domain binds to the same epitope as IMC-A1. In another embodiment, the antibody fragment or domain competitively inhibits binding of IMC-A1 to FGFR1. In an additional embodiment, the antibody fragment or domain competitively inhibits binding of FP-1039 (Five Prime) to an FGF ligand of FGFR1. In another embodiment, the antibody fragment or domain in the MRD-ELP fusion protein is an antibody fragment or domain that specifically binds to FGFR2 (e.g., FGFR2-IIIB and FGFR2-IIIC). In a further embodiment, the antibody fragment or domain in the MRD-ELP fusion protein is an antibody fragment or domain that specifically binds to FGFR3. In a specific embodiment, the antibody is IMC-A1 (Imclone). In one embodiment, the antibody fragment or domain binds to the same epitope as PRO-001 (ProChon Biotech), R3Mab (Genentech), or 1A6 (Genentech). In another embodiment, the antibody fragment or domain competitively inhibits binding of PRO-001 (ProChon Biotech), R3Mab (Genentech), or 1A6 (Genentech).

In one embodiment, the antibody fragment or domain and/or the MMM and/or the MMM complex (e.g., ELP-MRD fusion protein) comprising an antibody fragment or domain binds VEGFR2. In a specific embodiment, the antibody fragment or domain and/or the MMM and/or the MMM complex (e.g., ELP-MRD fusion protein) comprising an antibody fragment or domain binds to the same epitope as ramucirumab (e.g., IMC1121B and IMC1C11, ImClone). In another embodiment, the antibody fragment or domain and/or the MMM and/or the MMM complex (e.g., ELP-MRD fusion protein) comprising an antibody fragment or domain competitively inhibits binding of ramucirumab to VEGFR2. In an additional embodiment, the antibody fragment or domain and/or the MMM and/or the MMM complex (e.g., ELP-MRD fusion protein) comprising an antibody fragment or domain inhibits VEGFR2 dimerization. MMM complexes having 1, 2, 3, 4, 5, 6, or more antibody fragments or domains (e.g., ScFv) that bind to the same antigen as ramucirumab are also encompassed by the invention. MMM complexes having 1, 2, 3, 4, 5, 6, or more antibody fragments or domains that bind to the same epitope as, or competitively inhibit binding of, ramucirumab are also encompassed by the invention. MMM complexes having 1, 2, 3, 4, 5, 6, or more antibody fragments or domains that are fragments or domains of ramucirumab are also encompassed.

In one embodiment, the antibody fragment or domain and/or the MMM and/or the MMM complex (e.g., ELP-MRD fusion protein) comprising an antibody fragment or domain binds CD20. In one embodiment, the antibody fragment or domain and/or the MMM and/or the MMM complex (e.g., ELP-MRD fusion protein) comprising an antibody fragment or domain binds to the same epitope as rituximab (e.g., RITUXAN®/MABTHERA®, Genentech/Roche/Biogen Idec). In another embodiment, the antibody fragment or domain and/or the MMM and/or the MMM complex (e.g., ELP-MRD fusion protein) comprising an antibody fragment or domain competitively inhibits binding of rituximab to CD20. In one embodiment, the antibody fragment or domain and/or the MMM and/or the MMM complex (e.g., ELP-MRD fusion protein) comprising an antibody fragment or domain binds to the same epitope as GA-101 (Genentech). In another embodiment, the antibody fragment or domain and/or the MMM and/or the MMM complex (e.g., ELP-MRD fusion protein) comprising an antibody fragment or domain competitively inhibits binding of GA-101 to CD20. In another embodiment, the antibody fragment or domain and/or the MMM and/or the MMM complex (e.g., ELP-MRD fusion protein) comprising an antibody fragment or domain competitively inhibits binding of rituximab to CD20. In one embodiment, the antibody fragment or domain and/or the MMM and/or the MMM complex (e.g., ELP-MRD fusion protein) comprising an antibody fragment or domain binds to the same epitope as ocrelizumab (e.g., 2H7; Genentech/Roche/Biogen Idec). In another embodiment, the antibody fragment or domain and/or the MMM and/or the MMM complex (e.g., ELP-MRD fusion protein) comprising an antibody fragment or domain competitively inhibits binding of ocrelizumab to CD20. In another embodiment, the antibody fragment or domain and/or the MMM and/or the MMM complex (e.g., ELP-MRD fusion protein) comprising an antibody fragment or domain binds to the same epitope as an antibody selected from: obinutuzumab (e.g., GA101; Biogen Idec/Roche/Glycart), ofatumumab (e.g., ARZERRA® and HuMax-CD20Genmab), veltuzumab (e.g., IMMU-160, Immunomedics), AME-133 (Applied Molecular Evolution), SGN35 (Millennium), TG-20 (GTC Biotherapeutics), afutuzumab (Hoffman-La Roche), and PRO131921 (Genentech). In another embodiment, the antibody fragment or domain and/or the MMM and/or the MMM complex (e.g., ELP-MRD fusion protein) comprising an antibody fragment or domain competitively inhibits CD20 binding by an antibody selected from: obinutuzumab, ofatumumab, veltuzumab, AME-133, SGN35, TG-20 and PRO131921. MMM complexes having 1, 2, 3, 4, 5, 6, or more antibody fragments or domains (e.g., ScFv) that bind to the same antigen as the above antibodies are also encompassed by the invention. MMM complexes having 1, 2, 3, 4, 5, 6, or more antibody fragments or domains that bind to the same epitope as, or competitively inhibit binding of, one of the above antibodies are also encompassed by the invention. MMM complexes (e.g., ELP-MRD fusion proteins) having antibody fragments or domains that bind to the same epitope as, or competitively inhibit binding of, 1 or 2 of the above antibodies are additionally encompassed by the invention. MMM complexes (e.g., ELP-MRD fusion proteins) having 1, 2, 3, 4, 5, 6, or more antibody fragments or domains that are fragments or domains of the above antibodies are also encompassed.

In one embodiment, the antibody fragment or domain and/or the MMM and/or the MMM complex (e.g., ELP-MRD fusion protein) comprising an antibody fragment or domain binds IGF1R. In one embodiment, the antibody fragment or domain and/or the MMM and/or the MMM complex comprising an antibody fragment or domain binds to the same epitope as an antibody selected from: cixutumumab (e.g., IMC-A12, ImClone), figitumumab (e.g., CP-751,871, Pfizer), AMG479 (Amgen), BIIB022 (Biogen Idec), SCH 717454 (Schering-Pough), and R1507 (Hoffman La-Roche). In another embodiment, the antibody fragment or domain and/or the MMM and/or the MMM complex comprising an antibody fragment or domain competitively inhibits IGF1R binding by an antibody selected from: cixutumumab, figitumumab, AMG479, BIIB022, SCH 717454, and R1507. In another embodiment, the antibody fragment or domain and/or the MMM and/or the MMM comprising an antibody fragment or domain inhibits IGF1R dimerization. MMM complexes having 1, 2, 3, 4, 5, 6, or more antibody fragments or domains (e.g., ScFv) that bind to the same antigen as the above antibodies are also encompassed by the invention. MMM complexes having 1, 2, 3, 4, 5, 6, or more antibody fragments or domains that bind to the same epitope as, or competitively inhibit binding of, one of the above antibodies are also encompassed by the invention. MMM complexes having antibody fragments or domains that bind to the same epitope as, or competitively inhibit binding of, 1 or 2 of the above antibodies are additionally encompassed by the invention. MMM complexes having 1, 2, 3, 4, 5, 6, or more antibody fragments or domains that are fragments or domains of the above antibodies are also encompassed by the invention.

In additional embodiments, the MMM complex (e.g., ELP-MRD fusion protein) binds a target (e.g., ligand, receptor, or accessory protein) associated with an endogenous blood brain barrier (BBB) receptor mediated transport system (e.g., the insulin receptor, transferrin receptor, leptin receptor, lipoprotein receptor, and the IGF receptor mediated transport systems) and is capable of crossing to the brain side of the BBB. In some embodiments, the MMM complex (has 2 or more binding sites (i.e., is capable of multivalently binding) for a target antigen (e.g., ligand, receptor, or accessory protein) associated with an endogenous BBB receptor mediated transport system. In additional embodiments, the MMM complex has a single binding site for (i.e., monovalently binds) a target (e.g., ligand, receptor, or accessory protein) associated with an endogenous BBB receptor mediated transport system. In further embodiments, the MMM complex additionally binds 1, 2, 3, 4, 5, or more targets located on the brain (cerebrospinal fluid) side of the BBB. In particular embodiments, the MMM complex binds 1, 2, 3, 4, 5, or more targets associated with a neurological disease or disorder. In particular embodiments, the neurological disease or disorder is a member selected from: brain cancer, a neurodegenerative disease, schizophrenia, epilepsy, Alzheimer's disease, Parkinson's disease, Huntington's disease, ALS, multiple sclerosis, Neuromyelitis optica and Neuro-AIDS (e.g., HIV-associated dementia). Accordingly, the invention encompasses methods of treating a patient by administering a therapeutically effective amount of an MMM complex to treat a neurological disease or disorder selected from brain cancer, a neurodegenerative disease, schizophrenia, epilepsy, Alzheimer's disease, Parkinson's disease, Huntington's disease, ALS, multiple sclerosis, Neuromyelitis optica and Neuro-AIDS (e.g., HIV-associated dementia). In another embodiment, the MMM complex is administered to a patient to treat a brain cancer, metastatic cancer of the brain, or primary cancer of the brain. In additional embodiments, the MMM complex is administered to a patient to treat a neurological tumor such as, a glioma (e.g., a glioblastoma, glioblastoma multiforme (GBM), and astrocytoma), ependymoma, oligodendroglioma, neurofibroma, sarcoma, medulloblastoma, primitive neuroectodermal tumor, pituitary adenoma, neuroblastoma or cancer of the meninges (e.g., meningioma, meningiosarcoma and gliomatosis). In a further embodiment, the MMM complex is administered to a patient to treat brain injury, stroke, spinal cord injury, or to manage pain.

In some embodiments, the MMM complex (e.g., ELP-MRD fusion protein) binds an endogenous BBB receptor mediated transport system selected from the insulin receptor, transferrin receptor, leptin receptor, lipoprotein receptor, and the IGF receptor mediated transport systems.

In some embodiments, the MMM complex (e.g., ELP-MRD fusion protein) binds transferrin receptor. In additional embodiments, the MMM complex binds a target selected from: low-density lipoprotein receptor 1 (LRP-1), a LRP-1 ligand or a functional fragment or variant thereof that binds LRP-1, Low-density lipoprotein receptor 2 (LRP-2), a LRP-2 ligand or a functional fragment or variant thereof that binds LRP-1, a transferrin protein or a functional fragment or variant thereof, insulin receptor, TMEM30A, leptin receptor, IGF receptor, an IGFR ligand or a functional fragment or variant thereof, diphtheria receptor, a diphtheria receptor ligand or a functional fragment or variant thereof, choline transporter, a complex that binds choline receptor, an amino acid transporter (e.g., LAT1/CD98, SLC3A2, and SLC7A5), an amino acid transporter ligand or a functional fragment or variant thereof, RAGE, a RAGE ligand or a functional fragment or variant thereof, SLC2A1 and a SLC2A1 ligand or a functional fragment or variant thereof.

In additional embodiments, the MMM complex (e.g., ELP-MRD fusion protein) binds a target antigen associated with an endogenous blood brain barrier (BBB) receptor mediated transport system and also binds a target antigen selected from alpha-synuclein, RGM A, NOGO A, NgR, OMGp MAG, CSPG, neurite inhibiting semaphorins (e.g., Semaphorin 3A and Semaphorin 4) an ephrin, A-beta, AGE (S100 A, amphoterin), NGF, soluble A-B, aggrecan, midkine, neurocan, versican, phosphacan, Te38, and PGE2, IL-1, IL-1R, IL-6, IL6R, IL-12, IL-18, IL-23, TWEAK, CD40, CD40L, CD45RB, CD52, CD200, VEGF, VLA-4, TNF alpha, Interferon gamma, GMCSF, FGF, C5, CXCL13, CCR2, CB2, MIP 1a and MCP-1. In a further embodiment, the MMM complex (e.g., ELP-MRD fusion protein) has a single binding site for a target associated with an endogenous blood brain barrier (BBB) receptor mediated transport system and further binds a target selected from alpha-synuclein, RGM A, NOGO A, NgR, OMGp MAG, CSPG, neurite inhibiting semaphorins (e.g., Semaphorin 3A and Semaphorin 4) an ephrin, A-beta, AGE (S100 A, amphoterin), NGF, soluble A-B, aggrecan, midkine, neurocan, versican, phosphacan, Te38, and PGE2, IL-1, IL-1R, IL-6, IL6R, IL-12, IL-18, IL-23, TWEAK, CD40, CD40L, CD45RB, CD52, CD200, VEGF, VLA-4, TNF alpha, Interferon gamma, GMCSF, FGF, C5, CXCL13, CCR2, CB2, MIP 1a and MCP-1.

In additional embodiments, the MMM complex (e.g., ELP-MRD fusion protein) is administered to a patient to treat a neurological disease or disorder selected from brain cancer, a neurodegenerative disease, schizophrenia, epilepsy, Alzheimer's disease, Parkinson's disease, Huntington's disease, ALS, multiple sclerosis, Neuromyelitis optica and Neuro-AIDS (e.g., HIV-associated dementia). In one embodiment, the MMM complex contains 2 binding sites for 2 or more of the above targets. In a further embodiment, the MMM complex contains 2 binding sites for 3 or more targets. In additional embodiments, the targets bound by the MMM complex are associated with cancer. In a further embodiment the targets bound by the MMM complex are associated with 1, 2, 3, 4, 5 or more different signaling pathways or modes of action associated with cancer.

In one embodiment, the antibody fragment or domain and/or the MMM and/or the MMM complex (e.g., ELP-MRD fusion protein) comprising an antibody fragment or domain binds integrin. In a specific embodiment, the antibody fragment or domain and/or the MMM and/or the MMM complex (e.g., ELP-MRD fusion protein) comprising an antibody fragment or domain binds to the same epitope as an antibody selected from: MEDI-522 avb3 (VITAXIN®, MedImmune), CNTO 95 a5b3 (Centocor), JC7U αvβ3, and volociximab a5b1 (e.g., M200, PDL and Biogen Idec). In another embodiment, the antibody fragment or domain and/or the MMM and/or the MMM complex (e.g., ELP-MRD fusion protein) comprising an antibody fragment or domain binds to the same epitope as an antibody selected from: MEDI-522, CNTO 95, JC7U αvβ3, and volociximab. In another embodiment, the antibody fragment or domain and/or the MMM and/or the MMM complex (e.g., ELP-MRD fusion protein) comprising an antibody fragment or domain competitively inhibits integrin binding by an antibody selected from: MEDI-522, CNTO 95, JC7U, and M200. In one embodiment, the antibody fragment or domain and/or the MMM and/or the MMM complex (e.g., ELP-MRD fusion protein) comprising an antibody fragment or domain binds to the same epitope as natalizumab (e.g., TSABR1 ®, Biogen Idec). In another embodiment, the antibody fragment or domain and/or the MMM and/or the MMM complex (e.g., ELP-MRD fusion protein) comprising an antibody fragment or domain competitively inhibits integrin binding by natalizumab. MMM complexes (e.g., ELP-MRD fusion proteins) having 1, 2, 3, 4, 5, 6, or more antibody fragments or domains (e.g., ScFv) that bind to the same antigen as the above antibodies are also encompassed by the invention. MMM complexes (e.g., ELP, ELP-MRD fusion proteins) having 1, 2, 3, 4, 5, 6, or more antibody fragments or domains that bind to the same epitope as, or competitively inhibit binding of, one of the above antibodies are also encompassed by the invention. MMM complexes (e.g., ELP-MRD fusion proteins) having antibody fragments or domains that bind to the same epitope as, or competitively inhibit binding of, 1 or 2 of the above antibodies are additionally encompassed by the invention. MMM complexes (e.g., ELP, ELP-MRD fusion proteins) having 1, 2, 3, 4, 5, 6, or more antibody fragments or domains that are fragments or domains of the above antibodies are also encompassed.

In one embodiment, the antibody fragment or domain and/or the MMM and/or the MMM complex (e.g., ELP-MRD fusion protein) comprising an antibody fragment or domain binds cMet. In a specific embodiment, the antibody fragment or domain and/or the MMM and/or the MMM complex (e.g., ELP-MRD fusion protein) comprising an antibody fragment or domain binds to the same epitope as an antibody selected from: MetMab (OA-5D5, Genentech), AMG-102 (Amgen) and DN30. In another embodiment, the antibody fragment or domain and/or the MMM and/or the MMM complex (e.g., ELP-MRD fusion protein) comprising an antibody fragment or domain competitively inhibits cMET binding by an antibody selected from: MetMab (OA-5D5), AMG-102 and DN30. MMM complexes (e.g., ELP-MRD fusion proteins) having 1, 2, 3, 4, 5, 6, or more antibody fragments or domains (e.g., ScFv) that bind to the same antigen as the above antibodies are also encompassed by the invention. MMM complexes (e.g., ELP-MRD fusion proteins) having 1, 2, 3, 4, 5, 6, or more antibody fragments or domains that bind to the same epitope as, or competitively inhibit binding of, one of the above antibodies are also encompassed by the invention. MMM complexes (e.g., ELP-MRD fusion proteins) having antibody fragments or domains that bind to the same epitope as, or competitively inhibit binding of, 1 or 2 of the above antibodies are additionally encompassed by the invention. MMM complexes (e.g., ELP-MRD fusion proteins) having 1, 2, 3, 4, 5, 6, or more antibody fragments or domains that are fragments or domains of the above antibodies are also encompassed.

In other specific embodiments, the antibody fragment or domain and/or the MMM and/or the MMM complex (e.g., ELP-MRD fusion protein) comprising an antibody fragment or domain binds VEGF. In a specific embodiment, the antibody fragment or domain and/or the MMM and/or the MMM complex (e.g., ELP-MRD fusion protein) comprising an antibody fragment or domain binds to the same epitope as bevacizumab (e.g., AVASTIN®, Genentech. In another embodiment, the antibody fragment or domain and/or the MMM and/or the MMM complex (e.g., ELP-MRD fusion protein) comprising an antibody fragment or domain competitively inhibits binding of bevacizumab to VEGF. In another specific embodiment, the antibody fragment or domain and/or the MMM complex (e.g., ELP-MRD fusion protein) comprising an antibody fragment or domain binds to the same epitope as r84 (Peregrine) or 2C3 (Peregrine). In another embodiment, the antibody fragment or domain and/or the MMM complex (e.g., ELP-MRD fusion protein) comprising an antibody fragment or domain competitively inhibits VEGF binding by r84 or 2C3. MMM complexes (e.g., ELP-MRD fusion proteins) having 1, 2, 3, 4, 5, 6, or more antibody fragments or domains (e.g., ScFv) that bind to the same antigen as the above antibodies are also encompassed by the invention. MMM complexes (e.g., ELP-MRD fusion proteins) having 1, 2, 3, 4, 5, 6, or more antibody fragments or domains that bind to the same epitope as, or competitively inhibit binding of, one of the above antibodies are also encompassed by the invention. MMM complexes (e.g., ELP-MRD fusion proteins) having antibody fragments or domains that bind to the same epitope as, or competitively inhibit binding of, 1 or 2 of the above antibodies are additionally encompassed by the invention. MMM complexes (e.g., ELP-MRD fusion proteins) having 1, 2, 3, 4, 5, 6, or more antibody fragments or domains that are fragments or domains of the above antibodies are also encompassed.

In another embodiment, an antibody fragment or domain and/or the MMM complex (e.g., ELP-MRD fusion protein) comprising an antibody fragment or domain binds an antigen associated with an autoimmune disorder, inflammatory or other disorder of the immune system or an antigen associated with regulating an immune response.

In one embodiment, an MMM complex (e.g., an ELP-MRD fusion protein) comprising an antibody fragment or domain binds an immunoinhibitory target selected from: IL-1, IL-1b, IL-1Ra, L-5, IL6, IL-6R, CD26L, CD28, CD80, FcRn, or Fc Gamma RIIB MMM complexes (e.g., ELP-MRD fusion proteins) having 1, 2, 3, 4, 5, 6, or more antibody fragments or domains that bind to one or more of the above targets are also encompassed by the invention. MMM complexes (e.g., ELP-MRD fusion proteins) having 1, 2, 3, 4, 5, 6, or more antibody fragments or domains (e.g., ScFvs) that bind to one of the above antigens are also encompassed by the invention. ELP-MRD fusions having antibody fragments or domains that bind to 1, 2 or more of the above antigens are also encompassed.

In another embodiment an antibody fragment or domain and/or the MMM complex (e.g., ELP-MRD fusion protein) comprising an antibody fragment or domain binds an immunostimulatory antigen selected from: CD25, CD28, CTLA-4, PD1, PD11, B7-H1, B7-H4, IL-10, TGFbeta, TNFSF4 (OX40 Ligand), TNFRSF4 (OX40), TNFSF5 (CD40 Ligand), TNFRSF5 (CD40), TNFSF9 (41BB Ligand), TNFRSF9 (41BB, CD137), TNFSF14 (LIGHT, HVEM Ligand), TNFRSF14 (HVEM), TNFSF15 (TL1A), TNFRSF25 (DR3), TNFSF18 (GITR Ligand), and TNFRSF18 (GITR). MMM complexes (e.g., ELP-MRD fusion proteins) having 1, 2, 3, 4, 5, 6, or more antibody fragments or domains that bind to one or more of the above targets are also encompassed by the invention. MMM complexes (e.g., ELP-MRD fusion proteins) having 1, 2, 3, 4, 5, 6, or more antibody fragments or domains (e.g., ScFvs) that bind to one of the above antigens are also encompassed by the invention. ELP-MRD fusions having antibody fragments or domains that bind to 1, 2 or more of the above antigens are also encompassed.

In one embodiment, the antibody fragment or domain and/or the MMM complex (e.g., ELP-MRD fusion protein) comprising an antibody fragment or domain binds IL1Ra, IL1Rb, IL-2, IL-3, IL-4, IL-7, IL-10, IL-11, IL-15, IL-16, IL-17, IL-17A, IL-17F, IL-18, IL-19, IL-25, IL-32, IL-33, interferon beta, SCF, BCA1/CXCL13, CXCL1, CXCL2, CXCL6, CXCL13, CXCL16, C3AR, CSAR, CXCR1, CXCR2, CCR1, CCR3, CCR7, CCR8, CCR9, CCR10, ChemR23, CCL3, CCL5, CCL11, CCL13, CCL17, CCL18, CCL19, CCL20, CCL21, CCL22, CCL24, CCL25, CCL26, CCL27, MPL, GP130, TLR2, TLR3, TLR4, TLR5, TLR7, TLR8, TLR9, TREM1, TREM2, FcRn, Fc Gamma RIIB, oncostatin M, lymphotoxin alpha (LTa), integrin beta 7 subunit, CD49a (integrin alpha 1), integrin a5b3, MIF, ESM1, WIF1, cathepsin B, cathepsin D, cathepsin K, cathepsin S, TNFSF2 (TNFa), TNFSF3 (LTb), TNFRSF3 (LTBR), TNFSF6 (Fas Ligand), TNFRSF6 (Fas, CD95), TNFRSF6B (DcR3), TNFSF8 (CD30 Ligand), TNFRSF8 (CD30), TNFSF9 (41BB Ligand), TNFRSF9 (41BB, CD137), TNFSF11 (RANKL), TNFRSF11A (RANK), TNFSF14 (LIGHT, HVEM Ligand), TNFRSF14 (HVEM), TNFRSF16 (NGFR), TNFSF18 (GITR Ligand), TNFRSF18 (GITR), TNFRSF19L (RELT), TNFRSF19 (TROY), TNFRSF21 (DR6), CD14, CD23 CD25, CD28, CD36, CD36L, CD39, CD52, CD91, CD137, CD153, CD164, CD200, CD200R, BTLA, B7-1 (CD80), B7-2 (CD86), B7h, ICOS, ICOSL, MHC, CD, B7-H2, B7-H3, B7-H4, B7x, SLAM, KIM-1, SLAMF2, SLAMF3, SLAMF4, SLAMF5, SLAMF6, or SLAMF7. MMM complexes (e.g., ELP-MRD fusion proteins) having 1, 2, 3, 4, 5, 6, or more antibody fragments or domains (e.g., ScFvs) that bind to one of the above antigens are also encompassed by the invention. MMM complexes (e.g., ELP-MRD fusion proteins) having antibody fragments or domains that bind to 1, 2, 3, 4, 5, 6, or more of the above antigens are also encompassed by the invention.

In another embodiment, an antibody fragment or domain and/or the MMM complex (e.g., ELP-MRD fusion protein) comprising an antibody fragment or domain binds TNFSF1A (TNF-alpha), TNFRSF1A (TNFR1, p55, p60), TNFRSF1B (TNFR2), TNFSF7 (CD27 Ligand, CD70), TNFRSF7 (CD27), TNFSF13B (BLYS), TNFSF13 (APRIL), TNFRSF13B (TACI), TNFRSF13C (BAFFR), TNFRSF17 (BCMA), TNFSF15 (TL1A), TNFRSF25 (DR3), TNFSF12 (TWEAK), TNFRSF12 (TWEAKR), TNFSF4 (OX40 Ligand), TNFRSF4 (OX40), TNFSF5 (CD40 Ligand), TNFRSF5 (CD40), IL-1, IL-1b, IL1R, IL-2R, IL4-Ra, IL-5, IL-5R, IL-6, IL6R, IL9, IL12, IL-13, IL-14, IL-15, IL-15R, IL-17f, IL-17R, IL-17Rb, IL-17RC, IL-20, IL-21, IL-22RA, IL-23, IL-23R, IL-31, TSLP, TSLPR, interferon alpha, interferon gamma, B7RP-1, cKit, GMCSF, GMCSFR, CTLA-4, CD2, CD3, CD4, CD11a, CD18, CD20, CD22, CD26L, CD30, CD40, CD80, CD86, CXCR3, CXCR4, CCR2, CCR4, CCR5, CCR8, CCL2, CXCL10, P1GF, PD1, B7-DC(PDL2), B7-H1 (PDLL), alpha4 integrin subunit, A4B7 integrin, C5, RhD, IgE, or Rh. MMM complexes (e.g., ELP-MRD fusion proteins) having 1, 2, 3, 4, 5, 6, or more antibody fragments or domains (e.g., ScFvs) that bind to one of the above antigens are also encompassed by the invention. MMM complexes (e.g., ELP-MRD fusion proteins) having antibody fragments or domains that bind to 1, 2, 3, 4, 5, 6, or more of the above antigens are also encompassed.

In particular embodiments, an antibody fragment or domain and/or the MMM complex (e.g., ELP-MRD fusion protein) comprising an antibody fragment or domain competes for binding with: SGN-70 CD70 (Seattle Genetics), SGN-75 CD70 (Seattle Genetics), Belimumab BLYS (e.g., BENLYSTA®, Human Genome Sciences/GlaxoSmithKline), Atacicept BLYS/APRIL (Merck/Serono), TWEAK (e.g., Biogen mAb), TL1a antibodies of CoGenesys/Teva (e.g., hum11D8, hum25B9, and humlB4 (U.S. Patent Application Publication 2009/0280116), OX40 mAb, humAb OX40L (Genentech), rilonacept IL1 trap (e.g., ARCALYST®, Regeneron), catumaxomab IL1b (e.g., REMOVAB®, Fresenius Biotech GmbH), Xoma052 IL1b (Lilly), canakinumab IL1beta (e.g., ILARIS® (Novartis) and ACZ885 (Novartis)), AMG108 IL1R (Amgen), daclizumab IL2Ra (e.g., ZENAPAX®, Hoffman-La Roche), basiliximab IL2Ra (e.g., SIMULECT®, Novartis), AMGN-317 IL-4-a (Amgen), pascolizumab IL-4 (PDL), mepolizumab IL5 (e.g., BOSATRIA®, GlaxoSmithKline), reslizumab IL5 (e.g., SCH55700, Ception Therapeutics), MEDI-563 IL-5R (MedImmune), BIW-8405, IL-5R (BioWa), etanercept TNFR2-fc (e.g., ENBREL®, Amgen), siltuximab IL6 (e.g., CNT0328, Centocor), CNTO-136 IL6 (Centocor), CDP-6038 IL6 (UCB), AMGN-220 IL6 (Amgen), REGN-88 IL6R (Regeneron), tocilizumab IL6R (e.g., ACTEMRA™/ROACTEMRA™, Chugai/Roche), MEDI-528 IL9 (MedImmune), briakinumab IL-12/13 (e.g., ABT-874, Abbott), ustekinumab IL-12, IL-23 (e.g., STELARA® and CNTO 1275, Centocor), TNX-650 IL-13 (Tanox), lebrikizumab IL-13 (Genentech), CAT354 IL-13 (Cambridge Antibody Technology), AMG714 IL-15 (Amgen), CRB-15 IL-15R (Hoffman La-Roche), AMG827 IL-17R (Amgen), IL-17RC antibody of Zymogenetics/Merck Serono, IL-20 antibody of Zymogenetics, IL-20 antibody of Novo Nordisk, IL-21 antibody of Novo Nordisk (e.g., NCT01038674), IL-21 antibody Zymogenetics (Zymogenetics), IL-22RA antibody of Zymogenetics, IL-31 antibody of Zymogenetics, AMG157 TSLP (Amgen), MEDI-545 interferon alpha (MedImmune), MEDI-546 interferon alpha pathway component (MedImmune), AMG811 interferon gamma (Amgen), INNO202 interferon gamma (Innogenetics/Advanced Biotherapy), HuZAF interferon-gamma (PDL), AMG557 B7RP1 (Amgen), AMG191 cKit (Amgen), MOR103GMCSF (MorphoSys), CAM-3001 GMCSFR (MedImmune), tremelimumab CTLA4 (e.g., CP-675,206, Pfizer), iplimumab CTLA4 (e.g., MDX-010, BMS/Medarex), alefacept CD2 (e.g., AMEVIVE®, Astellas), siplizumab CD2 (e.g., MEDI-507, MedImmune), otelixizumab CD3 (e.g., TRX₄, Tolerx/GlaxoSmithKline), teplizumab CD3 (e.g., MGA031, MacroGenics/Eli Lilly), visilizumab CD3 (e.g., NUVION®, PDL), muromonab-CD3 CD3 (Ortho), ibalizumab (e.g., TMB-355 and TNX-355, TaiMed Biologics), zanolimumab CD4 (e.g., HUMAX-CD4®, Genmab), cedelizumab CD4 (Euroasian Chemicals), keliximab CD4, priliximab CD4 (e.g., cMT412, Centocor), BT-061 CD4 (BioTest AG), efalizumab CD11a (e.g., RAPTIVA®/XANELIM™, Genentech/Roche/Merck-Serono), MLN01 CD18 (Millennium Pharmaceuticals), epratuzumab CD22 (e.g., Amgen antibody) and hLL2; (Immunomedics/UCB)), aselizumab CD26L, iratumumab CD30 (e.g., SGN30 (Seattle Genetics) and MDX-060 (Medarex), SGN40 CD40 (Seattle Genetics), ANTOVA® CD40 ligand (Biogen Idec), abatacept CD80 CD86 (e.g., ORENCIA®, Bristol-Myers Squibb), CT-011 PD1 (Cure Tech), AT010 CXCR3 (Affitech), MLN₁₂O₂ CCR2 (Millennium Pharmaceuticals), AMG-761 CCR4 (Amgen), HGS004 CCR5 (Human Genome Sciences), PRO140 (Progenics), MDX-1338 CXCR4 (Medarex), CNTO-888 CCL2 (Centocor), ABN912 CCL2 (Novartis), MDX-1100 CXCL10 (Medarex), TB-403 PlGF (BioInvent), natalizumab integrin Alpha4 subunit (e.g., TYSABR1 ®, Biogen Idec/Elan), vedolizumab integrin A4B7 (e.g., MLN2, Millennium Pharmaceuticals/Takeda), eculizumab C5 Compliment (e.g., SOLIRIS®, Alexion), pexelizumab C5 Compliment (Alexion), omalizumab IgE (e.g., XOLAIR®, Genentech/Roche/Novartis), talizumab (e.g., TNX-901, Tanox), toralizumab (IDEC 131, IDEC), bertilimumab eotaxin (e.g., iCo-008, iCo Therapeutics Inc.), ozrolimupab RhD (e.g., Sym001, Symphogen A/S), atorolimumab or morolimumab (Rh factor). MMM complexes (e.g., ELP-MRD fusion proteins) having 1, 2, 3, 4, 5, 6, or more antibody fragments or domains (e.g., ScFv) that bind to the same antigen as the above antibodies are also encompassed by the invention. MMM complexes (e.g., ELP-MRD fusion proteins) having 1, 2, 3, 4, 5, 6, or more antibody fragments or domains that bind to the same epitope as, or competitively inhibit binding of, one of the above antibodies are also encompassed by the invention. MMM complexes (e.g., ELP-MRD fusion proteins) having antibody fragments or domains that bind to the same epitope as, or competitively inhibit binding of, 1, 2, 3, 4, 5, 6, or more of the above antibodies are additionally encompassed by the invention. MMM complexes (e.g., ELP-MRD fusion proteins) having 1, 2, 3, 4, 5, 6, or more antibody fragments or domains that are fragments or domains of the above antibodies are also encompassed.

In one embodiment, an antibody fragment or domain and/or the MMM complex (e.g., ELP-MRD fusion protein) comprising an antibody fragment or domain binds TNF. In another embodiment, the antibody fragment or domain and/or the MMM complex (e.g., ELP-MRD fusion protein) comprising an antibody fragment or domain binds to the same epitope as adalimumab (e.g., HUMIRA®/TRUDEXA®, Abbott). In another embodiment, the antibody fragment or domain and/or the MMM complex (e.g., ELP-MRD fusion protein) comprising an antibody fragment or domain competitively inhibits binding of adalimumab to TNF. In another embodiment, the antibody fragment or domain and/or the MMM complex (e.g., ELP-MRD fusion protein) comprising an antibody fragment or domain binds to the same epitope as infliximab. In another embodiment, the antibody fragment or domain and/or the MMM complex (e.g., ELP-MRD fusion protein) comprising an antibody fragment or domain competitively inhibits binding of infliximab to TNF. In another embodiment, the antibody fragment or domain and/or the MMM complex (e.g., ELP-MRD fusion protein) comprising an antibody fragment or domain competitively inhibits binding of: certolizumab (e.g., CIMZIA®, UCB), golimumab (e.g., SIMPONI™, Centocor), or AME-527 (Applied Molecular Evolution) to TNF. In an additional embodiment, the antibody fragment or domain and/or the MMM complex (e.g., ELP-MRD fusion protein) comprising an antibody fragment or domain binds to the same epitope as certolizumab (e.g., CIMZIA®, UCB), golimumab (e.g., SIMPONI™, Centocor), or AME-527 (Applied Molecular Evolution). In another embodiment, the antibody fragment or domain and/or the MMM complex (e.g., ELP-MRD fusion protein) comprising an antibody fragment or domain competitively inhibits binding of certolizumab, golimumab, or AME-527, to TNF. MMM complexes (e.g., ELP-MRD fusion proteins) having 1, 2, 3, 4, 5, 6, or more antibody fragments or domains (e.g., ScFv) that bind to the same antigen as the above antibodies are also encompassed by the invention. MMM complexes (e.g., ELP-MRD fusion proteins) having 1, 2, 3, 4, 5, 6, or more antibody fragments or domains that bind to the same epitope as, or competitively inhibit binding of, one of the above antibodies are also encompassed by the invention. MMM complexes (e.g., ELP-MRD fusion proteins) having antibody fragments or domains that bind to the same epitope as, or competitively inhibit binding of, 1 or 2 of the above antibodies are additionally encompassed by the invention. MMM complexes (e.g., ELP-MRD fusion proteins) having 1, 2, 3, 4, 5, 6, or more antibody fragments or domains that are fragments or domains of the above antibodies are also encompassed.

Thus, in some embodiments, an antibody fragment or domain and/or the MMM complex (e.g., ELP-MRD fusion protein) comprising an antibody fragment or domain comprises one or more of the CDRs of the anti-TNF antibody adalimumab. The CDR, VH, and VL sequences of adaliumumab are provided in Table 3.

TABLE 3

| CDR | Sequence |
|---|---|
| VL- | RASQGIRNYLA |
| CDR1 | (SEQ ID NO: 80) |
| VL- | AASTLQS |
| CDR2 | (SEQ ID NO: 81) |
| VL- | QRYNRAPYT |
| CDR3 | (SEQ ID NO: 82) |
| VH- | DYAMH |
| CDR1 | (SEQ ID NO: 83) |
| VH- | AITWNSGHIDYADSVEG |
| CDR2 | (SEQ ID NO: 84) |
| VH- | VSYLSTASSLDY |
| CDR3 | (SEQ ID NO: 85) |
| VL | DIQMTQSPSSLSASVGDRVTITCRASQGIRNYLAWYQQKPGKAPKL |
| | LIYAASTLQSGVPSRFSGSGSGTDFTLTISSLQPEDVATYYCQRYN |
| | RAPYTFGQGTKVEIKR |
| | (SEQ ID NO: 86) |
| VH | EVQLVESGGGLVQPGRSLRLSCAASGFTFDDYAMHWVRQAPGKG |
| | LEWVSAITWNSGHIDYADSVEGRFTISRDNAKNSLYLQMNSLRAE |
| | DTAVYYCAKVSYLSTASSLDYWGQGTLVTVSS |
| | (SEQ ID NO: 87) |

In particular embodiments, the antibody fragment or domain and/or the MMM complex (e.g., ELP-MRD fusion protein) comprising an antibody fragment or domain binds: amyloid beta (Abeta), beta amyloid, complement factor D, PLP, ROBO4, ROBO, GDNF, NGF, LINGO, or myostatin. In specific embodiments, the antibody fragment or domain and/or the MMM complex (e.g., ELP-MRD fusion protein) comprising an antibody fragment or domain binds to the same epitope as gantenerumab (e.g., R1450, Hoffman La-Roche), bapineuzumab beta amyloid 9 (Elan and Wyeth), solanezumab beta amyloid 9 (Lilly), tanezumab NGF (e.g., RN624, Pfizer), BIIB033 LINGO (Biogen Idec), or stamulumab myostatin (Wyeth). In another embodiment, the antibody fragment or domain and/or the MMM complex (e.g., ELP-MRD fusion protein) comprising an antibody fragment or domain competitively inhibits binding of gantenerumab, bapineuzumab, solarezumab, tanezumab, BIIB033, or stamulumab. MMM complexes (e.g., ELP-MRD fusion proteins) having 1, 2, 3, 4, 5, 6, or more antibody fragments or domains (e.g., ScFv) that bind to the same antigen as the above antibodies are also encompassed by the invention. MMM complexes (e.g., ELP-MRD fusion proteins) having 1, 2, 3, 4, 5, 6, or more antibody fragments or domains that bind to the same epitope as, or competitively inhibit binding of, one of the above antibodies are also encompassed by the invention. MMM complexes (e.g., ELP-MRD fusion proteins) having antibody fragments or domains that bind to the same epitope as, or competitively inhibit binding of, 1, 2, 3, 4, 5, 6, or more of the above antibodies are additionally encompassed by the invention. MMM complexes (e.g., ELP-MRD fusion proteins) having 1, 2, 3, 4, 5, 6, or more antibody fragments or domains that are fragments or domains of the above antibodies are also encompassed.

In another embodiment, an antibody fragment or domain and/or the MMM complex (e.g., ELP-MRD fusion protein) comprising an antibody fragment or domain binds: oxidized LDL, gpIIB, gpIIIa, PCSK9, Factor VIII, integrin a2bB3, AOC3, or mesothelin. In another embodiment, the antibody fragment or domain and/or the MMM complex (e.g., ELP-MRD fusion protein) comprising an antibody fragment or domain binds to the same epitope as BI-204, abciximab, AMG-145, TB-402, or tadocizumab. In another embodiment, the antibody fragment or domain and/or the MMM complex (e.g., ELP-MRD fusion protein) comprising an antibody fragment or domain competitively inhibits binding of BI-204, abciximab, AMG-145, TB-402, vapaliximab, or tadocizumab. MMM complexes (e.g., ELP-MRD fusion proteins) having 1, 2, 3, 4, 5, 6, or more antibody fragments or domains (e.g., ScFv) that bind to the same antigen as the above antibodies are also encompassed by the invention. MMM complexes (e.g., ELP-MRD fusion proteins) having 1, 2, 3, 4, 5, 6, or more antibody fragments or domains that bind to the same epitope as, or competitively inhibit binding of, one of the above antibodies are also encompassed by the invention. MMM complexes (e.g., ELP-MRD fusion proteins) having antibody fragments or domains that bind to the same epitope as, or competitively inhibit binding of, 1, 2, 3, 4, 5, 6, or more of the above antibodies are additionally encompassed by the invention. MMM complexes (e.g., ELP-MRD fusion proteins) having 1, 2, 3, 4, 5, 6, or more antibody fragments or domains that are fragments or domains of the above antibodies are also encompassed.

In other embodiments, the antibody fragment or domain and/or the MMM complex (e.g., ELP-MRD fusion protein) comprising an antibody fragment or domain binds an antigen associated with bone growth and/or metabolism. In certain embodiments, the antibody fragment or domain and/or the MMM complex (e.g., ELP-MRD fusion protein) comprising an antibody fragment or domain binds TNFSF11 (RANKL). In a specific embodiment, the antibody fragment or domain and/or the MMM complex (e.g., ELP-MRD fusion protein) comprising an antibody fragment or domain binds to the same epitope as denosumab (e.g., AMG-162, Amgen). In another embodiment, the antibody fragment or domain and/or the MMM complex (e.g., ELP-MRD fusion protein) comprising an antibody fragment or domain competitively inhibits binding by denosumab. In other embodiments, the antibody fragment or domain and/or the MMM complex (e.g., ELP-MRD fusion protein) comprising an antibody fragment or domain binds: DKK1, osteopontin, cathepsin K, TNFRSF19L (RELT), TNFRSF19 (TROY), or sclerostin (CDP-7851 UCB Celltech). In other embodiments, the antibody fragment or domain and/or the MMM complex (e.g., ELP-MRD fusion protein) comprising an antibody fragment or domain binds to the same epitope as AMG617 or AMG785 (e.g., CDP7851, Amgen). In another embodiment, the antibody fragment or domain and/or the MMM complex (e.g., ELP-MRD fusion protein) comprising an antibody fragment or domain competitively inhibits target binding of AMG617 or AMG785 (e.g., CDP7851, Amgen). In another embodiment, the antibody fragment or domain and/or the MMM complex (e.g., ELP-MRD fusion protein) comprising an antibody fragment or domain competitively inhibits binding of sclerostin by AMG617 or AMG785. MMM complexes (e.g., ELP-MRD fusion proteins) having 1, 2, 3, 4, 5, 6, or more antibody fragments or domains (e.g., ScFv) that bind to the same antigen as the above antibodies are also encompassed by the invention. MMM complexes (e.g., ELP-MRD fusion proteins) having 1, 2, 3, 4, 5, 6, or more antibody fragments or domains that bind to the same epitope as, or competitively inhibit binding of, one of the above antibodies are also encompassed by the invention. MMM complexes (e.g., ELP-MRD fusion proteins) having antibody fragments or domains that bind to the same epitope as, or competitively inhibit binding of, 1, 2, 3, 4, 5, 6, or more of the above antibodies are additionally encompassed by the invention. MMM complexes (e.g., ELP-MRD fusion proteins) having 1, 2, 3, 4, 5, 6, or more antibody fragments or domains that are fragments or domains of the above antibodies are also encompassed.

In additional embodiments, the antibody fragment or domain and/or the MMM complex (e.g., ELP-MRD fusion protein) comprising an antibody fragment or domain binds a bacterial antigen, a viral antigen, a mycoplasm antigen, a prion antigen, or a parasite antigen (e.g., one infecting a mammal). MMM complexes (e.g., ELP-MRD fusion proteins) having 1, 2, 3, 4, 5, 6, or more antibody fragments or domains (e.g., ScFvs) that bind to one of the above antigens are also encompassed by the invention. ELP-MRD fusions having antibody fragments or domains that bind to 1, 2 or more of the above antigens are also encompassed.

In other embodiments, the antibody fragment or domain and/or the MMM complex (e.g., ELP-MRD fusion protein) comprising an antibody fragment or domain binds a viral antigen. In one embodiment, the antibody fragment or domain and/or the MMM complex (e.g., ELP-MRD fusion protein) comprising an antibody fragment or domain binds anthrax, hepatitis b, rabies, Nipah virus, west nile virus, a mengititis virus, or CMV. In other embodiments, the antibody fragment or domain and/or the MMM complex (e.g., ELP-MRD fusion protein) comprising an antibody fragment or domain competitively inhibits binding of ABTHRAX® (Human Genome Sciences), exbivirumab, foravirumab, libivirumab, rafivirumab, regavirumab, sevirumab (e.g., MSL-109, Protovir), tuvirumab, raxibacumab, Nipah virus M102.4, or MGAWN1® (MacroGenics). MMM complexes (e.g., ELP-MRD fusion proteins) having 1, 2, 3, 4, 5, 6, or more antibody fragments or domains (e.g., ScFv) that bind to the same antigen as the above antibodies are also encompassed by the invention. MMM complexes (e.g., ELP-MRD fusion proteins) having 1, 2, 3, 4, 5, 6, or more antibody fragments or domains that bind to the same epitope as, or competitively inhibit binding of, one of the above antibodies are also encompassed by the invention. MMM complexes (e.g., ELP-MRD fusion proteins) having antibody fragments or domains that bind to the same epitope as, or competitively inhibit binding of, 1, 2, 3, 4, 5, 6, or more of the above antibodies are additionally encompassed by the invention. MMM complexes (e.g., ELP-MRD fusion proteins) having 1, 2, 3, 4, 5, 6, or more antibody fragments or domains that are fragments or domains of the above antibodies are also encompassed.

In other embodiments, the antibody fragment or domain and/or the MMM complex (e.g., ELP-MRD fusion protein) comprising an antibody fragment or domain binds RSV. In other embodiments, the antibody fragment or domain is a fragment or domain of motavizumab (e.g., NUMAX®, MEDI-577; Medlmmune) or palivizumab RSV fusion f protein (e.g., SYNAGIS®, Medlmmune). In other embodiments, the antibody fragment or domain and/or the MMM complex (e.g., ELP-MRD fusion protein) comprising an antibody fragment or domain competitively inhibits binding of motavizumab (e.g., NUMAX®, MEDI-577; Medlmmune) or palivizumab RSV fusion f protein (e.g., SYNAGIS®, Medlmmune). In other embodiments, the antibody fragment or domain and/or the MMM complex (e.g., ELP-MRD fusion protein) comprising an antibody fragment or domain competitively inhibits binding of felvizumab. In other embodiments, the antibody fragment or domain and/or the MMM complex (e.g., ELP-MRD fusion protein) comprising an antibody fragment or domain competitively inhibits binding of felvizumab. MMM complexes (e.g., ELP-MRD fusion proteins) having 1, 2, 3, 4, 5, 6, or more antibody fragments or domains (e.g., ScFv) that bind to the same antigen as the above antibodies are also encompassed by the invention. MMM complexes (e.g., ELP-MRD fusion proteins) having 1, 2, 3, 4, 5, 6, or more antibody fragments or domains that bind to the same epitope as, or competitively inhibit binding of, one of the above antibodies are also encompassed by the invention. MMM complexes (e.g., ELP-MRD fusion proteins) having antibody fragments or domains that bind to the same epitope as, or competitively inhibit binding of, 1, 2, 3, 4, 5, 6, or more of the above antibodies are additionally encompassed by the invention. MMM complexes (e.g., ELP-MRD fusion proteins) having 1, 2, 3, 4, 5, 6, or more antibody fragments or domains that are fragments or domains of the above antibodies are also encompassed.

In other embodiments, the antibody fragment or domain and/or the MMM complex (e.g., ELP-MRD fusion protein) comprising an antibody fragment or domain binds a bacterial or fungal antigen. In other embodiments, the antibody fragment or domain and/or the MMM complex (e.g., ELP-MRD fusion protein) comprising an antibody fragment or domain binds to the same epitope as nebacumab, edobacomab (e.g., E5), tefibazumab (Inhibitex), panobacumab (e.g., KBPA101, Kenta), pagibaximab (e.g., BSYX-A110, Biosynexus), urtoxazumab, or efungumab (e.g., MYCOGRAB®, Novartis). In other embodiments, the antibody fragment or domain and/or the MMM complex (e.g., ELP-MRD fusion protein) comprising an antibody fragment or domain competitively inhibits binding by nebacumab, edobacomab, tefibazumab, panobacumab, pagibaximab, urtoxazumab, or efungumab. MMM complexes (e.g., ELP-MRD fusion proteins) having 1, 2, 3, 4, 5, 6, or more antibody fragments or domains (e.g., ScFv) that bind to the same antigen as the above antibodies are also encompassed by the invention. MMM complexes (e.g., ELP-MRD fusion proteins) having 1, 2, 3, 4, 5, 6, or more antibody fragments or domains that bind to the same epitope as, or competitively inhibit binding of, one of the above antibodies are also encompassed by the invention. MMM complexes (e.g., ELP-MRD fusion proteins) having antibody fragments or domains that bind to the same epitope as, or competitively inhibit binding of, 1, 2, 3, 4, 5, 6, or more of the above antibodies are additionally encompassed by the invention. MMM complexes (e.g., ELP-MRD fusion proteins) having 1, 2, 3, 4, 5, 6, or more antibody fragments or domains that are fragments or domains of the above antibodies are also encompassed.

In another embodiment, the antibody fragment or domain and/or the MMM complex (e.g., ELP-MRD fusion protein) comprising an antibody fragment or domain binds to the same epitope as 38C2. In another embodiment, the antibody fragment or domain and/or the MMM complex (e.g., ELP-MRD fusion protein) comprising an antibody fragment or domain competitively inhibits 38C2 binding.

In another embodiment, the antibody fragment or domain and/or the MMM complex (e.g., ELP-MRD fusion protein) comprising an antibody fragment or domain binds to A33 antigen.

In some embodiments, one or more antibody variable domain fragments contained in MMM complex (e.g., ELP-MRD fusion protein) encompassed by some embodiments, of the invention bind to their target with a dissociation constant or Kd of less than 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M. In one embodiment, an antibody variable domain fragment component of an MMM complex (e.g., an ELP-MRD fusion protein) has a dissociation constant or Kd of less than 5×10⁻⁵ M. In another embodiment, an antibody variable domain fragment component of an MMM complex (e.g., an ELP-MRD fusion protein) has a dissociation constant or Kd of less than 5×10⁻⁸ M. In another embodiment, an antibody variable domain fragment component of an MMM complex (e.g., an ELP-MRD fusion protein) has a dissociation constant or Kd of less than less than 5×10⁻⁹ M. In another embodiment, the antibody variable domain fragment component of the MMM complexes (e.g., ELP-MRD fusion proteins) has dissociation constant or Kd of less than 5×10⁻¹⁰ M. In another embodiment, the antibody variable domain fragment component of the MMM complex (e.g., ELP-MRD fusion protein) has a dissociation constant or Kd of less than 5×10⁻¹¹ M. In another embodiment, the antibody fragment or domain of the MMM complex (e.g., ELP-MRD fusion protein) has a dissociation constant or Kd of less than 5×10⁻¹² M.

In specific embodiments, the antibody variable domain fragment component of the MMM complex (e.g., ELP-MRD fusion protein) binds its target with an off rate (k_{off}) of less than 5×10⁻² sec⁻¹, 10⁻² sec⁻¹, 5×10⁻³ sec⁻¹, or 10⁻³ sec⁻¹. More preferably, the antibody variable domain fragment component of the MMM complex (e.g., ELP-MRD fusion protein) binds its target with an off rate (k_{off}) of less than 5×10⁴ sec⁻¹, 10⁻⁴ sec⁻¹, 5×10⁻⁵ sec⁻¹, or 10⁻⁵ sec⁻¹, 5×10⁻⁶ sec⁻¹, 10⁻⁶ sec⁻¹, 5×10⁻⁷ sec⁻¹, or 10⁻⁷ sec⁻¹.

In other specific embodiments, the antibody fragment or domain of the MMM complex (e.g., ELP-MRD fusion protein) binds its target with an on rate (kₒₙ) of greater than 10³ M⁻¹ sec⁻¹, 5×10³ M⁻¹ sec⁻¹, 10⁴ M⁻¹ sec⁻¹, or 5×10⁴ M⁻¹ sec⁻¹. More preferably, the antibody fragment or domain of the MMM complex (e.g., ELP-MRD fusion protein) binds its target with an on rate (kₘ) of greater than 10⁵ M⁻¹ sec⁻¹, 5×10⁵ M⁻¹ sec⁻¹, 10⁶ M⁻¹ sec⁻¹, or 5×10⁶ M⁻¹ sec⁻¹, or 10⁷ M⁻¹ sec⁻¹.

Affinity maturation strategies and chain shuffling strategies (see, e.g., Marks et al., Bio/Technology 10:779-783 (1992), which is herein incorporated by reference) are known in the art and can be employed to generate high affinity antibody variable domain fragments that can be used in the MMM complexes (e.g., ELP-MRD fusion proteins) described herein.

Advantageously, the antibodies antibody variable domain fragment(s) contained in MMM complexes (e.g., ELP-MRD fusion proteins) of the invention can also include variants and derivatives that improve antibody function and/or desirable pharmacodynamic properties.

MMM complexes (e.g., ELP-MRD fusion proteins) used according to the methods of the invention also include derivatives that are modified, e.g., by the covalent attachment of any type of molecule to the antibody fragment such that covalent attachment does not prevent the antibody from specifically binding to its cognate epitope. For example, but not by way of limitation, the antibody fragment derivatives include antibody fragments that have been modified, e.g., by glycosylation, acetylation, pegylation, phosphorylation, amidation, or derivatization by known protecting/blocking groups. Any of numerous chemical modifications can be carried out by known techniques, including, but not limited to acetylation, formylation, etc. Additionally, the derivative may contain one or more non-classical amino acids.

In preferred embodiments, the MMM complex (e.g., ELP-MRD fusion protein) containing an antibody fragment or domain retains activities of the parent antibody. Thus, in certain embodiments, the MMM complex (e.g., ELP-MRD fusion protein) containing an antibody fragment or domain is capable of inducing complement dependent cytotoxicity. In certain embodiments, the MMM complex (e.g., ELP-MRD fusion protein) containing an antibody fragment or domain is capable of inducing antibody dependent cell mediated cytotoxicity (ADCC). In additional embodiments, the MMM complex (e.g., ELP-MRD fusion protein) containing an antibody fragment or domain is capable of inducing apoptosis. In additional embodiments, the MMM complex (e.g., ELP-MRD fusion protein) containing an antibody fragment or domain is capable of reducing tumor volume. In additional embodiments, the MMM complex (e.g., ELP-MRD fusion protein) containing an antibody fragment or domain is capable of inhibiting tumor growth.

B. Cytotoxic Agents and Other Modular Components

The present invention further provides MMM complexes (e.g., ELP-MRD fusion proteins) that comprise one or more therapeutic agents. Therapeutic agents that can be complexed with and/or recombinantly fused to the MMM complex (e.g., ELP-MRD fusion protein) of the invention include, but are not limited to one or more therapeutic components disclosed in WO 08/030,968 and WO 09/158,704 (each of these therapeutic compounds are excluded from the definition of an MRD herein), which is herein incorporated by reference.

In additional embodiments, the invention encompasses an MMM complex (e.g., an ELP-MRD fusion protein) that is covalently or otherwise associated with a cytotoxic agent (payload). According to some embodiments, the cytoxic agent is covalently attached to an MMM complex (e.g., an ELP-MRD fusion protein) by a linker. According to some embodiments, the linker attaching the MMM complex and the cytotoxic agent is cleavable by a protease. In additional embodiments, the cytotoxic agent is a chemotherapeutic agent, growth inhibitory agent, toxin (e.g., an enzymatically active toxin of bacterial, fungal, plant, or animal origin, or fragments or variants thereof), a radioactive isotope (i.e., a radioconjugate) or a prodrug. Methods of using MMM-Drug complexes (e.g., ELP-MRD fusion protein-drug fusion proteins) are also encompassed by the invention.

Cytotoxic agents that can be covalently or otherwise associated with MMM complexes (e.g., an MMM complex (e.g., an ELP-MRD fusion protein) include, but are not limited to any agent that is detrimental to (e.g., kills) cells. Cytotoxic agents useful in the compositions and methods of the invention include, inter alia, alkylating agents, intercalating agents, antiproliferative agents, anti-mititotic agents, tubulin binding agents, vinca alkaloids, enediynes, trichothecenes, podophyllotoxins or podophyllotoxin derivatives, the pteridine family of drugs, taxanes, anthracyclines (e.g., daunorubicin (formerly daunomycin) and doxorubicin), antibiotics (e.g., dactinomycin (formerly actinomycin, dolastatins (e.g., dolastatin 10, dolastatin 11, and dolastatin 15)), topoiosomerase inhibitors, and platinum complex chemotherapeutic agents (e.g., cis-platinum).

In some embodiments, compositions of the invention include a cytoxic agent that is a tubulin depolymerizing agent. Thus, in some embodiments, compositions of the invention include an auristatin or an auristatin derivative or analog. In one embodiment, compositions of the invention contain monomethyl auristatin E (MMAE). In another embodiment, compositions of the invention contain monomethyl auristatin F (MMAF). In additional embodiments, an MMM complex of the invention contains dolastatin or a dolastatin peptidic analog or derivative, e.g., an auristatin (U.S. Pat. Nos. 5,635,483; 5,780,588, 5,663,149)

In additional embodiments, complexes of the invention include a maytansinoid molecule. Maytansinoids are mitototic inhibitors which act by inhibiting tubulin polymerization. Methods of making maytansinoids and their therapeutic use are disclosed, for example, in U.S. Pat. Nos. 5,208,020; 5,416,064; 6,441,163 and European Patent EP 0 425 235 B1; each of which is herein incorporated by reference in its entirety.

Thus, in some embodiments, the cytotoxin is a maytansinoid or a maytansinoid derivative or analog. Maytansinoid drug moieties are attractive drug moieties in ELP-drug conjugates because they are: (i) relatively accessible to prepare by fermentation or chemical modification or derivatization of fermentation products, (ii) amenable to derivatization with functional groups suitable for conjugation through non-disulfide linkers to ELP, (iii) stable in plasma, and (iv) effective against a variety of tumor cell lines. Maytansine compounds suitable for use as maytansinoid drug moieties are well known in the art, and can be isolated from natural sources according to known methods, produced using genetic engineering techniques (see Yu et al PNAS 99:7968-7973 (2002)), or maytansinol and maytansinol analogues can be prepared synthetically according to known methods.

In particular embodiments, complexes of the invention include the maytansinoid DM1 (N(2′)-deacetyl-N(2′)-(3-mercapto-1-oxopropyl)-maytansine). In other particular embodiments, complexes of the invention include the maytansinoid DM2. In additional embodiments, complexes of the invention include the maytansinoid DM3 (N(2)-deacetyl-N2-(4-mercapto-1-oxopentyl)-maytansine) or DM4 (N(2′)-deacetyl-N2-(4-mercapto-4-methyl-1-oxopentyl)-maytansine).

In some embodiments, complexes of the invention include a cytoxic agent that is an alkylating agent. In particular embodiments, the cytotoxic agent is a member selected from: mechlorethamine, thiotepa, thioepa chlorambucil, melphalan, carmustine (BSNU), BCNU lomustine (CCNU), cyclothosphamide, busulfan, dibromomannitol, and streptozoicin.

In other embodiments, compositions of the invention include a cytoxic agent that is an antimetabolite. In particular embodiments, the cytotoxic agent is a member selected from: methotrexate, dichloromethotrexate, 6-mercaptopurine, 6-thioguanine, cytarabine, 5-fluorouracil and 5-fluorouracil decarbazine.

In additional embodiments, the multivalent and mulitspecific complex-drug conjugate (e.g., ELP-MRD fusion protein—drug (cytotoxic agent) conjugate) is capable of producing double-stranded DNA breaks. In further embodiments, the MMM complex (e.g., ELP-MRD fusion protein-drug conjugate contains a member of the calicheamicin family of antibiotics capable of producing double-stranded DNA breaks at sub-picomolar concentrations. In further embodiments, a multivalent and mulitspecific complex-drug conjugate (e.g., ELP-MRD fusion protein-drug conjugate) contains calicheamycin. For the preparation of conjugates of the calicheamicin family, see e.g., U.S. Pat. Nos. 5,712,374, 5,714,586, 5,739,116, 5,767,285, 5,770,701, 5,770,710, 5,773,001, and 5,877,296 (all to American Cyanamid Company). Structural analogues of calicheamicin which can be contained in the multivalent and mulitspecific complex-drug conjugate (e.g., ELP-MRD fusion protein-drug conjugate) of the invention include, but are not Cancer Research 53:3336-3342 (1993), and Lode et al., Cancer Research 58:2925-2928 (1998).

In other embodiments, multivalent and mulitspecific complex-drug conjugate (e.g., ELP-MRD fusion protein)-drug conjugate) compositions of the invention include a cytoxic agent selected from adriamicin, doxorubicin, mitomycin C, busulfan, cytoxin, chlorambucil, etoposide, etoposide phosphate, CC-1065, duocarmycin, KW-2189, CC1065, taxotere (docetaxel), methopterin, aminopterin, topotecan, camptothecin, porfiromycin, bleomycin, teniposide, esperamicins, mithramycin, anthramycin (AMC), fludarabine, tamoxifen, taxotere (docetaxel), cytosine arabinoside (Ara-C), adenosine arabinoside, cisplatin, carboplatin, cis-dichlorodiamine platinum (II) (DDP) cisplatin, chloroquine, cyclosporin A, docetaxel, paclitaxel, taxol, vinorelbine, vindesine, cytochalasin B, gramicidin D, ethidium bromide, emetine, mitomycin, ifosfamide, cyclophosphamide, tenoposide, caminomycin, porfiromycin, dihydroxy anthracin dione, mitoxantrone, mithramycin, dactinomycin, actinomycin D, puromycin 1-dehydrotestosterone, adriamycin, glucocorticoids, procaine, tetracaine, lidocaine, propranolol, epithiolone, QFA, combretastatin, combretastatin A4 phosphate, vinblastine, vincristine, colchicine, geldanamycin, doxorubicinchlorambucil, Auristatin F phenylene diamine (AFP)), monomethylauristatin, the family of agents known collectively LL-E33288 complex described in U.S. Pat. Nos. 5,053,394, 5,770,710, as well as esperamicins (U.S. Pat. No. 5,877,296) or a derivative or analog thereof and derivatives and analog thereof.

Additional suitable toxins and chemotherapeutic agents are described in Remington's Pharmaceutical Sciences, 19th Ed. (Mack Publishing Co. 1995), and in Goodman and Gilman's The Pharmacological Basis of Therapeutics, 7th Ed. (MacMillan Publishing Co. 1985). Moreover, for further discussion of types of cytotoxins, linkers and other methods that can be use or routinely adapted to conjugate therapeutic agents to the MRD-comprising ELP complex, see e.g., Intl. Appl. Publ. WO 2007/059404; Saito et al., Adv. Drug Deliv. Rev. 55:199-215 (2003); Trail et al. Cancer Immunol Immunother. 52:328-337 (2003); Payne, Cancer Cell 3:207-212 (2003); Allen, Nat. Rev. Cancer 2:750-763 (2002); Pastan et al., Curr. Opin. Investig. Drugs 3:1089-1091 (2002); and Senter et al., Adv. Drug Deliv. Rev. 53:247-264 (2001), each of which is hereby incorporated by reference in its entirety.

Cytotoxic agents that can be used in the MMM complexes of the invention (e.g., ELP-MRD fusion proteins-drug conjugates) include poisonous lectins and plant or other toxins (e.g., ricin, abrin, modeccin, botulina, and diphtheria toxins). It is envisioned that multiple copies of a toxin or combinations of various toxins can optionally be coupled to an MMM complex (e.g., an ELP-MRD fusion protein) thereby providing additional cytotoxicity. Enzymatically active toxins and fragments and variants thereof that can be used in compositions of the invention include, but are not limited to diphtheria A chain, nonbinding active fragments of diphtheria toxin, exotoxin A chain (from Pseudomonas aeruginosa), Pseudomonas exotoxin, Pseudomonas endotoxin, ricin A chain, abrin A chain, modeccin A chain, alpha-sarcin, Aleurites fordii proteins, ribonuclease, DNase I, Staphylococcal enterotoxin-A, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. See, for example, Pastan et al., Cell 47:641 (1986), Goldenberg et al., Cancer Journal for Clinicians 44:43 (1994) and Intl Appl. Publ. WO 93/21232 and WO 93/21232, each of which is herein incorporated by reference in its entirety.

Typically, peptide-based drug moieties can be prepared by forming a peptide bond between two or more amino acids and/or peptide fragments. Such peptide bonds can be prepared, for example, according to the liquid phase synthesis method (see E. Schroder and K. Lubke, “The Peptides”, volume 1, pp. 76-136, 1965, Academic Press) that is well known in the field of peptide chemistry. The auristatin/dolastatin drug moieties can be prepared according to the methods of: U.S. Pat. No. 5,635,483; U.S. Pat. No. 5,780,588; Pettit et., J. Am. Chem. Soc. 111:5463-5465 al (1989); Pettit et al., Anti-Cancer Drug Design 13:243-277 (1998); Pettit et al., Synthesis 719-725 (1996); Pettit et al J. Chem. Soc. Perkin Trans. 15:859-863 (1996); and Doronina Nat Biotechnol 21(7):778-784 (2003).

According to some embodiments, the MMM compositions of the invention comprise a highly radioactive atom. A variety of radioactive isotopes are available for the production of radioconjugated MMM complexes (e.g., ELP-MRD fusion proteins). Examples include At²¹¹, I¹²³, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the conjugate is used for detection, it may comprise a radioactive atom for scintigraphic studies, for example tc^{99m} or I¹²³, or a spin label for nuclear magnetic resonance (NMR) imaging (also known as magnetic resonance imaging, mri), such as I¹²³, I¹³¹, In¹¹¹, F¹⁹, C¹³, N¹⁵ O¹⁷, Gd, Fe, or Mn.

The radio- or other labels can be incorporated in the conjugate using techniques known in the art. For example, the peptide can be biosynthesized or can be synthesized by chemical amino acid synthesis using suitable amino acid precursors involving, for example, F¹⁹ in place of hydrogen. Labels such as tc^{99m} or I¹²³, Re¹⁸⁶, Re¹⁸⁸ and In¹¹¹ can be attached via a cysteine residue in the peptide. Y⁹⁰ can be attached via a lysine residue. The IODOGEN method (Fraker et al Biochem. Biophys. Res. Commun. 80: 49-57 (1978)) can be used to incorporate I¹²³. “Monoclonal Antibodies in Immunoscintigraphy” (Chatal, CRC Press 1989) describes in detail other methods that can be routinely applied to label the complexes of the invention.

A linker can be a “cleavable linker,” facilitating release of a drug in the cell. For example, an acid-labile linker (e.g., hydrazone), protease-sensitive (e.g., peptidase-sensitive) linker, photolabile linker, dimethyl linker or disulfide-containing linker (Chari et al., Cancer Research 52:127-131 (1992); U.S. Pat. No. 5,208,020, U.S. Pat. Appl. Publ. No. 20110293513) can be used. Thus, the invention encompasses MMM complexes containing one or more linkers that can contain any of a variety of groups as part of its chain that will cleave in vivo, e.g., in a cell, at a rate which is enhanced relative to that of constructs that lack such groups. Also provided are conjugates of the linker arms with therapeutic and diagnostic agents. The linkers are useful to form prodrug analogs of therapeutic agents and to reversibly link a therapeutic or diagnostic agent (e.g., cytotoxic agents and MRDs) to a targeting agent, a detectable label, or a solid support. The linkers can be stable in plasma so as not to release an MRD or cytotoxic agent. In the case of cytotoxic agents the linkers can be stable in plasma and labile once internalized so as to release the cytotoxic agent in an active form.

MRDs and/or cytotoxic agents are optionally attached to one another or to the MMM complex (e.g., ELP-MRD fusion protein) of the invention with a linker as described herein or otherwise known in the art. Conjugates of the MMM complex (e.g., ELP-MRD fusion protein) with an MRD or a cytotoxic agent can be made using a variety of bifunctional protein coupling agents known in the art, including, but not limited to, coupling agents containing a group selected from: 6-maleimidocaproyl (MC), maleimidocaproyl-polyethylene glycol (“MC(PEG)6-OH” (amenable to attachment to antibody cysteines)), maleimidopropanoyl (MP), MPBH, valine-citrulline (val-cit (exemplary dipeptide in a protease cleavable linker)), methyl-valine-citrulline (“Me-Val-CitN,” a linker in which a peptide bond has been modified to prevent its cleavage by cathepsin B) alanine-phenylalanine (ala-phe), p-aminobenzyloxycarbonyl (PAB (an example of a “self immolative” linker component)), valine-citrullin-p-aminobenzyloxycaronyl (“vc-PAB”), N-Succinimidyl 4-(2-pyridylthio) pentanoate (SPP), N-succinimidyl 4-(N-maleimidomethyl)cyclohexane-1 carboxylate (SMCC), LC-SMCC, N-Succinimidyl (4-iodo-acetyl) aminobenzoate (STAB), IT (iminothiolane), SPDP (N-succinimidyl-3-(2-pyridyldithio) propionate), 6-maleimidocaproyl-valine-citrulline-p-aminobenzyloxycarbonyl (MC-vc-PAB), ethyleneoxy —CH₂CH₂O— as one or more repeating units (“EO” or “PEO”), BMPS, EMCS, GMBS, HBVS, MBS, SBAP, SIA, SMPB, SMPH, sulfo-EMCS, sulfo-GMBS, sulfo-KMUS, sulfo-MBS, sulfo-SMCC, sulfo-SIAB, sulfo-SMPB, SVSB (succinimidyl-(4-vinylsulfone)benzoate), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Additional linker components are known in the art and some are described herein.

In some embodiments, the MMM complex (e.g., an ELP-MRD fusion protein) is covalently attached to a cytotoxic agent via a linker at 1-5, 5-10, 1-10, or 1-20 sites on the MMM complex. According to additional embodiments, the MMM complex is covalently attached to a cytotoxic agent via a linker at more than 2, 5 or 10 sites on the MMM complex.

In additional embodiments, the MMM complex (e.g., ELP-MRD fusion protein) is associated with a prodrug. The term “prodrug” as used herein, refers to precursor or derivative forms of pharmaceutically active substances that are less cytotoxic to tumor cells compared to their corresponding parent drugs and are capable of being enzymatically activated or converted into the more active parent form. Prodrugs encompassed by the invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, substituted phenoxyacetamide-containing prodrugs, substituted phenylacetamide-containing prodrugs, 5-fluorocytosine and other 5-fluorouridine prodrugs that can be converted into a more active free drug. Examples of cytotoxic drugs that can be derivatized into a prodrug form for use in this invention include, but are not limited to, those chemotherapeutic agents described herein. Prodrug synthesis, chemical linkage to antibodies, and pharmacodynamic properties are known in the art and can routinely be applied to make and use MMM complexes of the invention that contain prodrugs, such as, MMM-Drug (prodrug) complexes (e.g., ELP-MRD-Drug (prodrug) fusion proteins). See, e.g., Intl. Publ. No. WO 96/05863 and in U.S. Pat. No. 5,962,216, each of which is herein incorpatated by reference in it entirety.

Alternatively, a fusion protein comprising an ELP and a cytotoxic agent can be made, e.g., by recombinant techniques or peptide synthesis. A recombinant DNA molecule can comprise regions encoding the ELP and cytotoxic portions of the conjugate either adjacent to one another or separated by a region encoding a linker peptide which does not destroy the desired properties of the conjugate.

The MMM complex (e.g., ELP-MRD fusion protein) composition of the invention also can be conjugated to a radioactive isotope to generate cytotoxic radiopharmaceuticals, also referred to as radioMMM complexs. Examples of radioactive isotopes that can be conjugated to MMM complexes (e.g., MRD containing antibodies) for use diagnostically or therapeutically include, but are not limited to, iodine¹³¹, indium¹¹¹, yttrium⁹⁰, and lutetium¹⁷⁷.

In some embodiments, an MMM complex of the invention comprises a cytotoxic agent (e.g., an ELP-MRD fusion protein-cytotoxic agent conjugate) and may generally be referred to herein as an MMM complex. In some embodiments, an MMM complex of the invention binds a cell surface target that is internalized into the cell. In further embodiments, the binding of an MMM complex of the invention (e.g., an ELP-MRD fusion protein-cytotoxic agent conjugate) to a cell surface target results in the internalization of the MMM complex into the cell in vitro. In further embodiments, the binding of MMM complex to a cell surface target results in the internalization of the composition into the cell in vivo. Methods for treating a patient described herein can comprise: administering to the patient a therapeutically effective amount of an MMM complex (e.g., an ELP-MRD fusion protein) of the invention wherein the MMM complex comprises a cytotoxic agent, (e.g., an ELP-MRD fusion protein-cytotoxic agent conjugate) and wherein the MMM complex (e.g., an ELP-MRD fusion protein-cytotoxic agent conjugate) binds a target that is internalized into a cell. In some embodiments, the MMM complex comprises a cytotoxic agent disclosed herein. In additional embodiments, the MMM complex comprises a cytotoxic agent selected from an alkylating agent, antiproliferative agent, tubulin binding agent, vinca alkaloid, enediyne, podophyllotoxin, podophyllotoxin derivative, a member of the pteridine family of drugs, taxane, a dolastatin, topoiosomerase inhibitor, or a platinum complex chemotherapeutic agent. In further embodiments, the cytoxic agent is a maytansinoid or a maytansinoid derivative or analog. In specific embodiments, the cytoxic agent is the maytansinoid DM1, DM2, or DM3. In additional embodiments, the cytotoxic agent is auristatin or an auristatin derivative or analog. In specific embodiments, the cytoxic agent is MMAE or MMAF. The cytotoxic agents are optionally attached to the other components of the MMM complex by a linker. In some embodiments, the cytotoxic agent is attached to the other components of the MMM complex by an enzyme cleavable linker. In additional embodiments, the cytotoxic agent is attached to the other components of the MMM complex by an acid-labile linker.

In further embodiments, the cytoxic agent of the MMM complex (e.g., an ELP-MRD fusion protein) has a free drug potency of less than 10⁻⁷M, 10⁻⁸M, or 10⁻⁹M. In additional embodiments, the cytoxin has a free drug potency of 10⁻⁸ to 10⁻¹¹M.

In some embodiments, a target bound by the MMM complex (e.g., an ELP-MRD fusion protein) is a member selected from: CD19, CD22, CD30, CD33, CD56, CD70, CD79a, CD80, CD83, CD95, CD126, CD133, CD138, PSMA, EphA2, ErbB2 (CD340), SLC44A4, MN (carbonic anhydrase IX), GPNMB (glycoprotein non-metastatic melanoma protein), Cripto, and aV integrin. In additional embodiments, a target bound by the MMM complex (e.g., an ELP-MRD fusion protein) is a member selected from: CD1, CD1a, CD2, CD3, CD4, CD5, CD8, CD11A, CD14, CD15, CD16, CD18, CD19, CD20, CD25, CD40, CD64, CD74, CD79, CD105, CD174, CD205, CD227, CD326, CD340, MUC16, EGP-1, EGP-2, EGF receptor (ErbB1), ErbB2, ErbB3, Factor H, FHL-1, Flt-3, folate receptor, Ga 733, GROB, HMGB-1, hypoxia inducible factor (HIF), HM1.24, HER-2/neu, insulin-like growth factor (ILGF), IFN-gamma, IFN-.alpha, IFN-beta, IL-2R, IL-4R, IL-6R, IL-13R, IL-15R, IL-17R, IL-18R, IL-2, IL-6, IL-8, IL-12, IL-15, IL-17, IL-18, IL-25, IP-10, IGF-1R, Ia, HM1.24, HCG, HLA-DR, ED-B, TMEFF2, EphB2, FAP (fibroblast activation protein), mesothelin, EGFR, TAG-72, GD2 (encoded by the B4GALNT1 gene), and 5T4.

In some embodiments, a target bound by the MMM complex (e.g., an ELP-MRD fusion protein) is a receptor in the Tumor Necrosis Factor (TNF) receptor superfamily. In additional embodiments, a target bound by the MMM complex is selected from: TNFRSF10A (TRAIL R1 DR4), TNFRSF10B (TRAIL R2DR5), TNRSF10C (DcR1), and TNRSF10D (DcR3). In additional embodiments, a target bound by the MMM complex is selected from: TNFRSF21 (DR6), TNFRSF25 (DR3), TNFRSF1A, TNFRSF1B, TNFRSF4, TNFRSF9, TNFRSF12A, TNFRSF13B, TNFRSF13C, TNFRSF14 and TNFRSF18. In further embodiments, a target bound by the MMM complex is TNFRSF11A or TNFRSF11B.

In additional embodiments, a target bound by the MMM complex (e.g., an ELP-MRD fusion protein) is a myeloid and hematopoietic target selected from CD33, CD64, CD40, CD56, and CD138. In further embodiments, a target bound by the MMM complex is a carcinoma target selected from EpCam, GD2, EGFR, CD74, CD227, CD340, MUC16, GD2, GPNMB, PSMA, crypto, TMEFF2, EphB2, 5t4, mesothelin, TAG-72 and MN.

In other embodiments, a target bound by the MMM complex (e.g., an ELP-MRD fusion protein) is a B cell target selected from: CD19/CD21, CD20, CD22, CD40, CD70, CD79a, CD79b, and CD205.

In additional embodiments, a target bound by the MMM complex (e.g., an ELP-MRD fusion protein) is a T cell target selected from CD25, CD30, CD40, CD70, and CD205. In further embodiments, a target bound by the endothelial cell target CD105, the stromal cell target FAP and the vascular target ED-B.

Alternatively, a fusion protein comprising the antibody and cytotoxic agent can be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

MMM. In additional embodiments, the MMM complex of the invention has in vitro or in vivo cell killing activity. In one embodiment, the linker is attached to the ELP through a thiol group on the ELP. In one embodiment, the linker is cleavable by a protease. In one embodiment, the linker comprises a val-cit dipeptide. In one embodiment, the linker comprises a p-aminobenzyl unit. In one embodiment, the p-aminobenzyl unit is disposed between the drug and a protease cleavage site in the linker. In one embodiment, the p-aminobenzyl unit is p-aminobenzyloxycarbonyl (PAB). In one embodiment, the linker comprises 6-maleimidocaproyl. In one embodiment, the 6-maleimidocaproyl is disposed between the antibody and a protease cleavage site in the linker. The above embodiments, may occur singly or in any combination with one another.

The MMM complex (e.g., ELP-MRD fusion protein) of the present invention may also be conjugating to a prodrug-activating enzyme which converts a prodrug (e.g., a peptidyl chemotherapeutic agent, see e.g., WO81/01145) to an active anti-cancer drug. See, for example, WO 88/07378 and U.S. Pat. No. 4,975,278 the contents of which are herein incorporated by reference in its entirety. The enzyme component of the MMM complex (e.g., an ELP-MRD fusion protein) is preferably capable of acting on a prodrug in such a way so as to convert it into its more active, cytotoxic form. See, for example, Pastan et al., Cell, 47:641 (1986), and Goldenberg et al., Cancer Journal for Clinicians, 44:43 (1994). Enzymatically active toxins and fragments thereof which can be used include diphtheria A chain, non-binding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232.

In some embodiments, the MMM complexes of the invention (e.g., ELP-MRD fusion proteins) are conjugated to a radioisotope, such as ⁹⁰Y, ¹²⁵I, ¹³¹I, ¹²³I, ¹¹¹In, ¹⁰⁵Rh ¹⁵³Sm, ⁶⁷Cu, ⁶⁷Ga, ¹⁶⁶Ho, ¹⁷⁷Lu, ¹⁸⁶Re and ¹⁸⁸Re using anyone of a number of well-known chelators or direct labeling. In other embodiments, the MMM complex (e.g., ELP-MRD fusion protein) is coupled to drugs, prodrugs or lymphokines such as interferon. Compositions of the invention can be labeled with ligand reagents that bind, chelate or otherwise complex a radioisotope metal where the reagent is reactive with the engineered cysteine thiol of the ELP, using techniques known in the art such as, those described in Current Protocols in Immunology, Volumes 1 and 2, Coligen et al, Ed. Wiley-Interscience, New York, N.Y., Pubs. (1991). Chelating ligands which may complex a metal ion and that may have use in the compositions and methods of the invention include DOTA, DOTP, DOTMA, DTPA and TETA (Macrocyclics, Dallas, Tex.). Radionuclides can be targeted via complexation with the ELP-drug conjugates of the invention (Wu et al Nature Biotechnology 23(9): 1137-1146 (2005)). Linker reagents such as DOTA-maleimide (4-maleimidobutyramidobenzyl-DOTA) can be prepared by the reaction of aminobenzyl-DOTA with 4-maleimidobutyric acid (Fluka) activated with isopropylchloroformate (Aldrich), following the procedure of Axworthy et al., Proc. Natl. Acad. Sci. USA 97(4):1802-1807 (2000)). DOTA-maleimide reagents react with the free cysteine amino acids of the cysteine engineered antibodies and provide a metal complexing ligand on the antibody (Lewis et al., Bioconj. Chem. 9:72-86 (1998)). Chelating linker labeling reagents such as DOTA-NHS (1,4,7,10-tetraazacyclododecane-1,4,7,10-tetraacetic acid mono (N-hydroxysuccinimide ester) are commercially available (Macrocyclics, Dallas, Tex.).

Conjugates of the MMM complexes of the invention (e.g., ELP-MRD fusion proteins) and cytotoxin can routinely be made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). In specific embodiments, the toxin is conjugate to an MMM complex (e.g., an ELP-MRD fusion protein) through an enzyme-cleavable linker system (e.g., such as that present in SGN-35). Conjugates of an MMM complex (e.g., an ELP-MRD fusion protein) and one or more small molecule toxins, such as a calicheamicin, maytansinoids, a trichothene, and CC1065, and the derivatives of these toxins that have toxin activity, can also be used. In some embodiments, the MMM complex (e.g., ELP-MRD fusion protein).

In some embodiments, the MMM complex (e.g., ELP-MRD fusion protein) can be complexed, or have MRDs that bind with other immunologically active ligands (e.g., chemokines, cytokines, and antibodies or fragments thereof) wherein the resulting molecule binds to the neoplastic cell or other target as well as the chemokine, cytokine, or an effector cell such as a T cell. In certain embodiments, these conjugates can be generated as fusion proteins. The enzymes of this invention can be covalently bound to the antibody by techniques well-known in the art such as the use of the heterobifunctional crosslinking reagents discussed above.

In additional embodiments, an MRD and/or the MMM complex (e.g., ELP-MRD fusion protein) comprises one or more amino acid sequences that allow the MMM complex (e.g., ELP-MRD fusion protein) to cross the blood brain barrier. In particular examples, said one or more amino acid sequences that allow the MMM complex (e.g., ELP-MRD fusion protein) to cross the blood brain barrier are selected from FC44 or FC5 (see e.g., WO 02/057445: FC44 and FC5, which is herein incorporated by reference).

The further amino acid sequences can also be a signal sequence or leader sequence that directs secretion of the ELP-MRD fusion from a host cell upon synthesis, for example to provide a pre-, pro- or prepro-form of the polypeptide of the invention, depending on the host cell used to express the polypeptide.

The further amino acid sequence can also form a sequence or signal that allows the polypeptide of the invention to be directed towards and/or to penetrate or enter into specific organs, tissues, cells, or parts or compartments of cells, and/or that allows the polypeptide of the invention to penetrate or cross a biological barrier such as a cell membrane, a cell layer such as a layer of epithelial cells, a tumor including solid tumors, or the blood-brain-barrier. Suitable examples of such amino acid sequences will be clear to the skilled person, and for example include, but are not limited to, the sequences described by Cardinale et al. and the amino acid sequences and antibody fragments known per se that can be used to express or produce the Nanobodies and polypeptides as so-called “intrabodies”, for example as described in WO 94/02610, WO 95/22618, U.S. Pat. No. 6,004,940, WO 03/014960, WO 99/07414; WO-05/01690; EP 1 512 696; and in Cattaneo, A. & Biocca, S. (1997) Intracellular Antibodies: Development and Applications. Landes and Springer-Verlag; and in Kontermann, Methods 34, 163-170 (2004), and the further references described therein.

### III. Linkers

MMM complexes (e.g., ELP-MRD fusion proteins) can contain a single linker, multiple linkers, or no linkers. Thus, an MRD or other modular component can be operably attached (linked) to the ELP directly (i.e. without a linker peptide), or operably attached through an optional linker peptide. Similarly, a MRD or other modular component can be operably attached to one or more MRD(s) directly, or operably attached to one or more MRD(s) through one or more optional linker peptide(s). In one emobidment, an MRD or other modular component of an ELP-MRD fusion is directly (i.e. without a linker) attached. In another embodiment, an MRD or other modular component of an ELP-MRD fusion is attached through a linker.

In one embodiment, an ELP-MRD fusion comprises an ELP and MRD operably linked through a linker peptide. In one embodiment, the linker comprises a sequence selected from the group consisting of SEQ ID NO:1, SEQ ID NO:2, and SEQ ID NO:19. In one embodiment, an ELP-MRD fusion comprises at least 2, at least 3, at least 4, or at least 5 MRDs operably linked to another component of the ELP-MRD fusion through a linker peptide.

In one embodiment, an ELP-MRD fusion comprises an ELP directly (i.e. without a linker) attached to an MRD. In one embodiment, an ELP-MRD fusion comprises an MRD directly (i.e. without a linker) attached to another component of an ELP-MRD fusion. In one embodiment, an ELP-MRD fusion comprises at least 2, at least 3, at leat 4, or at least 5 MRDs directly (i.e. without a linker) attached to another component of the ELP-MRD fusion.

Linkers can be of any size or composition so long as they are able to operably attach an MRD, an ELP, or other ELP-MRD fusion component to an MRD, ELP, or other MMM complex (e.g., ELP-MRD fusion protein) component such that the MRD enables the MMM complex (e.g., ELP-MRD fusion protein) to bind an MRD target.

In some embodiments, linkers have about 1 to 20 amino acids, about 1 to 15 amino acids, about 1 to 10 amino acids, about 1 to 5 amino acids, about 2 to 20 amino acids, about 2 to 15 amino acids, about 2 to 10 amino acids, or about 2 to 5 amino acids. In additional embodiments, the linkers have about 4 to 15 amino acids. In certain embodiments, the linker peptide contains a short linker peptide with the sequence GGGS (SEQ ID NO:1), a medium linker peptide with the sequence SSGGGGSGGGGGGSS (SEQ ID NO:2), or a long linker peptide with the sequence SSGGGGSGGGGGGSSRSS (SEQ ID NO:19). In another embodiment, an MRD is inserted into the fourth loop in the light chain constant region. For example, an MRD can be inserted between the underlined letters in the following amino acid sequence: RTVAAPSVFIFPPSDEQLKSGTASVVCLLNNFYPREAKVQWKVDKLGTNSQESVTEQDSKDSTYSLSSTLTLSKADYEKHKVYACEVTHQGLSLPVTKSFNRGEC (SEQ ID NO:102).

The linker can also be a non-peptide linker such as an alkyl linker, or a PEG linker. For example, alkyl linkers such as —NH—(CH₂)s-C(O)—, wherein s=2-20 can be used. These alkyl linkers may further be substituted by any non-sterically hindering group such as lower alkyl (e.g., C₁ C₆) lower acyl, halogen (e.g., Cl, Br), CN, NH₂, phenyl, etc. An exemplary non-peptide linker is a PEG linker. In certain embodiments, the PEG linker has a molecular weight of about 100 to 5000 kDa, or about 100 to 500 kDa.

In some embodiments, the linker is a “cleavable linker” facilitating release of an MRD or cytotoxic agent in the cell. For example, an acid-labile linker (e.g., hydrazone), protease-sensitive (e.g., peptidase-sensitive) linker, photolabile linker, dimethyl linker or disulfide-containing linker (Chari et al., Cancer Research 52:127-131 (1992); U.S. Pat. No. 5,208,020; U.S. Appl. Pub. No. 20090110753) can be used wherein it is desrirable that the covalent attachment between an MRD or a cytoxic agent and the MMM complex (e.g., ELP-MRD fusion protein) is intracellularly cleaved when the composition is internalized into the cell. The terms “intracellularly cleaved” and “intracellular cleavage” refer to a metabolic process or reaction inside a cell on an antibody-drug conjugate (ADC) whereby the covalent attachment, i.e., linked via a linker between the MRD and cytotoxic agent, MRD and ELP, ELP and cytotoxic agent, or between two MRDs is broken, resulting in the free MRD and/or cytotoxic agent dissociated from the ELP inside the cell. The cleaved moieties of the zybody-ADC are thus intracellular metabolites.

In additional embodiments, one or more of the linkers in the MMM complex (e.g., ELP-MRD fusion protein) is cleavable. Examples of cleavable linkers include, without limitation, a peptide sequence recognized by proteases (in vitro or in vivo) of varying type, such as Tev, thrombin, factor Xa, plasmin (blood proteases), metalloproteases, cathepsins (e.g., GFLG, etc.), and proteases found in other corporeal compartments. In some embodiments, one or more functionalities of the MMM complex (e.g., ELP-MRD fusion protein) is activated, or rendered more active upon cleavage of a cleavable linker. In other embodiments, one or more functionalities of the MMM complex (e.g., ELP-MRD fusion protein) is activated, or rendered more active upon cleavage of a cleavable linker in vivo.

Linker optimization can be evaluated using the techniques described herein and techniques otherwise known in the art. In some embodiments, linkers do not disrupt the ability of an MRD to bind a target molecule and/or an antibody domain or fragment to bind an antigen.

### VI. MMM Complexes

An ELP and MRD can be combined to form a single molecule that is an MMM complex (e.g., an ELP-MRD fusion protein). These MMM complexes (e.g., ELP-MRD fusion proteins) can additionally contain other optional components such as the linkers and other modular components described herein. MRDs, antibody fragments or domains (e.g., antibody variable domains, ScFvs and domains), therapeutic proteins and other components of ELP-MRD fusions can be operably linked to one another and/or to the amino terminus or carboxy terminus of the ELP directly or through a linker. In one embodiment, an MRD of an MMM complex (e.g., an ELP-MRD fusion protein) is operably linked to the carboxy-terminus of an ELP. In another embodiment, an MRD of an MMM complex (e.g., an ELP-MRD fusion protein) is operably linked to the amino-terminus of an ELP. In additional embodiments, MRDs of an MMM complex (e.g., an ELP-MRD fusion protein) are operably linked to the amino terminus and carboxy terminus of an ELP. In additional embodiments, 2 or more MRDs of an MMM complex (e.g., ELP-MRD fusion protein) are operably linked to the carboxy-terminus of an ELP. In other embodiments, embodiments, 2 or more MRDs are operably linked to the amino-terminus of an ELP.

An MMM complex (e.g., ELP-MRD fusion protein) can be “monospecific” or “multispecific.” Whether an MMM complex (e.g., an ELP-MRD fusion protein) is “monospecific” or “multispecific,” (e.g., bispecific, trispecific, and tetraspecific) refers to the number of different epitopes that the MMM complex (e.g., ELP-MRD fusion protein) binds. An MMM complex (e.g., an ELP-MRD fusion protein) that is “multispecific” (e.g., bispecific, trispecific tetraspecific, pentaspecific or of greater multispecificity) recognizes and binds to 2 or more different epitopes present on one or more different molecules (e.g., proteins, solid support structures, etc.).

In some embodiments, an MMM complex (e.g., an ELP-MRD fusion protein) contains multiple MRDS that bind to the same epitope. In other embodiments, an MMM complex (e.g., an ELP-MRD fusion protein) contains at least one MRD and at least one other modular component, e.g., an antibody fragment or binding domain, that bind to the same epitope.

The present invention contemplates the preparation of mono-, bi-, tri-, tetra-, and penta-specific MMM complexes (e.g., ELP-MRD fusion proteins) as well as MMM complexes (e.g., ELP-MRD fusion proteins) of greater multispecificity. A multispecific MMM complex (e.g., ELP-MRD fusion protein) can contain at least 2 MRDs that bind to at least 2 different epitopes on a single target polypeptide. A multispecific MMM complex (e.g., ELP-MRD fusion protein) can also contain at least one MRD that binds to an epitope on a target polypeptide and at least one other modular component, e.g., an antibody fragment or domain, that binds to a different epitope on the same polypeptide. A multispecific MMM complex (e.g., ELP-MRD fusion protein) can also contain at least one MRD that binds to an epitope on a target polypeptide and at least one MRD that binds to an epitope on a different target polypeptide. A multispecific MMM complex (e.g., ELP-MRD fusion protein) can also contain at least one MRD that binds to an epitope on a target polypeptide and at least one other modular component, e.g., an antibody fragment or domain, that binds to an epitope on a different target polypeptide. In other embodiments, an MMM complex (e.g., an ELP-MRD fusion protein) contains at least one MRD or other modular component, e.g., an antibody fragment or domain, that binds to a polypeptide and at least one other MRD or other modular component, e.g., an antibody fragment or domain, that binds to a solid support material.

In one embodiment, the MMM complex (e.g., ELP-MRD fusion protein) binds 2 different epitopes. In an additional embodiment the MMM complex (e.g., ELP-MRD fusion protein) binds 2 different epitopes simultaneously. In another embodiment, the MMM complex (e.g., ELP-MRD fusion protein) binds 3 different epitopes. In an additional embodiment the MMM complex (e.g., ELP-MRD fusion protein) binds 3 different epitopes simultaneously. In another embodiment, the MMM complex (e.g., an ELP-MRD fusion protein) binds 4 different epitopes. In an additional embodiment the MMM complex (e.g., an ELP-MRD fusion protein) binds 4 different epitopes simultaneously. In another embodiment, the MMM complex (e.g., an ELP-MRD fusion protein) binds 5 different epitopes. In an additional embodiment the MMM complex (e.g., an ELP-MRD fusion protein) binds 5 different epitopes simultaneously.

In other embodiments, 2 MRDs of the MMM complex (e.g., an ELP-MRD fusion protein) bind the same target. In other embodiments, 3, 4, 5, 6, 7, 8, 9, or 10 MRDs of the MMM complex (e.g., an ELP-MRD fusion protein) bind the same target. In other embodiments, at least 2 MRDs of the MMM complex (e.g., an MMM complex (e.g., an ELP-MRD fusion protein) bind the same target. In other embodiments, at least 3, 4, 5, 6, 7, 8, 9, or 10 MRDs of the MMM complex (e.g., ELP-MRD fusion protein) bind the same target. In other embodiments, 2 MRDs of the MMM complex (e.g., an ELP-MRD fusion protein) bind the same epitope. In other embodiments, embodiments, 3, 4, 5, 6, 7, 8, 9, or 10 MRDs of the MMM complex (e.g., an ELP-MRD fusion protein) bind the same epitope. In other embodiments, embodiments, at least 2 MRDs of the MMM complex (e.g., an ELP-MRD fusion protein) bind the same epitope. In other embodiments, embodiments, at least 3, 4, 5, 6, 7, 8, 9, or 10 MRDs of the MMM complex (e.g., an ELP-MRD fusion protein) bind the same epitope. It is envisioned that these MRDs can be the same or different. In addition, any combination of MRD number and linkages can be used. The MMM complex (e.g., an ELP-MRD fusion protein) can contain 1, 2, 3, 4, 5, 6, 7, 8, 9, 10 ore more than 10 MRDs.

In one embodiment, MMM complex (e.g., an ELP-MRD fusion protein) contains at least 1 MRD. In preferred embodiments, the MMM complex (e.g., an ELP-MRD fusion protein) contains at least 2 MRDs. In another embodiment, the MMM complex (e.g., an ELP-MRD fusion protein) contains at least 3 MRDs. In another embodiment, the MMM complex (e.g., an ELP-MRD fusion protein) contains at least 4 MRDs. In another embodiment, the MMM complex (e.g., an ELP-MRD fusion protein) contains at least 5 MRDs. In another embodiment, the MMM complex (e.g., an ELP-MRD fusion protein) contains at least 6 MRDs.

In another embodiment, the MMM complex (e.g., an ELP-MRD fusion protein) contains 2 different MRDs. In another embodiment, the MMM complex (e.g., an ELP-MRD fusion protein) contains 3 different MRDs. In another embodiment, the MMM complex (e.g., an ELP-MRD fusion protein) contains 4 different MRDs. In another embodiment, the MMM complex (e.g., an ELP-MRD fusion protein) contains 5 different MRDs. In another embodiment, the MMM complex (e.g., an ELP-MRD fusion protein) contains 6 different MRDs. In an additional embodiment, the MMM complex (e.g., ELP-MRD fusion protein) contains between 2 and 10 different MRDs.

In another embodiment, the MMM complex (e.g., an ELP-MRD fusion protein) contains at least 2 different MRDs. In another embodiment, the MMM complex (e.g., an ELP-MRD fusion protein) contains at least 3 different MRDs. In another embodiment, the MMM complex (e.g., an ELP-MRD fusion protein) contains at least 4 different MRDs. In another embodiment, the MMM complex (e.g., ELP-MRD fusion protein) contains at least 5 different MRDs. In another embodiment, the MMM complex (e.g., ELP-MRD fusion protein) contains at least 6 different MRDs.

Thus, the MMM complexes (e.g., ELP-MRD fusion proteins) can be MRD monomeric (i.e., containing one MRD) or MRD multimeric (i.e., containing more than 1 MRD in tandem optionally connected by a linker). The multimeric MMM complexes (e.g., ELP-MRD fusion proteins) can be homo-multimeric (i.e., containing more than 1 of the same MRD in tandem optionally connected by linker(s) (e.g., homodimers, homotrimers, homotetramers etc.)) or hetero-multimeric (i.e., containing 2 or more MRDs in which there are at least 2 different MRDs. Moreover, multiple tandem components can contain the same or different MRDs. In additional embodiments, embodiments, MRDs are released by proteolysis of one or more spacer moieties separating 1 or more tandem MMM complex (e.g., ELP-MRD fusion protein) components. In some embodiments, one or more MRD components is active in the fused state. Alternatively, in some embodiments, one or more of MRD components of the MMM complex (e.g., an ELP-MRD fusion protein) is inactive in the fused state, and becomes active upon proteolytic release from the MMM complex (e.g., an ELP-MRD fusion protein).

The multiple MRDs in MMM complexes (e.g., ELP-MRD fusion proteins) can target the same target binding-site, or 2 or more different target-binding sites. Where MRDs bind to different target-binding sites, the binding sites can be on the same or different target molecules.

In some embodiments, the MMM complexes (e.g., ELP-MRD fusion proteins) bind at least 2 targets simultaneously. In one embodiment, each MRD in an MMM complex (e.g., an ELP-MRD fusion protein) can bind to its target simultaneously. Therefore, in some embodiments, the MMM complex (e.g., an ELP-MRD fusion protein) binds 2, 3, 4, 5, 6, 7, 8, 9, 10 or more target molecules simultaneously.

The ability of an MMM complex (e.g., an ELP-MRD fusion protein) to bind to multiple targets simultaneously can be assayed using methods known in the art, including, for example, those methods described in the examples below.

In additional embodiments, the MMM complexes (e.g., ELP-MRD fusion proteins) of the invention have a single binding site for (i.e., monovalently bind) a target.

It is envisioned that in some embodiments, the MMM complexes (e.g., ELP-MRD fusion proteins) of the invention have a single binding site for (i.e., monovalently bind) a target. In some embodiments, the single binding site is an MRD. Thus, the MMM complexes of the invention encompass (and can be routinely engineered to include) MMM complexes (e.g., ELP-MRD fusion proteins) that contain 1, 2, 3, 4 or more single binding sites for a target. In further embodiments, the MMM complex (e.g., an ELP-MRD fusion proteins) has a single binding site for (i.e., monovalently binds) a cell surface target that forms multimers (e.g., homomers or heteromers). In some embodiments, the single binding site binds a cell surface target that requires multimerization for signaling. In some embodiments, the MMM complex (e.g., ELP-MRD fusion proteins) has a single binding site that binds a cell surface target and inhibits binding of another molecule (such as a ligand) to the cell surface target. In other embodiments, binding of the single binding site inhibits multimerization of the target (e.g., homomeric and heteromeric multimerization). In additional embodiments, the complex has single binding sites for different targets (i.e., monovalently binds more than one different target). In some embodiments, the multiple single binding sites of the complex bind targets on the same cell. In additional embodiments, the multiple single binding sites of the complex bind targets on different cells. Numerous receptors are known in the art that require multimerization for affecting their normal function. Such receptors are envisioned to be targets of single binding sites in the MMM complexes (e.g., ELP-MRD fusion proteins) of the invention. In some embodiments, the complex has a single binding site for a receptor tyrosine kinase. In some embodiments, the complex has a single binding site for a growth factor receptor. In additional embodiments, embodiments, the complex ion has a single binding site for a G protein coupled receptor. In additional embodiments, embodiments, the complex has a single binding site for a chemokine receptor. In other embodiments, the complex has a single binding site for a TNF receptor superfamily member. In particular embodiments, the complex has a single binding site for a receptor selected from: RAGE, c-Met, ErbB2, VEGFR1, VEGFR2, VEGFR3, FGFR1 (e.g., FGFR1-IIIC), FGFR2 (e.g., FGFR2-IIIa, FGFR2-IIIb, and FGFR2-IIIc), FGFR3, PDGFRA, PDGFRB, netrin, CD28, TNFRSF1A (TNFR1, p55, p60), TNFRSF1B (TNFR2), TNFSF6 (Fas Ligand), TNFRSF6 (Fas, CD95), TNFRSF21 or TNFRSF25, TNFRSF7 (CD27), TNFSF8 (CD30 Ligand), TNFRSF8 (CD30), TNFSF11 (RANKL), TNFRSF11A (RANK), TNFRSF21 (DR6), TNFRSF25 (DR3), and LRP6.

In additional embodiments, the MMM complex (e.g., an ELP-MRD fusion protein) has a single binding site for (i.e., monovalently binds) a cell surface target that forms a multimer and multiple sites (i.e., multivalently binds) for 2 or more different targets. In other embodiments, the MMM complex has a single binding site for a cell surface target and multiple binding sites for 1, 2, 3, 4, 5 or more different targets. In further embodiments, at least 1, 2, 3, 4, 5 or more of the targets bound by the MMM complex are located on a cell surface. In other embodiments, at least 1, 2, 3, 4, 5 or more of the targets bound by the MMM complex are soluble targets (e.g., chemokines, cytokines, and growth factors). In additional embodiments, the composition binds 1, 2, 3, 4, 5 or more of the targets described herein. In further embodiments, the targets bound by the composition are tumor antigens (including tumor antigens and tumor associated antigens). In additional embodiments, a target bound by the composition is associated with a disease or disorder of the immune system. In further embodiments, a targets bound by the composition is associated with a disease or disorder of the skeletal system (e.g., osteoporosis), cardiovascular system, nervous system, or an infectious disease.

In one embodiment, an MMM complex (e.g., an ELP-MRD fusion protein) binds TNF alpha and additionally binds a target selected from: Te38, IL-12, IL-12p40, IL-13, IL-15, IL-17, IL-18, IL-1beta, IL-23, MIF, PEG2, PGE4, VEGF, TNFSF11 (RANKL), TNFSF13B (BLYS), GP130, and CD-22 and CTLA-4. In another embodiment, an MMM complex (e.g., an ELP-MRD fusion protein) binds TNF alpha, IL-6 and TNFSF13B (BLYS).

In one embodiment, an MMM complex (e.g., an ELP-MRD fusion protein) binds IL-1 alpha and IL-1 beta. In another embodiment, an MMM complex (e.g., an ELP-MRD fusion protein) binds IL-12 and additionally binds IL-18 or TWEAK. In an additional embodiment, an MMM complex (e.g., an ELP-MRD fusion protein) binds CTLA-4 and additionally binds PDL-1 or BTNO2.

In an additional embodiment, an MMM complex (e.g., an ELP-MRD fusion protein) binds IL-13 and additionally binds a target selected from: IL-1beta, IL-4; IL-9, IL-13, IL-25, LHR agonist, MDC, MIF, PED2, SPRR2a, SPRR2b; TARC, TGF-beta and CL25.

In a further embodiment, an MMM complex (e.g., an ELP-MRD fusion protein) binds RGM A and additionally binds a target selected from: RGM B, MAG, NgR, NogoA, OMGp and CSPGs.

In another embodiment, an MMM complex (e.g., an ELP-MRD fusion protein) binds CD38 and additionally binds a target selected from CD20, CD40 and CD138.

In one embodiment, an MMM complex (e.g., an ELP-MRD fusion protein) binds

ErbB2, and IGF1R. In another embodiment, an MMM complex (e.g., an ELP-MRD fusion protein) binds ErbB2, Ang2, and IGF1R.

In another embodiment, an MMM complex (e.g., an ELP-MRD fusion protein) binds VEGFR1 and additionally binds an angiogenic target selected from: VEGFA, VEGFB, FGF1, FGF2, FGF4, FGF7, FGF8b, FGF19, FGFR1 (e.g., FGFR1-IIIC), FGFR2 (e.g., FGFR2-IIIa, FGFR2-IIIb, and FGFR2-IIIc), FGFR3, TNFSF2 (TNFa), FGFR3, EFNa1, EFNa₂, ANG1, ANG2, IL-6, IL-8, IL-18, HGF, PDGFA, P1GF, PDGFB, CXCL12, KIT, GCSF, CXCR4, PTPRC, TIE2, VEGFR2, VEGFR3, Notch 1, DLL4, EGFL7, a2131 integrin a4131 integrin, a5131 integrin, αvβ3 integrin, TGFb, MMP2, MMPI, MMP9, MMP12, PLAU, VCAM1, PDGFRA and PDGFRB. MMM complexes (e.g., ELP, ELP-MRD fusion proteins) that bind VEGFR1 and additionally bind 2, 3, 4, 5 or more of these targets are also encompassed by the invention.

In some embodiments, an MMM complex (e.g., an ELP-MRD fusion protein) binds ErbB2 and HER2/3. In further embodiments, an MMM complex (e.g., an ELP-MRD fusion protein) binds ErbB2 and HER2/3 simultaneously.

Angiogenesis inhibitors targeting the vascular endothelial growth factor (VEGF) signaling pathways have been observed to provide at best transitory therapeutic benefits followed by restoration of tumor growth and progression due to an apparent ability of angiogenic tumors to adapt the presence of these inhibitors. Without being bound by theory, it is believed that the monovalent and multivalent multispecific properties of MMM complexes (e.g., ELP, ELP-MRD fusion proteins) that bind an angiogenesis target provide these compounds with an ability to extend anti-angiogenic therapeutic benefits beyond those observed from for example, conventional monoclonal antibody therapies by binding multiple distinct angiogenesis related targets and thereby disrupting resistance mechanisms available to the angiogenic tumor.

In one embodiment, an MMM complex (e.g., an ELP-MRD fusion protein) binds 2 or more targets selected from: VEGFA, VEGFB, FGF1, FGF2, FGF4, FGF7, FGF8b, FGF19, FGFR1 (e.g., FGFR1-IIIC), FGFR2 (e.g., FGFR2-IIIa, FGFR2-IIIb, and FGFR2-IIIc), FGFR3, TNFSF2 (TNFa), FGFR3, EFNa1, EFNa2, ANG1, ANG2, IL-6, IL-8, IL-18, HGF, PDGFA, P1GF, PDGFB, CXCL12, KIT, GCSF, CXCR4, PTPRC, TIE2, VEGFR1, VEGFR2, VEGFR3, Notch 1, DLL4, EGFL7, α2β1 integrin α4β1 integrin, α5β1 integrin, αvβ3 integrin, TGFb, MMP2, MMP7, MMP9, MMP12, PLAU, VCAM1, PDGFRA and PDGFRB.

In one embodiment, an MMM complex (e.g., an ELP-MRD fusion protein) binds

VEGFA and additionally binds an angiogenic target selected from: VEGFB, FGF1, FGF2, FGF4, FGF7, FGF8b, FGF19, FGFR1 (e.g., FGFR1-IIIC), FGFR2 (e.g., FGFR2-IIIa, FGFR2-IIIb, and FGFR2-IIIc), FGFR3, TNFSF2 (TNFa), FGFR3, EFNa1, EFNa2, ANG1, ANG2, IL-6, IL-8, IL-18, HGF, PDGFA, P1GF, PDGFB, CXCL12, KIT, GCSF, CXCR4, PTPRC, TIE2, VEGFR1, VEGFR2, VEGFR3, Notch 1, DLL4, EGFL7, α2β1 integrin α4β1 integrin, α5β1 integrin, αvβ3 integrin, TGFb, MMP2, MMP7, MMP9, MMP12, PLAU, VCAM1, PDGFRA and PDGFRB. MMM complexes (e.g., ELP, ELP-MRD fusion proteins) that bind VEGFA and 2, 3, 4, 5 or more of these targets are also encompassed by the invention. In specific embodiments, the antibody component of the MMM complex (e.g., ELP-MRD fusion protein) binds VEGFA. In further embodiments, the antibody component of the MMM complex (e.g., ELP-MRD fusion protein) is bevacizumab.

In another embodiment, an ELP-MRD fusion binds VEGFR1 and additionally binds an angiogenic target selected from: VEGFAA, VEGFB, FGF1, FGF2, FGF4, FGF7, FGF8b, FGF19, FGFR1 (e.g., FGFR1-IIIC), FGFR2 (e.g., FGFR2-IIIa, FGFR2-IIIb, and FGFR2-IIIc), FGFR3, TNFSF2 (TNFa), FGFR3, EFNa1, EFNa2, ANG1, ANG2, IL-6, IL-8, IL-18, HGF, PDGFA, P1GF, PDGFB, CXCL12, KIT, GCSF, CXCR4, PTPRC, TIE2, VEGFR2, VEGFR3, Notch 1, DLL4, EGFL7, α2β1 integrin α4β1 integrin, α5β1 integrin, αvβ3 integrin, TGFb, MMP2, MMP7, MMP9, MMP12, PLAU, VCAM1, PDGFRA and PDGFRB. MMM complexes (e.g., ELP-MRD fusion proteins) that bind VEGFR1 and additionally bind 2, 3, 4, 5 or more of these targets are also encompassed by the invention.

In another embodiment, an MMM complex (e.g., an ELP-MRD fusion protein) binds VEGFR1 and additionally binds an angiogenic target selected from: VEGFA, VEGFB, FGF1, FGF2, FGF4, FGF7, FGF8b, FGF19, FGFR1 (e.g., FGFR1-IIIC), FGFR2 (e.g., FGFR2-IIIa, FGFR2-IIIb, and FGFR2-IIIc), FGFR3, TNFSF2 (TNFa), FGFR3, EFNa1, EFNa2, ANG1, ANG2, IL-6, IL-8, IL-18, HGF, PDGFA, P1GF, PDGFB, CXCL12, KIT, GCSF, CXCR4, PTPRC, TIE2, VEGFR2, VEGFR3, Notch 1, DLL4, EGFL7, α2β1 integrin α4β1 integrin, α5β1 integrin, αvβ3 integrin, TGFb, MMP2, MMP7, MMP9, MMP12, PLAU, VCAM1, PDGFRA and PDGFRB. MMM complexes (e.g., ELP, ELP-MRD fusion proteins) that bind VEGFR1 and additionally bind 2, 3, 4, 5 or more of these targets are also encompassed by the invention.

In another embodiment, an MMM complex (e.g., an ELP-MRD fusion protein) binds VEGFR2 and additionally binds a target selected from: VEGFA, VEGFB, FGF1, FGF2, FGF4, FGF7, FGF8b, FGF19, FGFR1 (e.g., FGFR1-IIIC), FGFR2 (e.g., FGFR2-IIIa, FGFR2-IIIb, and FGFR2-IIIc), FGFR3, TNFSF2 (TNFa), FGFR3, EFNa1, EFNa2, ANG1, ANG2, IL-6, IL-8, IL-18, HGF, PDGFA, P1GF, PDGFB, CXCL12, KIT, GCSF, CXCR4, PTPRC, TIE2, VEGFR1, VEGFR2, VEGFR3, Notch 1, DLL4, EGFL7, α2β1 integrin α4β1 integrin, α5β1 integrin, αvβ3 integrin, TGFb, MMP2, MMP7, MMP9, MMP12, PLAU, VCAM1, PDGFRA, and PDGFRB. MMM complexes (e.g., ELP, ELP-MRD fusion proteins) that bind VEGFR2 and additionally bind 2, 3, 4, 5 or more of these targets are also encompassed by the invention.

In another embodiment, an MMM complex (e.g., an ELP-MRD fusion protein) binds ANG2 and additionally binds an angiogenic target selected from: VEGFA, VEGFB, FGF1, FGF2, FGF4, FGF7, FGF8b, FGF19, FGFR1 (e.g., FGFR1-IIIC), FGFR2 (e.g., FGFR2-IIIa, FGFR2-IIIb, and FGFR2-IIIc), FGFR3, TNFSF2 (TNFa), FGFR3, EFNa1, EFNa2, ANG1, ANG2, IL-6, IL-8, IL-18, HGF, PDGFA, P1GF, PDGFB, CXCL12, KIT, GCSF, CXCR4, PTPRC, TIE2, VEGFR1, VEGFR2, VEGFR3, Notch 1, DLL4, EGFL7, α2β1 integrin α4β1 integrin, α5β1 integrin, αvβ3 integrin, TGFb, MMP2, MMPI, MMP9, MMP12, PLAU, VCAM1, PDGFRA and PDGFRB. MMM complexes (e.g., ELP, ELP-MRD fusion proteins) that bind VEGFR2 and additionally bind 2, 3, 4, 5 or more of these targets are also encompassed by the invention.

In additional embodiments, an MMM complex (e.g., an ELP-MRD fusion protein) binds to an anti-angiogenic and a metastatic or invasive cancer target. In on embodiment an MMM complex (e.g., an ELP-MRD fusion protein) binds to an angiogenic target and also bind a metastatic or invasive cancer target selected from: CXCL12, CXCR4 (e.g., CXCR4b), CCR7 (e.g., CXCR7b), CD44 (e.g., CD44v3 and CD44v6), α2β1 integrin α4β1 integrin, α5β1 integrin, αvβ1 integrin, αvβ3 integrin, TGFb, αvβ5 integrin, α9β1 integrin, α6β4 integrin, αMβ2 integrin; PD-1, HGF, cMET, MMP2, MMP-7, MMP-9, MMP-12, VEGFA, VEGFB, and IGF1. MMM complexes (e.g., ELP, ELP-MRD fusion proteins) that bind an angiogenic target and also bind 2, 3, 4, 5 or more of these metastatic or invasive cancer targets are also encompassed by the invention. In specific embodiments, the antibody component of the MMM complex (e.g., ELP-MRD fusion protein) binds VEGF. In further embodiments, the antibody component of the MMM complex (e.g., ELP-MRD fusion protein) is bevacizumab.

In one embodiment, an MMM complex (e.g., an ELP-MRD fusion protein) binds to 2 or more targets associated with distinct cell signaling pathways. In additional embodiments, an MMM complex (e.g., an ELP-MRD fusion protein) binds to 2 or more targets associated with redundant, overlapping or cross-talking signaling pathways. For example, in one embodiment an MMM complex (e.g., an ELP-MRD fusion protein) binds to 2 or more targets associated with a PI3K/AKT/mTOR signaling (e.g., ErbB2, EGFR, IGF1R, Notch, FGFR1 (e.g., FGFR1-IIIC), FGFR2 (e.g., FGFR2-IIIa, FGFR2-IIIb, and FGFR2-IIIb), FGFR3, FGFR4, GPCR, and/or c-MET)). In another embodiment an MMM complex (e.g., an ELP-MRD fusion protein) binds to 2 or more targets associated with receptor tyrosine Raf/MEK/MAPK signaling (e.g., VEGFR1, VEGFR2, VEGFR3, FGFR1 (e.g., FGFR1-IIIC), FGFR2 (e.g., FGFR2-IIIa, FGFR2-IIIb, and FGFR2-IIIb), FGFR3, FGFR4, CD28, RET, cMET, EGFR, ErbB2, Notch, Notch1, Notch3, Notch4, DLL1, DLL4, Jagged, Jagged1, Jagged2, and Jagged3.

In another embodiment an MMM complex (e.g., an ELP-MRD fusion protein) binds to 2 or more targets associated with SMAD signaling (e.g., Notch, TGFβ, TGFβR1, TGFβR2, BMPs).

In another embodiment an MMM complex (e.g., an ELP-MRD fusion protein) binds to 2 or more targets associated with JAK/STAT signaling (e.g., IFNgR1, IFNgR3, IFNG, IFN-AR2, IFN-AR1, INFalpha, IFNbeta, IL6a receptor (GP130), IL6, IL12RB1, IL-12, and EGFR). In another embodiment an MMM complex (e.g., an ELP-MRD fusion protein) binds to 2 or more targets associated with b cateninin signaling (e.g., WNT1, WNT2, WNT2b, WNT3, WNT3A, WNT4, WNT5A, WNT5B, WNT6, WNT7A, WNT7B, WNT8A, WNT8B, WNT9A, WNT9B, WNT10A, WNT10B, WNT11, WNT16, FZD1, FZD2, FZD4, FZD5, FZD6, FZD7, FZD8, Notch, Notch1, Notch3, Notch4, DLL1, DLL4, Jagged, Jagged1, Jagged2, and Jagged3).

In another embodiment an MMM complex (e.g., an ELP-MRD fusion protein) binds to 2 or more targets associated with NFkB signaling (e.g., BCR, TCR, IL-1R, IL1, FZD1, FZD2, FZD4, FZD5, FZD6, FZD7, FZD8, Notch, Notch1, Notch3, Notch4, DLL4, Jagged, Jagged1, Jagged2, Jagged3, TNFSF1 (TNFb, LTa), TNFRSF1A (TNFR1, p55, p60), TNFRSF1B (TNFR2), TNFSF6 (Fas Ligand), TNFRSF6 (Fas, CD95), TNFRSF6B (DcR3), TNFSF7 (CD27 Ligand, CD70), TNFRSF7 (CD27), TNFSF8 (CD30 Ligand), TNFRSF8 (CD30), TNFSF11 (RANKL), TNFRSF11A (RANK), TNFSF12 (TWEAK), TNFRSF12 (TWEAKR), TNFSF13 (APRIL), TNFSF13B (BLyS), TNFRSF13B (TALI), TNFRSF13C (BAFFR), TNFSF15 (TL1A), TNFRSF17 (BCMA), TNFRSF19L (RELT), TNFRSF19 (TROY), TNFRSF21 (DR6), TNFRSF25 (DR3), TNFSF5 (CD40 Ligand), TNFRSF5 (CD40), TNFSF2 (TNFa), TNFSF3 (LTb), TNFRSF3 (LTBR), TNFSF14 (LIGHT, HVEM Ligand), TNFRSF14 (HVEM), TNFSF18 (GITR Ligand), TNFRSF18 (GITR), TNFSF4 (OX40 Ligand), TNFRSF4 (OX40), TNFSF9 (41BB Ligand), TNFRSF9 (41BB, CD137), BMP, NGF, TGFalpha).

In another embodiment an MMM complex (e.g., an ELP-MRD fusion protein) binds to 2 or more targets associated with cell proliferation (e.g., FGF1, FGF2, FGF7, FGF4, FGF10, FGF 18b, FGF19, FGF23, FGFR1 (e.g., FGFR1-IIIC), FGFR2 (e.g., FGFRIIIB and FGFR-IIIC), FGFR3, FGFR4, TCR, CD40, TLR1, TLR2, TLR3, TLR 4, TLR5, and TLR6).

In another embodiment an MMM complex (e.g., an ELP-MRD fusion protein) binds to 2 or more targets associated with toll-like receptor signaling (e.g., TLR1, TLR2, TLR3, TLR 4, TLR5, and TLR6).

In another embodiment an MMM complex (e.g., an ELP-MRD fusion protein) binds to 2 or more targets associated with B cell signaling (e.g., mIg, Igα/Igβ (CD79a/CD79b) heterodimers (α/β), CD19, CD20, CD21, CD22, CD23, CD27, CD30, CD46, CD80, CD86, ICOSL, HLA-DR, (CD74,74), PD1, PDL1, TNFRSF1A (TNFR1, p55, p60), TNFRSF1B (TNFR2), TNFRSF13B (TACI), TNFRSF13C (BAFFR), TNFRSF17 (BCMA), BTLA, TNFRSFS (CD40), TLR4, TNFRSF14 (HVEM), FcgammaRIIB, IL-4R and CRAC. In a particular embodiment, the MMM complex (e.g., ELP-MRD fusion protein) binds to CD19 and CD20. In an additional embodiment, the MMM complex (e.g., ELP-MRD fusion protein) binds CD19, CD20, and CD22.

In a further embodiment an MMM complex (e.g., an ELP-MRD fusion protein) binds to 1 or more B cell surface markers selected from: CD10, CD24, CD37, CD53, CD72, CD75, CD77, CD79a, CD79b, CD81, CD82, CD83, CD84 (SLAMS), and CD85.

In another embodiment an MMM complex (e.g., an ELP-MRD fusion protein) binds to 2 or more targets associated with antigen presentingpresentation cell signaling (e.g., mIg, Igα/Igβ (CD79a/CD79b) heterodimers (α/β), CD19, CD20, CD21, CD22, CD23, CD27, CD28, CD30, CD30L, TNFSF14 (LIGHT, HVEM Ligand), CD70, ICOS, ICOSL, CTLA4, PD-1, PDL1 (B7-H1), B7-H4, B7-H3, PDL2 (B7-DC), BTLA, CD46, CD80 (B7-1), CD86 (B7-2), HLA-DR, CD74, PD1, TNFRSF4 (OX40), TNFRSF9 (41BB, CD137), TNFSF4 (OX40 Ligand), TNFSF9 (41BB Ligand), TNFRSF9 (41BB, CD137), TNFRSF1A (TNFR1, p55, p60), TNFRSF1B (TNFR2), TNFRSF13B (TACI), TNFRSF13C (BAFFR), TNFRSF17 (BCMA), BTLA, TNFRSF18 (GITR), MHC-1, TNFRSFS (CD40), TLR4, TNFRSF14 (HVEM), FcgammaRIIB, IL-4R and CRAC.

In another embodiment an MMM complex (e.g., an ELP-MRD fusion protein) binds to 2 or more targets associated with T cell receptor signaling (e.g., CD3, CD4, CD27, CD28, CD70, CD40L, IL-2R, LFA-1, C4, ICOS, CTLA-4, CD45 CD80, CD86, PG-1, TIM1, TIM2, TIM3, TIM4, galectin 9, TNFRSF1A (TNFR1, p55, p60), TNFRSF1B (TNFR2), TNFRSF21 (DR6), TNFRSF6 (Fas, CD95), TNFRSF25 (DR3), TNFRSF14 (HVEM), TNFSF18, TNFRSF18 (GITR), TNFRSF4 (OX40), TNFSF4 (OX40 Ligand), PD1, PDL1, CTLA4, TNFSF9 (41BB Ligand), TNFRSF9 (41BB, CD137), TNFSF14 (LIGHT, HVEM Ligand), TNFSFS (CD40 Ligand), BTLA, and CRAC).

In additional embodiments, an MMM complex (e.g., an ELP-MRD fusion protein) binds to a therapeutic target and a target associated with an escape pathway for resisting therapeutic effect resulting from targeting therapeutic target. For example, in one embodiment an MMM complex (e.g., an ELP-MRD fusion protein) binds to EGFR and a target selected from MDR1, cMET, Notch, Notch1, Notch3, Notch4, DLL1, DLL4, Jagged, Jagged1, Jagged2, and Jagged3).3.

MMM Complexes that Redirect Effector Cell Function

The invention also encompasses MMM complexes such as, ELP-MRD fusion proteins, that are capable of juxtaposing host effector cells with cells that are desired to be eliminated (e.g., immune cells, cancer cells, diseased cells, infectious agents, and cells infected with infectious agents). The monovalent and MMM functionalities of the complexes of the invention are particularly well suited for redirecting host immune responses and provide numerous advantages over alternative multispecific complex platforms under development. In one embodiment, the MMM complex (e.g., an ELP-MRD fusion protein) binds (1) a target on a cell, tissue, or infectious agent of interest (e.g., an immune cell or a tumor antigen on a tumor cell) and (2) a target on an effector cell so as to direct an immune response to the cell, tissue, or infectious agent of interest. The target(s) to which the MMM complex binds can be monomeric or multimeric. Moreover, the mulitimeric target to which an MMM complex binds can be homomultimeric or heteromultimeric. In additional embodiments, the MMM complex binds at least 2, 3, 4, or 5 targets on the cell, tissue, or infectious agent of interest. In additional embodiments, one or more targets bound by the MMM complex is a tumor antigen (e.g., tumor antigens and tumor/cancer associated antigens). The MMM complexes also have applications in treating diseases and disorders including, but not limited to, diseases of the immune system, skeletal system, cardiovascular system, and nervous system, as well as infectious disease. Thus, in some embodiments, 1, 2, 3, 4, 5 or more targets bound by the MMM complex is associated with a disease or disorder of the immune system (for example, a disease or disorder of the immune system disclosed herein, such as inflammation or an autoimmune disease (e.g., rheumatoid arthritis)). In additional embodiments, 1, 2, 3, 4, 5 or more targets bound by the MMM complex is associated with a disease or disorder of the skeletal system (e.g., osteoporosis or another disease or disorder of the skeletal system as disclosed herein). In additional embodiments, 1, 2, 3, 4, 5 or more targets bound by the MMM complex is associated with a disease or disorder of the cardiovascular system (e.g., a disease or disorder of the cardiovascular system disclosed herein). In additional embodiments, 1, 2, 3, 4, 5 or more targets bound by the MMM complex is associated with a disease or disorder of the nervous system (e.g., a disease or disorder of the nervous system disclosed herein). In additional embodiments, 1, 2, 3, 4, 5 or more targets bound by the MMM complex is associated with an infectious agent or disease (e.g., an infectious disease or agent disclosed herein).

Effector cells that can be bound by an MMM complex (e.g., an ELP-MRD fusion protein) of the invention include, but are not limited to, T cells, monocytes/macrophages, and natural killer cells.

In one embodiment, the target on a cell to which an MMM complex (e.g., an ELP-MRD fusion protein) directs an immune response is a tumor antigen. The MMM complexes of the invention (e.g., ELP-MRD fusion proteins) are envisioned to be capable of binding virtually any type of tumor and any type of tumor antigen. Exemplary types of tumors that can be targeted include, but are not limited to, one or more cancers selected from the group: colorectal cancer, esophageal, gastric, head and neck cancer, thyroid cancer, multiple myeloma, renal cancer, pancreatic cancer, lung cancer, biliary cancer, glioma, melanoma, liver cancer, prostate cancer, and urinary bladder cancer breast cancer, ovarian cancer, cervical cancer, and endometrial cancer. Exemplary types of tumors that can be targeted include hematological cancers. Hematological cancers that can be targeted include, but are not limited to, one or more cancers selected from the group Hodgkin's lymphoma, medullary non-Hodgkin's lymphoma, acute lymphoblastic leukemia, lymphocytic leukemia, and chronic myelogenous leukemia, acute myelogenous leukemia.

Exemplary tumor antigens include TNFRSF6B (DcR3), ErbB1, ErbB2, ErbB3, VEGFR1, VEGFR2, EGFRvIII, CD16, CD19, CD20, oncostatin M, PSA, PSMA, integrin avb6, ADAM9, CD22, CD23, CD25, CD28, CD36, CD45, CD46, CD56, CD79a/CD79b, CD103, JAM-3, gp100, ALCAM, PIPA, A33, carboxypeptidease M, E-cadherin, CA125, CDK4, CEA, CTLA-4, RAAG10, transferrin receptor, p-15, GD2, MUM-IMAGE-1, MAGE-3, KSA, MOC31, MIC-1, EphA2, GAGE-1, GAGE-2, MART, KID31, CD44v3, CD44v6, and ROR1.

In one embodiment, the target on a cell to which an MMM complex (e.g., an ELP-MRD fusion protein) directs an immune response is an immune cell or an inflammatory cell.

In some embodiments, the invention encompasses an MMM complex that binds a tumor antigen that is not expressed on tumor cells themselves, but rather on the surrounding tumor supporting, non-malignant cells comprising the tumor stroma (i.e., tumor associated antigens). The tumor stroma comprises endothelial cells forming new blood vessels and stromal fibroblasts surrounding the tumor vasculature. In one embodiment, an MMM complex binds a tumor associated antigen on an endothelial cell. In an additional embodiment, an MMM complex binds a tumor antigen and also binds a tumor associated antigen on a fibroblast cell. In a further embodiment, an MMM complex binds a tumor antigen and also binds fibroblast activation protein (FAP).

Infectious agents to which an MMM complex (e.g., an ELP-MRD fusion protein) can direct an immune response include, but are not limited to, prokaryotic and eukaryotic cells, viruses (including bacteriophage), foreign objects (e.g., toxins), and infectious organisms such as funghi, and parasites (e.g., mammalian parasites), as described herein and infectious agents associated with infectious diseases described herein. The term infectious agents is also intended to encompass other prokaryotic and eukaryotic cells, viruses (including bacteriophage), foreign objects (e.g., toxins), and infectious organisms such as funghi, and parasites otherwise known in the art.

In further embodiments, the MMM complex (e.g., an ELP-MRD fusion protein) binds (1) a target on a cell, tissue, or infectious agent of interest (e.g., a tumor antigen on a tumor cell) and (2) has a single binding site for a target on an effector cell so as to direct an immune response to the cell, tissue, or infectious agent of interest. In some embodiments, embodiments, the single binding site is an MRD. In other embodiments, the single binding site is an antibody antigen binding domain. In further embodiments, binding of the MMM complex does not elicit a signal when the composition binds a target on an effector cell. In additional embodiments, the MMM complex binds at least 2, 3, 4, or 5 targets on the cell, tissue, or infectious agent of interest. According to some embodiments, at least 1, 2, 3, 4, 5 or more of the targets of the MMM complex are located on a cell surface. In additional embodiments, 1, 2, 3, 4, 5 or more targets bound by the MMM complex is a tumor antigen (e.g., tumor antigens and tumor/cancer associated antigens). In additional embodiments, one or more targets bound by the MMM complex are associated with a disease or disorder of the immune system. In additional embodiments, one or more targets bound by the MMM complex are associated with a disease or disorder of the skeletal system (e.g., osteoporosis), cardiovascular system, nervous system, or an infectious disease.

In additional embodiments, the MMM complex (e.g., an ELP-MRD fusion protein) binds (1) a target on a cell, tissue, or infectious agent of interest (e.g., a tumor antigen on a tumor cell) and (2) a target on a leukocyte so as to direct an immune response to the cell, tissue, or infectious agent of interest. In additional embodiments, the MMM complex binds at least 2, 3, 4, or 5 targets on the cell, tissue, or infectious agent of interest. According to some embodiments, at least 1, 2, 3, 4, 5 or more of the targets of the MMM complex are located on a cell surface. In additional embodiments, embodiments, the MMM complex binds 1, 2, 3, 4, 5 or more targets described herein. In additional embodiments, 1, 2, 3, 4, 5 or more targets bound by the MMM complex are a tumor antigen (e.g., tumor antigens and tumor/cancer associated antigens). In additional embodiments, one or more targets bound by the MMM complex are associated with a disease or disorder of the immune system. In additional embodiments, one or more targets bound by the MMM complex are associated with a disease or disorder of the skeletal system (e.g., osteoporosis), cardiovascular system, nervous system, or an infectious disease.

The invention also encompasses MMM complexes (e.g., ELP, ELP-MRD fusion proteins) that bind a target expressed on a leukocyte. In some embodiments, the MMM complex (e.g., an ELP-MRD fusion protein) binds (1) a target on a cell, tissue, or infectious agent of interest (e.g., a tumor antigen on a tumor cell) and (2) has a single binding site for a target on a leukocyte so as to direct an immune response to the cell, tissue, or infectious agent of interest. In additional embodiments, the MMM complex binds at least 2, 3, 4, or 5 targets on the cell, tissue, or infectious agent of interest. According to some embodiments, at least 1, 2, 3, 4, 5 or more of the targets of the MMM complex are located on a cell surface. In additional embodiments, 1, 2, 3, 4, 5 or more targets bound by the MMM complex is a tumor antigen (e.g., tumor antigens and tumor/cancer associated antigens). In additional embodiments, 1, 2, 3, 4, 5 or more targets bound by the MMM complex are associated with a disease or disorder of the immune system. In additional embodiments, 1, 2, 3, 4, 5 or more targets bound by the MMM complex are associated with a disease or disorder of the skeletal system (e.g., osteoporosis), cardiovascular system, nervous system, or an infectious disease.

In one embodiment, the MMM complex (e.g., an MMM complex (e.g., an ELP-MRD fusion protein) binds a target expressed on a T cell. In some embodiments, the MMM complex (e.g., an ELP-MRD fusion protein) binds (1) a target on a cell, tissue, or infectious agent of interest (e.g., a tumor antigen on a tumor cell) and (2) a target on a T cell so as to juxtapose myeloid cells with the cell, tissue, or infectious agent of interest. In some embodiments, the MMM complex has multiple binding sites for (i.e., multivalently binds) a target on a T cell. In other embodiments, the MMM complex has a single binding site for (i.e., monovalently binds) a target on a T cell. In some embodiments, embodiments, the single binding site is an MRD. In other embodiments, the single binding site is an antibody antigen binding domain. In further embodiments, binding of the MMM complex does not elicit a signal when the composition binds a target on a T cell. In other embodiments, the binding of the MMM complex does not result in lysis of the T cell expressing the target. In some embodiments, the MMM complex binds a target selected from: CD2, CD3, CD4, CD8, CD161, a chemokine receptor, CD95, and CCR5. In additional embodiments, the MMM complex binds at least 2, 3, 4, or 5 targets on the cell, tissue, or infectious agent of interest. According to some embodiments, at least 1, 2, 3, 4, 5 or more of the targets of the MMM complex are located on a cell surface. In additional embodiments, 1, 2, 3, 4, 5 or more targets bound by the MMM complex is a tumor antigen (e.g., tumor antigens and tumor/cancer associated antigens). In additional embodiments, 1, 2, 3, 4, 5 or more targets bound by the MMM complex are associated with a disease or disorder of the immune system. In additional embodiments, 1, 2, 3, 4, 5 or more targets bound by the MMM complex are associated with a disease or disorder of the skeletal system (e.g., osteoporosis), cardiovascular system, nervous system, or an infectious disease.

In further embodiments, the MMM complex (e.g., an MMM complex (e.g., an ELP-MRD fusion protein) contains a fusion protein containing one or more peptides that bind to a protein on the surface of a cell, such as a T cell. In additional embodiments, MMM complex bind target membrane proximal protein sequences on a cell and inhibit the cross-linking of the target protein or its associated proteins. In a particular embodiment, the MMM complex binds to a T cell and inhibits the cross-linking of the cell protein or its associated proteins. For example, in one embodiment, the MMM ELP comprises the amino terminal 27 amino acids of mature CD3 epsilon. In another embodiment, the MMM complex comprises a fusion protein containing one or more proteins corresponding to the G Domain of a CD3 protein (e.g., CD3 epsilon, CD3 gamma, CD3 alpha (TCRA) or CD3 beta (TCRB). Thus, in some embodiments, the fusion protein comprises a polypeptide having an amino acid sequence selected from: GYYVCYPRGSKPEDANFYLYLRARVC (SEQ ID NO:133), YLYLRAR (SEQ ID NO:134), YRCNGTDIYKDKESTVQVHYRMC (SEQ ID NO:135), and DKESTVQVH (SEQ ID NO:136). In additional embodiments, the composition comprises a fusion protein containing one or more proteins corresponding to a portion of the extracellular domain of a CD3 protein (e.g., CD3 epsilon, CD3 gamma, CD3 alpha (TCRA) or CD3 beta (TCRB)) that is able to bind CD3, or a CD3 multimer. Thus, in some embodiments, the fusion protein comprises a portion of a CD3 protein that is able to bind CD3 or a CD3 multimer wherein the portion comprises a CD3 binding fragment of a polypeptide having an amino acid sequence selected from: KIPIEELEDRVFVNCNTSITWVEGTVGTLLSDITRLDLGKRILDPRGIYRCNGTDIYKDKESTVQVHYRMCQSCVELD (human CD3 delta mature ECD, SEQ ID NO:137), QSIKGNHLVKVYDYQEDGSVLLTCDAEAKNITWFKDGKMIGFLTEDKKKWNLGSNAKDPRGMYQCKGSQNKSKPLQVYYRMCQNCIELN (human CD3 gamma mature ECD, Ig-like domain highlighted; SEQ ID NO:138), GNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVM (human CD3 epsilon mature ECD, Ig-like domain highlighted, SEQ ID NO:139), and QSFGLLDPK (human CD3 zeta mature ECD, SEQ ID NO:140), In alternative embodiments, the fusion protein comprises a chemokine fragment that binds a target on the cell surface. In some embodiments, the chemokine fragment is a portion of a chemokine selected from: CCL20 (LARC/Ckβ4), CCL25 (TECK/Ckβ15), CXCL12 (SDF-1), CXCL13 (BCA-1), CXCL16 (SRPSOX), and CX3CL1 (Fractalkine) In some embodiments, the chemokine fragment is a portion of a chemokine selected from: CCL5 (RANTES), CCL8 (MCP-2), CXCL9 (MIG/CRG-10), CXCL10 (IP-10/CRG-2) and CXCL11 (TAC/IP-9). In some embodiments, the chemokine fragment is a portion of a chemokine selected from CCL3 (MIP-1a) and CCL4 (MIP-1β).

In specific embodiments, the MMM complex (e.g., an ELP-MRD fusion protein) binds CD3. In particular embodiments, the composition binds a CD3 target selected from CD3 delta, CD3 epsilon, CD3 gamma, CD3 zeta, TCR alpha, TCR beta, the TCR complex, or a heteromeric or homomultimeric combination thereof. In a further embodiment, the composition binds CD3 epsilon. In additional embodiments, the MMM complex binds CD3 and multiple binding sites for 1, 2, 3, 4, 5 or more different targets (e.g., a tumor antigen as disclosed herein or otherwise known in the art). In additional embodiments, the MMM complex has a single binding site for (i.e., monovalently binds) CD3. In further embodiments, the MMM complex has a single MRD that binds CD3 and multiple binding sites for 1, 2, 3, 4, 5 or more different targets (e.g., a tumor antigen as disclosed herein or otherwise known in the art). In further embodiments, the MMM complex has a single antibody antigen binding domain that binds CD3 and multiple binding sites for 1, 2, 3, 4, 5 or more different targets (e.g., a tumor antigen as disclosed herein or otherwise known in the art). In particular embodiments, the CD3 binding compositions of the invention are not single chain antibodies.

In some embodiments, the MMM complex (e.g., an ELP-MRD fusion protein) binds human CD3 and a CD3 ortholog from another organism. In additional embodiments, the MMM complex binds human CD3 and a CD3 ortholog from another primate. In further embodiments, the MMM complex binds human CD3 and a CD3 ortholog from cynomolgus monkey or rhesus monkey. In further embodiments, the MMM complex binds human CD3 and a CD3 ortholog from cynomolgus monkey and rat or mouse. In other embodiments, the MMM complex binds human CD3 and a CD3 ortholog from a primate selected from macaque falpricana, Saguinus Oedipus and Callithrix jacchus).

According to one embodiment, the MMM complex (e.g., an ELP-MRD fusion protein) binds human CD3 epsilon. In a particular embodiment, the, MMM complex binds human CD3 epsilon protein having the sequence of amino acids 23-207 set forth in NCBI Ref. Seq, No. NP_{—}000724. In another embodiment, the MMM complex binds a polypeptide having the amino acid sequence of QDGNEEMGGITQTPYKVSISGTTVILT (SEQ ID NO:141). In an additional embodiment, the MMM complex binds a polypeptide having the amino acid sequence of QDGNEEMGGI (SEQ ID NO:142). In a further embodiment, the MMM complex binds a polypeptide having the amino acid sequence of QDGNEEMGG (SEQ ID NO:143). In particular embodiments, the human CD3 epsilon binding compositions of the invention are not single chain antibodies.

In some embodiments, an MMM complex (e.g., an ELP-MRD fusion protein) has a single binding site for CD3 epsilon (i.e., monovalently binds CD3 epsilon) and multiple binding sites for 1, 2, 3, 4, 5 or more different targets (e.g., a B cell or other target disclosed herein). In further embodiments, the MMM complex (e.g., an ELP-MRD fusion protein) competes for binding to CD3 with an antibody selected from: OKT-3, otelixizumab, teplizumab, visilizumab, muromonab, X35-3, VIT3, BMA030 (BW264/56), CLB-T3/3, CRIS7, YTH12.5, F111409, CLB-T3.4.2, TR-66, WT31, WT32, SPv-T3b, 11D8, XIII-141, XII146, XIII-87, 12F6, T3/RW2-8C8, T3/RW24B6, OKT3D, M-T301, SMC2 and F101.01. In additional embodiments, an MRD of an MMM complex (e.g., an ELP-MRD fusion protein) competes for binding to CD3 with an antibody selected from: OKT-3, otelixizumab, teplizumab, visilizumab, muromonab X35-3, VIT3, BMA030 (BW264/56), CLB-T3/3, CRIS7, YTH12.5, F111409, CLB-T3.4.2, TR-66, WT31, WT32, SPv-T3b, 11D8, XIII-141, XII146, XIII-87, 12F6, T3/RW2-8C8, T3/RW24B6, OKT3D, M-T301, SMC2 and F101.01. In further embodiments, the MMM complex (e.g., ELP-MRD fusion protein) competes for binding to CD3 with a CD3 binding composition disclosed in Int. Appl. Pubs. WO 2004/106380 and WO 99/54440; Tunnacliffe et al., Int. Immunol. 1:546-550 (1989); Kjer-Nielsen, PNAS 101: 7675-7680 (2004); or Salmeron et al., J. Immunol. 147: 3047-3052 (1991).

In additional embodiments, the MMM complex (e.g., an ELP-MRD fusion protein) binds human CD3 epsilon and a CD3 epsilon ortholog from another organism. In some embodiments, the MMM complex (e.g., an ELP-MRD fusion protein) binds human CD3 epsilon and a CD3 epsilon ortholog from another primate. In additional embodiments, the MMM complex binds human CD3 epsilon and a CD3 epsilon ortholog from cynomolgus monkey or rhesus monkey. In additional embodiments, the MMM complex binds human CD3 epsilon and a CD3 epsilon ortholog from a primate selected from macaque falpricana, Saguinus Oedipus and Callithrix jacchus. In particular embodiments, an MRD of the MMM complex binds CD3 epsilon.

In another embodiment the MMM complex (e.g., an ELP-MRD fusion protein) binds human CD3 delta. In a particular embodiment, the, MMM complex binds human CD3 delta having the sequence of amino acids 22-171 set forth in NCBI Ref. Seq, No.: NP_{—}000723. In particular embodiments, an MRD of the MMM complex binds CD3 delta. In other embodiments, an antibody antigen binding domain of the MMM complex binds CD3 delta. In particular embodiments, the human CD3 epsilon binding compositions of the invention are not single chain antibodies.

In an additional embodiment, the MMM complex (e.g., an ELP-MRD fusion protein) binds human CD3 gamma protein having the sequence of amino acids 23-182 set forth in NCBI Ref. Seq, No.: NP_{—}000064. In particular embodiments, an MRD of the MMM complex binds CD3 gamma. In particular embodiments, an MRD of the MMM complex binds CD3 gamma. In other embodiments, an antibody antigen binding domain of the MMM complex binds CD3 gamma. In particular embodiments, the human CD3 gamma binding compositions of the invention are not single chain antibodies.

In an additional embodiment, the MMM complex (e.g., an ELP-MRD fusion protein) binds human CD3 zeta protein having the sequence of amino acids 22-164 set forth in NCBI Ref. Seq, No.: NP_{—}932170. In particular embodiments, an MRD of the MMM complex binds CD3 zeta. In other embodiments, an antibody antigen binding domain of the MMM complex binds CD3 zeta. In particular embodiments, the human CD3 zeta binding compositions of the invention are not single chain antibodies.

The invention also encompasses MMM complexes that bind a target expressed on a natural killer cell. In some embodiments, the MMM complex (e.g., an ELP-MRD fusion protein) binds (1) a target on a cell, tissue, or infectious agent of interest (e.g., a tumor antigen on a tumor cell) and (2) a target on a natural killer cell. In some embodiments, the MMM complex has multiple binding sites for (i.e., multivalently binds) a target on a natural killer cell. In other embodiments, the MMM complex has a single binding site for (i.e., monovalently binds) a target on a natural killer cell. In some embodiments, embodiments, the single binding site is an MRD. In other embodiments, the single binding site is an antibody antigen binding domain. In further embodiments, binding of the MMM complex does not elicit a signal when the composition binds a target on a natural killer cell. In some embodiments, the MMM complex binds a target selected from: KLRD1, KLRK1, KLRB1, 2B4 (CD244), KIR2D4, KIR2D5, and KIR3DL1. In other embodiments, the MMM complex binds a target selected from: CD56, CD2, and CD161. In additional embodiments, the MMM complex binds at least 2, 3, 4, or 5 targets on the cell, tissue, or infectious agent of interest. According to some embodiments, at least 1, 2, 3, 4, 5 or more of the targets of the MMM complex are located on a cell surface. In additional embodiments, 1, 2, 3, 4, 5 or more targets bound by the MMM complex are a tumor antigen (e.g., tumor antigens and tumor/cancer associated antigens). In additional embodiments, 1, 2, 3, 4, 5 or more targets bound by the MMM complex are associated with a disease or disorder of the immune system. In additional embodiments, 1, 2, 3, 4, 5 or more targets bound by the MMM complex are associated with a disease or disorder of the skeletal system (e.g., osteoporosis), cardiovascular system, nervous system, or an infectious disease.

In specific embodiments, the MMM complex (e.g., an ELP-MRD fusion protein) binds CD2. According to one embodiment, the MMM complex (e.g., an ELP-MRD fusion protein) binds human CD2. In a particular embodiment, the MMM complex binds human CD2 protein having the sequence of amino acids 25-209 set forth in NCBI Ref. Seq. No. NP_{—}001758. In some embodiments, the MMM complex has multiple binding sites for CD2. In some embodiments, embodiments, the single binding site is an MRD. In other embodiments, the MMM complex has a single binding site for CD2. In further embodiments, binding of the MMM complex to CD2 does not elicit a signal by the cell on which CD2 is expressed. In additional embodiments, the MMM complex binds CD2 and 1, 2, 3, 4, 5 or more different targets (e.g., a tumor antigen as disclosed herein or otherwise known in the art).

In some embodiments, the MMM complex (e.g., an ELP-MRD fusion protein) binds human CD2 and a CD2 ortholog from another organism. In additional embodiments, the MMM complex binds human CD2 and a CD2 ortholog from another primate. In further embodiments, the MMM complex binds human CD2 and a CD2 ortholog from cynomolgus monkey or rhesus monkey.

In some embodiments, the MMM complex (e.g., an ELP-MRD fusion protein) binds a target on a myeloid cell. In some embodiments, the MMM complex (e.g., an ELP-MRD fusion protein) binds (1) a target on a cell, tissue, or infectious agent of interest (e.g., a tumor antigen on a tumor cell) and (2) a target on an immune accessory cell (e.g., myeloid cell) so as to juxtapose myeloid cells with the cell, tissue, or infectious agent of interest. In some embodiments, the MMM complex has multiple binding sites for (i.e., multivalently binds) a target on a myeloid cell. In other embodiments, the MMM complex has a single binding site for (i.e., monovalently binds) a target on an accessory cell (e.g., myeloid cell). In some embodiments, embodiments, the single binding site is an MRD. In further embodiments, binding of the MMM complex does not elicit a signal when the composition binds a target on a myeloid cell. In some embodiments, the MMM complex binds an Fc gamma receptor selected from CD16 (i.e., Fc gamma RIII), CD64 (i.e., Fc gamma R1), and CD32 (i.e., Fc gamma R11). In particular embodiments, the MMM complex binds CD64 (i.e., Fc gamma R1). In some embodiments, the MMM complex binds a target selected from, MHC class 2 and its invariant chain, TLR1, TLR2, TLR4, TLR5 and TLR6. In additional embodiments, the MMM complex binds at least 2, 3, 4, or 5 targets on the cell, tissue, or infectious agent of interest. According to some embodiments, at least 1, 2, 3, 4, 5 or more of the targets of the MMM complex are located on a cell surface. In additional embodiments, 1, 2, 3, 4, 5 or more targets bound by the MMM complex are a tumor antigen (e.g., tumor antigens and tumor/cancer associated antigens). In additional embodiments, 1, 2, 3, 4, 5 or more targets bound by the MMM complex are associated with a disease or disorder of the immune system. In additional embodiments, 1, 2, 3, 4, 5 or more targets bound by the MMM complex are associated with a disease or disorder of the skeletal system (e.g., osteoporosis), cardiovascular system, nervous system, or an infectious disease.

In some embodiments, the MMM complex (e.g., an ELP-MRD fusion protein) binds a target of interest on a cancer cell. In additional embodiments, the MMM complex binds a target of interest on an immune cell. In further embodiments, the MMM complex binds a target of interest on a diseased cell. In other embodiments, the MMM complex (e.g., an ELP-MRD fusion protein) binds a target of interest on an infectious agent (e.g., a bacterial cell or a virus).

In further embodiments, the invention encompasses a method of treating a disease or disorder by administering to a patient in need thereof, a therapeutically effective amount of an MMM complex of the invention to a patient in need thereof. Particular embodiments, embodiments, are directed to a method of treating a disease or disorder by administering to a patient in need thereof, a therapeutically effective amount an MMM complex (e.g., an ELP-MRD fusion protein) that has a single binding site for a target (i.e., that monovalently binds a target). In some embodiments, the administered MMM complex has a single binding site for a target on a leukocyte, such as a T-cell (e.g., CD3). In additional embodiments, the administered MMM complex has a single binding site for a target on a leukocyte, such as a T-cell (e.g., CD3) and multiple binding sites for (i.e., is capable of multivalently binding) a target located on a cell or tissue of interest (e.g., a tumor antigen on a tumor cell).

In further embodiments, the invention is directed to treating a disease or disorder by administering to a patient a therapeutically effective amount of an MMM complex (e.g., an ELP-MRD fusion protein) that has a single binding site for a target (i.e., that monovalently binds a target) and multiple binding sites for 1, 2, 3, 4, 5 or more different targets.

In additional embodiments, the invention is directed to treating a disease or disorder by administering to a patient in need thereof, a therapeutically effective amount of an MMM complex (e.g., an ELP-MRD fusion protein) that has a single binding site for CD3 (e.g., CD3 epsilon) that monovalently binds CD3 and multiple binding sites for 1, 2, 3, 4, 5 or more different targets.

According to some embodiments, the tumor cell is from a cancer selected from breast cancer, colorectal cancer, endometrial cancer, kidney (renal cell) cancer, lung cancer, melanoma, Non-Hodgkin Lymphoma, leukemia, prostate cancer, bladder cancer, pancreatic cancer, and thyroid cancer. In additional embodiments, the MMM complex has multiple binding sites for a target on a neurological tumor. In particular embodiments, the neurological tumor is a glioma (e.g., a glioblastoma, glioblastoma multiforme (GBM), and astrocytoma), ependymoma, oligodendroglioma, neurofibroma, sarcoma, medulloblastoma, primitive neuroectodermal tumor, pituitary adenoma, neuroblastoma or cancer of the meninges (e.g., meningioma, meningiosarcoma and gliomatosis).

In some embodiments, the MMM complex (e.g., an ELP-MRD fusion protein) binds a cytokine or chemokine. In some embodiments, the MMM complex (e.g., ELP-MRD fusion protein) is capable of binding at least 1, at least 2, at least 3, at least 4, or at least 5 cytokines or chemokines In some embodiments, the MMM complex (e.g., ELP-MRD fusion protein) is capable of binding at least 1, at least 2, at least 3, at least 4, or at least 5 cytokines or chemokines simultaneously. In some embodiments, the MMM complex (e.g., ELP-MRD fusion protein) is capable of binding at least 1, at least 2, at least 3, at least 4, or at least 5 molecules of the same cytokine or chemokine. In some embodiments, the MMM complex (e.g., ELP-MRD fusion protein) is capable of binding at least 1, at least 2, at least 3, at least 4, or at least 5 molecules of the same cytokine or chemokine simultaneously. In some embodiments, the MMM complex (e.g., ELP-MRD fusion protein) is capable of binding at least 1, at least 2, at least 3, at least 4, or at least 5 different epitopes of a cytokine or chemokine. In some embodiments, the MMM complex (e.g., ELP-MRD fusion protein) is capable of binding at least 1, at least 2, at least 3, at least 4, or at least 5 different epitopes of a cytokine or chemokine simultaneously. In some embodiments, the MMM complex (e.g., ELP-MRD fusion protein) is capable of binding at least 1, at least 2, at least 3, at least 4, or at least 5 different cytokines or chemokines. In some embodiments, the MMM complex (e.g., ELP-MRD fusion protein) is capable of binding at least 1, at least 2, at least 3, at least 4, or at least 5 different cytokines or chemokines simultaneously.

In some embodiments, the MMM complex (e.g., ELP-MRD fusion protein) binds a cancer antigen. In some embodiments, the MMM complex (e.g., ELP-MRD fusion protein) is capable of binding at least 1, at least 2, at least 3, at least 4, or at least 5 cancer antigens. In some embodiments, the MMM complex (e.g., ELP-MRD fusion protein) is capable of binding at least 1, at least 2, at least 3, at least 4, or at least 5 cancer antigens, simultaneously. In some embodiments, the MMM complex (e.g., ELP-MRD fusion protein) is capable of binding at least 1, at least 2, at least 3, at least 4, or at least 5 molecules of the same cancer antigen. In some embodiments, the MMM complex (e.g., ELP-MRD fusion protein) is capable of binding at least 1, at least 2, at least 3, at least 4, or at least 5 molecules of the same cancer antigen simultaneously. In some embodiments, the MMM complex (e.g., ELP-MRD fusion protein) is capable of binding at least 1, at least 2, at least 3, at least 4, or at least 5 different epitopes of the same cancer antigen. In some embodiments, the MMM complex (e.g., ELP-MRD fusion protein) is capable of binding at least 1, at least 2, at least 3, at least 4, or at least 5 different epitopes of the same cancer antigen, simultaneously. In some embodiments, the MMM complex (e.g., ELP-MRD fusion protein) is capable of binding at least 1, at least 2, at least 3, at least 4, or at least 5 different cancer antigens. In some embodiments, the MMM complex (e.g., ELP-MRD fusion protein) is capable of binding at least 1, at least 2, at least 3, at least 4, or at least 5 different cancer antigens, simultaneously.

In some embodiments, the MMM complex (e.g., ELP-MRD fusion protein) binds an antigen associated with a disorder of the immune system. In some embodiments, the MMM complex (e.g., ELP-MRD fusion protein) is capable of binding at least 1, at least 2, at least 3, at least 4, or at least 5 antigens associated with a disorder of the immune system. In some embodiments, the MMM complex (e.g., ELP-MRD fusion protein) is capable of binding at least 1, at least 2, at least 3, at least 4, or at least 5 antigens associated with a disorder of the immune system, simultaneously. In some embodiments, the MMM complex (e.g., ELP-MRD fusion protein) is capable of binding at least 1, at least 2, at least 3, at least 4, or at least 5 molecules of the same antigen associated with a disorder of the immune system. In some embodiments, the MMM complex (e.g., ELP-MRD fusion protein) is capable of binding at least 1, at least 2, at least 3, at least 4, or at least 5 molecules of the same antigens associated with a disorder of the immune system, simultaneously. In some embodiments, the MMM complex (e.g., ELP-MRD fusion protein) is capable of binding at least 1, at least 2, at least 3, at least 4, or at least 5 different epitopes of the same antigen associated with a disorder of the immune system. In some embodiments, the MMM complex (e.g., ELP-MRD fusion protein) is capable of binding at least 1, at least 2, at least 3, at least 4, or at least 5 different epitopes of the same antigen associated with a disorder of the immune system, simultaneously. In some embodiments, the MMM complex (e.g., ELP-MRD fusion protein) is capable of binding at least 1, at least 2, at least 3, at least 4, or at least 5 different antigens associated with a disorder of the immune system. In some embodiments, the MMM complex (e.g., ELP-MRD fusion protein) is capable of binding at least 1, at least 2, at least 3, at least 4, or at least 5 different antigens associated with a disorder of the immune system, simultaneously.

In some embodiments, one or more of an MRD(s), or the collective MMM complex (e.g., ELP-MRD fusion protein) is an antagonist of their respective target molecules. In other embodiments, one or more of an MRD(s), or the collective MMM complex (e.g., ELP-MRD fusion protein) is an agonist of the respective target molecules. In yet other embodiments, at least one MRD in the MMM complex (e.g., ELP-MRD fusion protein) is an antagonist of its target molecule and a second MRD or the collective MMM complex (e.g., ELP-MRD fusion protein) is an agonist of a different target molecule. In yet another embodiment, at least one MRD in the MMM complex (e.g., ELP-MRD fusion protein) is an agonist of its target molecule, and a second MRD or the collective MMM complex (e.g., ELP-MRD fusion protein) is an antagonist of a different target molecule. In some embodiments, at least 1, at least 2, at least 3, at least 4, or at least 5

MRD(s) in the MMM complex (e.g., ELP-MRD fusion protein) bind to soluble factors. In some embodiments, at least 1, at least 2, at least 3, at least 4, or at least 5 MRD(s) in the MMM complex (e.g., ELP-MRD fusion protein) bind to cell surface molecules. In some embodiments, at least 1, at least 2, at least 3, at least 4, or at least 5 MRD(s) in the MMM complex (e.g., ELP-MRD fusion protein) binds to a cell surface molecule and at least 1, at least 2, at least 3, at least 4, or at least 5 MRD(s) in the MMM complex (e.g., ELP-MRD fusion protein) binds to a soluble factor.

In preferred embodiments, the MMM complex (e.g., an ELP-MRD fusion protein) is capable of inducing complement dependent cytotoxicity. In certain embodiments, the MMM complex (e.g., an ELP-MRD fusion protein) is capable of inducing antibody dependent cell mediated cytotoxicity (ADCC). In additional embodiments, the MMM complex (e.g., an ELP-MRD fusion protein) is capable of inducing apoptosis. In additional embodiments, the MMM complex (e.g., an ELP-MRD fusion protein) is capable of reducing tumor volume. In additional embodiments, the MMM complexes (e.g., ELP-MRD fusion proteins) are capable of inhibiting tumor growth.

An improved MMM complex (e.g., ELP-MRD fusion protein) that binds a desired target or targets can also be prepared based on a previously known MRD or antibody variable domain fragment containing MMM complex (e.g., ELP-MRD fusion protein). For example, 1, 2, 3, 4, 5, 6, 7, 8, 9, 10, 10-20, 20-30, 30-50, 50-100, 100-150 or more than 150 amino acid substitutions, deletions or insertions can be introduced into an MRD or MMM complex (e.g., ELP-MRD fusion protein) sequence and the resulting MRD or MMM complex (e.g., ELP-MRD fusion protein) can be screened for binding to the desired target or targets, for antagonizing target activity, or for agonizing target activity as described in the examples or using techniques known in the art.

Additional peptide sequences can be added, for example, to enhance the in vivo stability of an MRD or affinity of an MRD for its target.

In some embodiments, the ELP-MRDs contain at least one reactive residue residue. Reactive residues are useful, for example, as sites for the attachment of conjugates such as chemotherapeutic drugs. The reactive residue can be, for example, a cysteine, a lysine, or another reactive residue. The cysteine, lysine, or other reactive residue can be located between components of an ELP-MRD fusion, e.g. between an ELP and an MRD, linker, or other component of an ELP-MRD fusion, between an MRD and an ELP, linker, or other component of an ELP-MRD fusion, or between a linker and an ELP, MRD, or other component of an ELP-MRD fusion. The cysteine, lysine, or other reactive residue can also be located within the sequence of an ELP, MRD, linker, or other component of the ELP-MRD fusion. Thus, a cysteine, lysine, or other reactive residue can be added within the sequence of an ELP, MRD, linker or other component of the ELP-MRD fusion and/or a cysteine, lysine, or other reactive residue can be substituted for another amino acid in the sequence of an ELP, MRD, linker, or other component of an ELP-MRD fusion. In some embodiments, an MMM complex (e.g., an ELP-MRD fusion protein) contains at least 1, at least 2, at least 3, at least 4, or at least 5 reactive residues. In some embodiments, an MMM complex (e.g., an ELP-MRD fusion protein) contains at least 1, at least 2, at least 3, at least 4, or at least 5 cysteines.

In other embodiments, the MMM complexes (e.g., ELP-MRD fusion proteins) have one or more of the following effects: inhibit proliferation of tumor cells, reduce the tumorigenicity of a tumor, inhibit tumor growth, increase subject survival, trigger cell death of tumor cells, differentiate tumorigenic cells to a non-tumorigenic state, or prevent metastasis of tumor cells.

In some embodiments, the MMM complex (e.g., ELP-MRD fusion protein) can bind to multiple molecules of the same target and induce homo-multimerization. In some embodiments, the MMM complex (e.g., ELP-MRD fusion protein) can bind to multiple molecules that are different and induce hetero-multimerization.

In some embodiments, the MMM complex (e.g., ELP-MRD fusion protein) can bind multiple targets on the surface of a target cell. In some embodiments, the MMM complex (e.g., ELP-MRD fusion protein) can bind multiple targets on the surface of a target cell, simultaneously. The multiple targets on the surface of the target cell can be the same target molecule or can be different target molecules. In some embodiments, the MMM complex (e.g., ELP-MRD fusion protein) can bind multiple targets on the surface of a target cell to agonize cell signaling. In some embodiments, the MMM complex (e.g., ELP-MRD fusion protein) can bind multiple targets on the surface of a target cell to antagonize cell signaling.

In some embodiments, an MMM complex (e.g., an ELP-MRD fusion protein) binds to a family of receptors. For example, an MMM complex (e.g., an ELP-MRD fusion protein) can bind to growth factor receptors, to TNF family receptors, to G-protein-coupled receptors, and/or chemokine receptors. Thus, for example, and MMM complex (e.g., ELP-MRD fusion protein) can bind to multiple TNF receptors (e.g. TRAIL-R1 and TRAIL-R2). An MMM complex (e.g., an ELP-MRD fusion protein) can bind to different families of receptors as well. Thus, for example, an MMM complex (e.g., an ELP-MRD fusion protein) can bind to a growth factor receptor and a TNF receptor or a G-protein-coupled receptor and a chemokine receptor.

In some embodiments, the MMM complex (e.g., ELP-MRD fusion protein) can bind multiple targets on the surface of different target cells. In some embodiments, the MMM complex (e.g., ELP-MRD fusion protein) can bind multiple targets on the surface of the target cells, simultaneously. The target cells can be the same type of target cell or can be different types of target cells. The multiple targets on the surface of the target cells can be the same target molecule or can be different target molecules. In some embodiments, the MMM complex (e.g., ELP-MRD fusion protein) can bring target cells together by binding to targets on the surface of the target cells.

In some embodiments, the MMM complex (e.g., ELP-MRD fusion protein) can bind to different targets associated with a disease or disorder, wherein the different targets are associated with different modes of action in connection with the disease or disorder. For example, an MMM complex (e.g., an ELP-MRD fusion protein) can bind to a target in a pathway that influences cell proliferation and a target that in a pathway that influences angiogenesis. Thus, In some embodiments, the MMM complex (e.g., ELP-MRD fusion protein) binds to at least 2, at least 3, at least 4, or at least 5 targets from at least 2, at least 3, at least 4, or at least 5 pathways or mechanisms of action associated with a disease or disorder. For example, an MMM complex (e.g., an ELP-MRD fusion protein) can bind to targets that regulate angiogenesis, proliferation, survival, apoptosis, adhesion, metastasis, cell cycle, DNA repair, senescence, trafficking, metabolism, autophagy, inflammation and/or immunosurveillance. In some embodiments, an ELP-MRD fusion binds to targets that influence at least 2, at least 3, at least 4, or at least 5 mechanisms of action selected from the group consisting of: angiogenesis, proliferation, survival, apoptosis, adhesion, metastasis, cell cycle, DNA repair, senescence, trafficking, metabolism, autophagy, inflammation and/or immunosurveillance.

In specific embodiments, the MMM complex (e.g., ELP-MRD fusion protein) binds ErbB2 and an angiogenic factor. In specific embodiments, the MMM complex (e.g., ELP-MRD fusion protein) binds ErbB2 and IGF1R. In another embodiment, the MMM complex (e.g., ELP-MRD fusion protein) binds ErbB2, and an angiogenic factor and/or IGF1R. In one embodiment, the MMM complex (e.g., ELP-MRD fusion protein) binds to the same ErbB2 epitope as trastuzumab and an angiogenic factor and/or IGF1R. In an additional embodiment, the MMM complex (e.g., ELP-MRD fusion protein) competitively inhibits trastuzumab binding to ErbB2 and an angiogenic factor and/or IGF1R. In additional embodiments, an MMM complex (e.g., an ELP-MRD fusion protein) comprises the sequences of SEQ ID NOS:59-64 and an angiogenic factor and/or IGF1R. In additional embodiments, the MMM complex (e.g., ELP-MRD fusion protein) binds ErbB2 and an angiogenic factor and/or IGF1R.

In some embodiments, the MMM complex (e.g., ELP-MRD fusion protein) binds ErbB2 and Ang2. In some embodiments, the MMM complex (e.g., ELP-MRD fusion protein) binds ErbB2 and the same Ang2 epitope as an MRD comprising the sequence of SEQ ID NO:8. In some embodiments, the MMM complex (e.g., ELP-MRD fusion protein) binds to ErbB2 and competitively inhibits binding of Ang2 binding by an MRD comprising the sequence of SEQ ID NO:8. In some embodiments, the MMM complex binds to ErbB2 and comprises the sequence of SEQ ID NO:8.

In some embodiments, the MMM complex (e.g., ELP-MRD fusion protein) binds to ErbB2 and IGF1R. In some embodiments, the MMM complex (e.g., ELP-MRD fusion protein) binds to ErbB2 and binds to the same IGF epitope as an MRD comprising the sequence of SEQ ID NO:14. In some embodiments, the MMM complex (e.g., ELP-MRD fusion protein) binds to ErbB2 and competitively inhibits IGF1R binding of an MRD comprising the sequence of SEQ ID NO:14. In some embodiments, the MMM complex (e.g., ELP-MRD fusion protein) binds to ErbB2 and comprises the sequence of SEQ ID NO:14. In some embodiments, the MMM complex (e.g., ELP-MRD fusion protein) binds ErbB2 and comprises the sequence SLFVPRPERK (SEQ ID NO:103). In some embodiments, the MMM complex (e.g., ELP-MRD fusion protein) binds to ErbB2 and comprises the sequence ESDVLHFTST (SEQ ID NO:104). In some embodiments, the MMM complex (e.g., ELP-MRD fusion protein) binds to ErbB2 and comprises the sequence LRKYADGTL (SEQ ID NO:105).

In some embodiments, the MMM complex (e.g., ELP-MRD fusion protein) targets ErbB2, Ang2, and IGF1R.

In some embodiments, the MMM complex (e.g., ELP-MRD fusion protein) binds to both ErbB2 and Ang2 simultaneously. In some embodiments, the MMM complex (e.g., ELP-MRD fusion protein) binds to both ErbB2 and IGF simultaneously. In some embodiments, the MMM complex (e.g., ELP-MRD fusion protein) binds to ErbB2, Ang2, and IGF1R simultaneously. In some embodiments, the MMM complex (e.g., ELP-MRD fusion protein) binds to both ErbB2 and Ang2 simultaneously and exhibits ADCC activity. In some embodiments, the MMM complex (e.g., ELP-MRD fusion protein) binds to ErbB2, Ang2, and IGF1R simultaneously and exhibits ADCC activity. In additional embodiments, the MMM complex (e.g., ELP-MRD fusion protein) binds ErbB2, and Ang2 and/or IGF1R binding MRD(s) and down-regulates Akt signaling. In additional embodiments, the MMM complex (e.g., ELP-MRD fusion protein) binds ErbB2 and Ang2 and inhibits Ang2 binding to Tie2. In additional embodiments, the MMM complex (e.g., ELP-MRD fusion protein) binds ErbB2 and Ang2 and/or IGF and down-regulates IGF signaling. In additional embodiments, the MMM complex (e.g., ELP-MRD fusion protein) binds ErbB2, and Ang2 and/or IGF1R and inhibits cell proliferation. In additional embodiments, the MMM complex (e.g., ELP-MRD fusion protein) binds ErbB2 and Ang2 and/or IGF1R and inhibits tumor growth.

In specific embodiments, the MMM complex (e.g., ELP-MRD fusion protein) binds VEGF and an angiogenic factor. In specific embodiments, the MMM complex (e.g., ELP-MRD fusion protein) targets VEGF and IGF1R. In another embodiment, the MMM complex (e.g., ELP-MRD fusion protein) targets VEGF, and at least one MRD targets an angiogenic factor and/or IGF1R. In one embodiment, an MMM complex (e.g., an ELP-MRD fusion protein) that binds to the same VEGF epitope as bevacizumab is operably linked to at least one MRD that targets an angiogenic factor and/or IGF1R. In an additional embodiment, an MRD or antibody variable domain fragment that competitively inhibits bevacizumab binding is operably linked to at least one MRD that targets an angiogenic factor and/or IGF1R. In additional embodiments, MMM complex (e.g., ELP-MRD fusion protein) comprises the sequences of SEQ ID NOS:78-79 operably linked to at least one MRD that targets an angiogenic factor and/or IGF1R. In additional embodiments, an MMM complex (e.g., an ELP-MRD fusion protein) that comprises the sequences of SEQ ID NOS:78-79 operably linked to at least one MRD that targets an angiogenic factor and/or IGF1R.

In some embodiments, the MMM complex (e.g., ELP-MRD fusion protein) binds VEGF and comprises an MRD that binds Ang2. In some embodiments, the MMM complex (e.g., ELP-MRD fusion protein) binds VEGF and comprises an Ang2 binding MRD that binds to the same Ang2 epitope as an MRD comprising the sequence of SEQ ID NO:8. In some embodiments, the MMM complex (e.g., ELP-MRD fusion protein) binds VEGF and comprises and MRD that competitively inhibits an MRD comprising the sequence of SEQ ID NO:8. In some embodiments, MMM complex (e.g., ELP-MRD fusion protein) binds VEGF and comprises an MRD comprising the sequence of SEQ ID NO:8.

In some embodiments, the MMM complex (e.g., ELP-MRD fusion protein) binds VEGF and comprises an MRD that binds IGF1R. In some embodiments, the MMM complex (e.g., ELP-MRD fusion protein) binds VEGF and comprises an MRD that binds to the same IGF1R epitope as an MRD comprising the sequence of SEQ ID NO:14. In some embodiments, the MMM complex (e.g., ELP-MRD fusion protein) binds VEGF and comprises an IGF binding MRD that competitively inhibits binding of an MRD comprising the sequence of SEQ ID NO:14. In some embodiments, the MMM complex (e.g., ELP-MRD fusion protein) binds VEGF and contains an MRD comprising the sequence of SEQ ID NO:14. In some embodiments, the MMM complex (e.g., ELP-MRD fusion protein) binds VEGF and comprises an MRD encoding the sequence SLFVPRPERK (SEQ ID NO:103). In some embodiments, the MMM complex (e.g., ELP-MRD fusion protein) binds VEGF and comprises an MRD encoding the sequence ESDVLHFTST (SEQ ID NO:104). In some embodiments, the MMM complex (e.g., ELP-MRD fusion protein) binds VEGF and comprises an MRD encoding the sequence LRKYADGTL (SEQ ID NO:105).

In some embodiments, the MMM complex (e.g., ELP-MRD fusion protein) binds VEGF, Ang2, and IGF1R. In some embodiments, the MMM complex (e.g., ELP-MRD fusion protein) comprises an antibody variable domain fragment that binds VEGF, and MRDs that bind Ang2, and IGF1R.

In some embodiments, the MMM complex (e.g., ELP-MRD fusion protein) binds to both VEGF and Ang2 simultaneously. In some embodiments, the MMM complex (e.g., ELP-MRD fusion protein) binds to VEGF and IGFR1 simultaneously. In some embodiments, the MMM complex (e.g., ELP-MRD fusion protein) binds to VEGF, Ang2, and IGF1R simultaneously. In some embodiments, the MMM complex (e.g., ELP-MRD fusion protein) binds VEGF, and Ang2 and/or IGF1R and exhibits ADCC activity. In additional embodiments, the MMM complex (e.g., ELP-MRD fusion protein) binds VEGF, and Ang2 and/or IGF1R and down-regulates VEGF signaling. In additional embodiments, MMM complex (e.g., ELP-MRD fusion protein) binds VEGF and Tie2 and inhibits Ang2 binding to Tie2. In additional embodiments, the MMM complex (e.g., ELP-MRD fusion protein) binds VEGF and IGF1R and inhibits IGF1R signaling. In additional embodiments, the MMM complex (e.g., ELP-MRD fusion protein) binds VEGF and Ang2 and/or IGF and inhibits cell proliferation. In additional embodiments, the MMM complex (e.g., ELP-MRD fusion protein) binds VEGF and Ang2 and/or IGF and inhibits tumor growth.

In some embodiments, the MMM complex (e.g., ELP-MRD fusion protein) binds ErbB2 and competitively inhibits binding of pertuzumab to ErbB2. In some embodiments, the MMM complex (e.g., ELP-MRD fusion protein) binds ErbB2, and Ang2. and competitively inhibits binding of pertuzumab to ErbB2. In some embodiments, the MMM complex (e.g., ELP-MRD fusion protein) binds ErbB2, and IGF and competitively inhibits binding of pertuzumab to ErbB2. In some embodiments, the MMM complex (e.g., ELP-MRD fusion protein) binds ErbB2, Ang2 and IGF1R and competitively inhibits binding of pertuzumab to ErbB2. In some embodiments, the MMM complex (e.g., ELP-MRD fusion protein) binds ErbB2, VEGF, and Ang2 or IGF1R and competitively inhibits the binding of pertuzumab to ErbB2.

In additional embodiments, the MMM complex (e.g., ELP-MRD fusion protein) binds TNF and an angiogenic factor. In one embodiment, the MMM complex (e.g., ELP-MRD fusion protein) binds to the same TNF epitope as adalimumab and binds an angiogenic factor. In an additional embodiment, an MMM complex (e.g., an ELP-MRD fusion protein) competitively inhibits adalimumab binding to TNF and binds an angiogenic factor. In additional embodiments, an ELP-MRD fusion comprises the sequences of SEQ ID NOS:80-85 and binds an angiogenic factor. In one embodiment, an MMM complex (e.g., an ELP-MRD fusion protein) binds to the same TNF epitope as golimumab and also binds an angiogenic factor. In an additional embodiment, an MMM complex (e.g., an ELP-MRD fusion protein) competitively inhibits golimumab binding to TNF and binds an angiogenic factor.

In some embodiments, an MMM complex (e.g., an ELP-MRD fusion protein) binds to TNF and Ang2. In some embodiments, the MMM complex (e.g., ELP-MRD fusion protein) binds to TNF and also binds to the same Ang2 epitope as an MRD comprising the sequence of SEQ ID NO:8. In some embodiments, the MMM complex (e.g., ELP-MRD fusion protein) binds to TNF and also competitively inhibits binding of Ang-2 by an MRD comprising the sequence of SEQ ID NO:8. In some embodiments, the MMM complex (e.g., ELP-MRD fusion protein) binds to TNF and comprises an MRD having the sequence of SEQ ID NO:8.

In some embodiments, the MMM complex (e.g., ELP-MRD fusion protein) binds to TNF and Ang2 simultaneously. In some embodiments, the MMM complex (e.g., ELP-MRD fusion protein) binds to TNF and Ang2 and exhibits ADCC activity. In additional embodiments, the MMM complex (e.g., ELP-MRD fusion protein) binds to TNF and Ang2 and inhibits binding of TNF to the p55 and p75 cell surface TNF receptors. In additional embodiments, the MMM complex (e.g., ELP-MRD fusion protein) binds to TNF binds and Ang2 and also lyses surface TNF-expressing cells in vitro in the presence of complement. In additional embodiments, the MMM complex (e.g., ELP-MRD fusion protein) binds to TNF and Ang2 and competitively inhibits Ang2 binding to Tie2. In additional embodiments, the MMM complex (e.g., ELP-MRD fusion protein) binds to TNF binds and Ang2 and reduces the signs and symptoms of arthritis.

In some embodiments, embodiments, the MMM complex (e.g., ELP-MRD fusion protein) does not undergo reversible inverse phase transition at a biologically relevant onset temperature of phase transition (Tt). In additional embodiments, the Tt of the MMM complex (e.g., ELP-MRD fusion protein) is less than about 30° C., 25° C., 20° C., 15° C., 10° C., 5° C., or 3° C. In additional embodiments, the onset temperature of phase transition (Tt) for the MMM complex (e.g., ELP-MRD fusion protein) is between about 3-30° C., 3-25° C., 3-20° C. or 3-15° C.

In particular embodiments, the MMM complexes (e.g., ELP-MRD fusion proteins) bind to one or more biological targets at temperatures below the phase transition (i.e., when MMM complex (e.g., ELP-MRD fusion protein) is in a hydrophilic state). In some embodiments, the MMM complex (e.g., ELP-MRD fusion protein) binds to a cell receptor at temperatures below the phase transition state. In some embodiments, the MMM complex (e.g., ELP-MRD fusion protein) binds to a cell receptor at temperatures below 30° C., 25° C., 20° C., 15° C., or 10° C. In additional embodiments, the MMM complex (e.g., ELP-MRD fusion protein) is multivalent at temperatures below the phase transition for the fusion protein. In further embodiments, the MMM complex (e.g., ELP-MRD fusion protein) is multivalent and binds to a cell receptor at temperatures below the onset temperature of phase transition (Tt) for the fusion protein. In further embodiments, the MMM complex (e.g., ELP-MRD fusion protein) is multivalent and binds more than one different cell receptors and/or soluble ligand at temperatures below the onset temperature of phase transition (Tt) for the fusion protein. In additional embodiments, the MMM complex (e.g., ELP-MRD fusion protein) is multivalent and binds to one or more different cell receptors at temperatures below 30° C., 25° C., 20° C., 15° C., or 10° C.

In other embodiments, the onset temperature of phase transition (Tt) for the MMM complex (e.g., ELP-MRD fusion protein) is more than about 33° C., 35° C., 40° C., 45° C., 50° C., 55° C., 60° C., or 65° C. In additional embodiments, the onset temperature of phase transition (Tt) for the MMM complex (e.g., ELP-MRD fusion protein) is between about 30-50° C., 30-40° C., or 30-35° C.

In certain embodiments, the MMM complex (e.g., ELP-MRD fusion protein) does not undergo reversible inverse phase transition at a biologically relevant Tt and the physiological properties of the fusion protein are independent of phase transition. In some embodiments, the MMM complex (e.g., ELP-MRD fusion protein) does not undergo reversible phase transition at a biologically relevant Tt, but the phase transition properties are useful for recovery and/or purification of the ELP-MRD fusion protein).

For example, the ELP forms insoluble polymers when reaching sufficient size, which can be readily removed and isolated from solution by centrifugation. Such phase transition is reversible, and isolated insoluble ELPs can be completely resolubilized in buffer solution when the temperature is returned below the Tt of the ELPs. Thus, ELP-MRD fusions can, in some embodiments, be separated from other contaminating proteins to high purity using inverse transition cycling procedures, e.g., utilizing the temperature-dependent solubility of therapeutic agent, or salt addition to the medium. Successive inverse phase transition cycles can be used to obtain a high degree of purity. In addition to temperature and ionic strength, other environmental variables useful for modulating the inverse transition of therapeutic agents include pH, the addition of inorganic and organic solutes and solvents, side-chain ionization or chemical modification, and pressure.

In some embodiments, the MMM complex (e.g., ELP-MRD fusion protein) circulates or exists in the body in a soluble form, and escapes filtration by the kidney thereby persisting in the body in an active form. In some embodiments, the MMM complexes (e.g., ELP-MRD fusion proteins) have a molecular weight of less than the generally recognized cut-off for filtration through the kidney, such as less than about 60 kD, or alternatively, in some embodiments, less than about 55, 50, 45, 40, 30, or 20 kDa, and persist in the body by at least 2-fold, 3-fold, 4-fold, 5-fold, 10-fold, 20-fold, or 100-fold longer than an uncoupled (e.g., unfused or unconjugated) MRD.

### VI. Methods of Making ELP-MRD Fusions

MMM complexes (e.g., ELP-MRD fusion proteins) of the invention are highly tunable proteins containing modular functionalities and properties that are amenable to rapid rational design, production and optimization. The knowledge and level of skill relating to recombinant technology is such that one can readily exploit the ability to control the sequence, molecular weight, and thermal properties of ELPs (e.g., by guest residue selections of the ELP repeat units) and other components of the MMM complexes (e.g., ELP-MRD fusion proteins) to design MMM complexes (e.g., ELP-MRD fusion proteins) demonstrating desired functionalities.

MRDs and/or the MMM complex (e.g., ELP-MRD fusion protein) of the invention can be prepared by any method known in the art. For example, MMM complexes (e.g., ELP-MRD fusion proteins) “recombinantly produced,” i.e., produced using recombinant DNA technology. For example, recombinant methods available for synthesizing the MMM complexes (e.g., ELP-MRD fusion proteins) of the invention, include, but are not limited to polymerase chain reaction (PCR) based synthesis, concatemerization, seamless cloning, and recursive directional ligation (RDL) (see, e.g., Meyer et al., Biomacromolecules 3:357-367 (2002), Kurihara et al., Biotechnol. Lett. 27:665-670 (2005), Haider et al., Mol. Pharm. 2:139-150 (2005); and McMillan et al., 32:3643-3646 (1999), each of which are herein incorporated by reference).

Moreover, the genetic engineering of the components of the MMM complexes (e.g., ELP-MRD fusion proteins) also provides a facile method to introduce residues for conjugation of therapeutics and/or a variety of labile linkers to control the release of free drug from an ELP-drug conjugate. For example, in one embodiment, the inclusion of an N-terminal lysine on the MMM complex (e.g., ELP-MRD fusion protein) confers the ability to conjugate doxorubicin (Dox), a commonly used chemotherapeutic, through a pH sensitive hydrazone linker to the MMM complex (e.g., ELP-MRD fusion protein). This lysine residue can be functionalized by reaction with succinimidyl-4-(N-maleimidomethyl)cyclohexane-1-carboxylate (SMCC), to form a reactive maleimide group that can then be reacted with Dox-hydrazone, and thereby conjugate Dox to MMM complex (e.g., ELP-MRD fusion protein). According to this embodiment, endosomal uptake of the MMM complex (e.g., ELP-MRD fusion protein) leads to a change in pH and the release of free Dox from the pH labile hydrazone linker in the acidic lysosomal compartments of the targeted cells.

A. ELP-MRD Purification

MRD and/or the MMM complexes (e.g., ELP-MRD fusion proteins) of the invention can be purified by any methods and technologies known in the art. Nonetheless, the temperature (or other stimulus) phase transition responsiveness of the ELP fusion proteins can be exploited to isolate and purify the MMM complexes (e.g., ELP-MRD fusion proteins) using methods that have clear advantages over conventional chromatographic techniques. More particularly, the ability to induce MMM complex (e.g., ELP-MRD fusion protein) aggregation (e.g., by changing temperature or ionic strength), allows for the use of inverse transition cycling (ITC) to rapidly purify the protein. According to this method, the addition of, for example, heat or salt, triggers phase transition leading to aggregation of the ELP-MRD fusion and the aggregated ELP-MRD fusion is then separated from the cell lysate by centrifugation. (see, e.g., Meyer et al., Nat. Biotechnol. 17:1112-1115 (1999), herein incorporated by reference). After discarding the supernatant, the pellet containing the aggregated MMM complex (e.g., ELP-MRD fusion protein) is redissolved in cold buffer. Subsequent centrifugation of the resolubilized MMM complex (e.g., ELP-MRD fusion protein) containing solution below the Tt provides a means by which to eliminate contaminating insoluble proteins in the MMM complex (e.g., ELP-MRD fusion protein) containing pellet. This cycle is than optionally repeated at least 1, 2, 3, 4, 5, or more times to increase the purity of the MMM complex (e.g., ELP-MRD fusion protein). In some embodiments, elastin or elastin-like peptide is added to the cell lysate to increase the purification efficiency of the ITC method (see, e.g., Christensen et al., Anal. Biochem. 360:166-168 (2007), and Ge et al., Biomacromolecules 7:2475-2478 (2006), both of which are herein incorporated by reference).

In another embodiment, the MMM complexes (e.g., ELP-MRD fusion proteins) are isolated by indirect ITC. This process combines ITC with affinity capture methods in which an ELP or another component of the MMM complex (e.g., ELP-MRD fusion protein) is attached to a polypeptide capture agent that binds to a target protein. Following binding of the target with the capture agent-ELP fusion in solution, purification of the fusion protein is carried out via ITC. In an alternative embodiment using the indirect ITC approach, metal binding domains are incorporated into the MMM complex (e.g., ELP-MRD fusion protein) and are bound to Ni2+ and the Ni2+-MMM complex (e.g., ELP-MRD fusion protein) is purified using an oligohistidine sequence by metal affinity capture.

In additional embodiments, the MMM complexes are optionally fused to heterologous polypeptide sequences described herein or otherwise known in the art to facilitate purification. More particularly, it is envisioned that ligands (e.g., antibodies and other affinity matrices) for MRDs or other components of the MMM complexes can be used in affinity columns for affinity purification and that optionally, the MRDs or other components of the MMM complex that are bound by these ligands are removed from the composition prior to final preparation of the MMM complexes using techniques known in the art.

B. Polynucleotides, Vectors, and Host Cells

In another embodiment, the invention provides polynucleotides comprising a nucleotide sequence encoding and MRD and/or the MMM complex (e.g., ELP-MRD fusion protein) of the invention. Such polynucleotides further comprise, in addition to sequences encoding an MRD and/or the MMM complex (e.g., ELP-MRD fusion protein), one or more expression control elements. For example, the polynucleotide, may comprise one or more promoters or transcriptional enhancers, ribosomal binding sites, transcription termination signals, and polyadenylation signals, as expression control elements. The polynucleotide can be inserted within any suitable vector, which can be contained within any suitable host cell for expression.

A vector comprising the polynucleotide can be introduced into a cell for expression of MRD and/or the MMM complex (e.g., ELP-MRD fusion protein). The vector can remain episomal or become chromosomally integrated, as long as the insert encoding therapeutic agent can be transcribed. Vectors can be constructed by standard recombinant DNA technology. Vectors can be plasmids, phages, cosmids, phagemids, viruses, or any other types known in the art, which are used for replication and expression in prokaryotic or eukaryotic cells. It will be appreciated by one of skill in the art that a wide variety of components known in the art (such as expression control elements) can be included in such vectors, including a wide variety of transcription signals, such as promoters and other sequences that regulate the binding of RNA polymerase onto the promoter. Any promoter known to be effective in the cells in which the vector will be expressed can be used to initiate expression of MRD and/or the MMM complex (e.g., ELP-MRD fusion protein). Suitable promoters can be inducible or constitutive. Examples of suitable promoters include the SV40 early promoter region, the promoter contained in the 3′ long terminal repeat of Rous sarcoma virus, the HSV-1 (herpes simplex virus-1) thymidine kinase promoter, the regulatory sequences of the metallothionein gene, etc., as well as the following animal transcriptional control regions, which exhibit tissue specificity and have been utilized in transgenic animals: elastase I gene control region which is active in pancreatic acinar cells; insulin gene control region which is active in pancreatic beta cells, immunoglobulin gene control region which is active in lymphoid cells, mouse mammary tumor virus control region which is active in testicular, breast, lymphoid and mast cells, albumin gene control region which is active in liver, alpha-fetoprotein gene control region which is active in liver, alpha 1-antitrypsin gene control region which is active in the liver, beta-globin gene control region which is active in erythroid cells, myelin basic protein gene control region which is active in oligodendrocyte cells in the brain, myosin light chain-2 gene control region which is active in skeletal muscle, and gonadotropin releasing hormone gene control region which is active in the hypothalamus.

When prepared as recombinant fusions, the MMM complex (e.g., ELP-MRD fusion protein) can be prepared by recombinant expression techniques known in the art. For example, to recombinantly produce the MMM complex (e.g., ELP-MRD fusion protein), a nucleic acid sequence encoding the MMM complex (e.g., ELP-MRD fusion protein) is operatively linked to a suitable promoter sequence such that the nucleic acid sequence encoding the fusion protein is transcribed and/or translated into the desired fusion protein in the host cells. Promoters useful for expression in E. coli, include but are not limited to, the T7 promoter. Any commonly used expression system can be used to produce the MMM complexes (e.g., ELP-MRD fusion proteins), including eukaryotic or prokaryotic systems. Specific examples include yeast (e.g., Saccharomyces spp., Pichia spp.), baculovirus, mammalian, and bacterial systems, such as E. coli, and Caulobacter.

The invention also provides for expression vectors and/or host cells that comprises one or more polynucleotides encoding an MRD and/or the MMM complex (e.g., ELP-MRD fusion protein) of the invention. In additional embodiments, the invention provides methods of producing an MMM complex (e.g., an ELP-MRD fusion protein), comprising: culturing a host cell comprising one or more polynucleotides encoding an MRD and/or the MMM complex (e.g., ELP-MRD fusion protein) or an expression vector comprising one or more isolated polynucleotides encoding an MRD and/or the MMM complex (e.g., ELP-MRD fusion protein) in a medium under conditions allowing the expression of said one or more MRD and/or the MMM complex (e.g., ELP-MRD fusion proteins); and recovering said MRD and/or the MMM complex (e.g., ELP-MRD fusion proteins).

Prokaryotes useful as host cells in producing the compositions of the invention (e.g., ELP-MRD fusion proteins) include gram negative or gram positive organisms such as E. coli and B. subtilis. Expression vectors for use in prokaryotic host cells generally contain one or more phenotypic selectable marker genes (e.g., genes encoding proteins that confers antibiotic resistance or that supply an autotrophic requirement). Examples of useful prokaryotic host expression vectors include the pKK223-3 (Pharmacia, Uppsala, Sweden), pGEM1 (Promega, Wis., USA), pET (Novagen, Wis., USA) and pRSET (Invitrogen, Calif., USA) series of vectors (see, e.g., Studier, J. Mol. Biol. 219:37 (1991) and Schoepfer, Gene 124:83 (1993)). Exemplary promoter sequences frequently used in prokaryotic host cell expression vectors include T7, (Rosenberg et al., Gene 56: 125-135 (1987)), beta-lactamase (penicillinase), lactose promoter system (Chang et al., Nature 275:615 (1978)); and Goeddel et al., Nature 281:544 (1979)), tryptophan (trp) promoter system (Goeddel et al., Nucl. Acids Res. 8:4057, (1980)), and tac promoter (Sambrook et al., 1990, Molecular Cloning, A Laboratory Manual, 2d Ed., Cold Spring Harbor Laboratory, Cold Spring Harbor, N.Y.).

In alternative embodiments, eukaryotic host cell systems can be used, including yeast cells transformed with recombinant yeast expression vectors containing the coding sequence of an MMM complex (e.g., an ELP-MRD fusion protein) fusion protein of the present invention, such as the expression systems taught in U.S. Pat. Appl. No. 60/344,169 and WO 03/056914 (methods for producing human-like glycoprotein in a non-human eukaryotic host cell) (the contents of each of which are incorporated by reference in their entirety). Exemplary yeast that can be used to produce compositions of the invention, such as MRDs, include yeast from the genus Saccharomyces, Pichia, Actinomycetes and Kluyveromyces. Yeast vectors typically contain an origin of replication sequence from a 2 mu yeast plasmid, an autonomously replicating sequence (ARS), a promoter region, sequences for polyadenylation, sequences for transcription termination, and a selectable marker gene. Examples of promoter sequences in yeast expression constructs include, promoters from metallothionein, 3-phosphoglycerate kinase (Hitzeman et al., J. Biol. Chem. 255:2073, (1980)) and other glycolytic enzymes, such as, enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Additional suitable vectors and promoters for use in yeast expression as well as yeast transformation protocols are known in the art. See, e.g., Fleer et al., Gene, 107:285-195 (1991) and Hinnen et al., Proc. Natl. Acad. Sci., 75:1929 (1978).

Insect and plant host cell culture systems are also useful for producing the complexes of the invention. Such host cell systems include for example, insect cell systems infected with recombinant virus expression vectors (e.g., baculovirus) containing the coding sequence of an MMM complex (e.g., an ELP-MRD fusion protein); plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the coding sequence of an MMM complex (e.g., an ELP-MRD fusion protein), including, but not limited to, the expression systems taught in U.S. Pat. No. 6,815,184, WO 2004/057002, WO 2004/024927, U.S. Pat. Appl. Nos. 60/365,769, 60/368,047, and WO 2003/078614, the contents of each of which is herein incorporated by reference in its entirety.

In alternate embodiments, other eukaryotic host cell systems can be used, including animal cell systems infected with recombinant virus expression vectors (e.g., adenovirus, vaccinia virus) including cell lines engineered to contain multiple copies of the DNA encoding an MMM complex (e.g., an ELP-MRD fusion protein) either stably amplified (CHO/dhfr) or unstably amplified in double-minute chromosomes (e.g., murine cell lines). In one embodiment, the vector comprising the polynucleotide(s) encoding the MMM complex (e.g., ELP-MRD fusion protein) of the invention is polycistronic.

Exemplary mammalian cells useful for producing these compositions include 293 cells (e.g., 293T and 293F), CHO cells, BHK cells, NS0 cells, SP2/0 cells, YO myeloma cells, P3X63 mouse myeloma cells, PER cells, PER.C6 (Crucell, Netherlands) cells or hybridoma cells, other mammalian cells. Additional exemplary mammalian host cells that are useful in practicing the invention include but are not limited, to VERY, Hela, COS, MDCK, 3T3, W138, BT483, Hs578T, HTB2, BT20 and T47D, CRL7O3O and HsS78Bst cells. Some examples of expression systems and selection methods are described in the following references and references cited therein: Borth et al., Biotechnol. Bioen. 71(4):266-73 (2000-2001), in Werner et al., Arzneimittelforschung/Drug Res. 48(8):870-80 (1998), in Andersen and Krummen, Curr. Op. Biotechnol. 13:117-123 (2002), in Chadd and Chamow, Curr. Op. Biotechnol. 12:188-194 (2001), and in Giddings, Curr. Op. Biotechnol. 12: 450-454 (2001). Additional examples of expression systems and selection methods are described in Logan & Shenk, Proc. Natl. Acad. Sci. USA, 81:355-359 (1984), Bittner et al., Methods in Enzymol. 153:51-544 (1987)). Transcriptional and translational control sequences for mammalian host cell expression vectors are frequently derived from viral genomes. Commonly used promoter sequences and enhancer sequences in mammalian expression vectors include, sequences derived from Polyoma virus, Adenovirus 2, Simian Virus 40 (SV40), and human cytomegalovirus (CMV). Exemplary commercially available expression vectors for use in mammalian host cells include pCEP4 (Invitrogen®) and pcDNA3 (Invitrogen®).

A number of selection systems can be used in mammalian host-vector expression systems, including, but not limited to, the herpes simplex virus thymidine kinase, hypoxanthine-guanine phosphoribosyltransferase and adenine phosphoribosyltransferase (Lowy et al., Cell 22:817 (1980)) genes, which can be employed in tk, hgprt⁻ or aprt⁻ cells, respectively. Additionally, antimetabolite resistance can be used as the basis of selection for e.g., dhfr, gpt, neo, hygro, trpB, hisD, ODC (ornithine decarboxylase), and the glutamine synthase system.

Methods which are well known to those skilled in the art can be used to construct expression vectors containing the coding sequence of an MMM complex (e.g., an ELP-MRD fusion protein) along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989) and Ausubel et al., CURRENT PROTOCOLS IN MOLECULAR BIOLOGY, Greene Publishing Associates and Wiley Interscience, N.Y (1989).

A variety of host-expression vector systems can be utilized to express the coding sequence an MMM complex (e.g., an ELP-MRD fusion protein). A host cell strain can be chosen which modulates the expression of inserted antibody sequences, or modifies and processes the antibody gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the antibody or portion thereof expressed. To this end, eukaryotic host cells which possess the cellular machinery for proper processing of the primary transcript, glycosylation, and phosphorylation of the gene product can be used. Stable expression typically achieves more reproducible results than transient expression and also is more amenable to large-scale production; however, it is within the skill of one in the art to determine whether transient expression is better for a particular situation. Rather than using expression vectors which contain viral origins of replication, host cells can be transformed with the respective coding nucleic acids controlled by appropriate expression control elements (e.g., promoter, enhancer, sequences, transcription terminators, polyadenylation sites, etc.), and a selectable marker. Following the introduction of foreign DNA, engineered cells can be allowed to grow for 1-2 days in an enriched media, and then are switched to a selective media. The selectable marker in the recombinant plasmid confers resistance to the selection and allows selection of cells which have stably integrated the plasmid into their chromosomes and grow to form foci which in turn can be cloned and expanded into cell lines.

### VIII. Uses of ELP-MRD Fusions

The MMM complexes (e.g., ELP-MRD fusion proteins) described herein are useful in a variety of applications including, but not limited to, therapeutic treatment methods, such as the treatment of cancer. In certain embodiments, the MMM complexes (e.g., ELP-MRD fusion proteins) are useful for inhibiting tumor growth, reducing neovascularization, reducing angiogenesis, inducing differentiation, reducing tumor volume, and/or reducing the tumorigenicity of a tumor. The methods of use can be in vitro, ex vivo, or in vivo methods.

In one embodiment, the MMM complexes (e.g., ELP-MRD fusion proteins) are useful for detecting the presence of a factor or multiple factors (e.g., antigens or organisms) in a biological sample. The term “detecting” as used herein encompasses quantitative or qualitative detection. In certain embodiments, a biological sample comprises a cell or tissue. In certain embodiments, such tissues include normal and/or cancerous tissues.

The present invention contemplates therapeutic compositions useful for practicing therapeutic methods described herein. In one embodiment, therapeutic compositions of the present invention contain a physiologically tolerable carrier together with at least one species of ELP-MRD fusion as described herein, dissolved or dispersed therein as an active ingredient. In another embodiment, therapeutic compositions of the present invention contain a physiologically tolerable carrier together with at least one species of an MRD as described herein, dissolved or dispersed therein as an active ingredient. In a preferred embodiment, therapeutic composition is not immunogenic when administered to a human patient for therapeutic purposes.

The preparation of a pharmacological composition that contains active ingredients dissolved or dispersed therein is well understood in the art. Typically such compositions are prepared as sterile injectables either as liquid solutions or suspensions, aqueous or nonaqueous. However, solid forms suitable for solution, or suspensions, in liquid prior to use can also be prepared. The preparation can also be emulsified. Thus, an ELP-MRD containing composition can take the form of solutions, suspensions, tablets, capsules, sustained release formulations or powders, or other compositional forms.

In some embodiments, the MMM complexes of the invention (e.g., ELP-MRD fusion proteins) are formulated to ensure or optimize distribution in vivo. For example, the blood-brain barrier (BBB) excludes many highly hydrophilic compounds and if so desired, the compositions are prepared so as to increase transfer across the BBB, by for example, formulation in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., Ranade Clin. Pharmacol. 29:685 (1989)).

The active ingredient can be mixed with excipients which are pharmaceutically acceptable and compatible with the active ingredient and in amounts suitable for use in therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

Therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylarnine, 2-ethylamino ethanol, histidine, procaine and the like.

Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, propylene glycol, polyethylene glycol, and other solutes.

Liquid compositions can also contain liquid phases in addition to and to the exclusion of water.

Exemplary of such additional liquid phases are glycerin, vegetable oils such as cottonseed oil, organic esters such as ethyl oleate, and water-oil emulsions.

In one embodiment, a therapeutic composition contains an ELP-MRD fusion of the present invention, typically in an amount of at least 0.1 weight percent of ELP-MRD fusion per weight of total therapeutic composition. A weight percent is a ratio by weight of ELP-MRD fusion per total composition. Thus, for example, 0.1 weight percent is 0.1 grams of ELP-MRD per 100 grams of total composition.

The MMM complexes (e.g., MRD-ELP fusion proteins) are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The dosage schedule and amounts effective for therapeutic and prophylactic uses, i.e., the “dosing regimen”, will depend upon a variety of factors, including the cause, stage and severity of the disease or disorder, the health, physical status, age of the mammal being treated, and the site and mode of the delivery of the MMM complex. Therapeutic efficacy and toxicity of the complex and forumation can be determined by standard pharmaceutical, pharmacological, and toxicological procedures in cell cultures or experimental animals. Data obtained from these procedures caln likewise be used in formulating a range of dosages for human use. Moreover, therapeutic index (i.e., the dose therapeutically effective in 50 percent of the population divided by the dose lethal to 50 percent of the population (ED₅₀/LD₅₀)) can readily be determined using known procedures. The dosage is preferably within a range of concentrations that includes the ED₅₀ with little or no toxicity, and may vary within this range depending on the dosage form employed, sensitivity of the patient, and the route of administration.

The dosage regimen also takes into consideration pharmacokinetics parameters known in the art, such as drug absorption rate, bioavailability, metabolism and clearance (see, e.g., Hidalgo-Aragones, J. Steroid Biochem. Mol. Biol. 58:611-617 (1996); Groning et al., Pharmazie 51:337-341 (1996); Fotherby Contraception 54:59-69 (1996); and Johnson et al., J. Pharm. Sci. 84:1144-1146 (1995)). It is well within the state of the art for the clinician to determine the dosage regimen for each subject being treated. Moreover, single or multiple administrations of MMM complex containing compositions can be administered depending on the dosage and frequency as required and tolerated by the subject. The duration of prophylactic and therapeutic treatment will vary depending on the particular disease or condition being treated. Some diseases are amenable to acute treatment whereas others require long-term, chronic therapy. When treating with an additional therapeutic agent, MMM complex can be administered serially, or simultaneously with the additional therapeutic agent.

Therapeutically effective amounts of MMM complexes (e.g., MRD-ELP fusion proteins) of the invention vary according to, for example, the targets of the MMM c complex and the potency of conjugated cytotoxic agents encompassed by various embodiments of the invention Thus, for example therapeutically effective dose of an MMM complex that “mops up” a soluble ligand, such as TNF alpha, is expected to be higher than that for an MMM complex that redicrects T cell effector function to a target on a hematological malignancy. Likewise, therapeutically effective amounts of MMM complexes comprising a maytansinoid cytotoxic agent are likely to be lower then the dosage of an MMM complex comprising a less potent chemotherapeutic, such as taxol, or the counterpart MMM does not contain a cytotoxic agent.

According to one embodiment, a therapeutically effective dose of an MMM complex (e.g., MRD-ELP fusion protein) is an amount selected from about 0.00001 mg/kg to about 20 mg/kg, from about 0.00001 mg/kg to about 10 mg/kg, from about 0.00001 mg/kg to about 5 mg/kg, from about 0.0001 mg/kg to about 20 mg/kg, from about 0.0001 mg/kg to about 10 mg/kg, from about 0.0001 mg/kg to about 5 mg/kg, from about 0.001 mg/kg to about 20 mg/kg, from about 0.001 mg/kg to about 10 mg/kg, and from about 0.001 mg/kg to about 5 mg/kg of the patient's body weight, in one or more dose administrations daily, for one or several days.

According to another embodiment, a therapeutically effective amount of an MMM complex (e.g., MRD-ELP fusion protein) is an amount of MMM complex such that when administered in a physiologically tolerable composition is sufficient to achieve a plasma concentration of from about 0.1 microgram (μg) per milliliter (ml) to about 100 μg/ml, from about 1 μg/ml to about 5 μg/ml, and usually about 5 μg/ml. Stated differently, in another embodiment, the dosage can vary from about 0.1 mg/kg to about 300 mg/kg, from about 0.2 mg/kg to about 200 mg/kg, from about 0.5 mg/kg to about 20 mg/kg, in one or more dose administrations daily, for one or several days.

In some embodiments, the MMM complex (e.g., MRD-ELP fusion proteins) is administered at about 1 mg/kg to about 50 mg/kg, about 1 mg/kg to about 25 mg/kg, about 1 mg/kg to about 20 mg/kg, about 1 mg/kg to about 15 mg/kg, about 1 mg/kg to about 10 mg/kg, or about 1 mg/kg to about 5 mg/kg.

In one embodiment, the MMM complexes are administered in metronomic dosing regimens, either by continuous infusion or frequent administration without extended rest periods. Such metronomic administration can involve dosing at constant intervals without rest periods. According to this embodiment, MMM complexes, in those containing particular cytotoxic agents, are used at lower doses. Such dosing regimens encompass the chronic daily administration of relatively low doses for extended periods of time, which can minimize toxic side effects and eliminate rest periods. See, e.g., Kamat et al. Cancer Research 67:281-88 (2007). According to some embodiments, the MMM complex of the invention is delivered by chronic low-dose or continuous infusion ranging from about 24 hours to about 2 days, from about 24 hours to about 1 week, from about 24 hours to about 2 weeks, from about 24 hours to about 3 weeks, from about 24 hours to about 1 month, from about 24 hours to about 2 months, from about 24 hours to about 3 months, from about 24 hours to about 4 months, from about 24 hours to about 5 months, and from about 24 hours to about 6 months. In a particular embodiment, the MMM complex of the invention is delivered by chronic low-dose or continuous infusion ranging from 2 weeks to 6 weeks for 5 cycles. The scheduling of such dose regimens can be optimized by those of skill in the art.

An ELP-MRD fusion-containing therapeutic composition typically contains about 10 micrograms (μg) per milliliter (ml) to about 100 milligrams (mg) per ml of ELP-MRD fusion as active ingredient per volume of composition, and more preferably contains about 1 mg/ml to about 10 mg/ml (i.e., about 0.1 to 1 weight percent).

A therapeutic composition in another embodiment contains a polypeptide of the present invention, typically in an amount of at least 0.1 weight percent of polypeptide per weight of total therapeutic composition. A weight percent is a ratio by weight of polypeptide total composition. Thus, for example, 0.1 weight percent is 0.1 grams of polypeptide per 100 grams of total composition.

A polypeptide-containing therapeutic composition can contain about 10 micrograms (ug) per milliliter (ml) to about 100 milligrams (mg) per ml of polypeptide as active ingredient per volume of composition, and can contain about 1 mg/ml to about 10 mg/ml (i.e., about 0.1 to 1 weight percent).

The dosage ranges for the administration of the ELP-MRD molecule of the invention are those large enough to produce the desired effect in which the disease symptoms mediated by the target molecule are ameliorated. The dosage should not be so large as to cause adverse side effects, such as hyperviscosity syndromes, pulmonary edema, congestive heart failure, and the like. Generally, the dosage will vary with the age, condition, sex and extent of the disease in the patient and can be determined by one of skill in the art. The dosage can be adjusted by the individual physician in the event of any complication.

The MMM complexes (e.g., MRD-ELP fusion proteins) need not be, but is optionally formulated with one or more agents currently used to prevent or treat the disorder in question. The effective amount of such other agents depends on the amount of MMM multispecific complex present in the formulation, the type of disorder or treatment, and other factors discussed above.

As discussed above, the appropriate dosage of the MMM complex (e.g., MRD-ELP fusion protein) will depend on the type of disease to be treated, as defined above, the severity and course of the disease, previous therapy, the patient's clinical history, and the discretion of the attending physician. The MMM complex is suitably administered to the patient at one time or over a series of treatments. Preferably, the MMM complex is administered by intravenous infusion or by subcutaneous injections. According to some embodiments, the MMM complex is administered parenterally by injection or by gradual infusion over time. Although the target molecule can typically be accessed in the body by systemic administration and therefore most often treated by intravenous administration of therapeutic compositions, other tissues and delivery means are contemplated where there is likelihood that the tissue targeted contains the target molecule. Thus, the MMM complex can be administered intravenously, intraperitoneally, intramuscularly, subcutaneously, intracavity, transdermally, and can be delivered by peristaltic means. MMM complexes can also be delivered by aerosol to airways and lungs. In some embodiments, the MRD-ELP fusion protein is administered by intravenous infusion. In some embodiments, the antibody-MRD molecule is administered by subcutaneous injection.

Therapeutic compositions containing an MMM complex (e.g., MRD-ELP fusion protein) can conventionally be administered intravenously, as by injection of a unit dose, for example. The term “unit dose” when used in reference to a therapeutic complex of the present invention refers to physically discrete units suitable as unitary dosage for the patient, each unit containing a predetermined quantity of active material calculated to produce the desired therapeutic effect in association with the required diluent; i.e., carrier, or vehicle. In a specific embodiment, therapeutic complexes containing a human monoclonal antibody or a polypeptide are administered subcutaneously.

The complexes of the invention are administered in a manner compatible with the dosage formulation, and in a therapeutically effective amount. The quantity to be administered depends on the patient to be treated, capacity of the patient's system to utilize the active ingredient, and degree of therapeutic effect desired. Precise amounts of active ingredient required to be administered depend on the judgment of the practitioner and are peculiar to each individual. However, suitable dosage ranges for systemic application are disclosed herein and depend on the route of administration. Suitable regimes for administration are also variable, but are typified by an initial administration followed by repeated doses at one or more hour intervals by a subsequent injection or other administration. Alternatively, continuous intravenous infusion sufficient to maintain concentrations in the blood in the ranges specified for in vivo therapies are contemplated.

In other embodiments, the invention provides a method for treating or preventing a disease, disorder, or injury comprising administering a therapeutically effective amount or prophylactically effective amount of MRD-ELP fusion protein molecule to a patient in need thereof. In some embodiments, the disease, disorder or injury is cancer. In other embodiments, the disease, disorder or injury is a disease or disorder of the immune system, such as inflammation or an autoimmune disease.

According to one embodiment, an MRD-ELP fusion protein is administered in combination with a compound that promotes apoptosis, inhibits apoptosis, promotes cell survival, inhibits cell survival, promotes senescence of diseased or aberrant cells, inhibits cell senescence, promotes cell proliferation, inhibits cell proliferation, promotes cell differentiation, inhibits cell differentiation, promotes cell activation, inhibits cell activation, promotes cell metabolism, inhibits cell metabolism, promotes cell adhesion, inhibits cell adhesion, promotes cell cycling or cell division, inhibits cell cycling or cell division, promotes DNA replication or repair, inhibits DNA replication or repair, promotes transcription or translation, or inhibits transcription or translation.

A therapeutically effective amount of an ELP-MRD molecule of the invention can be an amount such that when administered in a physiologically tolerable composition is sufficient to achieve a plasma concentration of from about 0.1 microgram (μg) per milliliter (ml) to about 100 μg/ml, preferably from about 1 μg/ml to about 5 μg/ml, and usually about 5 μg/ml. Stated differently, the dosage can vary from about 0.1 mg/kg to about 300 mg/kg, preferably from about 0.2 mg/kg to about 200 mg/kg, most preferably from about 0.5 mg/kg to about 20 mg/kg, in one or more dose administrations daily, for one or several days.

The ELP-MRD molecule of the invention can be administered parenterally by injection or by gradual infusion over time. Although the target molecule can typically be accessed in the body by systemic administration and therefore most often treated by intravenous administration of therapeutic compositions, other tissues and delivery means are contemplated where there is a likelihood that the tissue targeted contains the target molecule. Thus, ELP-MRD molecules of the invention can be administered intravenously, intraperitoneally, intramuscularly, subcutaneously, intracavity, transdermally, and can be delivered by peristaltic means. MMM complexes (e.g., ELP-MRD fusion proteins) can also be delivered by aerosol to airways and lungs.

Therapeutic compositions containing an ELP-MRD molecule of this invention are conventionally administered intravenously, as by injection of a unit dose, for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent; i.e., carrier, or vehicle. In a specific embodiment, therapeutic compositions containing a ELP-MRD are administered subcutaneously.

The compositions of the invention are administered in a manner compatible with the dosage formulation, and in a therapeutically effective amount. The quantity to be administered depends on the subject to be treated, capacity of the subject's system to utilize the active ingredient, and degree of therapeutic effect desired. Precise amounts of active ingredient required to be administered depend on the judgment of the practitioner and are peculiar to each individual. However, suitable dosage ranges for systemic application are disclosed herein and depend on the route of administration. Suitable regimes for administration are also variable, but are typified by an initial administration followed by repeated doses at one or more hour intervals by a subsequent injection or other administration. Alternatively, continuous intravenous infusion sufficient to maintain concentrations in the blood in the ranges specified for in vivo therapies are contemplated.

In other embodiments, the invention provides a method for treating or preventing a disease, disorder, or injury comprising administering a therapeutically effective amount or prophylactically effective amount of ELP-MRD molecule to a subject in need thereof. In some embodiments, the disease, disorder or injury is cancer. In other embodiments, the disease, disorder or injury is a disease or disorder of the immune system, such as inflammation or an autoimmune disease. This beneficial activity can be demonstrated in vitro, in an in vivo animal model, or in human clinical trials.

In one embodiment, the invention provides a method of treating cancer comprising administering a therapeutically effective amount of a VEGFA or VEGFR binding MMM complex (e.g., ELP-MRD fusion protein) to a patient in need thereof. In a specific embodiment, the invention provides a method of treating cancer comprising administering a therapeutically effective amount of bevacizumab comprising at least one MRD to a patient in need thereof. In one embodiment, the invention provides a method of treating colorectal cancer by administering a therapeutically effective amount of bevacizumab comprising at least one MRD to a patient having colorectal cancer. In another embodiment, the invention provides a method of treating breast cancer by administering a therapeutically effective amount of bevacizumab comprising at least one MRD to a patient having breast cancer. In another embodiment, the invention provides a method of treating non-small cell lung carcinoma by administering a therapeutically effective amount of bevacizumab comprising at least one MRD to a patient having non-small cell lung carcinoma. In other embodiments, therapeutic effective amounts of bevacizumab comprising at least one MRD are administered to a patient to treat metastatic colorectal cancer, metastatic breast cancer, metastatic pancreatic cancer, or metastatic non-small cell lung carcinoma. In another embodiment, the invention provides a method of treating cancer by administering a therapeutically effective amount of bevacizumab comprising at least one MRD to a patient having renal cell carcinoma, glioblastoma muliforme, ovarian cancer, prostate cancer, liver cancer or pancreatic cancer.

Combination therapy and compositions including MMM complexes (e.g., ELP-MRD fusion proteins) of the invention and another therapeutic are also encompassed by the invention, as are methods of treatment using these compositions. In other embodiments, compositions of the invention are administered alone or in combination with one or more additional therapeutic agents. Combinations can be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. “Concurrent” administration, as used herein, refers to administration of two or more agents, where at least part of the administration overlaps in time. Accordingly, concurrent administration includes a dosing regimen when the administration of one or more agent(s) continues after discontinuing the administration of one or more other agent(s). Administration “in combination” further includes the separate administration of one of therapeutic compounds or agents given first, followed by the second. Accordingly, in one embodiment, a VEGFA or VEGFR binding MMM complex (e.g., ELP-MRD fusion protein) is administered in combination with 5-fluorouracil, carboplatin, paclitaxel, or interferon alpha. In another embodiment, bevacizumab comprising at least one MRD is administered in combination with 5-fluorouracil, carboplatin, paclitaxel, or interferon alpha.

In another embodiment, the invention provides a method of treating macular degeneration comprising administering a therapeutically effective amount of a VEGFA or VEGFR binding MMM complex (e.g., ELP-MRD fusion protein) to a patient in need thereof. In a specific embodiment, the invention provides a method of treating macular degeneration comprising administering a therapeutically effective amount of bevacizumab comprising at least one MRD to a patient in need thereof. In a specific embodiment, the invention provides a method of treating macular degeneration comprising administering a therapeutically effective amount of ranibizumab comprising at least one MRD to a patient in need thereof.

In another embodiment, the invention provides a method of treating cancer comprising administering a therapeutically effective amount of a ErbB2 (HER2) binding MMM complex (e.g., ELP-MRD fusion protein) to a patient in need thereof. In a specific embodiment, the invention provides a method of treating cancer comprising administering a therapeutically effective amount of trastuzumab comprising at least one MRD to a patient in need thereof. In one embodiment, the invention provides a method of treating breast cancer by administering a therapeutically effective amount of trastuzumab comprising at least one MRD to a patient having breast cancer. In other embodiments, therapeutic effective amounts of trastuzumab comprising at least one MRD are administered to a patient to treat metastatic breast cancer.

In another embodiment, an ErbB2(HER2) binding MMM complex (e.g., ELP-MRD fusion protein) is administered in combination with cyclophosphamide, paclitaxel, docetaxel, carboplatin, anthracycline, or a maytansinoid. In a specific embodiment, trastuzumab comprising at least one MRD is administered in combination with cyclophosphamide, paclitaxel, docetaxel, carboplatin, anthracycline, or a maytansinoid.

In another embodiment, the invention provides a method of treating cancer comprising administering a therapeutically effective amount of a CD20-binding MMM complex (e.g., ELP-MRD fusion protein) to a patient in need thereof. In a specific embodiment, the invention provides a method of treating a hematologic cancer comprising administering a therapeutically effective amount of rituximab comprising at least one MRD to a patient in need thereof. In one embodiment, the invention provides a method of treating CD20 positive NHL by administering a therapeutically effective amount of bevacizumab comprising at least one MRD to a patient having CD20 positive NHL. In one embodiment, the invention provides a method of treating CD20 positive CLL by administering a therapeutically effective amount of bevacizumab comprising at least one MRD to a patient having CD20 positive CLL.

In another embodiments, a therapeutically effective amount of a CD20-binding MMM complex (e.g., ELP-MRD fusion protein) is administered in combination with: ludarabine, cyclophosphamide, FC (fludarabine and cyclophosphamide), anthracycline based chemotherapy regimen (e.g., CHOP (cyclophosphamide, adriamycin, vincristine and prednisone)), or CVP (cyclophosphamide, prednisone, and vincristine) chemotherapy. In a specific embodiment, a therapeutically effective amount of bevacizumab comprising at least one MRD is administered in combination with: ludarabine, cyclophosphamide, FC (fludarabine and cyclophosphamide), anthracycline based chemotherapy regimen (e.g., CHOP (cyclophosphamide, adriamycin, vincristine and prednisone)), or CVP (cyclophosphamide, prednisone, and vincristine) chemotherapy.

In another embodiment, the invention provides a method of treating a disorder of the immune system comprising administering a therapeutically effective amount of a CD20-binding MMM complex (e.g., ELP-MRD fusion protein) to a patient in need thereof. In a specific embodiment, the invention provides a method of treating an autoimmune disease comprising administering a therapeutically effective amount of a CD20-binding MMM complex (e.g., ELP-MRD fusion protein) to a patient in need thereof. In one embodiment, the invention provides a method of treating an autoimmune disease comprising administering a therapeutically effective amount of a an MMM complex (e.g., an ELP-MRD fusion protein) that competes with Rituximab for binding to CD20a patient in need thereof. In another embodiment, the invention provides a method of treating rheumatoid arthritis comprising administering a therapeutically effective amount of a rituximab-MMM complex (e.g., ELP-MRD fusion protein) to a patient in need thereof. In another embodiment, the invention provides a method of treating systemic lupus erythematosus comprising administering a therapeutically effective amount of a rituximab-MMM complex (e.g., ELP-MRD fusion protein) to a patient in need thereof.

In another embodiment, the invention provides a method of treating a disorder of the immune system comprising administering a therapeutically effective amount of a TNF-binding MMM complex (e.g., ELP-MRD fusion protein) to a patient in need thereof. In a specific embodiment, the invention provides a method of treating a disorder of the immune system comprising administering a therapeutically effective amount of adalimumab comprising at least one MRD to a patient in need thereof. In one embodiment, the invention provides a method of treating an autoimmune disease by administering a therapeutically effective amount of adalimumab comprising at least one MRD to a patient in need thereof. In one embodiment, the invention provides a method of treating rheumatoid arthritis, by administering a therapeutically effective amount of adalimumab comprising at least one MRD to a patient in need thereof. In one embodiment, the invention provides a method of treating an inflammatory disorder, by administering a therapeutically effective amount of adalimumab comprising at least one MRD to a patient in need thereof. In another embodiment, the invention provides a method of treating Crohn's disease, by administering a therapeutically effective amount of adalimumab comprising at least one MRD to a patient in need thereof. In another embodiment, the invention provides a method of treating ulcerative colitis, by administering a therapeutically effective amount of adalimumab comprising at least one MRD to a patient in need thereof. In another embodiment, the invention provides a method of treating psoriatic arthritis, ankylosing spondylitis, psoriasis, or juvenile idiopathic arthritis by administering a therapeutically effective amount of adalimumab comprising at least one MRD to a patient in need thereof.

In a specific embodiment, the invention provides a method of treating a disorder of the immune system comprising administering a therapeutically effective amount of infliximab comprising at least one MRD to a patient in need thereof. In one embodiment, the invention provides a method of treating an inflammatory disorder, by administering a therapeutically effective amount of infliximab comprising at least one MRD to a patient in need thereof. In one embodiment, the invention provides a method of treating an autoimmune disease, by administering a therapeutically effective amount of infliximab comprising at least one MRD to a patient in need thereof. In one embodiment, the invention provides a method of treating rheumatoid arthritis, by administering a therapeutically effective amount of infliximab comprising at least one MRD to a patient in need thereof. In another embodiment, the invention provides a method of treating Crohn's disease, by administering a therapeutically effective amount of infliximab comprising at least one MRD to a patient in need thereof. In another embodiment, the invention provides a method of treating ulcerative colitis, by administering a therapeutically effective amount of infliximab comprising at least one MRD to a patient in need thereof. In another embodiment, the invention provides a method of treating psoriatic arthritis, ankylosing spondylitis, psoriasis, or juvenile idiopathic arthritis by administering a therapeutically effective amount of infliximab comprising at least one MRD to a patient in need thereof.

In another embodiment, the invention provides a method of treating cancer comprising administering a therapeutically effective amount of a EGFR-binding MMM complex (e.g., ELP-MRD fusion protein) to a patient in need thereof. In a specific embodiment, the invention provides a method of treating cancer comprising administering a therapeutically effective amount of cetuximab comprising at least one MRD to a patient in need thereof. In one embodiment, the invention provides a method of treating cancer by administering a therapeutically effective amount of cetuximab comprising at least one MRD to a patient having colorectal cancer. In another embodiment, therapeutic effective amounts of cetuximab comprising at least one MRD are administered to a patient to treat metastatic colorectal cancer, metastatic breast cancer, metastatic pancreatic cancer, or metastatic non-small cell lung carcinoma. In one embodiment, the invention provides a method of treating cancer by administering a therapeutically effective amount of cetuximab comprising at least one MRD to a patient having squamous cell carcinoma of the head and neck.

In another embodiment, a therapeutically effective amount of an EGFR-binding MMM complex (e.g., ELP-MRD fusion protein) is administered in combination with irinotecan, FOLFIR1, platinum-based chemotherapy, or radiation therapy. In a specific embodiment, a therapeutically effective amount of cetuximab comprising at least one MRD is administered in combination with irinotecan, FOLFIRI, platinum-based chemotherapy, or radiation therapy

In some embodiments, the MMM complexes (e.g ELP-MRD fusion proteins) described herein are useful for treating cancer. Thus, in some embodiments, the invention provides methods of treating cancer comprise administering a therapeutically effective amount of an MMM complex (e.g., an ELP-MRD fusion protein) to a subject (e.g., a subject in need of treatment). In certain embodiments, the cancer is a cancer selected from the group consisting of colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, and head and neck cancer. In certain embodiments, the cancer is breast cancer. In certain embodiments, the subject is a human.

Other examples of cancers or malignancies that can be treated with MMM complexes (e.g., ELP-MRD fusion proteins) and MRDs include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, and Wilms' Tumor.

In some embodiments, MMM complexes (e.g., ELP-MRD fusion proteins) are useful for inhibiting tumor growth. In certain embodiments, the method of inhibiting the tumor growth comprises contacting the cell with an MMM complex (e.g., an ELP-MRD fusion protein) in vitro. For example, an immortalized cell line or a cancer cell line that expresses an ELP-MRD fusion and/or MRD target is cultured in medium to which is added the MMM complex (e.g., ELP-MRD fusion protein) to inhibit tumor growth. In some embodiments, tumor cells are isolated from a patient sample such as, for example, a tissue biopsy, pleural effusion, or blood sample and cultured in medium to which is added an MMM complex (e.g., an ELP-MRD fusion protein) to inhibit tumor growth.

In some embodiments, the method of inhibiting tumor growth comprises contacting the tumor or tumor cells with a therapeutically effective amount of the MMM complex (e.g., ELP-MRD fusion protein) in vivo. In certain embodiments, contacting a tumor or tumor cell is undertaken in an animal model. For example, MMM complexes (e.g., ELP-MRD fusion proteins) can be administered to xenografts in immunocompromised mice (e.g., NOD/SCID mice) to inhibit tumor growth. In some embodiments, cancer stem cells are isolated from a patient sample such as, for example, a tissue biopsy, pleural effusion, or blood sample and injected into immunocompromised mice that are then administered an MMM complex (e.g., an ELP-MRD fusion protein) to inhibit tumor cell growth. In some embodiments, the MMM complex (e.g., ELP-MRD fusion protein) is administered at the same time or shortly after introduction of tumorigenic cells into the animal to prevent tumor growth. In some embodiments, the MMM complex (e.g., ELP-MRD fusion protein) is administered as a therapeutic after the tumorigenic cells have grown to a specified size.

In certain embodiments, the method of inhibiting tumor growth comprises administering to a subject a therapeutically effective amount of an MMM complex (e.g., an ELP-MRD fusion protein). In certain embodiments, the subject is a human. In certain embodiments, the subject has a tumor or has had a tumor removed. In certain embodiments, the tumor expresses an ELP-MRD and/or MRD target. In certain embodiments, the tumor overexpresses an MRD target and/or ELP-MRD target.

In certain embodiments, the inhibited tumor growth is a member selected from the group consisting of: brain tumor, colorectal tumor, pancreatic tumor, lung tumor, ovarian tumor, liver tumor, breast tumor, kidney tumor, prostate tumor, gastrointestinal tumor, melanoma, cervical tumor, bladder tumor, glioblastoma, and head and neck tumor. In certain embodiments, the tumor is a breast tumor.

In additional embodiments, MMM complexes (e.g., ELP-MRD fusion proteins) are useful for reducing tumorigenicity. Thus, in some embodiments, the method of reducing the tumorigenicity of a tumor in a subject, comprises administering a therapeutically effective amount of an MMM complex (e.g., an ELP-MRD fusion protein) to the subject. In certain embodiments, the tumor comprises cancer stem cells. In certain embodiments, the frequency of cancer stem cells in the tumor is reduced by administration of the agent.

In other embodiments, MMM complexes (e.g., ELP-MRD fusion proteins) are useful for diagnosing, treating or preventing a disorder of the immune system. In one embodiment, the disorder of the immune system is inflammation or a inflammatory disorder. In a more specific embodiment, the inflammatory disorder is a member selected from the group consisting of: asthma, allergic disorders, and rheumatoid arthritis.

In another embodiment, the disorder of the immune system is an autoimmune disease. Autoimmune disorders, diseases, or conditions that can be diagnosed, treated or prevented using MMM complexes (e.g., ELP-MRD fusion proteins) include, but are not limited to, autoimmune hemolytic anemia, autoimmune neonatal thrombocytopenia, idiopathic thrombocytopenia purpura, autoimmune neutiopenia, autoimmunocytopenia, hemolytic anemia, antiphospholipid syndrome, dermatitis, gluten-sensitive enteropathy, allergic encephalomyelitis, myocarditis, relapsing polychondritis, rheumatic heart disease, glomerulonephritis (e.g., IgA nephropathy), Multiple Sclerosis, Neuritis, Uveitis Ophthalmia, Polyendocrinopathies, Purpura (e.g., Henloch-Scoenlein purpura), Reiter's Disease, Stiff-Man Syndrome, Autoimmune Pulmonary Inflammation, myocarditis, IgA glomerulonephritis, dense deposit disease, rheumatic heart disease, Guillain-Barre Syndrome, insulin dependent diabetes mellitis, and autoimmune inflammatory eye, autoimmune thyroiditis, hypothyroidism (i.e., Hashimoto's thyroiditis, systemic lupus erhythematosus, discoid lupus, Goodpasture's syndrome, Pemphigus, Receptor autoimmunities such as, for example, (a) Graves' Disease, (b) Myasthenia Gravis, and (c) insulin resistance, autoimmune hemolytic anemia, autoimmune thrombocytopenic purpura, rheumatoid arthritis, schleroderma with anti-collagen antibodies, mixed connective tissue disease, polymyositis/dermatomyositis, pernicious anemia, idiopathic Addison's disease, infertility, glomerulonephriis such as primary glomerulonephriis and IgA nephropathy, bullous pemphigoid, Sjogren's syndrome, diabetes mellitus, and adrenergic drug resistance (including adrenergic drug resistance with asthma or cystic fibrosis), chronic active hepatitis, primary biliary cirrhosis, other endocrine gland failure, vitiligo, vasculitis, post-MI, cardiotomy syndrome, urticaria, atopic dermatitis, asthma, inflammatory myopathies, and other inflammatory, granulomatous, degenerative, and atrophic disorders.

In a further embodiment the disorder of the immune system diagnosed, treated or prevented using MMM complex es (e.g., ELP-MRD fusion proteins) is a member selected from: the group consisting of: Crohn's disease, Systemic lupus erythematosus (SLE), inflammatory bowel disease, psoriasis, diabetes, ulcerative colitis, multiple sclerosis, and rheumatoid arthritis. In a preferred embodiment, the autoimmune disease is rheumatoid arthritis.

In other embodiments, a therapeutically effective amount of an MMM complex (e.g., an ELP-MRD fusion protein) is administered to a patient to treat a metabolic disease or disorder.

In an additional embodiments, a therapeutically effective amount of an MMM complex (e.g., an ELP-MRD fusion protein) is administered to a patient to treat a cardiovascular disease or disorder. In one embodiment, the MMM complexes (e.g., ELP-MRD fusion proteins) is administered to a patient to to treat or prevent thrombosis, atheroschlerosis, heart attack, or stroke.

In another embodiment, a therapeutically effective amount of an MMM complex (e.g., an ELP-MRD fusion protein) is administered to a patient to treat a musculoskeletal disease or disorder.

In further embodiments, a therapeutically effective amount of an MMM complex (e.g., an ELP-MRD fusion protein) is administered to a patient to treat a skeletal disease or disorder. In one embodiment, aMMM complex (e.g., ELP-MRD fusion protein) is administered to a patient to treat osteoporosis.

In additional embodiments, the MMM complex binds (1) a target on a cell or tissue of interest (e.g., a tumor antigen on a tumor cell) and (2) a target on a leukocyte, such as a T-cell receptor molecule. According to one embodiment, the binding of one or more targets by the MMM complex is used to direct an immune response to an infectious agent, cell, tissue, or other location of interest in a patient. For example, in some embodiments, embodiments, an MRD of the MMM complex binds a target on the surface of an effector cell. Thus, in some embodiments, an MRD of the MMM complex binds a target on the surface of a T cell. In specific embodiments, embodiments, an MRD of the MMM complex binds CD3. In other embodiments, an MRD of the MMM complex binds CD2. In further embodiments, an MRD of the MMM complex binds one or more of the components of the T-cell receptor (TCR) complex. According to additional embodiments, an MRD of the MMM complex binds a target on the surface of a Natural Killer Cell. Thus, in some embodiments, an MRD of the MMM complex binds a NKG2D (Natural Killer Group 2D) receptor. In additional embodiments, an MRD of the MMM complex binds CD16 (i.e., Fc gamma RIII) CD64 (i.e., Fc gamma RI), or CD32 (i.e., Fc gamma RII). In additional embodiments, the mutispecific composition contains more than one monospecific binding site for different targets.

In further embodiments, the MMM complex has a single binding site (i.e., is monospecific) for a target. In some embodiments, the MMM complex has a single binding site (i.e., is monospecific) for a target on a leukocyte, such as a T-cell (e.g., CD3) and binds a target on a cell or tissue of interest (e.g., a tumor antigen on a tumor cell, such as a target disclosed herein).

In further embodiments, the invention is directed to treating a disease or disorder by administering a therapeutically effective amount of an MMM complex that has a single binding site (i.e., is monospecific) for a target. In some embodiments, the administered MMM complex has a single binding site (i.e., is monospecific) for a target on a leukocyte, such as a T-cell (e.g., CD3) and binds a target on a cell or tissue of interest (e.g., a tumor associated antigen on a tumor cell). In some embodiments, the tumor cell is from a cancer selected from breast cancer, colorectal cancer, endometrial cancer, kidney (renal cell) cancer, lung cancer, melanoma, Non-Hodgkin Lymphoma, leukemia, prostate cancer, bladder cancer, pancreatic cancer, and thyroid cancer. In additional embodiments, the MMM complex has multiple binding sites for a target on a neurological tumor. In particular embodiments, the neurological tumor is a glioma (e.g., a glioblastoma, glioblastoma multiforme (GBM), and astrocytoma), ependymoma, oligodendroglioma, neurofibroma, sarcoma, medulloblastoma, primitive neuroectodermal tumor, pituitary adenoma, neuroblastoma or cancer of the meninges (e.g., meningioma, meningiosarcoma and gliomatosis).

Additional emodiments are directed to administering a therapeutically effective amount of an MMM complex to treat a neurological disease or disorder selected from brain cancer, a neurodegenerative disease, schizophrenia, epilepsy, Alzheimer's disease, Parkinson's disease, Huntington's disease, ALS, multiple sclerosis, Neuromyelitis optica and Neuro-AIDS (e.g., HIV-associated dementia). In another embodiment, the MMM complex is administered to a patient to treat a brain cancer, metastatic cancer of the brain, or primary cancer of the brain. In a further embodiment, the MMM complex is administered to a patient to treat brain injury, stroke, spinal cord injury, or pain management. In further embodiments, the MMM complex is administered to a patient to treat brain injury, stroke, or spinal cord injury, or for pain management.

In one embodiment, a therapeutically effect amount of the MMM complex is administered to a patient to treat an infection or a symptom associated with an infection caused by an infectious agent. In some embodiments, the infection is caused by a member selected from apovavirus (e.g., JC polyomavirus), trypanosomes, West Nile virus, HIV, Streptococcus pneumoniae and Haemophilus influenzae, bovine spongiform encephalopathy, meningitis, Progressive multifocal leukoencephalopathy (PML), Late-stage neurological trypanosomiasis, Encephalitis, and rabies.

According to some embodiments, the MMM complex (e.g., ELP-MRD fusion protein) is able to cross the blood brain barrier (BBB) and bind a target located on the brain side of the BBB. In additional embodiments, the MMM complex has a single binding site that binds a target (e.g., ligand, receptor, or accessory protein) associated with an endogenous BBB receptor mediated transport system. In some embodiments, a single binding site of the composition is an MRD. In other embodiments, a single binding site of the composition is an antibody antigen binding domain. In some embodiments, the MMM complex contains 1, 2, 3, 4, 5, or more single binding sites (i.e., monovalently binds) for a target associated with an endogenous BBB receptor mediated transport system and the composition is able to cross to the cerebrospinal fluid side of the BBB. In additional embodiments, the MMM complex contains 1, 2, 3, 4, 5, or more multiple binding sites (i.e., multivalently binds) for a target associated with an endogenous BBB receptor mediated transport system and the composition is able to cross to the cerebrospinal fluid side of the BBB. In additional embodiments, a therapeutically effective amount of an MMM complex (e.g., an ELP-MRD fusion protein) is administered to a patient to treat a neurological disease or disorder selected from brain cancer, a neurodegenerative disease, schizophrenia, epilepsy, Alzheimer's disease, Parkinson's disease, Huntington's disease, ALS, multiple sclerosis, Neuromyelitis optica and Neuro-AIDS (e.g., HIV-associated dementia). In some embodiments, the MMM complex has a single binding site (i.e., is monovalent for binding a particular target (antigen))) or two or more binding sites (i.e., is monovalent for binding a particular target) for a target selected from alpha-synuclein, RGM A, NOGO A, NgR, OMGp MAG, CSPG, neurite inhibiting semaphorins (e.g., Semaphorin 3A and Semaphorin 4) an ephrin, A-beta, AGE (S100 A, amphoterin), NGF, soluble A-B, aggrecan, midkine, neurocan, versican, phosphacan, Te38, and PGE2. In some embodiments, the MMM complex additionally has a single binding site or multiple binding sites for a target selected from IL-1, IL-1R, IL-6, IL6R, IL-12, IL-18, IL-23, TWEAK, CD40, CD40L, CD45RB, CD52, CD200, VEGF, VLA-4, TNF alpha, Interferon gamma, GMCSF, FGF, C5, CXCL13, CCR2, CB2, MIP 1a and MCP-1.

In additional embodiments, the MMM complex is capable of transferring to the cerebrospinal fluid side of the BBB and is administered to a patient to treat a neurological disease or disorder selected from: brain cancer, a neurodegenerative disease, schizophrenia, epilepsy, Alzheimer's disease, Parkinson's disease, Huntington's disease, ALS, multiple sclerosis, Neuromyelitis optica and Neuro-AIDS (e.g., HIV-associated dementia). In further embodiments, the invention is directed to treating a disease or disorder by administering an MMM complex (e.g., an ELP-MRD fusion protein) that has a single binding site (i.e., is monospecific) for a target to a patient in need thereof. In some embodiments, the administered MMM complex (e.g., ELP-MRD fusion protein) has a single binding site (i.e., is monospecific) for a target on a leukocyte, such as a T-cell (e.g., CD3) and binds a target on a cell or tissue of interest (e.g., a tumor associated antigen on a tumor cell).

In some embodiments, the MMM complex is administered to a patient to treat a neurological disease or disorder selected from brain cancer, a neurodegenerative disease, schizophrenia, epilepsy, Alzheimer's disease, Parkinson's disease, Huntington's disease, ALS, multiple sclerosis, Neuromyelitis optica and Neuro-AIDS (e.g., HIV-associated dementia). In additional embodiments, the MMM complex is administered to a patient to treat a brain cancer, metastatic cancer of the brain, or primary cancer of the brain. In additional embodiments, the MMM complex is administered to a patient to treat a neurological tumor. In particular embodiments, the neurological tumor is a selected from: glioma (e.g., a glioblastoma, glioblastoma multiforme (GBM), and astrocytoma), ependymoma, oligodendroglioma, neurofibroma, sarcoma, medulloblastoma, primitive neuroectodermal tumor, pituitary adenoma, neuroblastoma or cancer of the meninges (e.g., meningioma, meningiosarcoma and gliomatosis).

In additional embodiments, the MMM complex is administered to a patient to treat brain injury, stroke, spinal cord injury, or pain. Thus, according to some embodiments, the disease, disorder, or injury treated or prevented with an MMM complex (e.g., an ELP-MRD fusion protein) or MRD of the invention is neurological. In one embodiment, the neurological disease, disorder or injury is associated with pain such as, acute pain or chronic pain.

In some embodiments the MMM complex binds 1, 2, 3, 4 or 5 targets associated with a neurological disease or disorder. In one embodiment, the MMM complex (e.g., ELP-MRD fusion protein) binds 1, 2, or all 3 of the targets RGM A; NgR, and NogoA. In another embodiment, the MMM complex binds 1, 2, 3, or all 4 of RGM A, RGM B, and Semaphorin 3A or Semaphorin 4. In a further embodiment, the MMM complex binds 1, 2, 3, 4 or 5 targets selected from aggrecan, midkine, neurocan, versican, phosphacan, Te38, TNF alpha, NogoA, RGM A, MAG, and OMGp. In another embodiment, the MMM complex binds 1, 2, 3, 4 or 5 targets selected from aggrecan, midkine, neurocan, versican, phosphacan, Te38 and TNF alpha. In an alternative embodiment, the MMM complex binds 1, 2, 3, 4 or 5 targets selected from NgR-p75, NgR-Troy, NgR-Nogo66 (Nogo), NgR-Lingo, Lingo-Troy, Lingo-p75, MAG and Omgp. In another embodiment, the MMM complex binds 1, 2, 3, 4 or 5 targets selected from NGF, PGE2, TNF-alpha, IL-1 beta, and IL-6R.

In an additional embodiment, the MMM complex binds 1, 2, 3, 4 or 5 targets selected from alpha-synuclein, RGM A and one or more pro-inflammatory mediators (e.g., TNF alpha, IL-1, and MCP-1). Such compositions have applications in, for example, treating neurodegenerative diseases such as, Parkinson's.

In another embodiment, the MMM complex binds and antagonizes (i.e., is an antagonist of) 1, 2, 3, 4 or 5 targets selected from RGM A, NOGO A, neurite inhibiting semaphorins (e.g., Semaphorin 3A and Semaphorin 4) and ephrins, and pro-inflammatory targets (e.g., IL-12, TWEAK, IL-23, CXCL13, CD40, CD40L, IL-18, VEGF, VLA-4, TNF alpha, CD45RB, CD200, Interferon gamma, GMCSF, FGF, C5, CD52, and CCR2). The complexes have applications in treating for example, inflammation, neuroregeneration and neurodegenerative disorders, such as MS. In another embodiment, the MMM complex binds and antagonizes (i.e., is an antagonist of) 1, 2, 3, 4 or 5 targets selected from AGE (S100 A, amphoterin), pro-inflammatory cytokines (e.g., IL-1, IL-6, and TNF), chemokines (e.g., MCP 1), and molecules that inhibit neural regeneration (e.g., Nogo and RGM A). These compositiosn have applications in treating, for example, chronic neurodegenerative diseases such as, Alzheimer's. In an additional embodiment, the complex of the invention binds 1, 2, 3, 4 or 5 targets that influence neural generation and survival including, for example, NGF agonists, IL1 or IL1R antagonists, and A-beta. These complexes have applications in treating, for example, chronic neurodegenerative diseases such as, Alzheimer's. In an additional embodiment, the complex of the invention binds to and antagonizes 1, 2, 3, 4, or 5 targets that targets that interfere with neural regeneration or recovery, including NogoA, OMgp MAG, RGM A, CSPG, one or more astrocyte inhibiting semaphorins (e.g., Semaphorin 3A and Semaphorin 4), ephrins, and pro-inflammatory cytokines (e.g., IL-1, IL-6, and TNF). These complexes have applications in treating neurodegenerative diseases and neural injury or trauma.

In additional embodiment, the MMM complex binds and antagonize (i.e., is an antagonist of) 1, 2, 3, 4, or 5 targets associated with pain, including, but not limted to, NGF and SCN9A/NAV1.7. Such complexes have applications in for example, treating or alleviating pain and pain associated conditions.

In additional embodiments, the targets bound by the complex of the invention binds and antagonizes 1, 2, 3, 4, 5 or more mediators and or soluble or cell surface targets implicated in the inhibition of neurite growth or recovery. In specific embodiments, compostions of the invention bind to and antagonizes 1, 2, 3, 4, 5 or more targets selected from Nogo, Ompg, MAG, RGM A, semaphorins, ephrins, soluble A-b, pro-inflammatory cytokines (e.g., IL-1 and TNF alpha), chemokines (e.g., MIP 1a).

In other embodiments, MMM complexes (e.g., ELP-MRD fusion proteins) are useful for treating or preventing an infectious disease. Infectious diseases that can be treated or prevented with MMM complexes (e.g., ELP-MRD fusion proteins) include, but are not limited to, diseases associated with yeast, fungal, viral and bacterial infections. Viruses causing viral infections which can be treated or prevented with MMM complexes (e.g., ELP-MRD fusion proteins) include, but are not limited to, retroviruses (e.g., human T-cell lymphotrophic virus (HTLV) types I and II and human immunodeficiency virus (HIV)), herpes viruses (e.g., herpes simplex virus (HSV) types I and II, Epstein-Barr virus, HHV6-HHV8, and cytomegalovirus), adrenoviruses (e.g., lassa fever virus), paramyxoviruses (e.g., morbilbivirus virus, human respiratory syncytial virus, mumps, and pneumovirus), adrenoviruses, bunyaviruses (e.g., hantavirus), cornaviruses, filoviruses (e.g., Ebola virus), flaviviruses (e.g., hepatitis C virus (HCV), yellow fever virus, and Japanese encephalitis virus), hepadnaviruses (e.g., hepatitis B viruses (HBV)), orthomyoviruses (e.g., influenza viruses A, B and C (including avian influenza, e.g., H5N1 subtype)), papovaviruses (e.g., papillomaviruses), picornaviruses (e.g., rhinoviruses, enteroviruses and hepatitis A viruses), poxviruses, reoviruses (e.g., rotaviruses), togaviruses (e.g., rubella virus), rhabdoviruses (e.g., rabies virus). Microbial pathogens causing bacterial infections include, but are not limited to, Streptococcus pyogenes, Streptococcus pneumoniae, Neisseria gonorrhoea, Neissetia meningitidis, Corynebacterium diphtheriae, Clostridium botulinum, Clostridium pefringens, Clostridium tetani, Haemophilus influenzae, Klebsiella pneumoniae, Klebsiella ozaenae, Klebsiella rhinoscleromotis, Staphylococcus aureus, Vibrio cholerae, Escherichia coli, Pseudomonas aeruginosa, Campylobacter (Vibrio) fetus, Campylobacter jejuni, Aeromonas hydrophila, Bacillus cereus, Edwardsiella tarda, Yersinia enterocolitica, Yersinia pestis, Yersinia pseudotuberculosis, Shigella dysenteriae, Shigella flexneri, Shigella sonnei, Salmonella typhimurium, Treponema pallidum, Treponema pertenue, Treponema carateneum, Borrelia vincentii, Borrelia burgdorferi, Leptospira icterohemorrhagiae, Mycobacterium tuberculosis, Toxoplasma gondii, Pneumocystis carinii, Francisella tularensis, Brucella abortus, Brucella suis, Brucella melitensis, Mycoplasma spp., Rickettsia prowazeki, Rickettsia Lsutsugumushi, Chlamydia spp., and Helicobacter pylori.

In one embodiment, the MMM complexes (e.g., ELP-MRD fusion proteins) are administered to treat or prevent human immunodeficiency virus (HIV) infection or AIDS, botulism, anthrax, or clostridium difficile.

In one embodiment the complex comprises an elastin-like peptide-modular recognition domain (ELP-MRD) fusion protein which comprises at least one elastin-like peptide (ELP) and at least one modular recognition domain (MRD) that binds a soluble ligand.

In an additional embodiment, the complex comprises an ELP-MRD fusion protein comprising at least 2, at least 3, at least 4, or at least 5 ELPs. In an additional embodiment, the complex comprises an ELP-MRD fusion protein comprising an ELP having repeat units containing the sequence (VPGXG)n (SEQ ID NO:119), where X is a natural or non-natural amino acid residue and optionally varies among repeats units, and where n is a number from 1 to 50. In an additional embodiment, n is a number from 1 to 18. In an additional embodiment, X is an amino acid residue selected from A, R, N, D, C, E, Q, G, H, I, L, K, M, F, S, T, W, Y and V.

In an additional embodiment, the MRDs are homomultimeric. In an additional embodiment, MRDs are heteromultimeric.

In an additional embodiment, the ELP-MRD fusion protein comprises at least 2, at least 3, at least 4, or at least 5 MRDs. In an additional embodiment, the ELP-MRD fusion protein comprises at least 2, at least 3, at least 4, or at least 5 MRDs that bind the same target. In an additional embodiment, the ELP-MRD fusion protein comprises at least 2, at least 3, at least 4, or at least 5 MRDs that bind different epitopes on the same target. In an additional embodiment, the ELP-MRD fusion protein comprises at least 2, at least 3, at least 4, or at least 5 MRDs that bind different targets. In an additional embodiment, the ELP-MRD fusion protein comprises at least 2, at least 3, at least 4, or at least 5 MRDs that bind a soluble ligand.

In an additional embodiment, the ELP-MRD fusion protein is capable of binding at least 2, at least 3, at least 4, or at least 5 targets. In an additional embodiment, the ELP-MRD fusion protein is capable of binding at least 2, at least 3, at least 4, or at least 5 targets simultaneously.

In an additional embodiment, the ELP-MRD fusion protein is capable of binding at least 2, at least 3, at least 4, or at least 5 molecules of the same target. In an additional embodiment, the ELP-MRD fusion protein is capable of binding at least 2, at least 3, at least 4, or at least 5 molecules of the same target simultaneously.

In an additional embodiment, the ELP-MRD fusion protein is capable of binding at least 2, at least 3, at least 4, or at least 5 different epitopes of the same target. In an additional embodiment, the ELP-MRD fusion protein is capable of binding at least 2, at least 3, at least 4, or at least 5 different epitopes of the same target simultaneously.

In an additional embodiment, the ELP-MRD fusion protein is capable of binding at least 2, at least 3, at least 4, or at least 5 different targets. In an additional embodiment, the ELP-MRD fusion protein is capable of binding at least 2, at least 3, at least 4, or at least 5 different targets simultaneously.

In an additional embodiment, the ELP-MRD fusion protein is an antagonist of at least 2, at least 3, at least 4, or at least 5 different targets. In an additional embodiment, the ELP-MRD fusion protein is an agonist of at least 2, at least 3, at least 4, or at least 5 different targets.

In an additional embodiment, the ELP-MRD fusion protein binds a membrane associated target. In an additional embodiment, the ELP-MRD fusion protein further comprises at least 1, at least 2, at least 3, at least 4, or at least 5 MRDs that bind a membrane associated target.

In an additional embodiment, the ELP-MRD fusion protein binds a cytokine or a chemokine. In an additional embodiment, the ELP-MRD fusion protein is capable of binding at least 2, at least 3, at least 4, or at least 5 cytokines or chemokines. In an additional embodiment, the ELP-MRD fusion protein is capable of binding at least 2, at least 3, at least 4, or at least 5 cytokines or chemokines simultaneously.

In an additional embodiment, the ELP-MRD fusion protein is capable of binding at least 2, at least 3, at least 4, or at least 5 molecules of the same cytokine or chemokine. In an additional embodiment, the ELP-MRD fusion protein is capable of binding at least 2, at least 3, at least 4, or at least 5 molecules of the same cytokine or chemokine simultaneously.

In an additional embodiment, the ELP-MRD fusion protein is capable of binding at least 2, at least 3, at least 4, or at least 5 different epitopes of a cytokine or chemokine. In an additional embodiment, the ELP-MRD fusion protein is capable of binding at least 2, at least 3, at least 4, or at least 5 different epitopes of a cytokine or chemokine simultaneously.

In an additional embodiment, the ELP-MRD fusion protein is capable of binding at least 2, at least 3, at least 4, or at least 5 different cytokines or chemokines. In an additional embodiment, the ELP-MRD fusion protein is capable of binding at least 2, at least 3, at least 4, or at least 5 different cytokines or chemokines simultaneously.

In an additional embodiment, the ELP-MRD fusion protein binds VEGF. In an additional embodiment, the ELP-MRD fusion protein binds ANG2. In an additional embodiment, the ELP-MRD fusion protein binds TNF. In an additional embodiment, the ELP-MRD fusion protein binds RANKL. In an additional embodiment, the ELP-MRD fusion protein binds BLyS. In an additional embodiment, the ELP-MRD fusion protein binds TL1a. In an additional embodiment, the ELP-MRD fusion protein binds LIGHT. In an additional embodiment, the ELP-MRD fusion protein binds APRIL. In an additional embodiment, the ELP-MRD fusion protein binds IL-1. In an additional embodiment, the ELP-MRD fusion protein binds IL-1 beta. In an additional embodiment, the ELP-MRD fusion protein binds IL-6. In an additional embodiment, the ELP-MRD fusion protein binds IL-10. In an additional embodiment, the ELP-MRD fusion protein binds IL-17. In an additional embodiment, the ELP-MRD fusion protein binds IGF. In an additional embodiment, the ELP-MRD fusion protein binds NGF. In an additional embodiment, the ELP-MRD fusion protein binds CCL19. In an additional embodiment, the ELP-MRD fusion protein binds CCL21. In an additional embodiment, the ELP-MRD fusion protein binds interferon alpha.

In an additional embodiment, the ELP-MRD fusion protein binds a target selected from: TNFSF7 (CD27 Ligand, CD70), TNFSF12 (TWEAK), TNFSF4 (OX40 Ligand), TNFRSF4 (OX40), TNFSF5 (CD40 Ligand), IL-5, IL-9, IL-12, IL-13, IL-14, IL-15, IL-20, IL-21, IL-23, IL-31, Tsu), interferon alpha, interferon gamma, B7RP-1, GMCSF, CSF, CCL2, CCL17, CXCL8, CXCL10, P1GF, PD1, alpha4 C5, RhD, IgE, and Rh.

In an additional embodiment, the ELP-MRD fusion protein binds a target selected from: amyloid beta (Abeta), beta amyloid, complement factor D, PLP, GDNF, NGF, myostatin, oxidized LDL, PCSK9, Factor VIII, or mesothelin, DKK1, osteopontin, cathepsin K, and sclerostin,

In an additional embodiment, the ELP-MRD fusion protein binds a target selected from: TGFbeta 1, phosphatidlyserine, HGF, CRIPTO, TNFSF9 (41BB Ligand), TNFSF4 (OX40 Ligand) EGFL7, beta (Abeta), and complement factor D.

In an additional embodiment, the ELP-MRD fusion protein binds a serum protein.

In an additional embodiment, the ELP-MRD fusion protein comprises at least 2, at least 3, at least 4, or at least 5 MRDs that bind a serum protein. In an additional embodiment, the ELP-MRD fusion protein binds a serum protein selected from: serum albumin, thyroxin-binding protein, transferrin, fibrinogen, and an immunoglobulin.

In an additional embodiment, the ELP-MRD fusion protein binds human serum albumin. In an additional embodiment, the ELP-MRD fusion protein comprises an MRD that binds human serum albumin.

In an additional embodiment, the ELP-MRD fusion protein further comprises an antibody fragment or domain.

In an additional embodiment, the ELP-MRD fusion protein binds a human protein. In an additional embodiment, the ELP-MRD fusion protein binds a nonhuman protein.

In an additional embodiment, the ELP-MRD fusion protein binds a pathogenic antigen. In an additional embodiment, the ELP-MRD fusion protein is capable of binding a member selected from: a bacterial antigen, a viral antigen, a mycoplasm antigen, a prion antigen, or a parasite antigen. In an additional embodiment, the ELP-MRD fusion protein comprises at least 1, at least 2, at least 3, at least 4, or at least 5 MRDs capable of binding a member selected from: a bacterial antigen, a viral antigen, a mycoplasm antigen, a prion antigen, or a parasite antigen.

In an additional embodiment, the ELP-MRD fusion protein comprises an ELP and

MRD or other component of the complex operably linked through a linker peptide. In an additional embodiment, the linker comprises a sequence selected from the group consisting of: SEQ ID NO:1, SEQ ID NO:2, and SEQ ID NO:19. In an additional embodiment, the ELP-MRD fusion protein comprises at least 2, at least 3, at least 4, or at least 5 MRDs operably linked to within the fusion protein through a linker peptide. In an additional embodiment, the ELP-MRD fusion protein comprises an ELP and MRD or other component of the complex operably attached without a linker. In an additional embodiment, the ELP-MRD fusion protein comprises at least two ELPs and MRDs or other components of the complex operably attached without a linker.

In an additional embodiment, the invention encompasses a polynucleotide encoding the ELP-MRD fusion proteins of the invention. In an additional embodiment, the invention encompasses a vector comprising the polynucleotide encoding the ELP-MRD fusion proteins of the invention. In an additional embodiment, the invention encompasses a host cell comprising the vector comprising the polynucleotide encoding the ELP-MRD fusion proteins of the invention.

In an additional embodiment, the complex comprises an elastin-like peptide-modular recognition domain (ELP-MRD) fusion protein which comprises at least one elastin-like peptide (ELP) and at least one modular recognition domain (MRD) that binds a membrane associated target. In an additional embodiment, the ELP-MRD fusion protein comprises a continuous amino acid sequence containing at least 2, at least 3, at least 4, or at least 5 MRDs that bind a membrane associated target.

In an additional embodiment, the ELP-MRD fusion protein comprises at least 2, at least 3, at least 4, or at least 5 ELPs. In an additional embodiment, the ELP-MRD fusion protein comprises an ELP having repeat units containing the sequence (VPGXG)n (SEQ ID NO:119), where X is a natural or non-natural amino acid residue and optionally varies among repeats units, and wherein n is a number from 1 to 50. In an additional embodiment, n is a number from 1 to 18. In an additional embodiment, X is an amino acid residue selected from A, R, N, D, C, E, Q, G, H, I, L, K, M, F, S, T, W, Y and V.

In an additional embodiment, the MRDs are homomultimeric. In an additional embodiment, the MRDs are heteromultimeric.

In an additional embodiment, the ELP-MRD fusion protein comprises at least 2, at least 3, at least 4, or at least 5 MRDs. In an additional embodiment, the ELP-MRD fusion protein comprises at least 2, at least 3, at least 4, or at least 5 MRDs that bind the same target. In an additional embodiment, the ELP-MRD fusion protein comprises at least 2, at least 3, at least 4, or at least 5 MRDs that bind different epitopes on the same target. In an additional embodiment, the ELP-MRD fusion protein comprises at least 2, at least 3, at least 4, or at least 5 MRDs that bind different targets.

In an additional embodiment, the ELP-MRD fusion protein comprises at least 2, at least 3, at least 4, or at least 5 MRDs that bind a soluble ligand.

In an additional embodiment, the ELP-MRD fusion protein is capable of binding at least 2, at least 3, at least 4, or at least 5 targets. In an additional embodiment, the ELP-MRD fusion protein is capable of binding at least 2, at least 3, at least 4, or at least 5 targets simultaneously.

In an additional embodiment, the ELP-MRD fusion protein is capable of binding at least 2, at least 3, at least 4, or at least 5 molecules of a target. In an additional embodiment, the ELP-MRD fusion protein is capable of binding at least 2, at least 3, at least 4, or at least 5 molecules of the same target simultaneously.

In an additional embodiment, the ELP-MRD fusion protein is capable of binding at least 2, at least 3, at least 4, or at least 5 different epitopes of the same target. In an additional embodiment, the ELP-MRD fusion protein is capable of binding at least 2, at least 3, at least 4, or at least 5 different epitopes of the same target simultaneously.

In an additional embodiment, the ELP-MRD fusion protein is capable of binding at least 2, at least 3, at least 4, or at least 5 different targets. In an additional embodiment, the ELP-MRD fusion protein is capable of binding at least 2, at least 3, at least 4, or at least 5 different targets simultaneously.

In an additional embodiment, the ELP-MRD fusion protein is an antagonist of at least 2, at least 3, at least 4, or at least 5 different targets. In an additional embodiment, the ELP-MRD fusion protein is an agonist of at least 2, at least 3, at least 4, or at least 5 different targets.

In an additional embodiment, the ELP-MRD fusion protein further comprises at least 1, at least 2, at least 3, at least 4, or at least 5 MRDs that bind a soluble ligand. In an additional embodiment, the ELP-MRD fusion protein binds a soluble ligand.

In an additional embodiment, the ELP-MRD fusion protein binds a cancer antigen. In an additional embodiment, the ELP-MRD fusion protein is capable of binding at least 2, at least 3, at least 4, or at least 5 cancer antigens. In an additional embodiment, the ELP-MRD fusion protein is capable of binding at least 2, at least 3, at least 4, or at least 5 cancer antigens, simultaneously. In an additional embodiment, the ELP-MRD fusion protein is capable of binding at least 2, at least 3, at least 4, or at least 5 molecules of the same cancer antigen. In an additional embodiment, the ELP-MRD fusion protein is capable of binding at least 2, at least 3, at least 4, or at least 5 molecules of the same cancer antigen, simultaneously.

In an additional embodiment, the ELP-MRD fusion protein is capable of binding at least 2, at least 3, at least 4, or at least 5 different epitopes of the same cancer antigen. In an additional embodiment, the ELP-MRD fusion protein is capable of binding at least 2, at least 3, at least 4, or at least 5 different epitopes of the same cancer antigen, simultaneously.

In an additional embodiment, the ELP-MRD fusion protein is capable of binding at least 2, at least 3, at least 4, or at least 5 different cancer antigens. In an additional embodiment, the ELP-MRD fusion protein is capable of binding at least 2, at least 3, at least 4, or at least 5 different cell surface cancer antigens simultaneously.

In an additional embodiment, the ELP-MRD fusion protein is capable of binding an antigen associated with a disorder of the immune system. In an additional embodiment, the ELP-MRD fusion protein is capable of binding at least 2, at least 3, at least 4, or at least 5 antigens associated with a disorder of the immune system. In an additional embodiment, the ELP-MRD fusion protein is capable of binding at least 2, at least 3, at least 4, or at least 5 antigens associated with a disorder of the immune system, simultaneously. In an additional embodiment, the ELP-MRD fusion protein is capable of binding at least 2, at least 3, at least 4, or at least 5 molecules of the same antigen associated with a disorder of the immune system. In an additional embodiment, the ELP-MRD fusion protein is capable of binding at least 2, at least 3, at least 4, or at least 5 molecules of the same antigen associated with a disorder of the immune system, simultaneously. In an additional embodiment, the ELP-MRD fusion protein is capable of binding at least 2, at least 3, at least 4, or at least 5 different epitopes of the same antigen associated with a disorder of the immune system. In an additional embodiment, the ELP-MRD fusion protein is capable of binding at least 2, at least 3, at least 4, or at least 5 different antigens associated with a disorder of the immune system. In an additional embodiment, the ELP-MRD fusion protein is capable of binding at least 2, at least 3, at least 4, or at least 5 different antigens associated with a disorder of the immune system simultaneously.

In an additional embodiment, the ELP-MRD fusion protein binds EGFR. In an additional embodiment, the ELP-MRD fusion protein binds ErbB2. In an additional embodiment, the ELP-MRD fusion protein binds CD20. In an additional embodiment, the ELP-MRD fusion protein binds cMET. In an additional embodiment, the ELP-MRD fusion protein binds IGFR-1. In an additional embodiment, the ELP-MRD fusion protein binds CD19. In an additional embodiment, the ELP-MRD fusion protein binds IL6R. In an additional embodiment, the ELP-MRD fusion protein binds CCR5. In an additional embodiment, the ELP-MRD fusion protein binds CCR7. In an additional embodiment, the ELP-MRD fusion protein binds NAV1.7.

In an additional embodiment, the ELP-MRD fusion protein binds a target selected from: ErbB3, ErbB4, prostate specific membrane antigen, an integrin, VEGFR1, and VEGFR2. In an additional embodiment, the ELP-MRD fusion protein binds a target selected from: CD22, CD30, CD33, CD38, CD44v6, TNFSF5 (CD40 Ligand), TNFRSF5 (CD40), CD52, CD54 (ICAM), CD74, CD80, CD200, EPCAM (EGP2), neuropilin 1 (NRP1), TEM1, mesothelin, TGFbeta 1, TGFBR11, phosphatidlyserine, folate receptor alpha (FOLR1), TNFRSF10A (TRAIL R1DR4), TNFRSF10B (TRAIL R2DR5), CXCR4, CCR4, HGF, VLA5, TNFSF9 (41BB Ligand), TNFRSF9 (41BB, CD137), CTLA4, HLA-DR, TNFRSF4 (OX40), CanAg, ganglioside GD3, PDGFRa, CD117 (cKit), SLAMF7, carcinoembryonic antigen (CEA), FAP, MUC1, MUC18, mucin, LINGO, AOC3, ROBO, ROBO4, gpIIB, gpIIIa, integrin avb3, or integrin α5β3.

In an additional embodiment, the ELP-MRD fusion protein binds a target selected from: TNFRSF1A (TNFR1, p55, p60), TNFRSF1B (TNFR2), TNFRSF7 (CD27), TNFRSF13B (TACI), TNFRSF13C (BAFFR), TNFRSF17 (BCMA), TNFRSF25 (DR3), TNFRSF12 (TWEAKR), TNFRSF4 (OX40), TNFRSF5 (CD40), IL1Ra, IL-2R, IL4-Ra, IL-5R, IL-15R, IL-17R, 11-17Rb, IL-17RC, IL-22RA, IL-23R, TSLPR, 137R P-1, GMCSFR, CD2, CD3, CD4, CD11a, CD18, CD20, CD40, CD80, CD86, CXCR3, CCR2, CCR8, P1GF, PD1, B7-DC (PD1,2), B74-11 (PIM), alpha4 integrin subunit, A4B7 integrin, FcRn and FcGamma RIIB.

In an additional embodiment, the ELP-MRD fusion protein binds a serum protein. In an additional embodiment, the ELP-MRD fusion protein comprises at least 2, at least 3, at least 4, or at least 5 MRDs that bind a serum protein. In an additional embodiment, the ELP-MRD fusion protein binds a serum protein selected from: serum albumin, thyroxin-binding protein, transferrin, fibrinogen, and an immunoglobulin. In an additional embodiment, the ELP-MRD fusion protein binds human serum albumin. In an additional embodiment, the ELP-MRD fusion protein comprises an MRD that binds human serum albumin.

In an additional embodiment, the ELP-MRD fusion protein further comprises an antibody fragment or domain. In an additional embodiment, the ELP-MRD fusion protein binds a human protein. In an additional embodiment, the ELP-MRD fusion protein binds a nonhuman protein.

In an additional embodiment, the ELP-MRD fusion protein binds a pathogen. In an additional embodiment, the ELP-MRD fusion protein is capable of binding a member selected from: a bacteria, a virus, a mycoplasm, a prion, or a parasite. In an additional embodiment, the ELP-MRD fusion protein comprises at least 1, at least 2, at least 3, at least 4, or at least 5 MRDs capable of binding a member selected from: a bacteria, a virus, a mycoplasm, a prion, or a parasite.

In an additional embodiment, the ELP-MRD fusion protein comprises an ELP and MRD or another component of the complex operably linked through a linker peptide. In an additional embodiment, the linker comprises a sequence selected from the group consisting of: SEQ ID NO:1, SEQ ID NO:2, and SEQ ID NO:19.

In an additional embodiment, the ELP-MRD fusion protein comprises at least 2, at least 3, at least 4, or at least 5 MRDs operably linked to another component of the ELP-MRD fusion protein through a linker peptide. In an additional embodiment, the ELP-MRD fusion protein comprises an ELP and MRD or another component of the complex operably attached without a linker peptide.

In an additional embodiment, the ELP-MRD fusion protein comprises at least two ELPs and MRDs or other components of the complex operably attached without a linker.

In an additional embodiment, the ELP-MRD fusion protein comprises an effector domain capable of interacting with a host effector system. In an additional embodiment, the ELP-MRD fusion protein comprises an MRD comprising an amino acid sequence of an immunoglobulin effector domain.

In an additional embodiment, the complex has CDC activity. In an additional embodiment, the complex has ADCC activity.

In an additional embodiment, the ELP-MRD fusion protein is capable of binding CD3 and CD19 simultaneously.

In an additional embodiment, the invention encompasses a polynucleotide encoding the ELP-MRD fusion proteins of the invention. In an additional embodiment, the invention encompasses a vector comprising the polynucleotide encoding the ELP-MRD fusion proteins of the invention. In an additional embodiment, the invention encompasses a host cell comprising the vector comprising the polynucleotide encoding the ELP-MRD fusion proteins of the invention.

In an additional embodiment, the invention encompasses a method for producing a complex comprising an ELP-MRD fusion protein comprising culturing the host cell comprising the vector comprising the polynucleotide encoding the ELP-MRD fusion proteins of the invention under conditions wherein ELP-MRD fusion protein is expressed and recovering said fusion protein.

In an additional embodiment, the invention encompasses a method for purifying a complex comprising an ELP-MRD fusion protein complex of the invention comprising (i) forming a solution containing an ELP-MRD fusion protein complex of the invention (ii) inducing a phase transition and aggregation of ELP-MRD fusion protein complexes in the solution; and (iii) separating aggregated ELP-MRD fusion protein complexes from the solution.

In an additional embodiment, the phase transition and aggregation is induced by changing the solution temperature, ionic strength, or a combination thereof. In an additional embodiment, the aggregated ELP-MRD fusion protein complexes are separated from the solution by centrifugation. In an additional embodiment, the method further includes the steps of (iv) resolubilizing the separated ELP-MRD fusion protein in a buffer at conditions below the transition phase complexes; and (v) repeating steps (i)-(iv) at least one time, two times, three times, four times, or five times.

In an additional embodiment, the invention encompasses a pharmaceutical composition comprising a complex the invention; a polynucleotide of the invention; a vector of the invention; or a host cell of the invention.

In an additional embodiment, the invention encompasses a method for inhibiting the growth of a cell comprising contacting the cell with a complex of the invention; a polynucleotide of the invention; a vector of the invention; or a host cell of the invention.

In an additional embodiment, the invention encompasses a method for inhibiting angiogenesis in a patient comprising administering to said patient a therapeutically effective amount of a complex of the invention; a polynucleotide of the invention; a vector of the invention; or a host cell of the invention.

In an additional embodiment, the invention encompasses a method for treating a patient having an autoimmune disease comprising administering to said patient a therapeutically effective amount of a complex of the invention; a polynucleotide of the invention; a vector of the invention; or a host cell of the invention.

In an additional embodiment, the invention encompasses a method for treating a patient having cancer comprising administering to said patient a therapeutically effective amount a complex of the invention; a polynucleotide of the invention; a vector of the invention; or a host cell of the invention. In an additional embodiment, the method further comprises administering a second therapeutic agent to the patient.

In an additional embodiment, the invention encompasses a method for making a complex comprising an ELP operably linked to an MRD, the method comprising (i) identifying MRDs that bind a target, and optionally conducting a screen of sequence variants of the MRD, to identify an MRD variant with desirable altered binding or functional characteristics, and (ii) expressing or synthesizing the MRD or MRD variant as a ELP-MRD fusion protein complex wherein the MRD or MRD variant is optionally operably linked to other components of the fusion protein via a linker, and wherein ELP-MRD fusion protein containing the MRD or MRD variant retains the capability to bind the target.

In an additional embodiment, the invention encompasses a method for optimizing a complex comprising an ELP operably linked to an MRD, the method comprising (i) engineering constructs encoding an MRD that is operably linked to different locations within an ELP-MRD fusion protein and/or to substituted ELPs of different compositions, in the ELP-MRD fusion protein, wherein said linkage is optionally via linkers of the same length and composition, or of different lengths and compositions; (ii) expressing the construct to produce ELP-MRD complexes; (iii) screening the ELP-MRD complexes for target binding; (iv) identifying ELP-MRD complexes that bind a target, and optionally quantitating said target binding or comparing said target binding with a reference MRD, ELP-MRD fusion protein or ligand; and (v) selecting an ELP-MRD complex with desirable binding or functional characteristics.

In an additional embodiment, the invention encompasses a method for identifying an MRD, ELP-MRD complex, antibody, antibody fragment, or ligand that competes with a reference compound for binding to a target, the method comprising (i) contacting the MRD, ELP-MRD complex, antibody, antibody fragment, or ligand with said target in the presence and absence of said reference compound, wherein said reference compound is selected from an antibody, MRD, cognate ligand or other target ligand that binds to said target, and (ii) determining target binding of the MRD, ELP-MRD complex, antibody, antibody fragment, or ligand in the presence and absence of said reference compound, wherein a lower level of target binding in the presence of the reference compound as compared to the absence of said reference compound indicates that the antibody, MRD, or ELP-MRD complex competes with said reference compound for binding to said target.

A complex comprising an elastin-like peptide-modular recognition domain (ELP-MRD) fusion protein, wherein the fusion protein comprises at least one elastin-like peptide (ELP) and (a) at least one modular recognition domain (MRD) that binds a soluble ligand or (b) at least two MRDs that bind membrane associated targets is provided herein.

In an additional embodiment, the ELP-MRD fusion protein comprises an ELP comprising the sequence (VPGXG)n (SEQ ID NO:119), wherein X is a natural or non-natural amino acid residue and optionally varies among repeats units, and where n is a number from 1 to 200. In an additional embodiment, X is an amino acid residue selected from A, R, N, D, C, E, Q, G, H, I, L, K, M, F, S, T, W, Y and V.

In an additional embodiment, the ELP-MRD fusion protein comprises an ELP and MRD or other component of the complex operably linked through a linker peptide.

In an additional embodiment, the ELP-MRD fusion protein further comprises an antibody fragment or domain. In an additional embodiment, the ELP-MRD fusion protein further comprises a cytotoxic agent.

In an additional embodiment, the ELP-MRD fusion protein comprises at least 2, at least 3, at least 4, or at least 5 MRDs that bind the same target or different targets. In an additional embodiment, the ELP-MRD fusion protein comprises at least 1 MRD that binds a soluble ligand and at least one MRD that binds a membrane associated target. In an additional embodiment, the ELP-MRD fusion protein binds at least 2, at least 3, at least 4, or at least 5 cytokines, chemokines, or serum proteins. In an additional embodiment, the ELP-MRD fusion protein binds ANG2, VEGF, TNF, TNFSF11, TNFSF13B, TNFSF15, TNFSF14,₌TNFSF13, IL-1, IGF, IL-1 beta, IL-6, IL-10, IL-17, NGF, CCL19, CCL21 or interferon alpha. In an additional embodiment, the ELP-MRD fusion protein binds serum albumin, thyroxin-binding protein, transferrin, fibrinogen, or an immunoglobulin. In an additional embodiment, the ELP-MRD fusion protein binds a target associated with an endogenous blood brain barrier receptor mediated transport system. In an additional embodiment, the ELP-MRD fusion protein binds a transferrin receptor. In an additional embodiment, the ELP-MRD fusion protein binds a cancer antigen, pathogenic antigen or an antigen associated with a disorder of the immune system. In an additional embodiment, the ELP-MRD fusion protein binds at least 2, at least 3, at least 4, or at least 5 different cancer antigens. In an additional embodiment, the ELP-MRD fusion protein binds at least 2, at least 3, at least 4, or at least 5 different pathogenic antigens. In an additional embodiment, the ELP-MRD fusion protein binds at least 2, at least 3, at least 4, or at least 5 different antigens associated with a disorder of the immune system. In an additional embodiment, the ELP-MRD fusion protein binds ErbB2, EGFR, CD20, cMET, IGFR1, CD19, CD20, IL6R, CCR5, CCR7, and NAV1.7. In an additional embodiment, the ELP-MRD fusion protein comprises an effector domain capable of interacting with a host effector system.

In an additional embodiment, the ELP-MRD fusion protein binds a target on a leukocyte. In an additional embodiment, the ELP-MRD fusion protein binds a target on a T cell or natural killer cell. In an additional embodiment, the ELP-MRD fusion protein binds CD3. In an additional embodiment, the ELP-MRD fusion protein binds CD3 epsilon.

In an additional embodiment, the ELP-MRD fusion protein binds a target on a diseased cell. In an additional embodiment, the ELP-MRD fusion protein binds a target on a tumor cell. In an additional embodiment, the ELP-MRD fusion protein binds a target on a leukocyte and a target on a tumor cell.

In an additional embodiment, the ELP-MRD fusion protein binds CD3 and CD19.

A polynucleotide encoding an ELP-MRD fusion protein is also provided. A vector comprising such a polynucleotide is also provided. A host cell comprising such a vector is also provided. A method for producing a complex comprising an ELP-MRD fusion protein comprising culturing such a host cell under conditions wherein the ELP-MRD fusion protein is expressed and recovering said fusion protein is also provided.

A pharmaceutical composition comprising an elastin-like peptide-modular recognition domain (ELP-MRD) fusion protein, a polynucleotide encoding an ELP-MRD fusion protein, a vector comprising such a polynucleotide, and a host cell comprising such a vector are also provided.

A composition comprising an elastin-like peptide-modular recognition domain (ELP-MRD) fusion protein, a polynucleotide encoding an ELP-MRD fusion protein, a vector comprising such a polynucleotide, and a host cell comprising such a vector can be used in a method of killing or inhibiting cells associated with cancer, an autoimmune disease, an infectious disease, or another disease or disorder, wherein the method comprises administering to a patient a therapeutically effective amount of the complex, the polynucleotide, the vector, or the host cell. In an additional embodiment, the method further comprises administering a second therapeutic agent to the patient.

A method for making a complex comprising an ELP operably linked to an MRD is also provided. In an additional embodiment, the method comprises (i) identifying MRDs that bind a target, and optionally conducting a screen of sequence variants of the MRD, to identify an MRD variant with desirable altered binding or functional characteristics, and (ii) expressing or synthesizing the MRD or MRD variant as an MMM complex (e.g., an ELP-MRD fusion protein) complex wherein the MRD or MRD variant is optionally operably linked to other components of the fusion protein via a linker, and wherein the MMM complex (e.g., ELP-MRD fusion protein) containing the MRD or MRD variant retains the capability to bind the target.

### EXAMPLES

### Example 1

### Creation of ELP-MRD Fusion Proteins

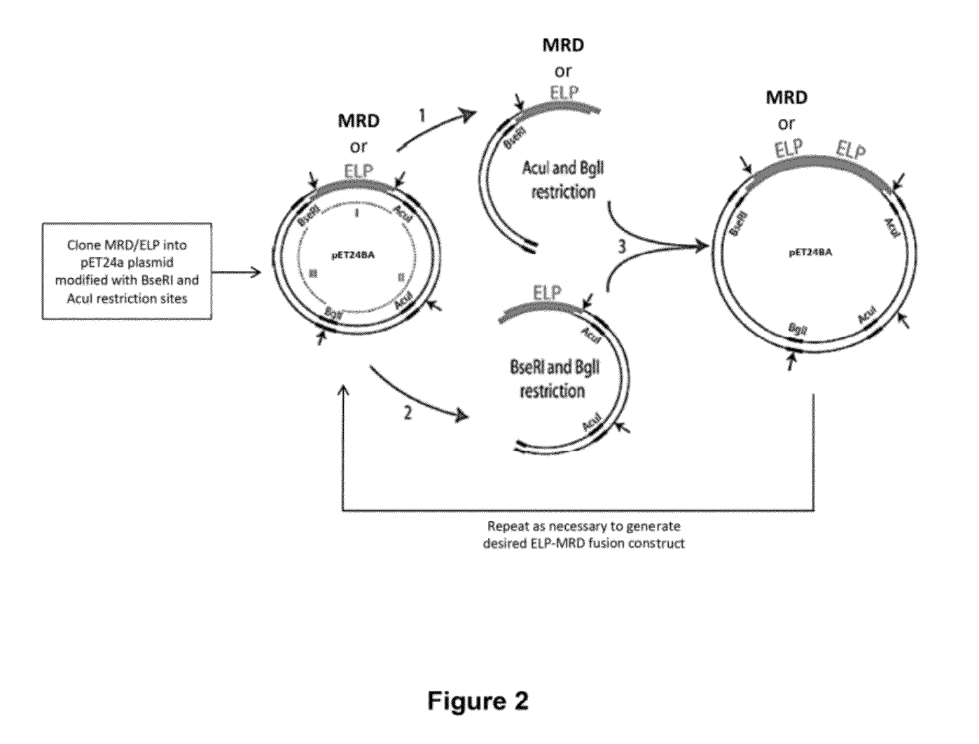

Recursive directional ligation was used to create vectors for expression of ELP-MRD fusion proteins. See FIG. 2. Unique BseRI and AcuI restriction sites were engineered into the pET-24a vector (Novagen/Merck). The pET-24a vector system is designed for T7 promoter driven expression of recombinant proteins in E. coli. MRD or ELP modules were then cloned into the region flanked by BseRI and AcuI. Fusion proteins were generated by plasmid reconstruction using the ELP/MRD-containing restriction fragments resulting from AcuI and BglI double digest (“leading” sequence) and BseRI and BglI double digest (“trailing” sequence) reactions. This fusion product was then used as the template for further directional ligation with itself or other MRD/ELP containing constructs in successive cycles to generate the desired ELP-MRD fusion protein (method adapted from McDaniel et al., Biomacromolecules, 11: 944-952 (2010)).

### Example 2

### Expression and Analysis of ELP-MRD Fusion Proteins

The following MMM complexes (e.g., ELP-MRD fusion protein) were expressed in E. coli. from the adapted pET-24a vectors:
(1) Construct 1: ANGa-ELP₂(40)-ANGa-ELP₂(40)-ANGa-ELP₂(40)-ANGa-ELP₂(40)-10×His construct;
(2) Construct 2: VEGFa-ELP₂(80)-ANGa-ELP₂(80)-10×His construct; and
(3) Construct 3: HER2a-ELP₂(80)-ANGa-ELP₂(80)-10×His construct.
   ANGa is a rhAng2-targeting MRD with the following sequence:

| | |
|---|---|
| GAQTNFMPMDDLEQRLYEQFILQQGLE. | (SEQ ID NO: 144) |

VEGFa is a rhVEGF165-targeting MRD with the following sequence:

| | |
|---|---|
| WCNGFPPNYPCY. | (SEQ ID NO: 145) |

HER2a is a HER2-targeting MRD with the following sequence:

(SEQ ID NO: 146)
VDNKFNKEMRNAYWEIALLPNLNNQQKRAFIRSLYDDPSQSANLLAEARK
LNDAQAPK.

ELP₂ is an ELP Pentamer Molecule with the sequence VPG(A/G)G (SEQ ID NO:147), wherein A and G alternate in a 1:1 ratio.
The numbers in parathesis indicate the number of consecutive ELP₂ sequences in the construct.

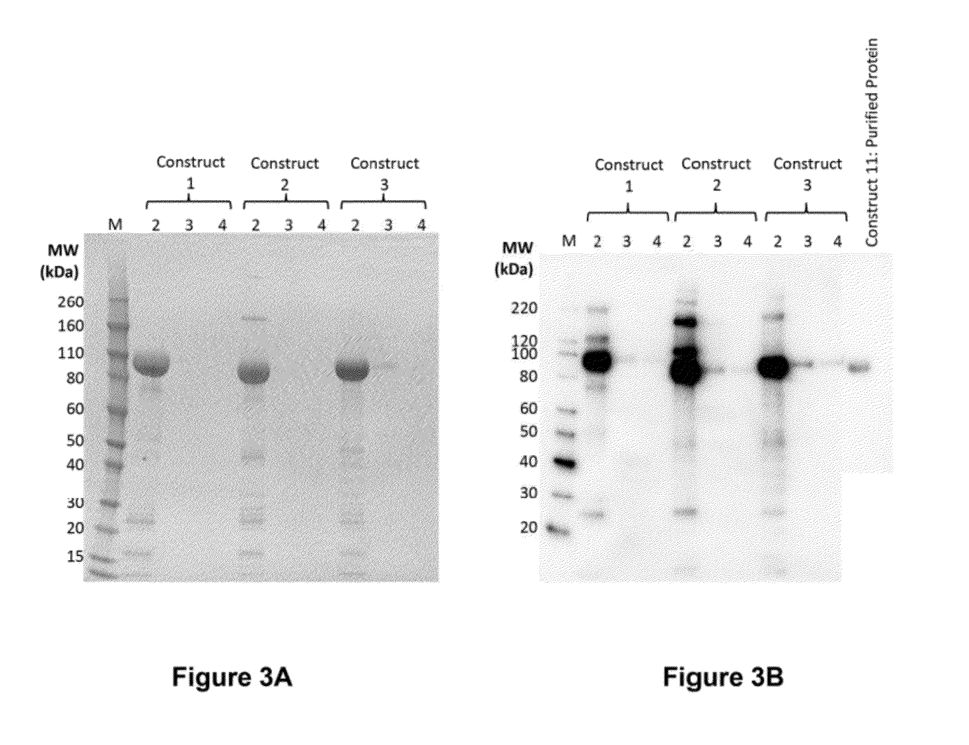

The resulting recombinant proteins were purified using His-tag purification and analyzed by non-reducing SDS-Page. Fractions 2-4 from each protein purification were prepared in non-reducing sample buffer and loaded onto a NuPAGE 4-12% Bis-Tris gel (Invitrogen). The results are shown in FIG. 3A.

The same purified protein fractions were also analyzed by Western blot. The fractions were characterized with a horseradish peroxidase (HRP) conjugated anti-6×His tag antibody (Abcam) under non-reducing conditions. Purified protein from Construct 11, another ELP-MRD fusion construct encoding ANGa-ELP₂(160)-10×His, was used as a positive control for the detection antibody. The results are shown in FIG. 3B.

### Example 3

### Purification and Analysis ELP-MRD Fusion Proteins

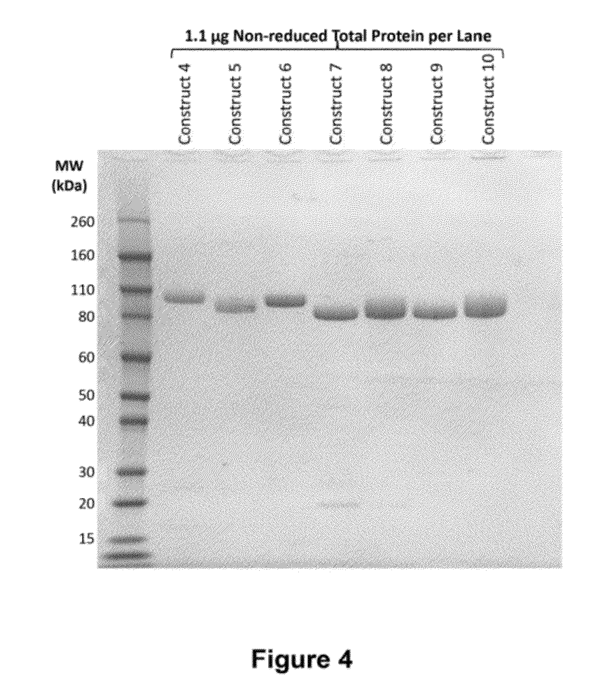

The ELP-MRD fusion proteins listed below in Table 4 were expressed and purified by His-tag purification. The isolated protein fractions were prepared in non-reducing sample buffer and loaded onto a NuPAGE 4-12% Bis-Tris gel (Invitrogen) with equal total protein content. ELP-MRD fusion protein containing fractions were pooled and buffer exchanged into phosphate buffered saline (PBS). The results are shown in FIG. 4.

TABLE 4
ELP-MRD fusion proteins

| MMM complex | |
|---|---|
| (e.g., ELP-MRD | |
| fusion protein) ID | Sequence (N → C) |
| Construct 4 | ANGa -ELP₂(40)- ANGa -ELP₂(40)- |
| | ANGa -ELP₂(40)- ANGa -ELP₂(40)-10xHis |
| Construct 5 | VEGFa-ELP₂(80)- ANGa -ELP₂(80)-10xHis |
| Construct 6 | HER2a-ELP₂(80)- ANGa -ELP₂(80)-10xHis |
| Construct 7 | ANGa -ELP₂(160)-10xHis |
| Construct 8 | ELP₂(80)- ANGa -ELP₂(80)-10xHis |
| Construct 9 | ELP₂(80)- ANGa -ELP₂(80)-10xHis |
| Construct 10 | ANGa -ELP₂(160)-10xHis |

### Example 4

### ELP-MRD Fusion Proteins Bind Target Proteins

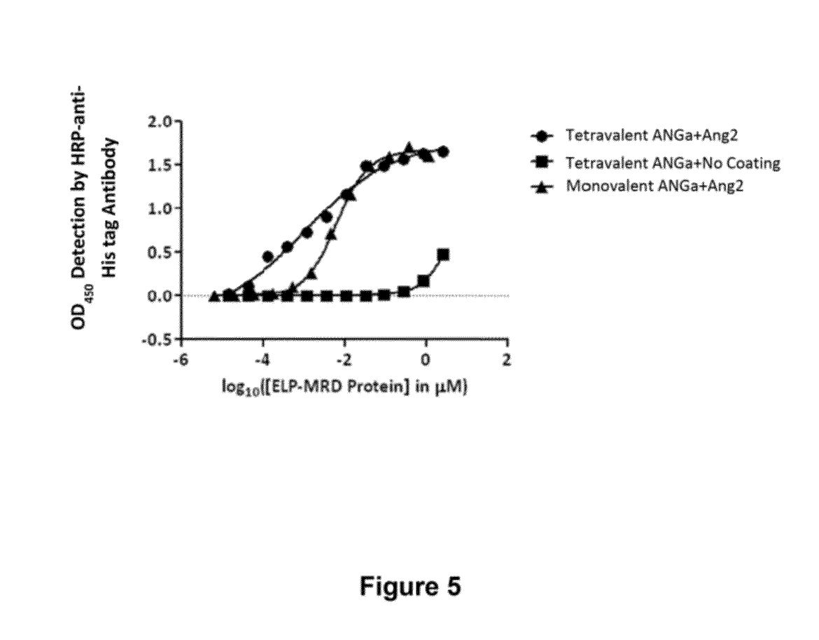

The binding of an MMM complex (e.g., an ELP-MRD fusion protein) containing 4 angiopoietin-2 (Ang2)-binding MRDs to Ang2 in an ELISA assay was compared to the binding of an ELP-MRD fusion protein containing one Ang-2 binding MRD. Recombinant human angiopoietin-2 (rhAng2) was coated on microplate wells, and the ability of Construct 7 and Construct 4 to bind was analyzed to determine qualitative EC₅₀ values and propensity for nonspecific binding. Uncoated microplate wells were used as a negative control. The results are shown in FIG. 5 and demonstrate that both the monovalent and tetravalent constructs bound to rhAng2, and the tetravalent construct bound more efficiently.

### Example 5

### ELP-MRD Fusion Proteins Bind Multiple Target Proteins

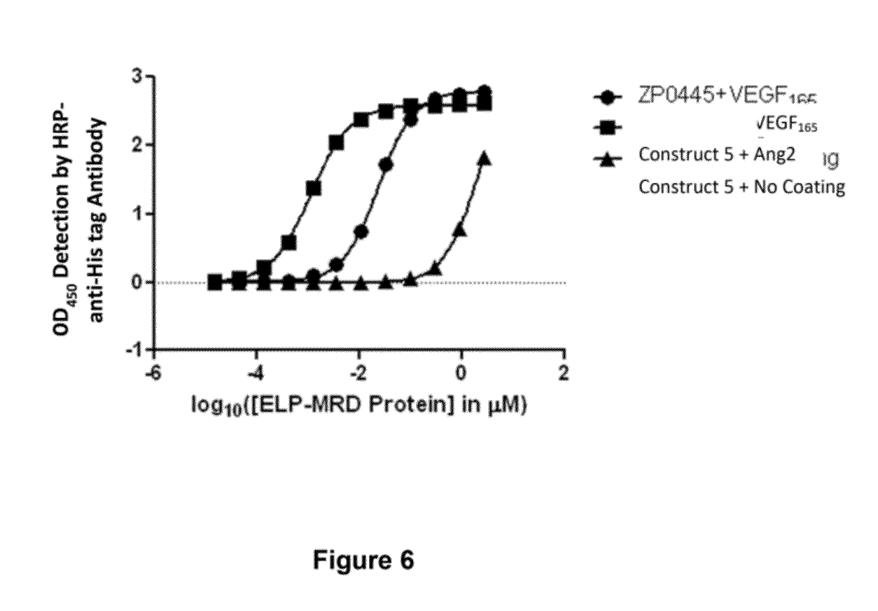

The ability of a bispecific ELP-MRD fusion protein was measured by ELISA on rhAng-2-coated, rhVEGF₁₆₅-coated, and uncoated wells. The bispecific ELP-MRD fusion protein was engineered with a rhVEGF₁₆₅-targeting MRD (VEGFa) and a rhAng2-targeting MRD (ANGa) along an ELP backbone. ELISAs were performed to determine the independent binding of the bispecific fusion protein, resulting in qualitative EC₅₀ values, for each of these targets as well as its propensity for nonspecific binding. The results, shown in FIG. 6, demonstrate that the ELP-MRD fusion protein bound to both rhAng2 and rhVEGF₁₆₅.

### Example 6

### Fusions Containing Internally Constrained MRDs Bind Target Proteins

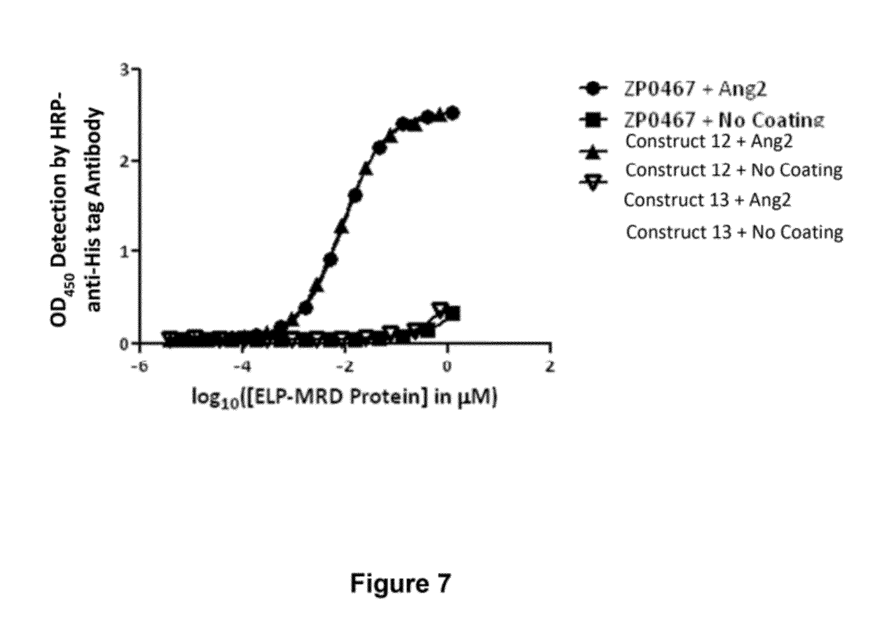

The ELP-MRD fusion proteins, Construct 12 and Construct 13, were engineered with an constrained rhAng2-targeting peptide (ANGd) sandwiched between ELP repeat modules. The rhAng2-targeting peptide was constrained via a disulfide bond between amino acids with the rhAng2-targeting peptide. Direct binding ELISAs to rhAng2 were performed to determine qualitative EC₅₀ values and propensity for nonspecific binding. The results are shown in FIG. 7 and demonstrate that the ELP-MRD fusion proteins containing a constraing rhAng2 MRD bound to rhAng2.

### Example 7

### ELP-MRD Fusions Bind Target Proteins on Cells

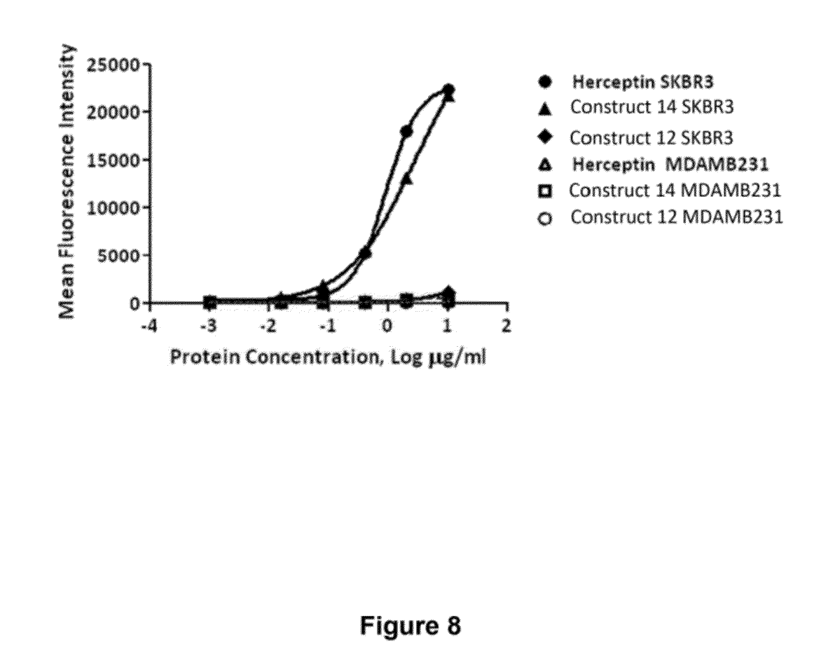

The ability of a HER2-targeted ELP-MRD fusion protein was measured by FACS analysis on SKBR3 (HER2+) and MDAMB231 (HER2−) cells. Construct 14 contains a HER2-targeting MRD. Construct 12, which contains an Ang-2 binding MRD and not a HER2-targeting MRD served as a negative control. The results are shown in FIG. 8 and demonstrate that Construct 14 binds to cells expressing HER2.

### Example 8

### Administration of ELP-MRD Fusion Proteins

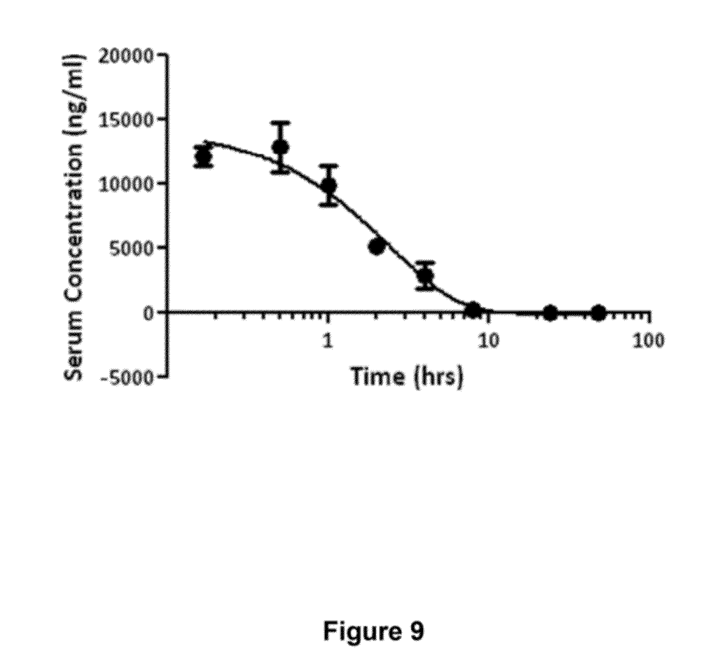

ELP-MRD fusion proteins were administered intravenously to mice at 1 mg/kg, and then serum was collected at the time points indicated in FIG. 9. The serum was analyzed by ELISA to determine the concentration of ANGa-ELP₂(160)-10×His. The pharmacokinetic data are shown in FIG. 9, and demonstrate that the fusion protein had a 1.7 hour half-life. Other target-binding molecules with similar half-lives have been shown to be efficacious.

### Example 9

### Construction of Bispecific ELP-MRD Fusions for Redirected T-Cell Killing and Expression in E. coli

ELP-MRD fusion proteins containing a CD19-targeting MRD at the N-terminus and a CD3-targeting MRD at the C-terminus (e.g., CD19a-ELP₂(160)-CD3a-10×His tag) or a CD3-targeting MRD at the N-terminus and CD19-targeting MRD at the C-terminus (e.g., CD3a-ELP₂(160)-CD19a-10×His tag) are generated by recursive directional ligation and plasmid reconstruction (RDL-PRe) as described in Example 1. The N-terminal MRD is cloned into the pET-24BA vector (e.g., pET-24a vector modified with unique BseRI and AcuI restriction sites) as a ‘leader’ sequence, and the C-terminal MRD is cloned into the same vector as a ‘trailer’ sequence. These MRDs are then ligated to the ELP₂(160) scaffold via successive directional ligation and plasmid reconstruction steps. The 10×His tag is then appended at the C-terminus of the resulting ELP-MRD fusion construct by RDL-PRe. The final ELP-MRD fusion construct is then used to chemically transform T7 Express (New England Biolabs) cells using the manufacturer's protocol, followed by plating on LB-kanamycin plates for overnight growth and selection. A single colony from the LB-kanamycin plate is used to inoculate 30 ml of Super Broth +50 μg/ml kanamycin in a 125-ml baffled Erlenmeyer flask for an overnight (−16 hr) culture at 37° C. in a shaking incubator set at 275 rpm. On the following day, the overnight culture is diluted 1:100 in 250 ml of Super Broth +50 μg/ml kanamycin and grown at 37° C. in a shaking incubator set at 275 rpm until the cell density as measured by the absorption at 600 nm (OD₆₀₀) reaches ˜0.8-1.2. Induction of fusion protein expression is achieved with IPTG at a final concentration of 0.4 mM. The culture is then grown under the same conditions as the initial growth phase for 3-4 hrs. Cells are then pelleted by centrifugation at 4700 rpm for 15 min, and the supernatant is decanted. The cell pellet is frozen at −80° C. overnight and thawed for cell lysis with Bugbuster Master Mix (EMD4Biosciences) per manufacturer's instructions.

Purification of ELP-MRD fusion proteins from cleared cell lysates is performed via the C-terminal His tag by affinity chromatography on a cobalt resin column. The ELP-MRD fusion proteins are eluted from the column with 150 mM imidazole. SDS-PAGE is then performed to analyze the protein content in column elutions with NuPAGE 4-12% Bis-Tris gel (Invitrogen) under non-reducing conditions. The gels are stained with SimplyBlue Safestain (Invitrogen) to visualize protein bands. The purified proteins are then buffer exchanged into PBS.

### Example 10

### Characterization of CD19×CD3 or CD3×CD19 Bispecific ELP-MRD Fusion Protein Binding Properties

Bispecific ELP-MRD fusion proteins to CD3 and CD19 are analyzed by flow cytometric analysis on CD3-positive Jurkat cells, human PBMCs and a number of different CD19-positive B cell lymphoma cell lines (e.g., SKW6.4, Blin I, BJAB, Daudi and Raji) to determine their specific binding affinities to each target. Since BL60 and the plasmacytoma cell lines NCI and L363 are negative for both surface molecules, CD3 and CD19, they are used as negative control cells to determine the specificity of ELP-MRD fusion protein interactions. CD3-negative Jurkat cells can also be used as a negative control cell population. Cell lines are cultured in complete RPMI 1640 (Invitrogen) with 10% FCS (GIBCO).

Cells are washed with PBS and blocked by resuspension in PBS with 10% human IgG (Innovative Research) and 0.1% NaN₃ (blocking buffer) for 30 min at 4° C. Cells are then pelleted by centrifugation (100×g for 5 min) followed by incubation with the bispecific ELP-MRD fusion protein in blocking buffer for 30 min at 4° C. The cells are washed three times with PBS. Detection of cell-surface bound ELP-MRD fusion proteins can be achieved by using a FITC-conjugated antibody against the His-tag (Abcam). The irrelevant ZHER2×CD3 or CD3×ZHER2 bispecific ELP-MRD fusion proteins, produced by the same expression system as CD19×CD3 or CD3×CD19 fusion proteins, or the His-tag antibody alone serve as negative controls. Flow cytometry can be performed with a BD FACScan.

### Example 11

### In Vitro Cytotoxicity Assay of the CD19×CD3 or CD3×CD19 ELP-MRD Fusion Proteins Against CD19-Positive Lymphoma Cells

The bispecific CD19×CD3 or CD3×CD19 ELP-MRD fusion proteins are assayed with respect to their abilities to induce redirected T-cell killing of CD19-positive lymphoma cells. Human peripheral blood mononuclear cells (PBMCs) are isolated as effector cells from fresh buffy coats of random donors using Lymphoprep™ (Nycomed/Axis-Shield PoC) gradient centrifugation with subsequent centrifugation at 100×g to remove platelets. CD19-positive B cells are depleted using Dynabeads® CD19 Pan B (Life Technologies). The PBMC populations are analyzed by flow cytometry before and after CD19-positive B cell depletion by labeling with FITC-conjugated mouse antibody against human CD19 and counter-labeled with a PE-conjugated anti-CD45 antibody. The PBMCs are incubated overnight at 37° C. under 5% CO₂. CD19-positive B cell lines (e.g., SKW6.4, Blin I, BJAB, Daudi and Raji) were used as target cells.

Target cells are incubated in 96-well plates using RPMI 1640 complete medium (Invitrogen) with 10% FCS (GIBCO) at different densities, such that addition of the same number of unstimulated PBMCs resulted in different effector-to-target cell (E:T) ratios. Various concentrations of bispecific ELP-MRD fusion proteins are then added to each well followed by the addition of unstimulated PBMCs. Plates are incubated at 37° C. under 5% CO₂ for 3 hrs. Cytotoxicity can be measured using the DELFIA® EuTDA cytotoxicity assay (PerkinElmer) in round-bottom 96-well-plates following manufacturer's instructions. Spontaneous cell death is measured by incubating the target cells without effector cells or ELP-MRD fusion proteins, and maximal cell death are determined by incubating the target cells with 10% Triton X-100. The fraction of specific cell lysis is calculated as the ratio between effector mediated cytotoxicity ([experimental cell death]−[spontaneous cell death]) and the maximum expected cytotoxicity ([maximal cell death]−[spontaneous cell death]).

### Example 12

### In Vivo Efficacy of CD19×CD3 or CD3×CD19 ELP-MRD Fusion Proteins in Human Xenograft Model of B-Cell Lymphoma

Raji B lymphoma cells are removed from routine cell culture, washed in PBS, and prepared as 1×10⁷ cells/ml. NOD/SCID mice are then inoculated subcutaneously with 1×10⁶ Raji cells with or without 5×10⁶ PBMCs (as prepared above) in a 50% Matrigel solution. CD19×CD3 or CD3×CD19 ELP-MRD fusion proteins, HER2a×CD3 or CD3×HER2a ELP-MRD fusion proteins, ELP₂(160)-CD3 MRD or CD3 MRD-ELP₂(160) fusion proteins, or PBS is administered intravenously 1 hr after lymphoma cell inoculation. ELP-MRD fusion proteins or PBS is administered once per day for four days after the initial dose. Subcutaneous tumors are measured by caliper to determine growth rate for each treatment group. Body weight of mice is also determined twice per week as an indicator of treatment tolerability.

All publications, patents, patent applications, internet sites, and accession numbers/database sequences (including both polynucleotide and polypeptide sequences) cited are herein incorporated by reference in their entirety for all purposes to the same extent as if each individual publication, patent, patent application, internet site, or accession number/database sequence were specifically and individually indicated to be so incorporated by reference.

1. A complex comprising an elastin-like peptide-modular recognition domain (ELP-MRD) fusion protein, wherein the fusion protein comprises at least one elastin-like peptide (ELP) and (a) at least one modular recognition domain (MRD) that binds a soluble ligand or (b) at least two MRDs that bind membrane associated targets.

2. The complex of claim 1, wherein the ELP-MRD fusion protein comprises an ELP comprising the sequence (VPGXG)n (SEQ ID NO:119), wherein X is a natural or non-natural amino acid residue and optionally varies among repeats units, and wherein n is a number from 1 to 200.

3. The complex of claim 1, wherein X is an amino acid residue selected from A, R, N, D, C, E, Q, G, H, I, L, K, M, F, S, T, W, Y and V.

4. (canceled)

5. (canceled)

6. (canceled)

7. The complex of claim 1, wherein the ELP-MRD fusion protein comprises at least two, at least three, at least four, or at least five MRDs that bind the same target.

8. The complex of claim 1, wherein the ELP-MRD fusion protein comprises at least two, at least three, at least four, or at least five MRDs that bind different targets.

9. The complex of claim 1, wherein the ELP-MRD fusion protein comprises at least one MRD that binds a soluble ligand.

10. The complex of claim 9, wherein the ELP-MRD fusion protein further comprises at least one MRD that binds a cell membrane associated target.

11. (canceled)

12. (canceled)

13. The complex of claim 10, wherein the ELP-MRD fusion protein binds at least one, at least two, at least three, at least four, or at least five cancer antigens.

14. (canceled)

15. (canceled)

16. (canceled)

17. The complex of claim 1, wherein the ELP-MRD fusion protein binds at least one, at least two, at least three, at least four, or at least five pathogenic antigens.

18. (canceled)

19. The complex of claim 1, wherein the ELP-MRD fusion protein binds at least one, at least two, at least three, at least four, or at least five antigens associated with a disease or disorder of the immune system.

20. (canceled)

21. (canceled)

22. (canceled)

23. The complex of claim 1, wherein the ELP-MRD fusion protein binds at least one, at least two, at least three, at least four, or at least five serum proteins.

24. (canceled)

25. The complex of claim 1, wherein the ELP-MRD fusion protein binds a target associated with an endogenous blood brain barrier receptor mediated transport system.

26. The complex of claim 25, wherein the ELP-MRD fusion protein binds a transferrin receptor.

27. (canceled)

28. The complex of claim 1, wherein the ELP-MRD fusion protein binds a target on a leukocyte.

29. (canceled)

30. The complex of claim 1, wherein the ELP-MRD fusion protein binds CD3.

31. (canceled)

32. (canceled)

33. The complex of claim 1, wherein the ELP-MRD fusion protein binds a target on a diseased cell.

34. The complex of claim 28 wherein the ELP-MRD fusion protein binds a target on a leukocyte and a target on a tumor cell.

35. The complex of claim 1, wherein the ELP-MRD fusion protein binds CD3 and CD19.

36. A pharmaceutical composition comprising the complex of claim 1.

37. A polynucleotide encoding the ELP-MRD fusion protein of claim 1.

38. A vector comprising the polynucleotide of claim 37.

39. A host cell comprising the vector of claim 38.

40. A method for producing a complex comprising an ELP-MRD fusion protein comprising culturing the host cell of claim 39 under conditions wherein the ELP-MRD fusion protein is expressed and recovering said fusion protein.

41. A method for treating a patient having a disease or disorder comprising administering to said patient a therapeutically effective amount of a complex comprising an elastin-like peptide-modular recognition domain (ELP-MRD) fusion protein, wherein the fusion protein comprises at least one elastin-like peptide (ELP) and (a) at least one modular recognition domain (MRD) that binds a soluble ligand or (b) at least two MRDs that bind membrane associated targets.

42. (canceled)

43. (canceled)

44. (canceled)

45. (canceled)

46. A method for making a complex comprising an ELP operably linked to an MRD, the method comprising
(i) identifying MRDs that bind a target, and optionally conducting a screen of sequence variants of the MRD, to identify an MRD variant with desirable altered binding or functional characteristics, and
(ii) expressing or synthesizing the MRD or MRD variant as an ELP-MRD fusion protein wherein the MRD or MRD variant is optionally operably linked to other components of the fusion protein via a linker, wherein the ELP-MRD fusion protein containing the MRD or MRD variant retains the capability to bind the target, and wherein the ELP-MRD fusion protein comprises (a) at least one ELP and at least one MRD that binds a soluble ligand or (b) at least two MRDs that bind membrane associated targets.


SEQUENCE LISTING (153 sequences)

SEQ ID NO: 1 - protein, 4 aa, artificial
  linker peptide
GGGS

SEQ ID NO: 2 - protein, 15 aa, artificial
  linker peptide
SSGGGGSGGGGGGSS

SEQ ID NO: 3 - protein, 7 aa, artificial
  integrin targeting MRD
YCRGDCT

SEQ ID NO: 4 - protein, 7 aa, artificial
  integrin targeting MRD
PCRGDCL

SEQ ID NO: 5 - protein, 7 aa, artificial
  integrin targeting MRD
TCRGDCY

SEQ ID NO: 6 - protein, 7 aa, artificial
  integrin targeting MRD
LCRGDCF

SEQ ID NO: 7 - protein, 28 aa, artificial
  angiogenic cytokine targeting MRD
MGAQTNFMPMDDLEQRLYEQFILQQGLE

SEQ ID NO: 8 - protein, 28 aa, artificial
  angiogenic cytokine targeting MRD
MGAQTNFMPMDNDELLLYEQFILQQGLE

SEQ ID NO: 9 - protein, 28 aa, artificial
  angiogenic cytokine targeting MRD
MGAQTNFMPMDATETRLYEQFILQQGLE

SEQ ID NO: 10 - protein, 54 aa, artificial
  angiogenic cytokine targeting MRD
AQQEECEWDPWTCEHMGSGSATGGSGSTASSGSGSATHQEECEWDPWTCEHMLE

SEQ ID NO: 11 - protein, 28 aa, artificial
  angiogenic cytokine targeting MRD
MGAQTNFMPMDNDELLNYEQFILQQGLE

SEQ ID NO: 12 - protein, 10 aa, artificial
  angiogenic cytokine targeting MRD
PXDNDXLLNY

SEQ ID NO: 13 - protein, 19 aa, artificial
  VEGF targeting MRD
VEPNCDIHVMWEWECFERL

SEQ ID NO: 14 - protein, 27 aa, artificial
  MRD
SFYSCLESLVNGPAEKSRGQWDGCRKK

SEQ ID NO: 15 - protein, 11 aa, artificial
  vascular homing peptide MRD
ACDCRGDCFCG

SEQ ID NO: 16 - protein, 58 aa, artificial
  EGFR binding MRD
VDNKFNKELEKAYNEIRNLPNLNGWQMTAFIASLVDDPSQSANLLAEAKKLNDAQAPK

SEQ ID NO: 17 - protein, 58 aa, artificial
  EGFR binding MRD
VDNKFNKEMWIAWEEIRNLPNLNGWQMTAFIASLVDDPSQSANLLAEAKKLNDAQAPK

SEQ ID NO: 18 - protein, 58 aa, artificial
  ErbB2 targeting MRD
VDNKFNKEMRNAYWEIALLPNLNNQQKRAFIRSLYDDPSQSANLLAEAKKLNDAQAPK

SEQ ID NO: 19 - protein, 18 aa, artificial
  linker peptide
SSGGGGSGGGGGGSSRSS

SEQ ID NO: 20 - protein, 60 aa, artificial
  MRD
MGAQTNFMPMDNDELLLYEQFILQQGLEGGSGSTASSGSGSSLGAQTNFMPMDNDELLLY

SEQ ID NO: 21 - protein, 16 aa, artificial
  ConFA
AQQEECEFAPWTCEHM

SEQ ID NO: 22 - protein, 106 aa, artificial
  MRD
XXXXXXXXXXXXXXXXXXXXXXXXXXXXXXXXXXXXXXXXXXXXXXXXXXEFAPWTXXXXXXXXXXXXXXXXXXXXXXXXXXXXXXXXXXXXXXXXXXXXXXXXXX

SEQ ID NO: 23 - protein, 54 aa, artificial
  2xConFA
AQQEECEFAPWTCEHMGSGSATGGSGSTASSGSGSATHQEECEFAPWTCEHMLE

SEQ ID NO: 24 - protein, 16 aa, artificial
  ConLA
AQQEECELAPWTCEHM

SEQ ID NO: 25 - protein, 106 aa, artificial
  MRD
XXXXXXXXXXXXXXXXXXXXXXXXXXXXXXXXXXXXXXXXXXXXXXXXXXELAPWTXXXXXXXXXXXXXXXXXXXXXXXXXXXXXXXXXXXXXXXXXXXXXXXXXX

SEQ ID NO: 26 - protein, 54 aa, artificial
  2xConLA
AQQEECELAPWTCEHMGSGSATGGSGSTASSGSGSATHQEECELAPWTCEHMLE

SEQ ID NO: 27 - protein, 16 aa, artificial
  ConFS
AQQEECEFSPWTCEHM

SEQ ID NO: 28 - protein, 106 aa, artificial
  MRD
XXXXXXXXXXXXXXXXXXXXXXXXXXXXXXXXXXXXXXXXXXXXXXXXXXEFSPWTXXXXXXXXXXXXXXXXXXXXXXXXXXXXXXXXXXXXXXXXXXXXXXXXXX

SEQ ID NO: 29 - protein, 54 aa, artificial
  2xConFS
AQQEECEFSPWTCEHMGSGSATGGSGSTASSGSGSATHQEECEFSPWTCEHMLE

SEQ ID NO: 30 - protein, 16 aa, artificial
  ConLE
AQQEECELEPWTCEHM

SEQ ID NO: 31 - protein, 106 aa, artificial
  MRD
XXXXXXXXXXXXXXXXXXXXXXXXXXXXXXXXXXXXXXXXXXXXXXXXXXELEPWTXXXXXXXXXXXXXXXXXXXXXXXXXXXXXXXXXXXXXXXXXXXXXXXXXX

SEQ ID NO: 32 - protein, 54 aa, artificial
  2xConLE
AQQEECELEPWTCEHMGSGSATGGSGSTASSGSGSATHQEECELEPWTCEHMLE

SEQ ID NO: 33 - protein, 54 aa, artificial
  ConFA-LA heterodimer
AQQEECEFAPWTCEHMGSGSATGGSGSTASSGSGSATHQEECELAPWTCEHMLE

SEQ ID NO: 34 - protein, 54 aa, artificial
  ConFA-FS
AQQEECEFAPWTCEHMGSGSATGGSGSTASSGSGSATHQEECEFSPWTCEHMLE

SEQ ID NO: 35 - protein, 28 aa, artificial
  MRD
NFYQCIEMLASHPAEKSRGQWQECRTGG

SEQ ID NO: 36 - protein, 28 aa, artificial
  MRD
NFYQCIEQLALRPAEKSRGQWQECRTGG

SEQ ID NO: 37 - protein, 28 aa, artificial
  MRD
NFYQCIDLLMAYPAEKSRGQWQECRTGG

SEQ ID NO: 38 - protein, 28 aa, artificial
  MRD
NFYQCIERLVTGPAEKSRGQWQECRTGG

SEQ ID NO: 39 - protein, 28 aa, artificial
  MRD
NFYQCIEYLAMKPAEKSRGQWQECRTGG

SEQ ID NO: 40 - protein, 28 aa, artificial
  MRD
NFYQCIEALQSRPAEKSRGQWQECRTGG

SEQ ID NO: 41 - protein, 28 aa, artificial
  MRD
NFYQCIEALSRSPAEKSRGQWQECRTGG

SEQ ID NO: 42 - protein, 27 aa, artificial
  MRD
NFYQCIEHLSGSPAEKSRGQWQECRTG

SEQ ID NO: 43 - protein, 27 aa, artificial
  MRD
NFYQCIESLAGGPAEKSRGQWQECRTG

SEQ ID NO: 44 - protein, 27 aa, artificial
  MRD
NFYQCIEALVGVPAEKSRGQWQECRTG

SEQ ID NO: 45 - protein, 27 aa, artificial
  MRD
NFYQCIEMLSLPPAEKSRGQWQECRTG

SEQ ID NO: 46 - protein, 27 aa, artificial
  MRD
NFYQCIEVFWGRPAEKSRGQWQECRTG

SEQ ID NO: 47 - protein, 27 aa, artificial
  MRD
NFYQCIEQLSSGPAEKSRGQWQECRTG

SEQ ID NO: 48 - protein, 27 aa, artificial
  MRD
NFYQCIELLSARPAEKSRGQWAECRAG

SEQ ID NO: 49 - protein, 27 aa, artificial
  MRD
NFYQCIEALARTPAEKSRGQWVECRAP

SEQ ID NO: 50 - protein, 7 aa, artificial
  MRD
YCRGDCT

SEQ ID NO: 51 - protein, 7 aa, artificial
  MRD
PCRGDCL

SEQ ID NO: 52 - protein, 7 aa, artificial
  MRD
TCRGDCY

SEQ ID NO: 53 - protein, 7 aa, artificial
  MRD
LCRGDCF

SEQ ID NO: 54 - protein, 22 aa, artificial
  MRD
XXCXXXXXCXXFCXDWPXXXSC

SEQ ID NO: 55 - protein, 9 aa, artificial
  MRD
VCYXXXICF

SEQ ID NO: 56 - protein, 26 aa, artificial
  MRD
MGAQTNFMPMDXXXXXXXXXXXXXXX

SEQ ID NO: 57 - protein, 116 aa, artificial
  targeting MRD peptide
XXXXXXXXXXXXXXXXXXXXXXXXXXXXXXXXXXXXXXXXXXXXXXXXXXAQQEECEXXPWTCEHMXXXXXXXXXXXXXXXXXXXXXXXXXXXXXXXXXXXXXXXXXXXXXXXXXX

SEQ ID NO: 58 - protein, 28 aa, artificial
  MRD
NFYQCIXXLXXXPAEKSRGQWQECRTGG

SEQ ID NO: 59 - protein, 12 aa, artificial
  VL-CDR1
RASQDVNTAVAW

SEQ ID NO: 60 - protein, 7 aa, artificial
  VL-CDR2
SASFLYS

SEQ ID NO: 61 - protein, 9 aa, artificial
  VL-CDR3
QQHYTTPPT

SEQ ID NO: 62 - protein, 10 aa, artificial
  VH-CDR1
GRNIKDTYIH

SEQ ID NO: 63 - protein, 17 aa, artificial
  VH-CDR2
RIYPTNGYTRYADSVKG

SEQ ID NO: 64 - protein, 11 aa, artificial
  VH-CDR3
WGGDGFYAMDY

SEQ ID NO: 65 - protein, 109 aa, artificial
  VL
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRT

SEQ ID NO: 66 - protein, 120 aa, artificial [sequence omitted]

SEQ ID NO: 67 - protein, 27 aa, artificial
  Rm1-67
NFYQCIESLVNGPAEKSRGQWDGCRKK

SEQ ID NO: 68 - protein, 27 aa, artificial
  Rm2-2-218
NFYQCIESLVNGPAEKSRGQWVECRAP

SEQ ID NO: 69 - protein, 27 aa, artificial
  Rm2-2-316
NFYQCIESLVNGPAEKSRGQWAECRAG

SEQ ID NO: 70 - protein, 27 aa, artificial
  Rm2-2-319
NFYQCIESLVNGPAEKSRGQWQECRTG

SEQ ID NO: 71 - protein, 7 aa, artificial
  MRD
ATWLPPP

SEQ ID NO: 72 - protein, 11 aa, artificial
  VL-CDR1
SASQDISNYLN

SEQ ID NO: 73 - protein, 7 aa, artificial
  VL-CDR2
FTSSLHS

SEQ ID NO: 74 - protein, 9 aa, artificial
  VL-CDR3
QQYSTVPWT

SEQ ID NO: 75 - protein, 10 aa, artificial
  VH-CDR1
GYTFTNYGMN

SEQ ID NO: 76 - protein, 17 aa, artificial
  VH-CDR2
WINTYTGEPTYAADFKR

SEQ ID NO: 77 - protein, 14 aa, artificial
  VH-CDR3
YPHYYGSSHWYFDV

SEQ ID NO: 78 - protein, 108 aa, artificial
  VL
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKR

SEQ ID NO: 79 - protein, 123 aa, artificial [sequence omitted]

SEQ ID NO: 80 - protein, 11 aa, artificial
  VL-CDR1
RASQGIRNYLA

SEQ ID NO: 81 - protein, 7 aa, artificial
  VL-CDR2
AASTLQS

SEQ ID NO: 82 - protein, 9 aa, artificial
  VL-CDR3
QRYNRAPYT

SEQ ID NO: 83 - protein, 5 aa, artificial
  VH-CDR1
DYAMH

SEQ ID NO: 84 - protein, 17 aa, artificial
  VH-CDR2
AITWNSGHIDYADSVEG

SEQ ID NO: 85 - protein, 12 aa, artificial
  VH-CDR3
VSYLSTASSLDY

SEQ ID NO: 86 - protein, 108 aa, artificial
  VL
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKR

SEQ ID NO: 87 - protein, 121 aa, artificial [sequence omitted]

SEQ ID NO: 88 - protein, 7 aa, artificial
  MRD
CYXPGXC

SEQ ID NO: 89 - protein, 11 aa, artificial
  MRD
DXCLPXWGCLW

SEQ ID NO: 90 - protein, 11 aa, artificial
  MRD
XCLPRXWGCLW

SEQ ID NO: 91 - protein, 11 aa, artificial
  MRD
DLCLRDWGCLW

SEQ ID NO: 92 - protein, 11 aa, artificial
  MRD
DICLPRWGCLW

SEQ ID NO: 93 - protein, 20 aa, artificial
  MRD
QRLMEDICLPRWGCLWEDDE

SEQ ID NO: 94 - protein, 20 aa, artificial
  MRD
QGLIGDICLPRWGCLWGRSV

SEQ ID NO: 95 - protein, 21 aa, artificial
  MRD
QGLIGDICLPRWGCLWGRSVK

SEQ ID NO: 96 - protein, 15 aa, artificial
  MRD
EDICLPRWGCLWEDD

SEQ ID NO: 97 - protein, 18 aa, artificial
  MRD
RLMEDICLPRWGCLWEDD

SEQ ID NO: 98 - protein, 16 aa, artificial
  MRD
MEDICLPRWGCLWEDD

SEQ ID NO: 99 - protein, 15 aa, artificial
  MRD
MEDICLPRWGCLWED

SEQ ID NO: 100 - protein, 18 aa, artificial
  MRD
RLMEDICLARWGCLWEDD

SEQ ID NO: 101 - protein, 27 aa, artificial
  MRD
XXXXCXEXXXXXPAEKSRGQWXXCXXX

SEQ ID NO: 102 - protein, 105 aa, artificial
  Light Chain Insertion Site
RTVAAPSVFIFPPSDEQLKSGTASVVCLLNNFYPREAKVQWKVDKLGTNSQESVTEQDSKDSTYSLSSTLTLSKADYEKHKVYACEVTHQGLSLPVTKSFNRGEC

SEQ ID NO: 103 - protein, 10 aa, artificial
  MRD
SLFVPRPERK

SEQ ID NO: 104 - protein, 10 aa, artificial
  MRD
ESDVLHFTST

SEQ ID NO: 105 - protein, 9 aa, artificial
  MRD
LRKYADGTL

SEQ ID NO: 106 - protein, 20 aa, artificial
  MRD
EVRSFCTRWPAEKSCKPLRG

SEQ ID NO: 107 - protein, 20 aa, artificial
  MRD
RAPESFVCYWETICFERSEQ

SEQ ID NO: 108 - protein, 11 aa, artificial
  MRD
EMCYFPGICWM

SEQ ID NO: 109 - protein, 11 aa, artificial
  MRD
DXCLPXWGCLW

SEQ ID NO: 110 - protein, 11 aa, artificial
  MRD
FCXDWPXXXSC

SEQ ID NO: 111 - protein, 9 aa, artificial
  MRD
VCYXXXICF

SEQ ID NO: 112 - protein, 8 aa, artificial
  MRD
CYXPGXCX

SEQ ID NO: 113 - protein, 20 aa, artificial
  MRD
QRLMEDICLPRWGCLWEDDF

SEQ ID NO: 114 - protein, 15 aa, artificial
  MRD
MEDICLPRWGCLWGD

SEQ ID NO: 115 - protein, 13 aa, artificial
  MRD
DICLPRWGCLWED

SEQ ID NO: 116 - protein, 14 aa, artificial
  MRD
IEDICLPRWGCLWE

SEQ ID NO: 117 - protein, 11 aa, artificial
  MRD
DICLPRWGCLW

SEQ ID NO: 118 - protein, 10 aa, artificial
  MRD
DICLPRWGCL

SEQ ID NO: 119 - protein, 5 aa, artificial
  ELP
VPGXG

SEQ ID NO: 120 - protein, 5 aa, artificial
  ELP
IPGXG

SEQ ID NO: 121 - protein, 5 aa, artificial
  ELP
LPGXG

SEQ ID NO: 122 - protein, 4 aa, artificial
  ELP
VPGG

SEQ ID NO: 123 - protein, 4 aa, artificial
  ELP
IPGG

SEQ ID NO: 124 - protein, 5 aa, artificial
  ELP
AVGVP

SEQ ID NO: 125 - protein, 5 aa, artificial
  ELP
IPGXG

SEQ ID NO: 126 - protein, 5 aa, artificial
  ELP
IPGVG

SEQ ID NO: 127 - protein, 5 aa, artificial
  ELP
LPGXG

SEQ ID NO: 128 - protein, 5 aa, artificial
  ELP
LPGVG

SEQ ID NO: 129 - protein, 6 aa, artificial
  ELP
VAPGVG

SEQ ID NO: 130 - protein, 8 aa, artificial
  ELP
GVGVPGVG

SEQ ID NO: 131 - protein, 9 aa, artificial
  ELP
VPGFGVGAG

SEQ ID NO: 132 - protein, 9 aa, artificial
  ELP
VPGVGVPGG

SEQ ID NO: 133 - protein, 26 aa, artificial
  CD3 Fragment
GYYVCYPRGSKPEDANFYLYLRARVC

SEQ ID NO: 134 - protein, 7 aa, artificial
  CD3 Fragment
YLYLRAR

SEQ ID NO: 135 - protein, 23 aa, artificial
  CD3 Fragment
YRCNGTDIYKDKESTVQVHYRMC

SEQ ID NO: 136 - protein, 9 aa, artificial
  CD3 Fragment
DKESTVQVH

SEQ ID NO: 137 - protein, 78 aa, Homo sapiens
KIPIEELEDRVFVNCNTSITWVEGTVGTLLSDITRLDLGKRILDPRGIYRCNGTDIYKDKESTVQVHYRMCQSCVELD

SEQ ID NO: 138 - protein, 89 aa, Homo sapiens
QSIKGNHLVKVYDYQEDGSVLLTCDAEAKNITWFKDGKMIGFLTEDKKKWNLGSNAKDPRGMYQCKGSQNKSKPLQVYYRMCQNCIELN

SEQ ID NO: 139 - protein, 105 aa, Homo sapiens
GNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVM

SEQ ID NO: 140 - protein, 9 aa, Homo sapiens
QSFGLLDPK

SEQ ID NO: 141 - protein, 27 aa, artificial
  CD3 Fragment
QDGNEEMGGITQTPYKVSISGTTVILT

SEQ ID NO: 142 - protein, 10 aa, artificial
  CD3 Fragment
QDGNEEMGGI

SEQ ID NO: 143 - protein, 9 aa, artificial
  CD3 Fragment
QDGNEEMGG

SEQ ID NO: 144 - protein, 27 aa, artificial
  ANGa
GAQTNFMPMDDLEQRLYEQFILQQGLE

SEQ ID NO: 145 - protein, 12 aa, artificial
  VEGFa
WCNGFPPNYPCY

SEQ ID NO: 146 - protein, 58 aa, artificial
  HER2a
VDNKFNKEMRNAYWEIALLPNLNNQQKRAFIRSLYDDPSQSANLLAEARKLNDAQAPK

SEQ ID NO: 147 - protein, 5 aa, artificial
  ELP2
VPGXG

SEQ ID NO: 148 - protein, 18 aa, artificial
  ANGb
LWDDCYFFPNPPHCYNSP

SEQ ID NO: 149 - protein, 18 aa, artificial
  ANGc
LWDDCYSYPNPPHCYNSP

SEQ ID NO: 150 - protein, 18 aa, artificial
  ANGd
LWDDCYSFPNPPHCYNSP

SEQ ID NO: 151 - protein, 18 aa, artificial
  ANGe
DCAVYPNPPWCYKMEFGK

SEQ ID NO: 152 - protein, 18 aa, artificial
  ANGf
PHEECYFYPNPPHCYTMS

SEQ ID NO: 153 - protein, 18 aa, artificial
  ANGg
PHEECYSYPNPPHCYTMS